(19) United States
(12) Patent Application Publication
(10) Pub. No.: US 2010/0203211 A1
(43) Pub. Date: Aug. 12, 2010

(54) GENETIC BASIS FOR IMPROVED MILLING PERFORMANCE

(75) Inventors: Robert James Henry, Tuckombil (AU); Peter Christian Bundock, Alstonville (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: GRAIN FOODS CRC LTD., North Ryde, New South Wales (AU)

(21) Appl. No.: 12/669,284

(22) PCT Filed: Jul. 17, 2008
(86) PCT No.: PCT/AU08/01041

§ 371 Date: Apr. 26, 2010

(30) Foreign Application Priority Data
Jul. 17, 2007 (AU) 2007903866
Jul. 17, 2007 (AU) 2007903867

(51) Int. Cl. A23L 1/10; C12Q 1/68; C12N 15/82; A01H 1/00; A01H 5/00

(52) U.S. Cl. 426/443; 435/6; 435/320.1; 800/278; 800/320.3

(57) ABSTRACT

A method of selecting a grain or a grain-producing plant with improved millability by determining the relative amount of an isolated nucleic acid that is associated with or linked to improved millability to determine whether the grain or grain-producing plant has improved millability is provided therein. Typically, the grain or grain-producing plant is wheat. Also provided are genetic constructs, methods for the diagnosis of improved millability and methods for the production of grain or grain-producing plants with improved millability.

### FIELD OF THE INVENTION

THE present invention generally relates to plant genetics. More particularly, the present invention relates to methods for genetic selection of a plant for improved grain milling quality and flour yield.

### BACKGROUND TO THE INVENTION

Milling quality depends on three main characteristics that are in turn influenced to varying degrees by the genetic origin of wheat and environmental conditions during plant development: kernel hardness, considered to be the essential factor in wheat milling behaviour, the endosperm to bran ratio that should be as high as possible and ease of separation of bran (Haddad et al., 1999). Hard wheat is generally considered easier to mill since it gives readier separation of bran from endosperm after conditioning, and the liberated flour is more mobile and easier to sift.

Consequently the majority of research has focused on elucidating the genetic mechanisms for variation in hardness, hence milling performance, within the hard phenotype. Hardness or endosperm cohesion is thought to be mainly influenced by the particular puroindoline genotype and the distribution of puroindoline proteins (Greenwell & Schofield, 1986; Giroux et al. 2003; Capparelli et al. 2003; Hogg et al. 2004; Gedye et al. 2005; Day et al. 2006; Swan et al. 2006). The Pina-D1 and Pinb-D1 alleles, tightly linked to the Ha locus on the short arm of Chromosome 5D, determine the hardness phenotype (Turnball & Rahman, 2002). However, this does not fully account for the observed genetic variation in hardness, especially within each hardness class, and it is thought that additional modifying genes account for the range of hardness within hard or soft classes (Martin et al, 2001; Osborne et al., 2001; Turnball et al., 2000). Several research groups have studied the role of the puroindolines in explaining within-class variation in hardness. In hard wheats, the Pina-D1b allele was associated with harder texture than the Pina-D1b allele (Giroux & Morris, 1997). Martin et al. (2001) reported that the Pinb-D1b (softer texture) allele was associated with better flour yield in Hard Red Spring wheat.

### SUMMARY OF THE INVENTION

Conventional breeding strategies and milling technologies have reached a plateau in flour milling yield. Hence, even the smallest improvements in plant milling quality traits has potential to greatly influence commercial milling performance. As such, the inventors have identified a need for new and improved methods of determining the milling performance of various crops, including wheat.

The present invention is broadly directed to isolated nucleic acid sequences from a cereal seed which are associated with improved milling performance and are useful for selecting, predicting and/or engineering crops with improved millability.

In a first aspect, the invention provides a method of selecting a grain or a grain-producing plant with improved millability, including the step of determining a relative amount of an isolated nucleic acid associated with or linked to improved millability present in the grain or grain-producing plant to determine whether or not the grain or grain-producing plant has a predisposition to improved millability.

In a second aspect, the invention provides a method of determining whether a grain or a grain-producing plant is genetically predisposed to improved millability, including the step of detecting an isolated nucleic acid associated with or linked to improved millability.

In one preferred embodiment, the isolated nucleic acid associated with or linked to improved millability encodes a polypeptide comprising an amino acid sequence selected from the group consisting of SEQ ID NOS:49 to 56, SEQ ID NO:190, SEQ ID NO:284 and SEQ ID NOS:290 to 295, or a fragment thereof.

Preferably, the isolated nucleic acid associated with or linked to improved millability encodes a polypeptide comprising an amino acid sequence selected from the group consisting of SEQ ID NO:49 and SEQ ID NO:190, or a fragment thereof.

According to these embodiments, the fragment comprises an amino acid sequence selected from the group consisting of SEQ ID NOS: 107 to 117 and SEQ ID NOS: 235 to 251.

In another preferred embodiment, the isolated nucleic acid associated with or linked to improved millability comprises a nucleotide sequence selected from the group consisting of SEQ ID NO:15, SEQ ID NO:26, SEQ ID NOS:159 to 169, SEQ ID NOS:188 to 189, SEQ ID NOS:194 to 202, SEQ ID NOS:285 to 289 and SEQ ID NOS:296 to 301, or a fragment thereof.

More preferably, the isolated nucleic acid associated with or linked to improved millability comprises a nucleotide sequence selected from the group consisting of SEQ ID NO:26 and SEQ ID NO:285.

Preferably, the fragment comprises a nucleotide sequence selected from the group consisting of SEQ ID NOS: 57 to 60, SEQ ID NOS: 72 to 74, SEQ ID NOS: 95 to 99, SEQ ID NOS: 215 to 224, SEQ ID NO: 102, SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

In a preferred embodiment, the isolated nucleic acid associated with or linked to improved millability is a variant having at least 60% sequence identity to the isolated nucleic acids of the invention as hereinbefore described. Preferably, the variant has at least 70% sequence identity to a nucleotide sequence selected from the group consisting of SEQ ID NO:26 and SEQ ID NO:285.

Preferably, the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

Preferably, the grain or the grain-producing plant has a reduced relative amount of the isolated nucleic acid associated with or linked to improved millability when compared to a reference sample.

In a third aspect, the invention provides a method of milling flour including the step of selecting a millable grain or grain-producing plant according to the method of the first aspect for subsequent milling of said grain to produce a flour.

In a fourth aspect, the invention provides a method of identifying one or more plant genetic loci which is/are associated with improved millability of a grain or a grain-producing plant, including the step of determining whether one or more plant genetic loci is/are associated with or linked to flour milling yield.

Preferably, the one or more plant genetic loci is a polymorphism of a nucleotide sequence selected from the group consisting of SEQ ID NO:15 and SEQ ID NOS:159 to 169.

More preferably, the one or more plant genetic loci is a polymorphism of a nucleotide sequence selected from the group consisting of SEQ ID NO:15 and SEQ ID NOS:159.

In a fifth aspect, the invention provides a method of producing a grain-producing plant with improved millability, including the step of selectively modulating a gene associated with improved millability, so that the relative amount of said gene associated with or linked to improved millability is lower than in a grain-producing plant where said gene has not been modulated.

It is envisaged that in a particular embodiment, the gene associated with or linked to improved millability can be modulated by conventional plant breeding. In an alternative embodiment, modulation of the gene associated with or linked to improved millability can occur through recombinant DNA methodology to thereby generate a “genetically modified” or “transgenic” plant.

In a sixth aspect, the invention provides a grain-producing plant having improved grain millability produced according to the method of the fifth aspect.

In a seventh aspect, the invention relates to a method of milling flour including the step of obtaining a grain from a grain-producing plant produced according to method of the fifth aspect for subsequent milling to produce a flour.

In an eighth aspect, the invention provides a genetic construct for improving grain millability comprising an isolated nucleic acid associated with or linked to improved millability as hereinbefore described.

In a ninth aspect, the invention provides a grain-producing plant with improved millability wherein a gene associated with or linked to improved millability is selectively modulated to have a lower relative amount of the gene associated with or linked to improved millability than in a plant where the gene has not been modulated.

Preferably, the gene associated with or linked to improved millability encodes a polypeptide comprising an amino acid sequence selected from the group consisting of SEQ ID NO:49 to 56, SEQ ID NO:190, SEQ ID NO:284 and SEQ ID NOS:290 to 295, or a fragment thereof.

In one preferred embodiment, the gene associated with or linked to improved millability encodes a polypeptide comprising an amino acid sequence selected from the group consisting of SEQ ID NO:49 and SEQ ID NO:190, or a fragment thereof.

According to these embodiments, the fragment comprises an amino acid sequence selected from the group consisting of SEQ ID NOS: 107 to 117 and SEQ ID NOS: 235 to 251.

In another preferred embodiment, the gene associated with or linked to improved millability comprises a nucleotide sequence selected from the group consisting of SEQ ID NO:15, SEQ ID NO:26, SEQ ID NOS:159 to 169, SEQ ID NOS:188 to 189, SEQ ID NOS:193 to 202, SEQ ID NOS:285 to 289, SEQ ID NOS:296 to 301, or a fragment thereof.

More preferably, the gene associated with or linked to improved millability comprises a nucleotide sequence selected from the group consisting of SEQ ID NO:26 and SEQ ID NO:285.

Preferably, the fragment comprises a nucleotide sequence selected from the group consisting of SEQ ID NOS: 57 to 60, SEQ ID NOS: 72 to 74, SEQ ID NOS: 95 to 99, SEQ ID NOS: 215 to 224, SEQ ID NO: 102, SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

In a preferred embodiment, the gene associated with or linked to improved millability is a variant having at least 60% sequence identity to the isolated nucleic acids of the invention as hereinbefore described. Preferably, the variant has at least 70% sequence identity to a nucleotide sequence selected from the group consisting of SEQ ID NO:26 and SEQ ID NO:285.

Preferably, the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

Preferably, the grain or the grain-producing plant has a grain comprising at least an endosperm and a bran layer.

More preferably, the grain or grain-producing plant is wheat.

In a tenth aspect, the invention provides an isolated nucleic acid associated with or linked to improved millability comprising a nucleotide sequence selected from the group consisting of SEQ ID NO:15, SEQ ID NO:26, SEQ ID NOS:159 to 169, SEQ ID NOS:193 to 202, SEQ ID NOS:285 to 289 and SEQ ID NOS:296 to 301, or a variant thereof.

In an eleventh aspect, the invention provides an isolated polypeptide comprising an amino acid sequence selected from the group consisting of SEQ ID NO:49 to 56, SEQ ID NO:190, SEQ ID NO:284, and SEQ ID NOS:290 to 295, or a variant thereof.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

In order that the invention may be readily understood and put into practical effect, preferred embodiments will now be described by way of example with reference to the accompanying figures wherein like reference numerals refer to like parts and wherein:

FIG. 1 Distribution of flour yield as a percentage of seed weight amongst 71 wheat varieties harvested from two sites (N and B above) in 2005 from samples of developing wheat seed at 14 days post anthesis (dpa). Ten wheat varieties were selected for initial gene expression experiments based on flour yield at these sites—five from the low end and five from the high end of the distribution.

FIG. 2 Comparison of the yield of flour from wheat varieties grown at both Narrabri and Biloela from 14 dpa samples.

FIG. 3A Flour yield average for high and low yield wheat varieties from 14 dpa samples. Each yield class is composed of five wheat varieties from measurements of wheat harvested from two sites (Biloela, Qld. and Narrabri, NSW) in 2005. A 95% confidence interval for the mean of each yield class is indicated.

FIG. 3B Quality check for RNA for 14 dpa developing wheat seed, using Bioanalyser (Agilent Technologies, USA).

FIG. 3C Quality check for RNA for 30 dpa developing wheat seed, using Bioanalyser (Agilent Technologies, USA).

FIG. 4 Kernel density estimates of expression for each of the chips for 14 dpa samples.

FIG. 5 Box and whisker plots of expression for each of the chips for 14dpa samples.

FIG. 6 MA plots of gene expression for 14 dpa samples.

FIG. 7 MA plots of gene expression for 14 dpa samples.

FIG. 8 Principal components plot based on the gene expression of the wheat varieties for 14 dpa samples.

FIG. 9 Cluster dendogram based on the single linkage algorithm for 14 dpa samples.

FIG. 10 Cluster dendogram based on the average linkage algorithm for 14 dpa samples.

FIG. 11 Cluster dendogram based on the complete linkage algorithm for 14 dpa samples.

FIG. 12 ROC curves for low yield versus high yield varieties for 14 dpa samples.

FIG. 12A Non-overlapping (disjoint gene) gene expression between high and low flour yielding wheat varieties at 14pda.

FIG. 12B A virtual image showing non-overlapping (disjoint gene) gene expression signals between high and low flour yielding wheat varieties at 30dpa.

FIG. 13A Nucleotide sequence of EST TA.28688.1.A1_AT (SEQ ID NO: 1).

FIG. 13B Nucleotide sequence of target wheat: Ta.28688.1.A1_at; gb|BJ275807

FIG. 14 Amino acid sequence of a polypeptide translated from SEQ ID NO:1 (SEQ ID NO: 2).

FIG. 15 Distribution of flour yield as a percentage of seed weight amongst 71 wheat varieties harvested from two sites (N and B above) in 2005 from samples of developing wheat seed at 30 days post anthesis (dpa). Ten wheat varieties were selected for initial gene expression experiments based on flour yield at these sites—five from the low end and five from the high end of the distribution.

FIG. 16 Comparison of the yield of flour from wheat varieties grown at both Narrabri and Biloela for 30 dpa samples.

FIG. 17 Flour yield average for high and low yield wheat varieties. Each yield class is composed of five wheat varieties from measurements of wheat harvested from two sites (Biloela, Qld. and Narrabri, NSW) in 2005 from samples of developing wheat seed at 30 dpa. A 95% confidence interval for the mean of each yield class is indicated.

FIG. 18 Kernel density estimates of expression for each of the chips for 30 dpa samples.

FIG. 19 Box and whisker plots of expression for each of the chips for 30 dpa samples.

FIG. 20 MA plots of gene expression for 30 dpa samples.

FIG. 21 MA plots of gene expression for 30 dpa samples.

FIG. 22 Principal components plot based on the gene expression of the wheat varieties for 30 dpa samples.

FIG. 23 Cluster dendogram based on the single linkage algorithm for 30 dpa samples.

FIG. 24 Cluster dendogram based on the average linkage algorithm for 30 dpa samples.

FIG. 25 Cluster dendogram based on the complete linkage algorithm for 30 dpa samples.

FIG. 26 ROC curves for low yield versus high yield varieties for 30 dpa samples.

FIG. 27A Nucleotide sequence of TA.11743.1.A1.at (SEQ ID NO: 3) target sequence.

FIG. 27B Nucleotide sequence of wheat: WHEAT:TA.11743.1.A1_AT_target sequence (SEQ ID NO:3).

FIG. 28 Nucleotide sequence of EST which comprises TA.11743.1.A1.at target sequence (SEQ ID NO: 4).

FIG. 29 Details the gene sequence corresponding to target Ta.28688.1.A1_at as obtained through NetAffx website.

FIG. 30 Alignment of ESTs clustering with the target Ta.28688.1.A1_at.

FIG. 31 Consensus sequence from the alignment of the ESTs clustering to the target Ta.28688.1.A1_at (SEQ ID NO:49).

FIG. 32 Open reading frame of the predicted 14dpa gene based on the consensus sequence derived from the alignments of ESTs to target Ta.28688.1.A1_at.

FIG. 33 Putative exons on the open reading frame of the predicted 14 dpa gene sequence. The rice genomic sequence Locus NC_{—}08400 was used to determine the location of the exons.

FIG. 34 Primers designed to the consensus sequence derived from the alignment of the ESTs clustering to the target Ta.28688. These primers were used in a PCR to amplify the 14 dpa gene from wheat.

FIG. 35 PCR amplified fragments resolved on a 0.7% agarose gel. The putative 14 dpa gene fragment amplified by PCR is shown as a 1.4 kb fragment. Primer 14dpaF1 and Primer14dpaR1 was used in the PCR with genomic DNA isolated from wheat cv Bob white.

FIG. 36 Screening of white colonies to identify recombinant colonies containing the putative 14 dpa gene fragment amplified by PCR is shown as a 1.4 kb fragment that was amplified using primer 14dpaF1 and primed 4dpaR1 with genomic DNA isolated from wheat cv Bob white.

FIG. 37 The putative 14dpa fragment amplified by PCR using recombinant colonies C1, C2, C3, C4, C5, C6. C7, C9. These fragments were purified and sequenced.

FIG. 38 Sequences of the cloned fragments in recombinant clones C1, C2, C3. C4, C5, C6, C7 and C9 and containing the putative 14 dpa gene from wheat (SEQ ID NOS: 41 to 48).

FIG. 39 Alignment of the sequences of the cloned fragments in recombinant clones C1, C2, C3. C4, C5, C6, C7 and C9. The recombinant colonies contain the isolated putative 14 dpa gene from wheat. These sequences have both the exon and intron sequences.

FIG. 40 Alignment between the consensus sequence of EST to target Ta.28.688, and the open reading frame sequence of the 14 dpa gene and the 14 dpa gene sequence from clone 2. The alignment shows that the isolated PCR fragment in clone C2 is the 14 dpa gene. The alignment also shows an almost perfect match between the exons on the 14dpaClone2 sequence and the open reading frame sequence.

FIG. 41 Alignment between the 14 dpa coding sequences sequence from all recombinant clones C1, C2, C3, C4, C5, C6, C7 and C9. These sequences correspond to exon sequences.

FIG. 42 Alignment between the 14 dpa translated coding sequences sequence from all recombinant clones C1, C2, C3, C4, C5, C6, C7 and C9.

FIG. 43 BLAST searches for nr-DNA to the gene sequence corresponding to recombinant clone 2.

FIG. 44 BLAST searches for ESTs to the gene sequence corresponding to recombinant clone 2.

FIG. 45 BLAST searches for nr-DNA to the coding sequence corresponding to recombinant clone 2.

FIG. 46 BLAST searches for ESTs to the coding sequence corresponding to recombinant clone 2.

FIG. 47 BLAST searches for protein sequences to the translated sequence of corresponding to coding sequence of the recombinant clone 2.

FIG. 48 Details of the gene sequence corresponding to target TA.11743.1.A1_AT as obtained from NetAffx website.

FIG. 49 Nucleotide sequence of sequence of EST BQ170720.

FIG. 50 Alignment between EST BQ170720 and the target TA.11743.1.A1_AT.

FIG. 51 The target sequence Ta.11743.1 shows weak similarity to the transcribed locus in rice corresponding to locus NC_{—}008401 in the rice genome. The possible ORFs and their structure are indicated.

FIG. 52 Graphical representation of a contig generated using relevant ESTs and the target Ta.117431.1_AT (FIGS. 52A and B).

FIG. 53 Alignment between ESTs with locus ID BQ170720, BF482223 and CF133508 and the target Ta.117431.1_AT.

FIG. 54 Consensus sequence between ESTs with locus ID BQ170720, BF482223 and CF133508 and the target Ta.117431.1_AT. Location of primer and their sequence is indicated. These primers were used to amplify upstream sequences for the wheat genome.

FIG. 55 Nested GenomeWalker PCR products resolved in a 0.7% agarose gel. Wheat genomic DNA was used to amplify the region of DNA upstream from the target Ta.11743.1.A1.

FIG. 56 PCR screening of white colonies to identify recombinant colonies containing the Dra- and the Stu Fragments.

FIG. 57 Alignment of all GenomeWalker Dra-fragments representing the upstream region of the target Ta.117431.1_AT.

FIG. 58 Alignment between 30dpaDra-Fragment-1 and a contig of ESTs CF133508, BF482223 and BQ170720, and between target Ta.117431 sequence.

FIG. 59 Alignment between 30dpaDra-Fragment-2 and between a contig of ESTs CF133508, BF482223 and BQ170720, and between the target Ta.117431 sequence.

FIG. 60 ORFs found on the contig EST-BFBQ.

FIG. 61 Protein sequence and their alignments of two open reading frames (ORF) on the contig EST-BFBQ (BF482223 and BQ170720). ORF-1 and ORF-2 showed complete homology.

FIG. 62 Contig of ESTs CF133508, BF482223 and BQ170720, showing Open reading frames along the contigs. Note, most of the ORFs end at 817 bp.

FIG. 63 Protein sequence and their alignments of two open reading frames (ORF) on a contig of ESTs CF133508, BF482223 and BQ170720.

FIG. 64 Alignment showing protein sequence homology between ORFs on EST contigs. CF, CF133508; BF, BF482223; BQ, BQ170720.

FIG. 65 ORFs on 30dpa Dra fragment DraF2C1.

FIG. 66 Amino acid sequence of the ORF1-DraF2C1 on the 30dpa gene fragment DraF2C1 (SEQ ID NO:190).

FIG. 67 Alignment of amino acid sequence corresponding to ORF-1 of ORF-1/BFBQ, ORF-1/CFBFBQ and ORF-1/DraF2C1.

FIG. 68 Alignment of contig EST-CFBFBQ and 30dpa gene fragment DraF2C1 sequence. The indels in the contig EST-CFBFBQ are shown as a boxed region.

FIG. 69 Alignment of amino acid sequence between the 30dpa gene ORF1-DraF2C1 (DraF2C1 fragment), the ORF-1/CFBFBQ (contig EST-CFBFBQ with indels removed as indicated in FIG. 68) and the ORF-1/BFBQ (contig EST-BFBQ).

FIG. 70 Sequence of 30dpa-DraF2C3 fragment showing various open reading frames. The open reading frame ORF1-DraF2C3 and labelled as ORF-1 is similar in sequence to the ORF1-DraF2C1 that is located on the 30dpa DraF2C1 fragment.

FIG. 71 Sequence of 30dpa-DraF2C4 fragment showing various open reading frames. The open reading frame ORF1-DraF2C3 and labelled as ORF-1 is similar in sequence to the ORF1-DraF2C1 that is located on the 30dpa DraF2C1 fragment.

FIG. 72 Sequence variants of 30dpa gene fragment corresponding to DraF1 fragments.

FIG. 73 Sequence variants of 30dpa gene fragment corresponding to DraF2 fragments.

FIG. 74 Blast to nr-DNA of the 30-dpa gene fragment corresponding to the DraF2C1 fragment.

FIG. 75 Blast to EST of the 30-dpa gene fragment corresponding to the DraF2C1 fragment.

FIG. 76 Amino acid blast to nr-protin sequences with the 30-dpa gene fragment corresponding to the DraF2C1 fragment.

FIG. 77 Plasmid map of the gene construct pAHC25.

FIG. 78 Plasmid map of the gene construct pUbi.gfp.nos (pA53).

FIG. 79 ORF-1/ESTCFBFBQ sequence that was located on the contig EST CFBFBQ.

FIG. 80 ORF-1/DraF2C1 sequence that was located on the 30 dpa fragment DraF2C1 sequence (SEQ ID NO:285).

FIG. 81 ORF-1DraF2C3 sequence that was located on the 30 dpa fragment DraF2C3 sequence.

FIG. 82 Sequence alignment of the ORF-1 sequences that were located on the contig EST CFBFBQ, 30 dpa DraF2C1 and the 30 dpa DraF2C3 fragment.

FIG. 83 Protein sequence alignment of the ORF-1 sequences that were located on the contig EST CFBFBQ (with indels removed as indicated in FIGS. 68 and 69), 30 dpa DraF2C1 and the 30 dpa DraF2C3 fragment.

FIG. 84 Nucleotide and amino acid sequence of ORFs from DraF2C1, DraF2C3 and DraF2C4.

### BRIEF DESCRIPTION OF THE SEQUENCE LISTING

SEQ ID NO:1 Nucleotide sequence of TA.28688.1.A1_AT (14 dpa).

SEQ ID NO:2 Amino acid sequence of transcription elongation factor as translated from SEQ ID NO: 1.

SEQ ID NO:3 Nucleotide sequence of target EST TA.11743.1.A1.at (30 dpa).

SEQ ID NO:4 Nucleotide sequence of EST comprising TA.11743.1.A1.at.

SEQ ID NO:5 Affymetrix target sequence Ta.28688.1.A1_at.

SEQ ID NO:6 Nucleotide sequence of EST WHEAT:TA.28688.2.

SEQ ID NO:7 Nucleotide sequence of EST BJ24108.

SEQ ID NO:8 Nucleotide sequence of EST WHEAT:TA.28688.3.

SEQ ID NO:9 Nucleotide sequence of EST BJ27580.

SEQ ID NO:10 Nucleotide sequence of EST BJ270815.

SEQ ID NO:11 Nucleotide sequence of EST BJ297411.

SEQ ID NO:12 Nucleotide sequence of EST BJ235878.

SEQ ID NO:13 Nucleotide sequence of EST BJ290803.

SEQ ID NO:14 Nucleotide sequence of EST AL829370.

SEQ ID NO:15 Nucleotide sequence of the consensus sequence of Ta.28688.1.A1_at.

SEQ ID NO:16 Nucleotide sequence of ORF-1.

SEQ ID NO:17 Nucleotide sequence of rice Exon 1.

SEQ ID NO:18 Nucleotide sequence of rice Exon 2.

SEQ ID NO:19 Nucleotide sequence of rice Exon 3.

SEQ ID NO:20 Nucleotide sequence of rice Exon 4.

SEQ ID NO:21 Nucleotide sequence of wheat coding 1.

SEQ ID NO:22 Nucleotide sequence of wheat coding 2.

SEQ ID NO:23 Nucleotide sequence of wheat coding 3.

SEQ ID NO:24 Nucleotide sequence of wheat coding 4.

SEQ ID NO:25 Nucleotide sequence of start codon.

SEQ ID NO:26 Nucleotide sequence of ORF of consensus sequence of Ta.28688.1.A1_at.

SEQ ID NO:27 Nucleotide sequence of rice genomic sequence from locus NC_{—}008400.

SEQ ID NO:28-40 Miscellaneous Ta.28688.1.A1_at primer sequences.

SEQ ID NO:41 Nucleotide sequence of 14 dpa gene Clone 1.

SEQ ID NO:42 Nucleotide sequence of 14 dpa gene Clone 2.

SEQ ID NO:43 Nucleotide sequence of 14 dpa gene Clone 3.

SEQ ID NO:44 Nucleotide sequence of 14 dpa gene Clone 4.

SEQ ID NO:45 Nucleotide sequence of 14 dpa gene Clone 5.

SEQ ID NO:46 Nucleotide sequence of 14 dpa gene Clone 6.

SEQ ID NO:47 Nucleotide sequence of 14 dpa gene Clone 7.

SEQ ID NO:48 Nucleotide sequence of 14 dpa gene Clone 9.

SEQ ID NO:49 Amino acid sequence of 14 dpa gene Clone 1 ORF.

SEQ ID NO:50 Amino acid sequence of 14 dpa gene Clone 2 ORF.

SEQ ID NO:51 Amino acid sequence of 14 dpa gene Clone 3 ORF.

SEQ ID NO:52 Amino acid sequence of 14 dpa gene Clone 4 ORF.

SEQ ID NO:53 Amino acid sequence of 14 dpa gene Clone 5 ORF.

SEQ ID NO:54 Amino acid sequence of 14 dpa gene Clone 6 ORF.

SEQ ID NO:55 Amino acid sequence of 14 dpa gene Clone 7 ORF.

SEQ ID NO:56 Amino acid sequence of 14 dpa gene Clone 9 ORF.

SEQ ID NO:57 Nucleotide sequence of 14dpa gene clone1 fragment from position 1 to 115.

SEQ ID NO:58 Nucleotide sequence of 14dpa gene clone1 fragment from position 215 to 285.

SEQ ID NO:59 Nucleotide sequence of 14dpa gene clone1 fragment from position 981 to 1036.

SEQ ID NO:60 Nucleotide sequence of 14dpa gene clone1 fragment from position 864 to 886

SEQ ID NO:61 Nucleotide sequence of Hordeum vulgare subsp. vulgare cDNA clone: FLbaf30p05, mRNA sequence fragment from position 100 to 214.

SEQ ID NO:62 Nucleotide sequence of Hordeum vulgare subsp. vulgare cDNA clone: FLbaf30p05, mRNA sequence fragment from position 215 to 285.

SEQ ID NO:63 Nucleotide sequence of Hordeum vulgare subsp. vulgare cDNA clone: FLbaf30p05, mRNA sequence fragment from position 308 to 363.

SEQ ID NO:64 Nucleotide sequence of Hordeum vulgare subsp. vulgare cDNA clone: FLbaf30p05, mRNA sequence fragment from position 285 to 307.

SEQ ID NO:65 Nucleotide sequence of 14dpa gene clone1 fragment from position 1 to 115.

SEQ ID NO:66 Nucleotide sequence of 14dpa gene clone1 fragment from position 988 to 1027.

SEQ ID NO:67 Nucleotide sequence of Zea mays clone 93042 mRNA sequence fragment from position 139 to 253.

SEQ ID NO:68 Nucleotide sequence of Zea mays clone 12168 mRNA sequence fragment from position 108 to 222.

SEQ ID NO:69 Nucleotide sequence of Zea mays clone 12168 mRNA sequence fragment from position 323 to 362.

SEQ ID NO:70 Nucleotide sequence of Zea mays clone EL01N0552A10.c mRNA sequence fragment from position 332 to 446.

SEQ ID NO:71 Nucleotide sequence of Zea mays clone EL01N0552A10.c mRNA sequence fragment from position 547 to 586.

SEQ ID NO:72 Nucleotide sequence of 14dpa gene clone1 fragment from position 864 to 1045.

SEQ ID NO:73 Nucleotide sequence of 14dpa gene clone1 fragment from position 215 to 285.

SEQ ID NO:74 Nucleotide sequence of 14dpa gene clone1 fragment from position 51 to 115.

SEQ ID NO:75 Nucleotide sequence of wr1.pk015014 wr1 Triticum aestivum cDNA clone wr1.pk0150.f4 fragment from position 136 to 316.

SEQ ID NO:76 Nucleotide sequence of wr1.pk0150.f4 wr1 Triticum aestivum cDNA clone wr1.pk0150.f4 fragment from position 66 to 136.

SEQ ID NO:77 Nucleotide sequence of wr1.pk0150.f4 wr1 Triticum aestivum cDNA clone wr1.pk0150.f4 fragment from position 1 to 65.

SEQ ID NO:78 Nucleotide sequence of 14dpa gene clone1 fragment from position 981 to 1045.

SEQ ID NO:79 Nucleotide sequence of 14dpa gene clone1 fragment from position 215 to 283.

SEQ ID NO:80 Nucleotide sequence of 14dpa gene clone1 fragment from position 864 to 886.

SEQ ID NO:81 Nucleotide sequence of CJ655632 Y. Ogihara unpublished cDNA library Wh_GCPCDAM Triticum aestivum cDNA clone whgc3a04 5′, mRNA sequence fragment from position 60 to 174.

SEQ ID NO:82 Nucleotide sequence of CJ655632 Y. Ogihara unpublished cDNA library Wh_GCPCDAM Triticum aestivum cDNA clone whgc3a04 5′, mRNA sequence fragment from position 268 to 332.

SEQ ID NO:83 Nucleotide sequence of CJ655632 Y. Ogihara unpublished cDNA library Wh_GCPCDAM Triticum aestivum cDNA clone whgc3a04 5′, mRNA sequence fragment from position 175 to 243.

SEQ ID NO:84 Nucleotide sequence of CJ655632 Y. Ogihara unpublished cDNA library Wh_GCPCDAM Triticum aestivum cDNA clone whgc3a04 5′, mRNA sequence fragment from position 245 to 267.

SEQ ID NO:85 Nucleotide sequence of CJ547844 Y. Ogihara unpublished cDNA library Wh_GCPCDAM Triticum aestivum cDNA clone rwhgc3a04 3′, mRNA sequence fragment from position 644 to 530.

SEQ ID NO:86 Nucleotide sequence of CJ547844 Y. Ogihara unpublished cDNA library Wh_GCPCDAM Triticum aestivum cDNA clone rwhgc3a04 3′, mRNA sequence fragment from position 436 to 372.

SEQ ID NO:87 Nucleotide sequence of CJ547844 Y. Ogihara unpublished cDNA library Wh_GCPCDAM Triticum aestivum cDNA clone rwhgc3a04 3′, mRNA sequence fragment from position 529 to 461.

SEQ ID NO:88 Nucleotide sequence of CJ547844 Y. Ogihara unpublished cDNA library Wh_GCPCDAM Triticum aestivum cDNA clone rwhgc3a04 3′, mRNA sequence fragment from position 459 to 437.

SEQ ID NO:89 Nucleotide sequence of 14dpa gene clone1 fragment from position 215 to 285.

SEQ ID NO:90 Nucleotide sequence of 14dpa gene clone1 fragment from position 981 to 1045.

SEQ ID NO:91 Nucleotide sequence of 14dpa gene clone1 fragment from position 864 to 886.

SEQ ID NO:92 Nucleotide sequence of BJ290803 Y. Ogihara unpublished cDNA library, Wh_SL Triticum aestivum cDNA clone whs120e21 5′, mRNA sequence fragment from position 79 to 193.

SEQ ID NO:93 Nucleotide sequence of BJ290803 Y. Ogihara unpublished cDNA library, Wh_SL Triticum aestivum cDNA clone whs120e21 5′, mRNA sequence fragment from position 194 to 264.

SEQ ID NO:94 Nucleotide sequence of BJ290803 Y. Ogihara unpublished cDNA library, Wh_SL Triticum aestivum cDNA clone whs120e21 5′, mRNA sequence fragment from position 287 to 351.

SEQ ID NO:95 Nucleotide sequence of BJ290803 Y. Ogihara unpublished cDNA library, Wh_SL Triticum aestivum cDNA clone whs120e21 5′, mRNA sequence fragment from position 264 to 286.

SEQ ID NO:96 Nucleotide sequence of Coding 14dpa gene clone2 fragment from position 1 to 262.

SEQ ID NO:97 Nucleotide sequence of Coding 14dpa gene clone2 fragment from position 1 to 270.

SEQ ID NO:98 Nucleotide sequence of Coding 14dpa gene clone2 fragment from position 1 to 253.

SEQ ID NO:99 Nucleotide sequence of Hordeum vulgare subsp. vulgare cDNA clone: FLbaf30p05, mRNA sequence fragment from position 102 to 363.

SEQ ID NO:100 Nucleotide sequence of Hordeum vulgare subsp. vulgare cDNA clone: FLbaf83d21, mRNA sequence fragment from position 226 to 495.

SEQ ID NO:101 Nucleotide sequence of Zea mays clone 12168 mRNA sequence fragment from position 110 to 362.

SEQ ID NO:102 Nucleotide sequence of Coding 14dpa gene clone2 fragment from position 1 to 270.

SEQ ID NO:103 Nucleotide sequence of CJ655632 Y. Ogihara unpublished cDNA library Wh_GCPCDAM Triticum aestivum cDNA clone whgc3a04 5′, mRNA sequence fragment from position 62 to 331.

SEQ ID NO:104 Nucleotide sequence of CJ547844 Y. Ogihara unpublished cDNA library Wh_GCPCDAM Triticum aestivum cDNA clone rwhgc3a04 3′, mRNA sequence fragment from position 642 to 373.

SEQ ID NO:105 Nucleotide sequence of G356.110E18F010919 G356 Triticum aestivum cDNA clone G356110E18, mRNA sequence fragment from position 76 to 345.

SEQ ID NO:106 Nucleotide sequence of BJ311239 Y. Ogihara unpublished cDNA library, Wh_yd Triticum aestivum cDNA clone whyd26o07 3′, mRNA sequence fragment from position 503 to 234.

SEQ ID NO:107 Amino acid sequence of Protein coding 14dpa clone2 fragment from position 16 to 89.

SEQ ID NO:108 Amino acid sequence of Protein coding 14dpa clone2 fragment from position 16 to 86.

SEQ ID NO:109 Amino acid sequence of Protein coding 14dpa clone2 fragment from position 16 to 87.

SEQ ID NO:110 Amino acid sequence of Protein coding 14dpa clone2 fragment from position 17 to 81.

SEQ ID NO:111 Amino acid sequence of Protein coding 14dpa clone2 fragment from position 18 to 87.

SEQ ID NO:112 Amino acid sequence of Protein coding 14dpa clone2 fragment from position 32 to 86.

SEQ ID NO:113 Amino acid sequence of Protein coding 14dpa clone2 fragment from position 16 to 89.

SEQ ID NO:114 Amino acid sequence of Protein coding 14dpa clone2 fragment from position 19 to 78.

SEQ ID NO:115 Amino acid sequence of Protein coding 14dpa clone2 fragment from position 16 to 81.

SEQ ID NO:116 Amino acid sequence of Protein coding 14dpa clone2 fragment from position 17 to 88.

SEQ ID NO:117 Amino acid sequence of Protein coding 14dpa clone2 fragment from position 18 to 88.

SEQ ID NO:118 Amino acid sequence of Os07g0631100 [Oryza sativa Japonica Group] fragment from position 16 to 89.

SEQ ID NO:119 Amino acid sequence of unnamed protein product [Vitis vinifera] fragment from position 16 to 86.

SEQ ID NO:120 Amino acid sequence of unknown [Populus trichocarpa] and unknown [Populus trichocarpa×Populus deltoides] fragment from position 16 to 86.

SEQ ID NO:121 Amino acid sequence of gi|115444063|ref|NP_{—}001045811.1|Os02g0134300 [Oryza sativa (japonica cultivar-group)] fragment from 16 to 87.

SEQ ID NO:122 Amino acid sequence of gi|18422622|ref|NP_{—}568654.1| unknown protein [Arabidopsis thaliana] fragment from position 16 to 86.

SEQ ID NO:123 Amino acid sequence of gi|16791582|gb|ABK26033.1| unknown [Picea sitchensis] fragment from position 17 to 81.

SEQ ID NO:124 Amino acid sequence of gi|168025617|ref|XP_{—}001765330.1| predicted protein [Physcomitrella patens subsp. patens]; gi|162683383|gb|EDQ69793.1| predicted protein [Physcomitrella patens subsp. patens] fragment from position 17 to 81.

SEQ ID NO:125 Amino acid sequence of gi|168022079|ref|XP_{—}001763568.1| predicted protein [Physcomitrella patens subsp. patens] fragment from position 17 to 81.

SEQ ID NO:126 Amino acid sequence of gi|55296704|dbj|BAD69422.1| hypothetical protein [Oryza sativa Japonica Group] fragment from position 21 to 94.

SEQ ID NO:127 gi|55297459|dbj|BAD69310.1| hypothetical protein [Oryza sativa Japonica Group] fragment from position 21 to 94.

SEQ ID NO:128 gi|125554153|gb|EAY99758.1| hypothetical protein OsI_{—}020991 [Oryza sativa (indica cultivar-group)] fragment from position 21 to 94.

SEQ ID NO:129 gi|125596104|gb|EAZ35884.1| hypothetical protein OsJ_{—}019367 [Oryza sativa (japonica cultivar-group)] fragment from position 21 to 94.

SEQ ID NO:130 Amino acid sequence of gi|19757723|dbj|BAB08248.1| unnamed protein product [Arabidopsis thaliana] fragment from 146 to 204.

SEQ ID NO:131 Amino acid sequence of gi|68487892|ref|XP_{—}712163.1| hypothetical protein CaO019.13944 [Candida albicans SC5314] fragment from position 19 to 78.

SEQ ID NO:132 gi|68488889|ref|XP_{—}711689.1| hypothetical protein CaO19.6623 [Candida albicans SC5314] fragment from position 19 to 78.

SEQ ID NO:133 gi|46433010|gb|EAK92467.1| hypothetical protein CaO19.6623 [Candida albicans SC5314] fragment from position 19 to 78.

SEQ ID NO:134 gi|46433534|gb|EAK92970.1| hypothetical protein CaO19.13944 [Candida albicans SC5314] fragment from position 19 to 78.

SEQ ID NO:135 GENE ID: 3646195 CaO19.13944| similar to S. cerevisiae YKL160 W [Candida albicans SC5314] fragment from position 19 to 78.

SEQ ID NO:136 Amino acid sequence of gi|50287065|ref|XP_{—}445962.1| unnamed protein product [Candida glabrata] fragment from position 16 to 87.

SEQ ID NO:137 Amino acid sequence of gi|156841713|ref|XP_{—}001644228.1| hypothetical protein Kpol_{—}1051p19 [Vanderwaltozyma polyspora DSM 70294] fragment from position 16 to 81.

SEQ ID NO:138 Amino acid sequence of gi|6322689|ref|NP_{—}012762.1| Transcription elongation factor that contains a conserved zinc finger domain; implicated in the maintenance of proper chromatin structure in actively transcribed regions; deletion inhibits Brome mosaic virus (BMV) gene expression; Elf1p [Saccharomyces cerevisiae] fragment from position 16 to 91.

SEQ ID NO:139 Amino acid sequence of gi|151941650|gb|EDN60012.1| elongation factor [Saccharomyces cerevisiae YJM789] fragment from position 18 to 91.

SEQ ID NO:140-154 Miscellaneous Ta.11743.1.A1_at primer sequences.

SEQ ID NO:155 Nucleotide sequence of a contig CF133508 generated using ESTs and Ta.117431/1_AT.at.

SEQ ID NO:156 Nucleotide sequence of EST CF133508.

SEQ ID NO:157 Nucleotide sequence of EST BF482223.

SEQ ID NO:158 Nucleotide sequence of EST BQ170720.

SEQ ID NO:159 Nucleotide sequence of 30dpa DraF2C1.

SEQ ID NO:160 Nucleotide sequence of 30dpa DraF2C4.

SEQ ID NO:161 Nucleotide sequence of 30dpa DraF2C3.

SEQ ID NO:162 Nucleotide sequence of 30dpa DraF1C10.

SEQ ID NO:163 Nucleotide sequence of 30dpa DraF1C2.

SEQ ID NO:164 Nucleotide sequence of 30dpa DraF1C1.

SEQ ID NO:165 Nucleotide sequence of 30dpa DraF1C3.

SEQ ID NO:166 Nucleotide sequence of 30dpa DraF1C4.

SEQ ID NO:167 Nucleotide sequence of 30dpaDraF1C9.

SEQ ID NO:168 Nucleotide sequence of 30dpaDraF1C5.

SEQ ID NO:169 Nucleotide sequence of 30dpaDraF1C7.

SEQ ID NO:170 Nucleotide sequence of EST-BFBQ.

SEQ ID NO:171 Nucleotide sequence of ORF-1/EST-BFBQ.

SEQ ID NO:172 Nucleotide sequence of ORF-2/EST-BFBQ.

SEQ ID NO:173 Amino acid sequence of ORF-1 of EST-BFBQ.

SEQ ID NO:174 Amino acid sequence of ORF-2 of EST-BFBQ.

SEQ ID NO:175 Nucleotide sequence of ORF-1 of Contig of ESTs CF133508, BF482223 and BQ170720.

SEQ ID NO:176 Nucleotide sequence of ORF-2 of Contig of ESTs CF133508, BF482223 and BQ170720.

SEQ ID NO:177 Nucleotide sequence of ORF-3 of Contig of ESTs CF133508, BF482223 and BQ170720.

SEQ ID NO:178 Nucleotide sequence of ORF-4 of Contig of ESTs CF133508, BF482223 and BQ170720.

SEQ ID NO:179 Nucleotide sequence of ORF-5 of Contig of ESTs CF133508, BF482223 and BQ170720.

SEQ ID NO:180 Nucleotide sequence of ORF-6 of Contig of ESTs CF133508, BF482223 and BQ170720.

SEQ ID NO:181 Amino acid sequence of ORF-1/CFBFBQ.

SEQ ID NO:182 Amino acid sequence of ORF-5/CFBFBQ.

SEQ ID NO:183 Nucleotide sequence of ORF1 DraF2C1 common to BF482223 and BQ170720.

SEQ ID NO:184 Nucleotide sequence of ORF1 DraF2C1 common to CF133508 BF482223 BQ170720.

SEQ ID NO:185 Nucleotide sequence of ORF common to DraF2C2 contig.

SEQ ID NO:186 Nucleotide sequence of ORF2 DraF2C1 common to BF482223 and BQ170720.

SEQ ID NO:187 Nucleotide sequence of ORF2 DraF2C1 common to CF133508 BF482223 BQ170720.

SEQ ID NO:188 Nucleotide sequence of ORF7 of DraF2C1.

SEQ ID NO:189 Nucleotide sequence of ORF8 of DraF2C1.

SEQ ID NO:190 Amino acid sequence of the ORF1-DraF2C1 on the 30dpa gene fragment DraF2C1.

SEQ ID NO:191 Nucleotide sequence of 30dpa gene fragment DraF2C1 from position 1 to 1529.

SEQ ID NO:192 Amino acid sequence of ORF-1 EST-CFBFBQ mod.

SEQ ID NO:193 Nucleotide sequence of 30dpa-DraF2C3 fragment.

SEQ ID NO:194 Nucleotide sequence of ORF-1/30dpa-DraF2C3 fragment.

SEQ ID NO:195 Nucleotide sequence of ORF-2/30dpa-DraF2C3 fragment.

SEQ ID NO:196 Nucleotide sequence of ORF-3/30dpa-DraF2C3 fragment.

SEQ ID NO:197 Nucleotide sequence of ORF-4/30dpa-DraF2C3 fragment.

SEQ ID NO:198 Nucleotide sequence of ORF-5/30dpa-DraF2C3 fragment.

SEQ ID NO:199 Nucleotide sequence of ORF-6/30dpa-DraF2C3 fragment.

SEQ ID NO:200 Nucleotide sequence of ORF-7/30dpa-DraF2C3 fragment.

SEQ ID NO:201 Nucleotide sequence of ORF-8/30dpa-DraF2C3 fragment.

SEQ ID NO:202 Nucleotide sequence of ORF-9/30dpa-DraF2C3 fragment.

SEQ ID NO:203 Nucleotide sequence of ORF DraF2C3 common to CF133508 BF482223 BQ170720

SEQ ID NO:204 Nucleotide sequence of ORF DraF2C3 common to DraF2C1 contig.

SEQ ID NO:205 Nucleotide sequence of DraF2C1 contig fragment from position 220 to 316.

SEQ ID NO:206 Nucleotide sequence of DraF2C1 contig fragment from position 214 to 241.

SEQ ID NO:207 Nucleotide sequence of DraF2C1 contig fragment from position 289 to 316.

SEQ ID NO:208 Nucleotide sequence of gi|157863729|gb|EU159424.1| Triticum turgidum haplotype B DNA repair protein Rad50 gene, complete cds fragment from position 12214 to 12304.

SEQ ID NO:209 Nucleotide sequence of gi|157863729|gb|EU159424.1| Triticum turgidum haplotype B DNA repair protein Rad50 gene, complete cds fragment from position 12304 to 12277.

SEQ ID NO:210 Nucleotide sequence of gi|112361872|gb|DQ871219.1| Triticum turgidum subsp. dicoccoides clones BAC 409D13 and BAC 916017, complete sequence fragment from 106894 to 106990.

SEQ ID NO:211 Nucleotide sequence of gi|112361872|gb|DQ871219.1| Triticum turgidum subsp. dicoccoides clones BAC 409D13 and BAC 916017, complete sequence fragment from 106921 to 106894.

SEQ ID NO:212 Nucleotide sequence of gi|112361872|gb|DQ871219.1| Triticum turgidum subsp. dicoccoides clones BAC 409D13 and BAC 916017, complete sequence fragment from 106990 to 106963.

SEQ ID NO:213 Nucleotide sequence of gi|23476274|gb|AY133251.1|AY133250S2 Hordeum vulgare subsp. vulgare starch synthase II gene, exon 9 and complete cds fragment from position 905 to 1001.

SEQ ID NO:214 Nucleotide sequence of gi|23476274|gb|AY133251.1|AY133250S2 Hordeum vulgare subsp. vulgare starch synthase II gene, exon 9 and complete cds fragment from position 1001 to 974.

SEQ ID NO:215 Nucleotide sequence of DraF2C1 contig fragment from position 769 to 1327.

SEQ ID NO:216 Nucleotide sequence of DraF2C1 contig fragment from position 759 to 1260.

SEQ ID NO:217 Nucleotide sequence of DraF2C1 contig fragment from position 315 to 581.

SEQ ID NO:218 Nucleotide sequence of DraF2C1 contig fragment from position 1 to 215.

SEQ ID NO:219 Nucleotide sequence of DraF2C1 contig fragment from position 1311 to 1529.

SEQ ID NO:220 Nucleotide sequence of DraF2C1 contig fragment from position 611 to 1179.

SEQ ID NO:221 Nucleotide sequence of DraF2C1 contig fragment from position 864 to 1334.

SEQ ID NO:222 Nucleotide sequence of DraF2C1 contig fragment from position 446 to 881.

SEQ ID NO:223 Nucleotide sequence of DraF2C1 contig fragment from position 47 to 214.

SEQ ID NO:224 Nucleotide sequence of DraF2C1 contig fragment from position 336 to 384.

SEQ ID NO:225 Nucleotide sequence of gi|11565524|gb|BF482223.1| WHE1798_C04_F08ZS Wheat pre-anthesis spike cDNA library Triticum aestivum cDNA clone WHE1798_C04_F08, mRNA sequence fragment from position 1 to 563.

SEQ ID NO:226 Nucleotide sequence of gi|125204992|gb|CA626696.1| wl1n.pk0146.f10 wl1n Triticum aestivum cDNA clone wl1n.pk0146.f10 5′ end, mRNA sequence fragment from position 1 to 498.

SEQ ID NO:227 Nucleotide sequence of gi|70960540|gb|DR733736.1| FGAS079494 Triticum aestivum FGAS: Library 2 Gate 3 Triticum aestivum cDNA, mRNA sequence fragment from position 559 to 824.

SEQ ID NO:228 Nucleotide sequence of gi|70960540|gb|DR733736.1| FGAS079494 Triticum aestivum FGAS: Library 2 Gate 3 Triticum aestivum cDNA, mRNA sequence fragment from position 354 to 560.

SEQ ID NO:229 Nucleotide sequence of gi|20332543|gb|BQ170720.1| WHE1798_C04_F08ZT Wheat pre-anthesis spike cDNA library Triticum aestivum cDNA clone WHE1798_C04_F08, mRNA sequence fragment from position 450 to 235.

SEQ ID NO:230 Nucleotide sequence of gi|33217688|gb|CF133508.1| WHE4358_G06_N12ZT Wheat meiotic floret cDNA library Triticum aestivum cDNA clone WHE4358_G06_N12, mRNA sequence fragment from position 93 to 667.

SEQ ID NO:231 Nucleotide sequence of gi|93043667|dbj|CJ637246.1| CJ637246 Y. Ogihara unpublished cDNA library Wh_DPA20 Triticum aestivum cDNA clone whdp8n11 5′, mRNA sequence fragment from position 21 to 499.

SEQ ID NO:232 Nucleotide sequence of gi|143320161|dbj|CJ809854.1| J809854 Y. Ogihara unpublished cDNA library, whsct Triticum aestivum cDNA clone whsct9e04 5′, mRNA sequence fragment from position 267 to 702.

SEQ ID NO:233 Nucleotide sequence of gi|143320161|dbj|CJ809854.1| J809854 Y. Ogihara unpublished cDNA library, whsct Triticum aestivum cDNA clone whsct9e04 5′, mRNA sequence fragment from position 1 to 160.

SEQ ID NO:234 Nucleotide sequence of gi|143320161|dbj|CJ809854.1| J809854 Y. Ogihara unpublished cDNA library, whsct Triticum aestivum cDNA clone whsct9e04 5′, mRNA sequence fragment from position 181 to 229.

SEQ ID NO:235 Amino acid sequence of ORF-1 703 to 1332 bp Agnelo DraF2C1 contig (Region 1 to 209) fragment from position 1 to 169.

SEQ ID NO:236 Amino acid sequence of ORF-1 703 to 1332 bp Agnelo DraF2C1 contig (Region 1 to 209) fragment from position 1 to 204.

SEQ ID NO:237 Amino acid sequence of ORF-1 703 to 1332 bp Agnelo DraF2C1 contig (Region 1 to 209) fragment from position 1 to 201.

SEQ ID NO:238 Amino acid sequence of ORF-1 703 to 1332 bp Agnelo DraF2C1 contig (Region 1 to 209) fragment from position 15 to 204.

SEQ ID NO:239 Amino acid sequence of ORF-1 703 to 1332 bp Agnelo DraF2C1 contig (Region 1 to 209) fragment from position 2 to 200.

SEQ ID NO:240 Amino acid sequence of ORF-1 703 to 1332 bp Agnelo DraF2C1 contig (Region 1 to 209) fragment from position 3 to 173.

SEQ ID NO:241 Amino acid sequence of ORF-1 703 to 1332 bp Agnelo DraF2C1 contig (Region 1 to 209) fragment from position 2 to 127.

SEQ ID NO:242 Amino acid sequence of ORF-1 703 to 1332 bp Agnelo DraF2C1 contig (Region 1 to 209) fragment from position 1 to 172.

SEQ ID NO:243 Amino acid sequence of ORF-1 703 to 1332 bp Agnelo DraF2C1 contig (Region 1 to 209) fragment from position 4 to 200.

SEQ ID NO:244 Amino acid sequence of ORF-1 703 to 1332 bp Agnelo DraF2C1 contig (Region 1 to 209) fragment from position 4 to 124.

SEQ ID NO:245 Amino acid sequence of ORF-1 703 to 1332 bp Agnelo DraF2C1 contig (Region 1 to 209) fragment from position 3 to 67.

SEQ ID NO:246 Amino acid sequence of ORF-1 703 to 1332 bp Agnelo DraF2C1 contig (Region 1 to 209) fragment from position 1 to 111.

SEQ ID NO:247 Amino acid sequence of ORF-1 703 to 1332 bp Agnelo DraF2C1 contig (Region 1 to 209) fragment from position 1 to 158.

SEQ ID NO:248 Amino acid sequence of ORF-1 703 to 1332 bp Agnelo DraF2C1 contig (Region 1 to 209) fragment from position 1 to 67.

SEQ ID NO:249 Amino acid sequence of ORF-1 703 to 1332 bp Agnelo DraF2C1 contig (Region 1 to 209) fragment from position 112 to 163.

SEQ ID NO:250 Amino acid sequence of ORF-1 703 to 1332 bp Agnelo DraF2C1 contig (Region 1 to 209) fragment from position 126 to 202.

SEQ ID NO:251 Amino acid sequence of ORF-1 703 to 1332 bp Agnelo DraF2C1 contig (Region 1 to 209) fragment from position 13 to 91 SEQ ID NO:252 Amino acid sequence of gi|115476200|ref|NP_{—}001061696.1| Os08g0382800 [Oryza sativa (japonica cultivar-group)] from position 222 to 388.

SEQ ID NO:253 Amino acid sequence of gi|125575729|gb|EAZ17013.1| hypothetical protein OsJ_{—}031222 [Oryza sativa (japonica cultivar-group)] from position 216 to 418.

SEQ ID NO:254 Amino acid sequence of gi|115483508|ref|NP_{—}001065424.1| Os10g0566300 [Oryza sativa (japonica cultivar-group)] from position 243 to 445.

SEQ ID NO:255 Amino acid sequence of gi|115483534ref|NP_{—}001065437.1| Os10g0567900 [Oryza sativa (japonica cultivar-group)] from position 151 to 358.

SEQ ID NO:256 Amino acid sequence of gi|110289600|gb|ABG66270.1| F-box protein interaction domain containing protein, expressed [Oryza sativa (japonica cultivar-group)] from position 85 to 292.

SEQ ID NO:257 Amino acid sequence of gi|19224986|gb|AAL86462.1|AC077693_{—}1 putative transposase protein, 5′-partial [Oryza sativa (japonica cultivar-group)] from position 672 to 879.

SEQ ID NO:258 Amino acid sequence of gi|18854992|gb|AAL79684.1|AC087599_{—}3 putative transposase [Oryza sativa] from 190 to 422.

SEQ ID NO:259 Amino acid sequence of gi|125533008|gb|EAY79573.1| hypothetical protein OsI_{—}033532 [Oryza sativa (indica cultivar-group)] from 230 to 437.

SEQ ID NO:260 Amino acid sequence of gi|125532994|gb|EAY79559.1| hypothetical protein OsI_{—}033518 [Oryza sativa (indica cultivar-group)] from 215 to 401.

SEQ ID NO:261 Amino acid sequence of gi|125586371|gb|EAZ27035.1|hypothetical protein OsJ_{—}010518 [Oryza sativa (japonica cultivar-group)] from 190 to 422.

SEQ ID NO:262 Amino acid sequence of gi|108708334|gb|ABF96129.1| F-box domain containing protein [Oryza sativa (japonica cultivar-group)] from 267 to 499.

SEQ ID NO:263 Amino acid sequence of gi|125571320|gb|EAZ12835.1| hypothetical protein OsJ_{—}002660 [Oryza sativa (japonica cultivar-group)] from 171 to 337.

SEQ ID NO:264 Amino acid sequence of gi|125526988|gb|EAY75102.1| hypothetical protein OsI_{—}002949 [Oryza sativa (indica cultivar-group)] from 237 to 403.

SEQ ID NO:265 Amino acid sequence of gi|115438777|ref|NP_{—}001043668.1| Os01g0637100 [Oryza sativa (japonica cultivar-group)] from 217 to 383.

SEQ ID NO:266 Amino acid sequence of gi|125555027|gb|EAZ00633.11 hypothetical protein OsI_{—}021865 [Oryza sativa (indica cultivar-group)] from 53 to 182.

SEQ ID NO:267 Amino acid sequence of gi|125596957|gb|EAZ36737.1| hypothetical protein OsI_{—}020220 [Oryza sativa (japonica cultivar-group)] from 215 to 344.

SEQ ID NO:268 Amino acid sequence of gi|125582083|gb|EAZ23014.1| hypothetical protein OsJ_{—}006497 [Oryza sativa (japonica cultivar-group)] from 191 to 365.

SEQ ID NO:269 Amino acid sequence of gi|125539427|gb|EAY85822.1| hypothetical protein OsI_{—}007055 [Oryza sativa (indica cultivar-group)] from 191 to 365.

SEQ ID NO:270 Amino acid sequence of gi|125605903|gb|EAZ44939.1| hypothetical protein OsJ_{—}028422 [Oryza sativa (japonica cultivar-group)] from 294 to 389.

SEQ ID NO:271 Amino acid sequence of gi|125563939|gb|EAZ09319.1| hypothetical protein OsI_{—}030551 [Oryza sativa (indica cultivar-group)] from 243 to 483.

SEQ ID NO:272 Amino acid sequence of gi|125563928|gb|EAZ09308.1| hypothetical protein OsI_{—}030540 [Oryza sativa (indica cultivar-group)] from 189 to 301.

SEQ ID NO:273 Amino acid sequence of gi|115479445|ref|NP_{—}001063316.1| Os09g0448100 [Oryza sativa (japonica cultivar-group)] from 189 to 301.

SEQ ID NO:274 Amino acid sequence of gi|125579769|gb|EAZ20915.1| hypothetical protein OsJ_{—}035124 [Oryza sativa (japonica cultivar-group)] from 202 to 272.

SEQ ID NO:275 Amino acid sequence of gi|125543997|gb|EAY90136.1| hypothetical protein OsI_{—}011369 [Oryza sativa (indica cultivar-group)] from 272 to 367.

SEQ ID NO:276 Amino acid sequence of gi|63147802|gb|AAY34252.1| F-box like protein [Hordeum vulgare] from 204 to 322.

SEQ ID NO:277 Amino acid sequence of gi|77556844|gb|ABA99640.1| F-box domain containing protein [Oryza sativa (japonica cultivar-group)] from 202 to 273.

SEQ ID NO:278 Amino acid sequence of gi|147854091|emb|CAN83390.1| hypothetical protein [Vitis vinifera] from 129 to 290.

SEQ ID NO:279 Amino acid sequence of gi|125548041|gb|EAY93863.1| hypothetical protein OsI_{—}015096 [Oryza sativa (indica cultivar-group)] from 178 to 244.

SEQ ID NO:280 Amino acid sequence of gi|1066176|emb|CAA61663.1| virion protein [Canid herpesvirus 1] from 61 to 113.

SEQ ID NO:281 Amino acid sequence of gi|190622529|gb|EDV38053.1| GF11106 [Drosophila ananassae] from 252 to 533.

SEQ ID NO:282 Amino acid sequence of gi|125590154|gb|EAZ30504.1| hypothetical protein OsJ_{—}013987 [Oryza sativa (japonica cultivar-group)] from 212 to 292.

SEQ ID NO:283 Amino acid sequence of gi||38347475|emb|CAE05295.2| OSJNBa0084N21.13 [Oryza sativa (japonica cultivar-group)] from 212 to 292.

SEQ ID NO:284 Amino acid sequence of ORF-1DraF2C3.

SEQ ID NO:285 Nucleotide sequence of ORF-1 of DraF2C1 fragment.

SEQ ID NO:286 Nucleotide sequence of ORF-2 of DraF2C1 fragment.

SEQ ID NO:287 Nucleotide sequence of ORF-3 of DraF2C1 fragment.

SEQ ID NO:288 Nucleotide sequence if ORF-4 of DraF2C1 fragment.

SEQ ID NO:289 Nucleotide sequence of ORF-5 of DraF2C1 fragment.

SEQ ID NO:290 Amino acid sequence of ORF-3 on DraF2C2, DraF2C3 and DraF2C4.

SEQ ID NO:291 Amino acid sequence of ORF-4 on DraF2C2, DraF2C3 and DraF2C4.

SEQ ID NO:292 Amino acid sequence of ORF-5 on DraF2C2, DraF2C3 and DraF2C4.

SEQ ID NO:293 Amino acid sequence of ORF-6 on DraF2C3.

SEQ ID NO:294 Amino acid sequence of ORF-7 on DraF2C3.

SEQ ID NO:295 Amino acid sequence of ORF-8 on DraF2C3.

SEQ ID NO:296 Nucleotide sequence of ORF-3 on DraF2C2, DraF2C3 and DraF2C4.

SEQ ID NO:297 Nucleotide acid sequence of ORF-4 on DraF2C2, DraF2C3 and DraF2C4.

SEQ ID NO:298 Nucleotide sequence of ORF-5 on DraF2C2, DraF2C3 and DraF2C4.

SEQ ID NO:299 Nucleotide sequence of ORF-6 on DraF2C3.

SEQ ID NO:300 Nucleotide sequence of ORF-7 on DraF2C3.

SEQ ID NO:301 Nucleotide sequence of ORF-8 on DraF2C3.

SEQ ID NO:302 Nucleotide sequence of Contig CF BF BQ.

### DETAILED DESCRIPTION OF THE INVENTION

Pina-D1 and Pinb-D1 have not been associated with or linked to improved millability. The present invention is predicated on the discovery of differential patterns of gene expression between low flour yielding and high flour yielding wheat varieties during the development of wheat seed. From these results, it was established that low yielding wheat varieties express a disjoint set of genes when compared to high yielding wheat varieties during the early stages of wheat seed development. The inventors have concluded that improved millability is associated with or linked to expression distribution and patterns of certain nucleic acid sequences at different stages during wheat seed development. In particular, there is a striking disparity in expression of TA.28688.1.A1_AT at 14 dpa and TA.11743.1.A1.AT at 30 dpa in low flour yielding wheat varieties compared to high flour yielding wheat varieties, which indicates a genetic basis for the control of flour yield and therefore improved millability.

Throughout this specification, the terms “TA.11743.1.A1.at”, “30 dpa gene” and “30dpa sequence” will be used interchangeably to generally refer to the isolated nucleic acid associated with or linked to improved millability showing increased expression in low milling varieties at 30 dpa.

Similarly, the terms “TA.28688.1.A1_AT”, “14 dpa gene” and “14dpa sequence” will be used interchangeably to generally refer to the isolated nucleic acid associated with or linked to improved millability showing increased expression in low milling varieties at 14 dpa.

By utilising approaches such as genome walking and Expressed Sequence Tag (EST) database mining, a number of candidate open reading frames and/or protein coding sequences were characterised for each of the 14 dpa sequence and the 30 dpa sequence.

Based upon sequence alignment studies, the present inventors postulate that the nucleotide sequence of the 14 dpa sequence (or alternatively referred to as TA.28688.1.A1_AT) encodes a transcription elongation factor. Broadly, transcription elongation factors interact with RNA polymerase II to increase (positive transcription elongation factor) or reduce (negative transcription elongation factor) the rate of transcription elongation. Although not wishing to be bound by any particular theory, the translated product of TA.28688.1.A1_AT may regulate gene expression at a global level or alternatively, at a gene specific level. It is conceivable that the high levels of expression of TA.28688.1.A1_AT in wheat varieties with poor milling performance up-regulates or down-regulates expression of one or more other genes which in turn, has downstream negative effects on flour yields.

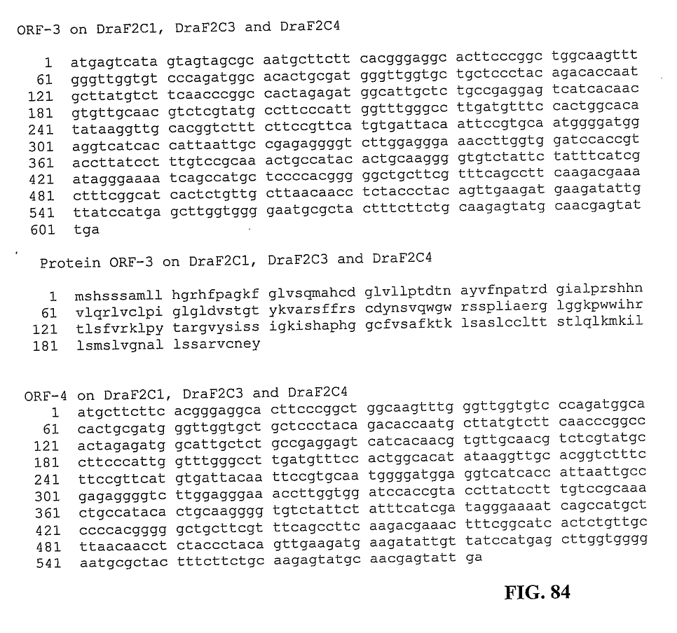

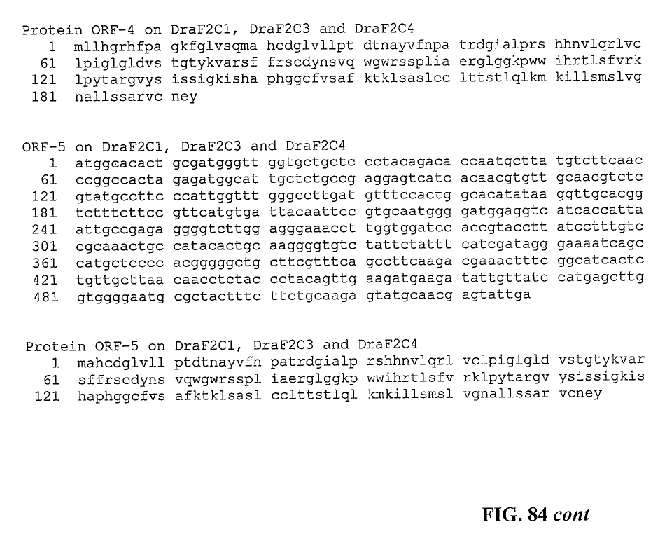

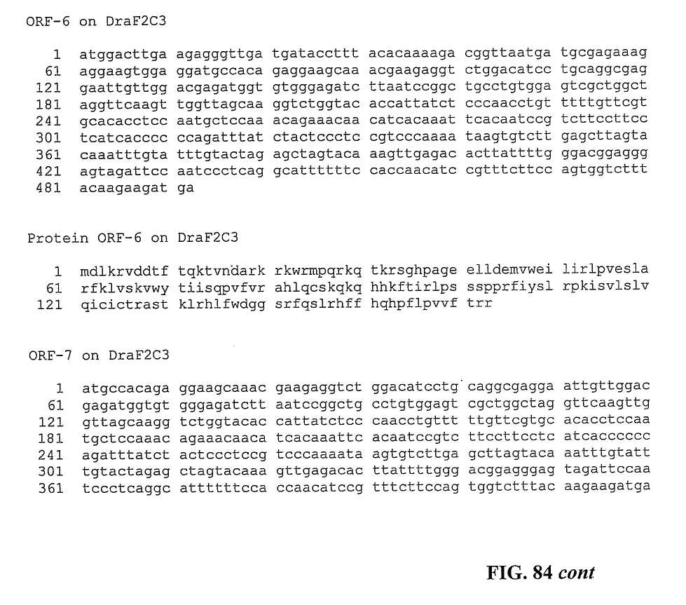

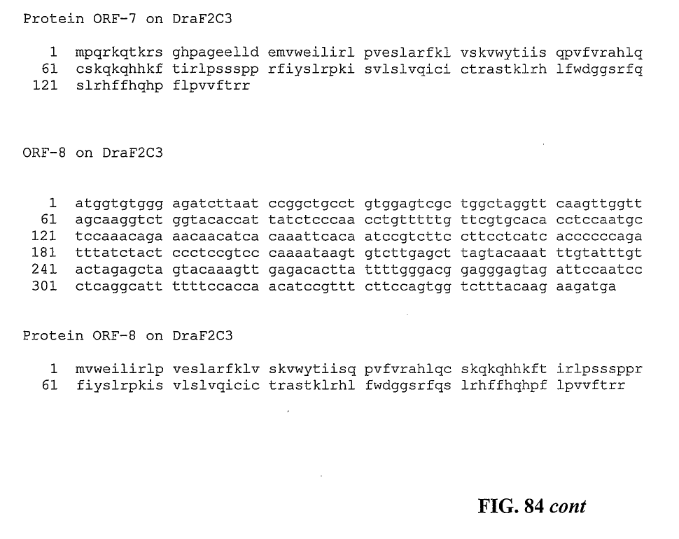

Although not wishing to be bound by any particular theory, the 30 dpa sequence may also broadly be involved in the control of gene expression, particularly at the stage of transcription elongation. The 30 dpa sequence as characterised by the present inventors has several open reading frames (ORFs) and/or protein coding regions as shown in FIG. 84. Preferably, the 30 dpa sequence ORF is a nucleotide sequence of an ORF on a nucleotide sequence selected from the group consisting of SEQ ID NOs:159 to 161. In preferred embodiments that relate to the 30dpa sequence, the isolated nucleic acid associated with or linked to improved millability encodes a polypeptide comprising an amino acid sequence selected from the group consisting of SEQ ID NO:190, SEQ ID NOS: 290 to 295 and SEQ ID NO:284. Preferably, the polypeptide has an amino acid sequence as set forth in SEQ ID NO:190.

Although not wishing to be bound by any particular theory, the polypeptide encoded by SEQ ID NO:190 is a cyclin-like F-box domain containing protein which has a potential role in control of gene expression and in particular transcription elongation, polyubiquitination, centromere binding and translational repression.

In other preferred embodiments relating to the 30dpa sequence, the ORF and/or protein coding region of the isolated nucleic acid associated with or linked to improved millability comprises a nucleotide sequence selected from the group consisting of SEQ ID NO: 188, SEQ ID NO: 189, SEQ ID NOS:193 to 202, SEQ ID NOS:285 to 289 and SEQ ID NOS:296 to 301.

Hence, the present invention broadly aims to utilise the observation that flour yield and milling performance, and in particular good milling performance, is under genetic control, to thereby provide methods of predicting, selecting and engineering improved commercial milling performance of a grain or grain-producing plant.

By “millability” is meant the capability of a grain or a grain-producing plant to be milled into a flour. The millability of a grain or a grain-producing plant is related to kernel hardness, the endosperm to bran ratio and ease of separation of the bran but is not limited thereto. Typically, although not exclusively, the milling process is more straightforward if the starting material exhibits a readier separation of bran from endosperm as the resultant flour is more mobile and easier to sift. Throughout this specification, millability will be used interchangeably with “milling performance”.

The term “improved” in the context of the present invention may relate to selection from a population of a grain or a grain-producing plant which is genetically predisposed to possessing superior or enhanced milling performance as a result of altered relative amounts of an isolated nucleic acid associated with or linked to improved millability. Alternatively, “improved” may relate to superior or enhanced millability by genetic-modification using conventional plant breeding or recombinant DNA methodologies.

Flour can be milled from a variety of crops, primarily cereals or other starchy food sources. Non-limiting examples are wheat, corn, maize and rye as well as other grasses and seed producing crops such as legumes and nuts.

Preferably, the crop is a cereal.

Even more preferably, the cereal is wheat.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

The term “nucleic acid” as used herein designates single- or double-stranded mRNA, RNA, cRNA and DNA inclusive of cDNA, genomic DNA and DNA-RNA hybrids.

One broad application of the present invention is a genetic-based method of analysing and/or predicting whether a grain or a grain-producing plant is likely to have improved milling performance. More particularly, methods of the invention are amenable for use in plant breeding programmes such as at the seedling stage. Such methods prove advantageous for accelerating and improving the efficiency of plant breeding and ultimately, improved milling performance.

In a particular aspect, the invention resides in a method for selecting a grain or a grain-producing plant which possesses the trait of improved millability by determining the relative amount of an isolated nucleic acid associated with or linked to improved millability to determine whether or not the grain or grain-producing plant has a predisposition to improved milling performance.

In a preferred embodiment, a grain or grain-producing plant will be selected for improved millability if the grain or grain-producing plant has a reduced relative amount of an isolated nucleic acid associated with or linked to improved millability when compared to a reference sample.

In another particular aspect, the invention resides in a method of determining the genetic predisposition of a grain or grain-producing plant for improved milling performance by detecting whether the grain or grain-producing plant has an isolated nucleic acid associated with or linked to improved millability of the present invention.

By “relative amount” is meant the relative level, proportion or otherwise quantity of an isolated nucleic acid associated with or linked to improved millability in a test sample when compared to the amount of the same isolated nucleic acid in a standard sample. In certain circumstances, it may be appropriate to predict improved millability in a grain or grain-producing plant relative to a standard sample such as a high flour yielding variety. It is also appropriate that the standard sample be a low flour yielding variety. It will be understood that by “relative amount” is meant not an absolute amount.

For the purpose of this invention, the terms “predisposed” or “predisposition” relate to the probability that a grain or a grain-producing plant will display improved flour yield potential as a result of an underlying genetic cause.

Preferably, the isolated nucleic acid associated with or linked to improved millability encodes a polypeptide comprising an amino acid sequence selected from the group consisting of SEQ ID NO:49, SEQ ID NO:190, SEQ ID NO:284 and SEQ ID NOS:290 to 295, or a variant thereof.

In one preferred embodiment, the isolated nucleic acid associated with or linked to improved millability encodes a polypeptide comprising an amino acid sequence selected from the group consisting of SEQ ID NO:49 and SEQ ID NO:190, or a fragment thereof.

In another preferred embodiment, the isolated nucleic acid associated with or linked to improved millability comprises a nucleotide sequence selected from the group consisting of SEQ ID NO:15, SEQ ID NO:26, SEQ ID NOS:159 to 169, SEQ ID NOS:188 to 189, SEQ ID NOS:194 to 202, SEQ ID NOS:285 to 289 and SEQ ID NOS:296 to 301, or a variant thereof.

In other preferred embodiments, the isolated nucleic acid associated with or linked to improved millability comprises a nucleotide sequence selected from the group consisting of SEQ ID NO:26 and SEQ ID NO:285, or a fragment thereof.

Genetic analysis methods as described herein could employ nucleic acid detection techniques as are well known in the art.

In principle, any nucleic acid sequence detection technique may be applicable, such as nucleic acid sequencing, northern and southern hybridization, nucleic acid sequence amplification and nucleic acid arrays.

For the purposes of detecting whether a grain or a grain-producing plant is predisposed to having improved millability, the invention contemplates particular embodiments of such methods which may be used alone or in combination.

In one general embodiment, a nucleic acid sequence amplification technique may be useful for rapid detection of said genetic loci which is indicative of improved millability, particularly where multiple samples are to be tested.

As used herein, a “nucleic acid sequence amplification technique” includes but is not limited to polymerase chain reaction (PCR) as for example described in Chapter 15 of CURRENT PROTOCOLS IN MOLECULAR BIOLOGY Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al. 1996, Proc. Natl. Acad. Sci. USA 93 5395 and helicase-dependent amplification as for example described in International Publication WO 2004/02025.

In this regard, it will be appreciated than an RNA copy of DNA corresponds to the DNA notwithstanding the presence of uracil bases rather than thymine bases.

Nucleic acid fragments in certain embodiments may have about 9, 12, 15, 20, 30 or up to 60 contiguous nucleotides (such as for a PCR primer) or have 100, 200, 300 or more contiguous nucleotides (such as for a probe).

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

A “polynucleotide” is a nucleic acid having eighty (80) or more contiguous nucleotides, while an “oligonucleotide” has less than eighty (80) contiguous nucleotides.

The terms “anneal”, “hybridize” and “hybridization” are used herein in relation to the formation of bimolecular complexes by base-pairing between complementary or partly-complementary nucleic acids in the sense commonly understood in the art. It should also be understood that these terms encompass base-pairing between modified purines and pyrimidines (for example, inosine, methylinosine and methyladenosine) and modified pyrimidines (for example thiouridine and methylcytosine) as well as between A, G, C, T and U purines and pyrimidines. Factors that influence hybridization such as temperature, ionic strength, duration and denaturing agents are well understood in the art, although a useful operational discussion of hybridization is provided in to Chapter 2 of CURRENT PROTOCOLS IN MOLECULAR BIOLOGY (Eds. Ausubel et al. John Wiley & Sons NY, 2000), particularly at sections 2.9 and 2.10.

The invention also contemplates using high-throughput diagnostic methods that utilize nucleic acid arrays for selection of a grain or a grain-producing plant that is genetically predisposed to improved millability.

In one embodiment, a library or array comprising one or more improved millability-associated nucleic acids, may be used to screen grain or grain-producing plant samples.

In another embodiment, screening using a library or array encompasses a combination of improved millability-associated nucleic acids as hereinbefore described and other improved millability associated traits such as hardness-associated nucleic acids, but is not limited thereto.

In one particular form of this embodiment, the invention provides a molecular library in the form of a nucleic acid array that comprises a substrate to which is immobilized, bound or otherwise coupled an improved millability-associated nucleic acid identified according to particular aspects of the invention, or a fragment thereof. Each immobilized, bound or otherwise coupled nucleic acid has an “address” on the array that signifies the location and identity of said nucleic acid.

Nucleic acid array technology has become well known in the art and examples of methods applicable to array technology are provided in Chapter 22 of CURRENT PROTOCOLS IN MOLECULAR BIOLOGY Eds. Ausubel et al. (John Wiley & Sons NY USA 1995-2001).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

It can be appreciated by a person of skill in the art that the invention also provides for a kit for the detection in a biological sample of isolated nucleic acids of the invention which are indicative of a predisposition to improved millability. The kit may be based on amplification of nucleic acids using PCR and may include primers for hybridizing with a known nucleic acid, reagents such as buffers and a thermostable DNA polymerase. It can also be appreciated that both DNA and mRNA can be detected using this kit. The enzyme used is dependent upon whether DNA or mRNA is to be detected. Detection of mRNA may be performed using a one-step coupled RT-PCR, which includes a mixture of a RNA-dependent DNA polymerase and a DNA-dependent DNA polymerase, with a buffer allowing maximal activity of the two enzymes in the same reaction mixture. Alternatively, detection kits for nucleic acids may be based on hybridization techniques common to the art such as Northern or Southern blotting using probes designed to detect the genetic region of interest. A nucleic acid may be detected using a variety of labels common in the art such as fluorescent dyes, radioactive labels such ³²P or ³⁵S, enzymes and metals, including gold.

With regard to the above, nucleic acid samples for genetic analysis may be isolated from any cell or tissue source, inclusive of endosperm tissue. For example such tissues may include but are not restricted to leaves, roots, stems and seeds.

In another general embodiment the methods of the invention may involve measuring expression levels of improved millability-associated nucleic acids of the invention, compared to a reference sample.

Methods for quantification for nucleic acids are well known in the art. Measurement of relative amounts of improved millability-associated nucleic acid levels (e.g. TA.28688.1.A1_AT and/or TA.11743.1.A1.at) compared to an expressed level of a reference nucleic acid may be conveniently performed using a nucleic acid array as hereinbefore described. Alternative methods include hybridisation techniques such as northern hybridisation, as are well known in the art.

In another particular form of this embodiment, quantitative or semi-quantitative PCR using primers corresponding to one or more improved millability-associated nucleic acids of the invention (eg. may be used to quantify relative expression levels of the or each nucleic acid to thereby determine whether a grain or a grain-producing plant is predisposed to improved millability). Exemplary primers comprise a nucleotide sequence selected from the group consisting of SEQ ID NOS: 28-40 in relation to embodiments encompassing Ta.28688.1.A1_at. In those general embodiments encompassing TA.11743.1.A1.at, exemplary primers comprise a nucleotide sequence selected from the group consisting of SEQ ID NO:140-154.

PCR amplification is not linear and hence end point analysis does not always allow for the accurate determination of nucleic acid expression levels. Real-time PCR analysis provides a high throughput means of measuring gene expression levels. It uses specific primers, an intercalating fluorescent dye such as SYBR Green I or ethidium bromide (EtBr) and fluorescence detection to measure the amount of product after each cycle. Hybridization probes utilise either quencher dyes or fluorescence directly to generate a signal. This method may be used to validate and quantify nucleic acid expression differences in cells or tissues obtained from a grain or a grain-producing plant with low flour yields compared to cells or tissues obtained from a grain or a grain-producing plant that produces high flour yields.

The invention also contemplates variants of the isolated nucleic acid associated with or linked to improved millability that share a relationship based upon homology between sequences.

“Homology” refers to the percentage number of nucleotides of a nucleotide sequence that are identical to a reference nucleotide sequence. Homology may be determined using sequence comparison programs such as BESTFIT (Deveraux et al. 1984, Nucleic Acids Research 12, 387-395) which is incorporated herein by reference. In this way sequences of a similar or substantially different length to those cited herein might be compared by insertion of gaps into the alignment, such gaps being determined, for example, by the comparison algorithm used by BESTFIT.

Terms used to describe sequence relationships between two or more nucleotide sequences include “reference sequence”, “comparison window”, “sequence identity”, “percentage of sequence identity” and “substantial identity”. A “reference sequence” is at least 6 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 6 to 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389, which is incorporated herein by reference. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis over a window of comparison. Thus, a “percentage of sequence identity” is calculated by comparing two optimally-aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software, which is incorporated herein by reference.

In one embodiment, nucleic acid variants share at least 50%, 55% or 60%, preferably at least 65%, 66%, 67%, 68%, 69% or 70%, 71%, 72%, 73%, 74%, more preferably at least 75%, 76%, 77%, 78%, 79%, 80%, 81%, 82%, 83%, 84%, 85%, 86%, 87%, 88% or 89%, and even more preferably at least 90%, 91%, 92%, 93%, 94%, 95%, 96%, 97%, 98%, 99% or 100% sequence identity with the isolated nucleic acids of the invention.

In a preferred embodiment, the nucleic acid variant is a variant of the nucleotide sequence of the 14 dpa sequence of the present invention. More preferably, the nucleotide sequence of a 14 dpa sequence variant is selected from the group consisting of SEQ ID NO: 41, SEQ ID NO: 42, SEQ ID NO:43, SEQ ID NO:44, SEQ ID NO:45, SEQ ID NO:46, SEQ ID NO:47 and SEQ ID NO:48.

In another preferred embodiment, the nucleic acid variant is a variant of the nucleotide sequence of 30 dpa sequence of the present invention. More preferably, the nucleotide sequence of the 30 dpa sequence variant as set forth in SEQ ID NO:194.

In another embodiment, nucleic acid variants hybridise to nucleic acids of the invention, including fragments, under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to low stringency conditions includes and encompasses:—
(i) from at least about 1% v/v to at least about 15% v/v formamide and from at least about 1 M to at least about 2 M salt for hybridisation at 42° C., and at least about 1 M to at least about 2 M salt for washing at 42° C.; and
(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:—
(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and
(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20, herein incorporated by reference.

Nucleic acid variants of the invention may be prepared according to the following procedure:
(i) obtaining a nucleic acid extract from a suitable host, for example a wheat species;
(ii) creating primers which are optionally degenerate wherein each comprises a fragment of a nucleotide sequence which corresponds to an isolated nucleic acid associated with or linked to improved millability such as SEQ ID NO:15, SEQ ID NO:26 and SEQ ID NO:285; and
(iii) using said primers to amplify, via nucleic acid amplification techniques, one or more amplification products from said nucleic acid extract.

As used herein, an “amplification product” refers to a nucleic acid product generated by nucleic acid amplification techniques.

The present invention also contemplates protein homologues or variant of the amino acid sequence as set forth in SEQ ID NO:49 and SEQ ID NO:190.

As generally used herein, a “protein homologue” shares a definable amino acid sequence relationship with a protein of the invention as the case may be.

“Protein homologues” share at least 60%, preferably at least 65%, 70%, 71%, 72%, 73%, 74%, 75%, 76%, 77%, 78%, 79% or 80% and more preferably at least 80%, 81%, 82%, 83%, 84%, 85%, 86%, 87%, 88%, 89%, 90%, 91%, 92%, 93%, 94%, 95%, 96%, 97%, 98% or 99% sequence identity with the amino acid sequences of proteins of the invention as hereinbefore described. It will be appreciated that a homolog comprises all integer values less than 100%, for example the percent value as set forth above and others.

The present invention further contemplates a method of identifying one or more plant genetic loci which is/are associated with improved millability of a grain or a grain-producing plant, including the step of determining whether one or more plant genetic loci is/are associated with or linked to flour milling yield.

By “genetic locus or loci” is meant the position of a gene in a linkage map or on a chromosome.

The term “gene” is used herein to describe a discrete nucleic acid locus, unit or region within a genome that may comprise one or more of introns, exons, splice sites, open reading frames and 5′ and/or 3′ non-coding regulatory sequences such as a a polyadenylation sequence.

In general embodiments, the invention contemplates identification of one or more polymorphisms of the nucleotide sequence of the 14dpa sequence and/or 30dpa sequence, wherein said variant may also be linked to or associated with improved millability. Preferably, the variant is a variant of a nucleotide sequence selected from the group consisting of SEQ ID NO:15, SEQ ID NO:26, SEQ ID NO:159 and SEQ ID NO:285. More preferably, the variant is selected from the group consisting of SEQ ID NO:15 and SEQ ID NO:159.

The term “polymorphism” is used herein to indicate any variation in an allelic form of a gene or its encoded protein that occurs in a grain or grain-producing plant population. This term encompasses mutation, insertion, deletion, variant and other like terms that indicate specific types of polymorphisms.

It is envisaged that particular polymorphisms, inclusive of single nucleotide polymorphisms (SNPs), splice variants and the like may be identified as being indicative of a predisposition to improved millability and will be useful for screening a grain or a grain-producing plant.

Such polymorphisms may be present in any nucleotide sequence of a gene, including but not limited to protein coding regions (e.g. exon sequences), non-coding intronic sequences, intergenic sequences, non-regulatory sequences upstream and downstream of the 5′ and 3 ′-UTRs respectively, regulatory regions including enhancers, polyadenylation signals, splice acceptor/donor sites and nucleotide sequences that affect mRNA processing, splicing, turnover and/or translation.

The skilled person will be aware of a variety of techniques whereby nucleic acid polymorphisms may be identified.

Typically, although not exclusively, nucleotide sequence polymorphisms may be identified by nucleotide sequencing as is well known in the art. Extensive methodology relating to nucleotide sequencing is provided in Chapter 7 of CURRENT PROTOCOLS IN MOLECULAR BIOLOGY Eds. Ausubel et al. John Wiley & Sons NY USA (1995-2002).

Therefore the present invention also contemplates identification of natural allelic variants of TA.28688.1.A1_AT and/or TA.11743.1.A1.at.

It is envisaged that a genetic loci associated with improved millability can be identified by any one of a number of other methods that are well known in the art. By way of example only, a genetic loci may be identified by construction and screening of either a genomic, Expressed Sequence Tag (EST) or cDNA library. Extensive methodology relating to library screening is provided in Chapters 5 and 6 of CURRENT PROTOCOLS IN MOLECULAR BIOLOGY Eds. Ausubel et al. John Wiley & Sons NY USA (1995-2002). A non-limiting example of genomic approach is genome walking using methods as are well known in the art. Approaches based on genome-wide expression data may also be employed. Non-limiting examples of potential methodologies include serial analysis of gene expression (SAGE), screening of EST libraries and hybridisation-based measures of global gene expression such as microarray analysis (see Chapters 10, 22 and 25 CURRENT PROTOCOLS IN MOLECULAR BIOLOGY Eds. Ausubel et al. John Wiley & Sons NY USA (1995-2002)). Other gene mapping techniques well known in the art such as, but not limited to, linkage analysis may be used to obtain a chromosomal location of the genetic loci associated with improved grain millability.

It will be appreciated that in particular embodiments, the present invention contemplates fragments of the 14dpa sequence or the 30dpa sequence or one or more other plant genetic loci associated with or linked to improved millability which can be identified as hereinbefore described. Typically, a fragment will constitute less than 100% of a genetic locus or at least 5%, 10%, 15%, 20%, 25%, 30%, 40%, 50%, 60%, 70%, 80%, up to 90%. In preferred embodiments relating to the 30dpa sequence, the fragment comprises a nucleotide sequence selected from the group consisting of SEQ ID NOS: 57 to 60, SEQ ID NOS: 72 to 74, SEQ ID NOS: 95 to 99, SEQ ID NOS: 215 to 224, SEQ ID NO: 102, SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

In one embodiment, the fragment may encompass a nucleotide sequence which encodes a protein that regulates improved milling performance by, for example, regulating gene expression such as transcription elongation or alternatively, starch synthesis and amyloplast division, but is not limited thereto.

In a particular embodiment, the fragment may also include a “biologically active” fragment, which retains biological activity of a given protein. In the context of the present invention, biological activity is broadly directed to the ability to regulate the millability of a grain or a grain producing plant.

In one embodiment, a “fragment” includes a protein comprising an amino acid sequence that constitutes less than 100% of an amino acid sequence of an entire protein. A fragment preferably comprises less than 99%, 98%, 97%, 96%, 95%, 94%, 93%, 92%, 91%, 90%, 85%, 80%, 75%, 70%, 60%, 50%, 40%, 30%, 20% or as little as even 10%, 5% or 3% of the entire protein.

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

It is envisaged that a further broad application of the invention is a method of producing a grain or a grain-producing plant with improved millability through manipulating the expression of a gene associated with or linked to improved millability. Preferably, manipulation is selective modulation.

In preferred embodiments relating to the 14 dpa sequence, the gene associated with or linked to improved millability encodes a polypeptide with an amino acid sequence as set forth in SEQ ID NO:49.

In other preferred embodiments relating to the 14 dpa sequence, the gene associated with or linked to improved millability comprises a nucleotide sequence selected from the group consisting of SEQ ID NO:15 and SEQ ID NO:26, or a variant thereof.

In preferred embodiments relating to the 30 dpa sequence, the gene associated with or linked to improved millability encodes a polypeptide with an amino acid sequence as set forth in SEQ ID NO:190, or a variant thereof.

In preferred embodiments relating to the 30 dpa sequence, the gene associated with or linked to improved millability comprises a nucleotide sequence selected from the group consisting of SEQ ID NOS:159 to 161, SEQ ID NOS:188 to 189, SEQ ID NOS:193 to 202, SEQ ID NOS:285 to 289 and SEQ ID NOS:296 to 301. Preferably, the gene is SEQ ID NO:285.

It will be appreciated by a person of skill in the art that the invention encompasses production grain or grain-producing plants with improved millability by genetic-modification through conventional plant breeding techniques or, alternatively, recombinant DNA methodology. Therefore in one aspect, the invention provides a method of producing a grain or grain-producing plant which includes the step of selectively modulating a gene associated with or linked to improved millability so that the relative amount of the gene associated with or linked t improved millability is lowever than in a grain-producing plant where said gene has not been modulated.

The term “genetically-modified” broadly refers to introduction of a heterologous nucleic acid into a plant. The heterologous nucleic acid may subsist in the organism by means of chromosomal integration into the host genome or alternatively, by episomal replication. Preferably, genetic-modification results in either a substantially reduced level of or, alternatively, zero expression of a gene associated with or linked to improved millability, when compared to a non-modified plant.

By “conventional plant breeding” is meant the creation of a new plant variety by hybridisation of two donor plants, one of which carries the trait of interest, followed by screening and field selection. This process is not reliant upon insertion of recombinant DNA in order to express a desired trait.

It will be appreciated by a person of skill in the art that a method for conventional plant breeding typically comprises identifying one or more parent plants which comprise at least one genetic component enhancing flour yield through the regulation of a gene associated with or linked to improved millability. By way of example only, conventional plant breeding methods may include the following steps:

(a) identifying a first parent plant and a second parent plant, wherein the first and second parent plants comprise at least one gene associated with or linked to improved millability, and wherein the first and second plants are capable of cross-pollination. Genetic screening methods well known to a person of skill in the art may be used to identify appropriate parents;

(b) pollinating the first parent plant with pollen from the second parent plant, or pollinating the second parent plant with pollen from the first parent plant;

(c) culturing the pollinated plant under conditions to produce progeny plants;

(d) selecting progeny plants that are homozygous for the quality trait using methods which are well known in the art.

It will be appreciated by those skilled in the art that once plants have been obtained which are heterozygous or homozygous for the improved millability enhancing element(s), those heterozygous or homozygous plants may be used in breeding programmes to transfer the ability to produce higher flour yields to plant varieties producing low flour yields.

It will be appreciated by a person skilled in the art that conventional plant breeding may include studying the genetic variability of the gene associated with or linked to improved millability and correlating the observed diversity with gene expression measurements. Those alleles with low expression would be selected for in breeding programs using nucleic acid based markers that distinguish between low and high expressing alleles of the genes. Knowledge of the contribution to gene expression in the developing seed, from the probable three loci from each of the A, B and D genomes of bread wheat for example may also be valuable for this approach.

The plants identified or produced by the methods of the invention may be used to produce any food product for which that organism is suitable. For example, cereal crops may be used to produce rice, flour and grains for use in the production of food products such as, for example, bread, beer and other fermented and non-fermented beverages.

It will be well understood by a person of skill in the art that selective modulation of the relative amount of a gene associated with or linked to improved millability may require down-regulation.

A person of skill in the art will readily appreciate that down-regulation of expression of one or more improved millability-associated genes in a plant can be effected by silencing. Silencing can be achieved by introduction of synthetic recombinant molecules or transgenes targeted to disrupt or degrade specific nucleotide sequences. Hence according to one embodiment, silencing can occur by construction of a knockout gene. Typically, although not exclusively, a gene knockout is created by homologous recombination of a foreign sequence into the gene of interest, to thereby disrupt the gene.

According to a further embodiment, the gene of the present invention may be silenced at the post-transcriptional level. By way of example only, the invention is well suited to a loss of function approach which employs introduction of one or more site-specific mutations into the nucleic acid. A person of skill in the art will recognise that an advantage to this approach is the ability to engineer precise mutations which have an effect the function of the protein. Hence, mutants can be artificially engineered using an assortment of recombinant techniques. Non-limiting examples of suitable techniques include oligonucleotide-mediated (or site-directed) mutagenesis, PCR mutagenesis and cassette mutagenesis.

Alternatively, loss of function mutants may be generated using random mutagenesis (e.g., transposon mutagenesis) to introduce mutations without a prior knowledge of their function.

According to yet a further embodiment, silencing may involve generation of an inhibitory RNA molecule (hereinafter referred to as “RNAi”). RNAi, and in particular siRNA (but not limited thereto), involves sequence specific cleavage of a cognate mRNA. Therefore the present invention contemplates generation of genetic reagents for RNAi wherein the genetic reagents comprises one or more nucleotide sequences capable of directing synthesis of an RNA molecule, said nucleotide sequence selected from the list comprising:—

(i) a nucleotide sequence transcribable to an RNA molecule comprising an RNA sequence which is substantially homologous to an RNA sequence encoded by a nucleotide sequence substantially homologous to or matching the nucleotide sequence of the present invention and preferably, as set forth in SEQ ID NO:26 or SEQ ID NO:285;

(ii) a reverse complement of the nucleotide sequence of (i);

(iii) a combination of the nucleotide sequences of (i) and (ii),

(iv) multiple copies of nucleotide sequences of (i), (ii) or (iii), optionally separated by a spacer sequence;

(v) a combination of the nucleotide sequences of (i) and (ii), wherein the nucleotide sequence of (ii) represents an inverted repeat of the nucleotide sequence of (i), separated by a spacer sequence; and

(vi) a combination as described in (v), wherein the spacer sequence comprises an intron sequence spliceable from said combination;

Where the nucleotide sequence comprises an inverted repeat separated by a non-intron spacer sequence, upon transcription, the presence of the non-intron spacer sequence facilitates the formation of a stem-loop structure by virtue of the binding of the inverted repeat sequences to each other. The presence of the non-intron spacer sequence causes the transcribed RNA sequence (also referred to herein as a “transcript”) so formed to remain substantially in one piece, in a form that may be referred to herein as a “hairpin”. Alternatively, where the nucleotide sequence comprises an inverted repeat wherein the spacer sequence comprises an intron sequence, upon transcription, the presence of intron/exon splice junction sequences on either side of the intron sequence facilitates the removal of what would otherwise form into a loop structure. The resulting transcript comprises a double-stranded RNA (dsRNA) molecule, optionally with overhanging 3′ sequences at one or both ends. Such a dsRNA transcript is referred to herein as a “perfect hairpin”. The RNA molecules may comprise a single hairpin or multiple hairpins including “bulges” of single-stranded DNA occurring in regions of double-stranded DNA sequences.

It can be foreseen that a reduction of the expression of the gene associated with or linked to improved millability of the present invention in grain or grain-producing plants such as wheat varieties that have low flour milling scores, can convert these varieties to high flour milling varieties. RNA silencing may be used as an approach to reduce the expression of the genes associated with or linked to improved millability in low milling varieties. For example, a wheat variety with low milling score may be transformed with RNAi vectors containing the isolated nucleic acids associated with or linked to improved millability to yield several independent genetically-modified wheat plants. Independent genetically-modified plants may be screened to identify those with reduced transcription followed by determining milling performance.

The present invention also contemplates creation of new alleles by mutagenesis which are low or non-expressing, or alternatively do not contribute to functional protein after expression. These new alleles could likewise be selected in breeding programs using specific DNA markers. This approach could lead to expression levels or levels of functional protein below that found in wheat varieties included in breeding trials. This could potentially increase the positive influence that these genes can have on flour yield beyond that found in existing breeding lines.

It is envisaged that low expressing or non-functional versions of the gene associated with or linked to improved millability may be identified from germplasm with induced mutations using the methods developed for TILLING (McCallum et al., Nat. Biotechnol. (2000) 18, 455-457; Till et al., Methods Mol. Biol. (2003) 236: 205-220; Till et al. Genome Res. (2003) 13: 524-530). This approach would likely involve targeting each of the loci on the three genomes separately in the one pool of mutants generated for TILLING. Judicious crossing and selection of resulting lines could result in plants with lower expression of functional gene product and hence the potential to improve flour yield beyond that possible with wild-type alleles. In any case the potential to produce new alleles with low expression or non-functional gene products could increase the possibilities for selection of appropriate alleles and creation of high flour yield varieties.

In alternative preferred embodiments that relate to methods for the generation of genetically-modified plants, it can also be foreseen the method further includes the step of increasing expression of the gene associated with or linked to improved millability of the present invention in desired wheat varieties that have high milling scores to convert these varieties into low milling varieties. A wheat variety with high milling scores will be selected and transformed with gene constructs designed to over-express a gene associated with or linked to improved millability, to yield several independent genetically-modified plants. Independent transgenic plants can be screened to identify those with increased transcription followed by determining their milling performance.

It will appreciated by the foregoing that the isolated nucleic acids discussed above are quite amenable for inclusion into a genetic construct for generation of a genetically-modified plant, wherein the genetic construct comprises one or more isolated nucleic acids associated with or linked to improved millability.

It can be readily appreciated by a person skilled in the art that a genetic construct is a nucleic acid comprising any one of a number of nucleotide sequence elements, the function of which depends upon the desired use of the construct. Uses range from vectors for the general manipulation and propagation of recombinant DNA to more complicated applications such as prokaryotic or eukaryotic expression of a heterologous nucleic acid and production of genetically-modified plants. Typically, although not exclusively, genetic constructs are designed to provide more than one application. By way of example only, a genetic construct whose intended end use is recombinant protein expression in a eukaryotic system may have incorporated nucleotide sequences for such functions as cloning and propagation in prokaryotes over and above sequences required for expression. An important consideration when designing and preparing such genetic constructs are the required nucleotide sequences for the intended application.

In view of the foregoing, it is evident to a person of skill in the art that genetic constructs are versatile tools that can be adapted for any one of a number of purposes.

In one preferred embodiment, the genetic construct may be suitable for plant transformation.

In another preferred embodiment, the genetic construct is suitable for particle bombardment in wheat. More preferably, the genetic construct comprises the nucleotide sequence of the vector pAHC25.

In alternative embodiments which contemplate co-bombardment, a mixture of a plurality of genetic constructs may be employed. In one preferred embodiment, one genetic construct such as pAHC25 may comprise the selectable marker genetic whilst another genetic construct based on a plasmid such as, but not limited to pGEM3zf, may comprise one or more isolated nucleic acid associated with or linked to improved millability.

By “vector” is meant a nucleic acid, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integratable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

### Additional Sequences

The genetic constructs of the present invention can further include enhancers, either translation or transcription enhancers, as may be required. These enhancer regions are well known to persons skilled in the art, and can include the ATG initiation codon and adjacent sequences. The initiation codon must be in phase with the reading frame of the coding sequence relating to the heterologous or endogenous DNA sequence to ensure translation of the entire sequence. The translation control signals and initiation codons can be of a variety of origins, both natural and synthetic. Translational initiation regions may be provided from the source of the transcriptional initiation region, or from the heterologous or endogenous DNA sequence. The sequence can also be derived from the source of the promoter selected to drive transcription, and can be specifically modified so as to increase translation of the mRNA.

Examples of transcriptional enhancers include, but are not restricted to, elements from the CaMV 35S promoter and octopine synthase genes as for example described by Last et al. (U.S. Pat. No. 5,290,924, which is incorporated herein by reference). It is proposed that the use of an enhancer element such as the ocs element, and particularly multiple copies of the element, will act to increase the level of transcription from adjacent promoters when applied in the context of plant transformation.

As the DNA sequence inserted between the transcription initiation site and the start of the coding sequence, i.e., the untranslated leader sequence, can influence gene expression, one can also employ a particular leader sequence. Preferred leader sequences include those that comprise sequences selected to direct optimum expression of the heterologous or endogenous DNA sequence. For example, such leader sequences include a preferred consensus sequence which can increase or maintain mRNA stability and prevent inappropriate initiation of translation as for example described by Joshi (1987, Nucl. Acid Res., 15:6643), which is incorporated herein by reference. However, other leader sequences, e.g., the leader sequence of RTBV, have a high degree of secondary structure that is expected to decrease mRNA stability and/or decrease translation of the mRNA. Thus, leader sequences (i) that do not have a high degree of secondary structure, (ii) that have a high degree of secondary structure where the secondary structure does not inhibit mRNA stability and/or decrease translation, or (iii) that are derived from genes that are highly expressed in plants, will be most preferred.

Regulatory elements such as the sucrose synthase intron as, for example, described by Vasil et al. (1989, Plant Physiol., 91:5175), the Adh intron I as, for example, described by Canis et al. (1987, Genes Develop., II), or the TMV omega element as, for example, described by Gallie et al. (1989, The Plant Cell, 1:301) can also be included where desired. Other such regulatory elements useful in the practice of the invention are known to those of skill in the art.

Additionally, targeting sequences may be employed to target a protein product of the heterologous or endogenous nucleotide sequence to an intracellular compartment within plant cells or to the extracellular environment. For example, a DNA sequence encoding a transit or signal peptide sequence may be operably linked to a sequence encoding a desired protein such that, when translated, the transit or signal peptide can transport the protein to a particular intracellular or extracellular destination, respectively, and can then be post-translationally removed. Transit or signal peptides act by facilitating the transport of proteins through intracellular membranes, e.g., vacuole, vesicle, plastid and mitochondrial membranes, whereas signal peptides direct proteins through the extracellular membrane. For example, the transit or signal peptide can direct a desired protein to a particular organelle such as a plastid (e.g., a chloroplast), rather than to the cytoplasm. For example, reference may be made to Heijne et al. (1989, Eur. J. Biochem., 180:535) and Keegstra et al. (1989, Ann. Rev. Plant Physiol. Plant Mol. Biol., 40:471), which are incorporated herein by reference.

An isolated nucleic acid of the present invention can also be introduced into a vector, such as a plasmid. Plasmid vectors include additional DNA sequences that provide for easy selection, amplification, and transformation of the expression cassette in prokaryotic and eukaryotic cells, e.g., pUC-derived vectors, pSK-derived vectors, pGEM-derived vectors, pSP-derived vectors, or pBS-derived vectors. Additional DNA sequences include origins of replication to provide for autonomous replication of the vector, selectable marker genes, preferably encoding antibiotic or herbicide resistance, unique multiple cloning sites providing for multiple sites to insert DNA sequences or genes encoded in the DNA construct, and sequences that enhance transformation of prokaryotic and eukaryotic cells.

The vector preferably contains an element(s) that permits stable integration of the vector into the host cell genome or autonomous replication of the vector in the cell independent of the genome of the cell. The vector may be integrated into the host cell genome when introduced into a host cell. For integration, the vector may rely on the heterologous or endogenous DNA sequence or any other element of the vector for stable integration of the vector into the genome by homologous recombination. Alternatively, the vector may contain additional nucleic acid sequences for directing integration by homologous recombination into the genome of the host cell. The additional nucleic acid sequences enable the vector to be integrated into the host cell genome at a precise location in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAM.beta.1 permitting replication in Bacillus. The origin of replication may be one having a mutation to make its function temperature-sensitive in a Bacillus cell (see, e.g., Ehrlich, 1978, Proc. Natl. Acad. Sci. USA 75:1433).

### Marker Genes

To facilitate identification of transformants, the genetic construct desirably comprises a selectable or screenable marker gene as, or in addition to, the expressible heterologous or endogenous nucleotide sequence. The actual choice of a marker is not crucial as long as it is functional (i.e., selective) in combination with the plant cells of choice. The marker gene and the heterologous or endogenous nucleotide sequence of interest do not have to be linked, since co-transformation of unlinked genes as, for example, described in U.S. Pat. No. 4,399,216 is also an efficient process in plant transformation.

Included within the terms selectable or screenable marker genes are genes that encode a “secretable marker” whose secretion can be detected as a means of identifying or selecting for transformed cells. Examples include markers that encode a secretable antigen that can be identified by antibody interaction, or secretable enzymes that can be detected by their catalytic activity. Secretable proteins include, but are not restricted to, proteins that are inserted or trapped in the cell wall (e.g., proteins that include a leader sequence such as that found in the expression unit of extensin or tobacco PR-S); small, diffusible proteins detectable, e.g. by ELISA; and small active enzymes detectable in extracellular solution (e.g., α-amylase, β-lactamase, phosphinothricin acetyltransferase).

### Selectable Markers

Examples of bacterial selectable markers are the dal genes from Bacillus subtilis or Bacillus licheniformis, or markers that confer antibiotic resistance such as ampicillin, kanamycin, erythromycin, chloramphenicol or tetracycline resistance. Exemplary selectable markers for selection of plant transformants include, but are not limited to, a hyg gene which encodes hygromycin B resistance; a neomycin phosphotransferase (neo) gene conferring resistance to kanamycin, paromomycin, G418 and the like as, for example, described by Potrykus et al. (1985, Mol. Gen. Genet. 199:183); a glutathione-S-transferase gene from rat liver conferring resistance to glutathione derived herbicides as, for example, described in EP-A 256 223; a glutamine synthetase gene conferring, upon overexpression, resistance to glutamine synthetase inhibitors such as phosphinothricin as, for example, described WO87/05327, an acetyl transferase gene from Streptomyces viridochromogenes conferring resistance to the selective agent phosphinothricin as, for example, described in EP-A 275 957, a gene encoding a 5-enolshikimate-3-phosphate synthase (EPSPS) conferring tolerance to N-phosphonomethylglycine as, for example, described by Hinchee et al. (1988, Biotech., 6:915), a bar gene conferring resistance against bialaphos as, for example, described in WO91/02071; a nitrilase gene such as bxn from Klebsiella ozaenae which confers resistance to bromoxynil (Stalker et al., 1988, Science, 242:419); a dihydrofolate reductase (DHFR) gene conferring resistance to methotrexate (Thillet et al., 1988, J. Biol. Chem., 263:12500); a mutant acetolactate synthase gene (ALS), which confers resistance to imidazolinone, sulfonylurea or other ALS-inhibiting chemicals (EP-A-154 204); a mutated anthranilate synthase gene that confers resistance to 5-methyl tryptophan; or a dalapon dehalogenase gene that confers resistance to the herbicide.

### Screenable Markers

Preferred screenable markers include, but are not limited to, a uidA gene encoding a β-glucuronidase (GUS) enzyme for which various chromogenic substrates are known; a β-galactosidase gene encoding an enzyme for which chromogenic substrates are known; an aequorin gene (Prasher et al., 1985, Biochem. Biophys. Res. Comm., 126:1259), which may be employed in calcium-sensitive bioluminescence detection; a green fluorescent protein gene (Niedz et al., 1995 Plant Cell Reports, 14:403); a luciferase (luc) gene (Ow et al., 1986, Science, 234:856), which allows for bioluminescence detection; a β-lactamase gene (Sutcliffe, 1978, Proc. Natl. Acad. Sci. USA 75:3737), which encodes an enzyme for which various chromogenic substrates are known (e.g., PADAC, a chromogenic cephalosporin); an R-locus gene, encoding a product that regulates the production of anthocyanin pigments (red color) in plant tissues (Dellaporta et al., 1988, in Chromosome Structure and Function, pp. 263-282); an α-amylase gene (Ikuta et al., 1990, Biotech., 8:241); a tyrosinase gene (Katz et al., 1983, J. Gen. Microbiol., 129:2703) which encodes an enzyme capable of oxidizing tyrosine to dopa and dopaquinone which in turn condenses to form the easily detectable compound melanin; or a xylE gene (Zukowsky et al., 1983, Proc. Natl. Acad. Sci. USA 80:1101), which encodes a catechol dioxygenase that can convert chromogenic catechols.

### Plant Transformation

The initial step in production of a genetically-modified plant is introduction of DNA into a plant host cell. A number of techniques are available for the introduction of DNA into a plant host cell. There are many plant transformation techniques well known to workers in the art, and new techniques are continually becoming known. The particular choice of a transformation technology will be determined by its efficiency to transform certain plant species as well as the experience and preference of the person practising the invention with a particular methodology of choice. It will be apparent to the skilled person that the particular choice of a transformation system to introduce a genetic construct into plant cells is not essential to or a limitation of the invention, provided it achieves an acceptable level of nucleic acid transfer. Guidance in the practical implementation of transformation systems for plant improvement is provided by Birch (1997, Annu. Rev. Plant Physiol. Plant Molec. Biol. 48: 297-326), which is incorporated herein by reference.

In one embodiment, transformation is by microprojectile bombardment, for example as described by Franks & Birch, 1991, Aust. J. Plant. Physiol., 18:471; Gambley et al., 1994, supra; and Bower et al., 1996, Molecular Breeding, 2:239, which are herein incorporated by reference.

In another embodiment, transformation is Agrobacterium-mediated. Examples of Agrobacterium-mediated transformation of monocots are provided in U.S. Pat. No. 6,037,522, Hiei et al., 1994, Plant Journal 6 271 and Ishida et al., 1996, Nature Biotechnol. 14 745 in relation to various cereals, Arencibia et al., 1998, Transgenic Res. 7 213.

Accordingly, persons skilled in the art will be aware that a variety of other transformation methods are applicable to the method of the invention such as liposome-mediated (Ahokas et al., 1987, Heriditas 106 129), laser-mediated (Guo et al., 1995, Physiologia Plantarum 93 19), silicon carbide or tungsten whiskers (U.S. Pat. No. 5,302,523; Kaeppler et al., 1992, Theor. Appl. Genet. 84 560), virus-mediated (Brisson et al., 1987, Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791).

Alternatively, a combination of different techniques may be employed to enhance the efficiency of the transformation process, e.g., bombardment with Agrobacterium coated microparticles (EP-A-486234) or microprojectile bombardment to induce wounding followed by co-cultivation with Agrobacterium (EP-A-486233).

### Plant Regeneration

The methods used to regenerate transformed cells into differentiated plants are not critical to this invention, and any method suitable for a target plant can be employed. Normally, a plant cell is regenerated to obtain a whole plant following a transformation process.

The term “regeneration” as used herein means growing a whole, differentiated plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible. Regeneration also occurs from plant callus, explants, organs or parts. Shoots that develop are excised from calli and transplanted to appropriate root-inducing selective medium. Rooted plantlets are transplanted to soil as soon as possible after roots appear. The plantlets can be repotted as required, until reaching maturity.

For example, wheat plants have been regenerated from embryogenic suspension culture by selecting only the aged compact and nodular embryogenic callus tissues for the establishment of the embryogenic suspension cultures (Vasil, 1990, Bio/Technol. 8:429-434). The combination with transformation systems for these crops enables the application of the present invention to monocots.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use.

In seed propagated crops, the mature transgenic plants can be self-crossed to produce a homozygous inbred plant. The inbred plant produces seed containing the newly introduced heterologous gene(s). These seeds can be grown to produce plants that would produce a selected phenotype, e.g., increased endosperm hardness.

Parts obtained from the regenerated plant, such as grains, flowers, seeds, leaves, branches, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

It will be appreciated that the literature describes numerous techniques for regenerating specific plant types and more are continually becoming known. Those of ordinary skill in the art can refer to the literature for details and select suitable techniques without undue experimentation.

### Characterization

To confirm the presence of the heterologous nucleic acid in the regenerating plants, a variety of assays may be performed. Such assays include, for example, “molecular biological” assays well known to those of skill in the art, such as Southern and Northern blotting and PCR; a protein expressed by the heterologous DNA may be analysed by western blotting, high performance liquid chromatography or ELISA (e.g., nptII) as is well known in the art.

Examples of various methods applicable to characterization of transgenic plants are provided in Chapters 9 and 11 of PLANT MOLECULAR BIOLOGY: A Laboratory Manual Ed. M. S. Clark (Springer-Verlag, Heidelberg, 1997), which chapters are herein incorporated by reference

So that the invention may be readily understood and put into practical effect, the following non-limiting Examples are provided.

### EXAMPLES

### Example 1

Measurements of flour yield made on wheat varieties at two sites for comparison with gene expression analysis experiments using DNA microarrays from samples of developing seed at 14 days post anthesis.

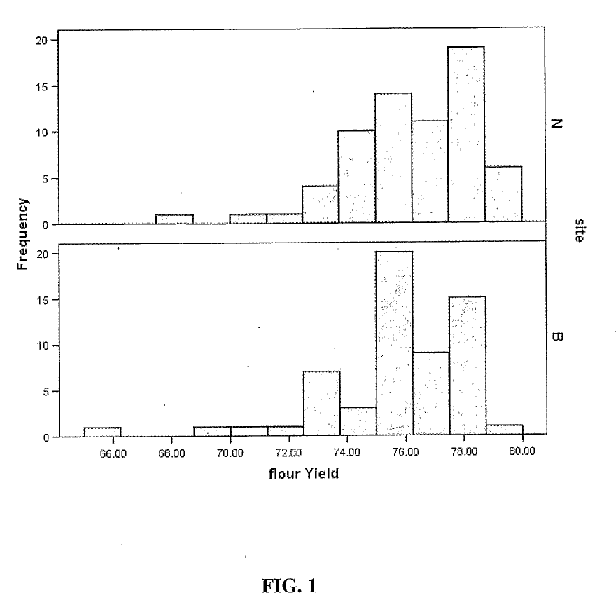

From 200 varieties of wheat grown in trials, 154 varieties provided sufficient seed material to enable small scale milling trials which provided data on flour yield from the site at Narrabri and 97 varieties at Biloela. Seventy one varieties classed as ‘hard wheats’ were selected for analysis for potential inclusion in microarray gene expression analysis studies. Fifty-five varieties provided flour yield data from both sites. The distribution of flour yield measurements is presented in FIG. 1.

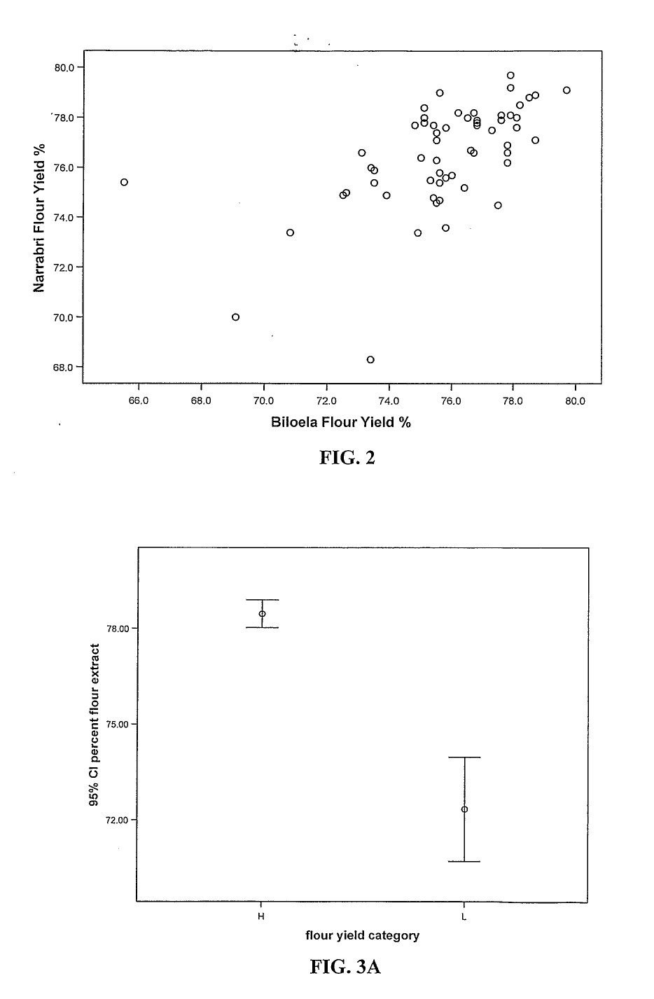

The flour yield from the wheat varieties was similar at the two sites with a correlation coefficient of r=0.59 between sites. A scatter plot comparing yield at the two sites is shown in FIG. 2. The difference in flour yield between sites (Table 1) was found to be statistically significant as was importantly the difference in flour yield between genotypes (varieties) (Table 2). However no significant interaction was found between these two factors (Table 2).

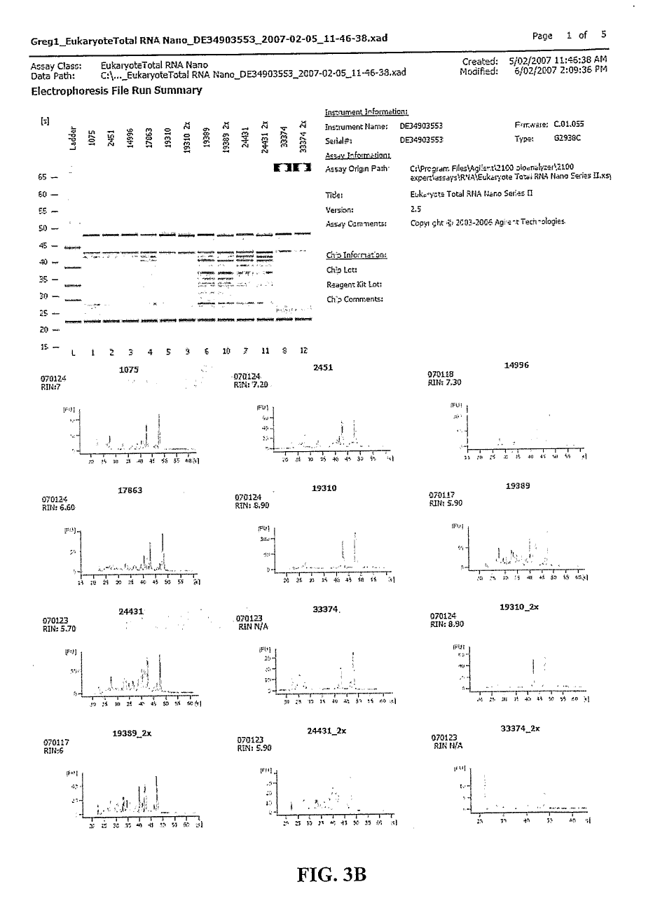

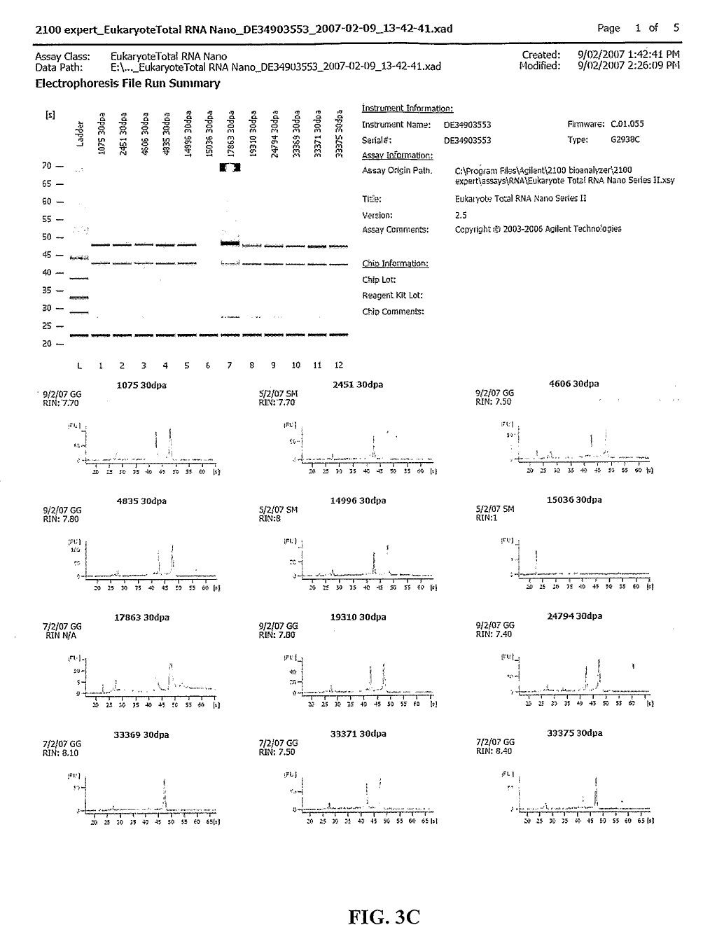

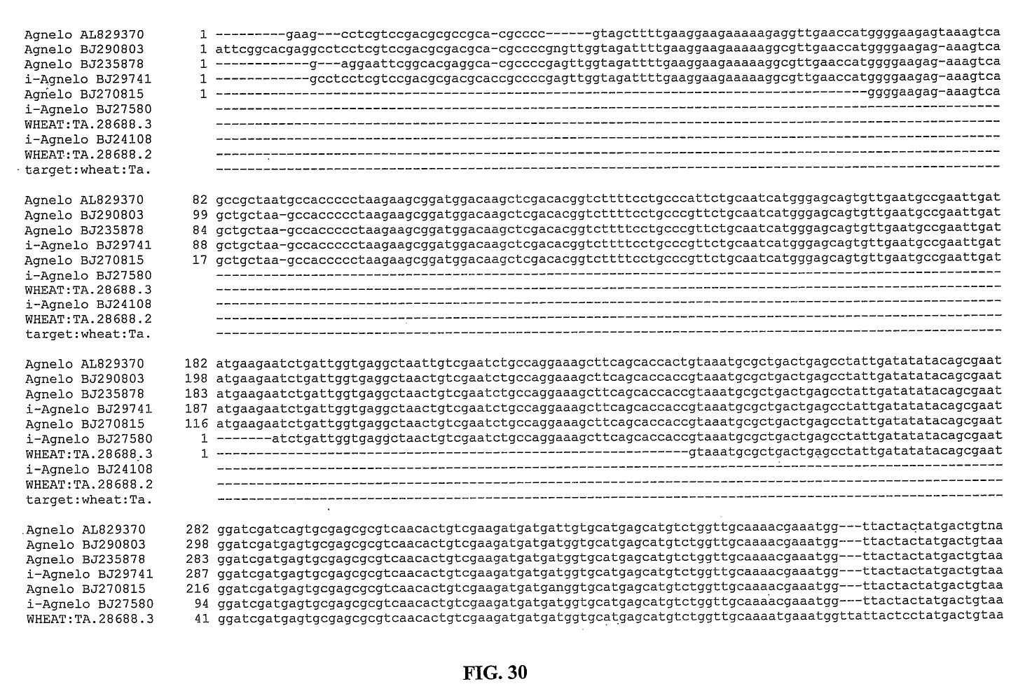

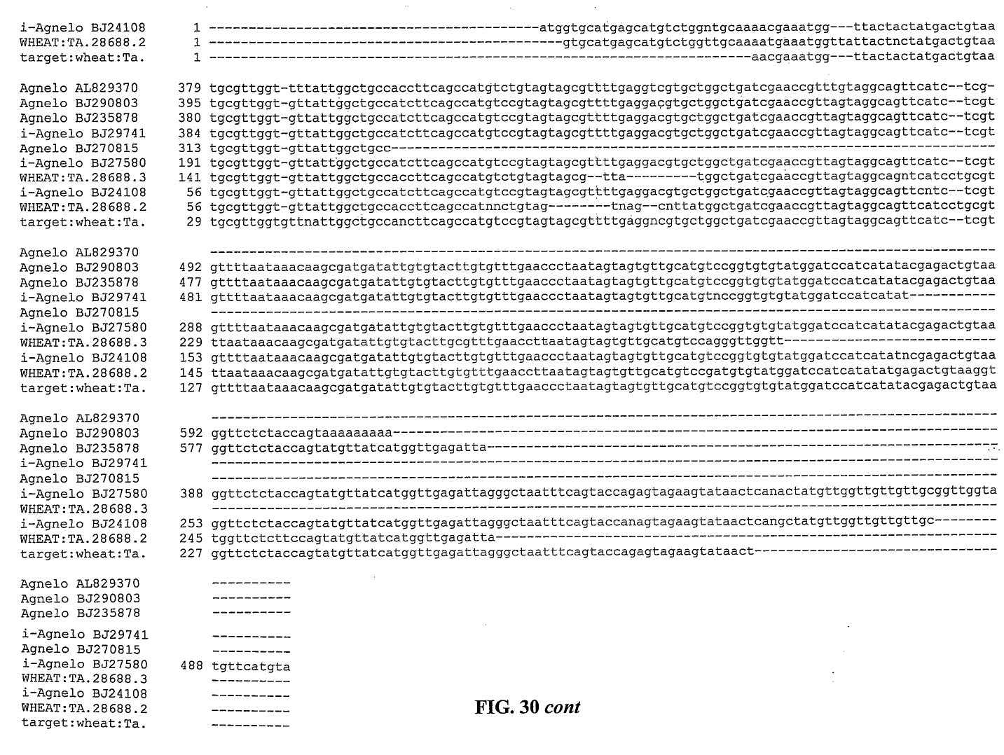

From the highest yielding and lowest yielding wheat varieties, RNA was extracted for gene expression analysis from samples of developing seed at 14 days post anthesis. Ten wheat varieties were selected that had RNA of sufficient quality and quantity and that came from the extremes of the distribution of flour yield. The yields from these varieties at the two experimental sites are indicated in Table 3. A 95% confidence interval about the mean of flour yield for the two classes of wheat (high—H and low—L) indicates that the two classes are widely separated in their flour yield properties and are thus likely to be quite distinct genetically for this trait (FIG. 3A). The RNA was extracted and purified from developing wheat seeds at 14 dpa (FIG. 3B) and 30 dpa (FIG. 3C) and the yield and quality checked by Bioanalyser.

### Example 2

A statistical analysis of expression differences between low yield and high yield wheat varieties from samples at 14 days post anthesis.

### Introduction

The present inventors have investigated gene expression in wheat seeds using Affymetrix wheat chips. This report provides a statistical analysis of these investigations.

The experiment was designed to compare gene expression between wheat varieties with low flour yield and high flour yield. Affymetrix expression data is available for ten varieties, five with low flour yield and five with high flour yield. An assessment of the quality of the data indicated that the data from each of the chips was suitable for analysis. A series of analyses were performed to investigate overall differences in expression between the low and high yield varieties as well as differences in individual genes. The results suggest that there are small differences in a large number of genes rather than large differences in a small number of genes. A set of disjoint genes, that may be suitable for further analysis, is identified.

### Data Screening

High quality data is essential for obtaining meaningful results from gene expression studies. Both RNA quality and the quality of the chip and its processing influence the final data. RNA quality can be assessed by the RNA Integrity Number (RIN) [5]. The RIN is a score produced by the Agilent bioanalyzer system that is designed to measure RNA degradation. The scores range from 1, indicating severe degradation to 10, indicating no degradation. The RINs were all greater than 5.5, making them suitable for analysis but not ideal.

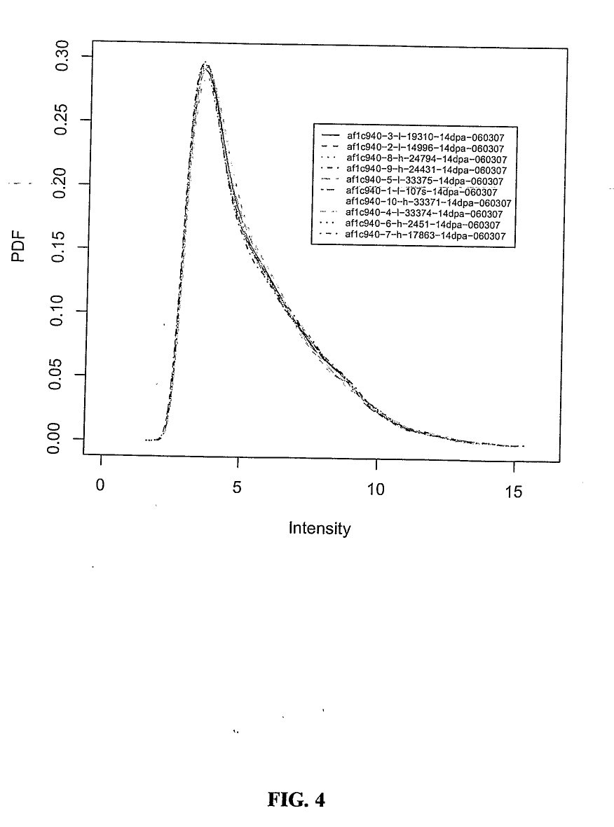

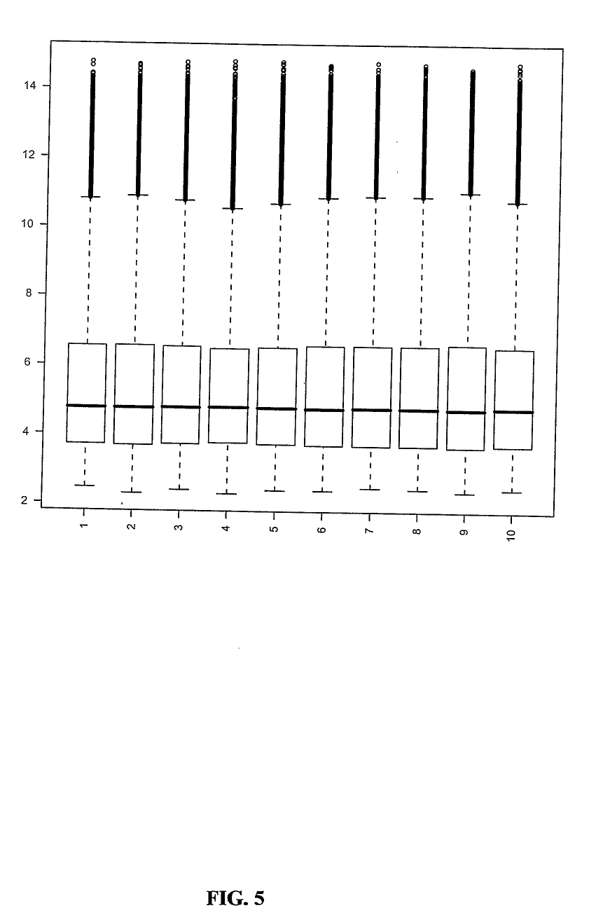

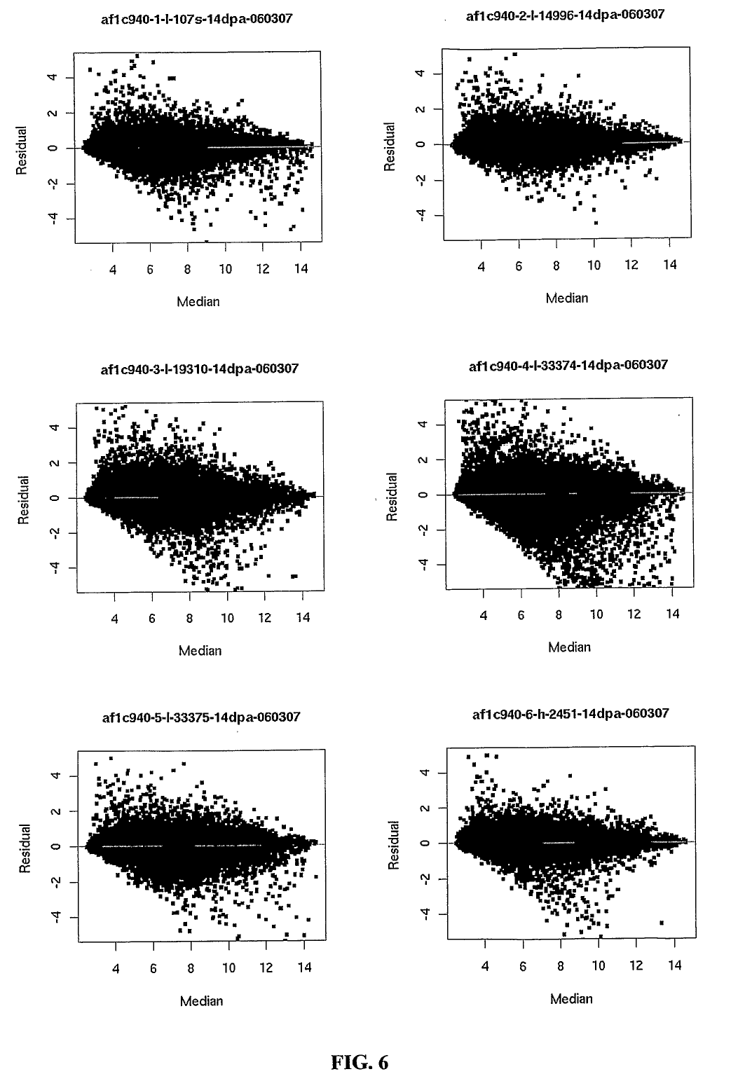

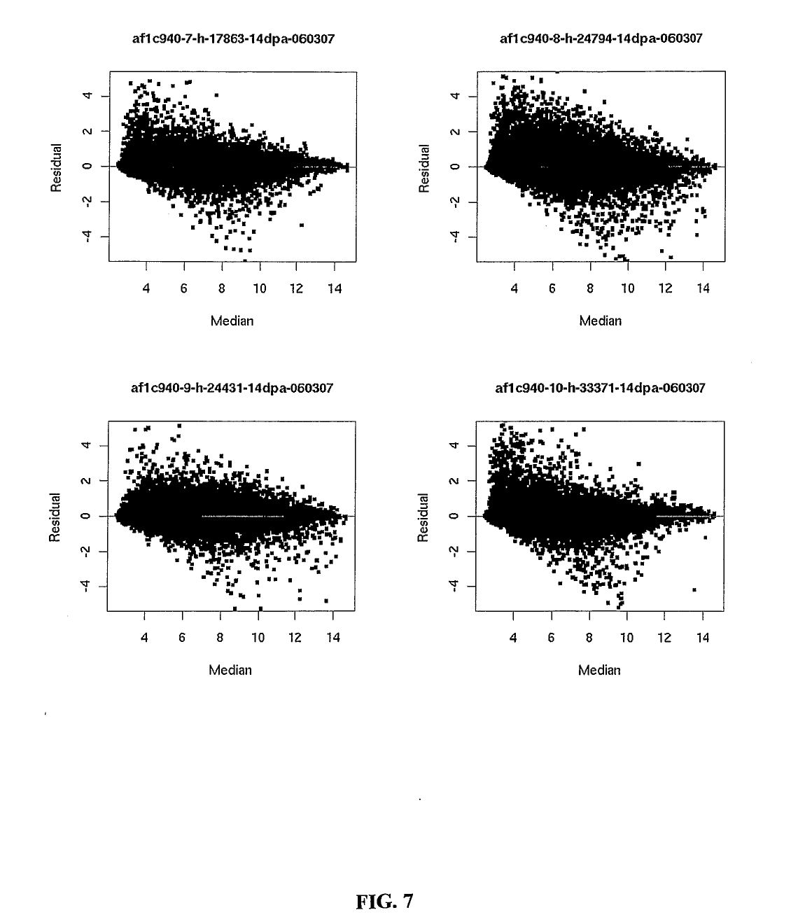

The distributions of the processed expression values can be examined for quality. After processing of the chips using the RMA algorithm [3], the kernel density estimates of expression (see FIG. 4) and the box and whisker plots (see FIG. 5) were used to display the distributions. Although RMA forces probe level expression estimates to have the same distribution on each chip, this is not the case for gene level expression estimates. Nevertheless, the distributions of gene level expression estimates for each of the chips are all very similar. No chip has an atypical distribution. MA plots were also used to examine the expression values (see FIGS. 6 and 7). The MA plot for a given chip is a comparison for each gene of the median across all chips with the difference between the expression of the chip and the median across all chips. Ideally, the points of the MA plot are evenly scattered about the horizontal line through zero. Although there is some evidence of non-linearity for chips 4 and 8, overall the MA plots indicate that the data is suitable for analysis.

### Data Analysis

### Methods

The first analyses were designed to examine any structure in the data. The varieties were plotted in the space of their first two principal components and cluster analysis was performed. The results of cluster analyses can be easily biased by the choice of clustering algorithm. To avoid bias, the clustering was conducted three times, each time using a different algorithm. Cluster dendograms based on the single linkage, average linkage and complete linkage algorithms were constructed.

The ability to use the gene expression data to distinguish between the low yield and high yield varieties is summarised by Receiver Operator Characteristic (ROC) curves. These curves illustrate the relationship between correctly identifying high yield samples as high yield and correctly identifying low yield samples as low yield. We will use the terms sensitivity and selectivity to describe this relationship. Usually, these terms are applied to positive and negative samples, e.g. samples that are either positive or negative for a disease. For the purposes of this report we will take the high yield varieties to be ‘positive’ and the low yield varieties to be ‘negative’. Thus the sensitivity is the percentage of high yield varieties correctly identified as high yield by the gene expression data and selectivity is the percentage of low yield varieties correctly identified as low yield by the gene expression data. ROC curves have received a great deal of attention in the biostatistical literature. Most techniques for the estimation of ROC curves have addressed problems in which diagnosis is based on a single measurement (for example an antibody titre). The techniques are based on estimates of the distribution of the measurement within the two groups to be differentiated, and a smoothly varying decision threshold.

All of this statistical apparatus is available to us, once the discriminant function score has been calculated. A decision process based on a varying threshold and fixed discriminant function scores can be motivated by the discussion of the effect of prior probability (see above). We consider a random variable X, which represents the value of the discriminant function score, and a threshold c such that observations with values of X below c are classified as negative, and observations with X greater than c are classified as positive. Now we may define the sensitivity and selectivity as functions of c as follows:

$$Sensitivity ( c ) = 1 - {\int }_{- ∞}^{o} {f}_{2} ( X ) d X , \\ Selectivity ( c ) = {\int }_{- ∞}^{o} {f}_{1} ( X ) d X ;$$

where f1(X) is the probability density function of X for normal individuals, and f2(X) is the probability density function of X for affected individuals.

Estimation of the ROC is then reduced to the problem of estimating the distribution of the discriminant function score for negative and positive subjects (f1(X) and f2(X) respectively). Two methods were used for this process:

A method based on the empirical distribution function of X, and which produces a raw ROC curve. This has the disadvantage that it is a step function—changing whenever the value of c crosses a value actually present in the data.

An approach using a kernel density estimate of the distribution of the score in each group, with a bandwidth chosen by the method of Lloyd. The Kernel density approach produces a smooth estimate of the ROC, but is not dependent on the assumption of normality. The values of the discriminant function scores used in this analysis were obtained using cross-validation. That is, rather than use the discriminant function scores obtained from a discriminant analysis with all the data, the scores for each observation were obtained by dropping that observation, fitting the discriminant function, and then calculating the discriminant function score for the dropped observation. This process is likely to be more conservative, and lead to more ‘honest’ estimates of the ROC.

As well as the analysis of overall differences between the high and low yield varieties, differences between the varieties for individual genes were also investigated. A linear model was constructed to compare gene expression between the low yield and high yield wheat varieties. The empirical Bayes procedure [4] was applied to this model and p-values of differential expression were calculated for each of the genes. The p-values were adjusted for multiple comparisons using two procedures. Holms method [2] was used to provide strong control of the Family Wise Type 1 Error Rate. In addition, the method of Benjamini and Hochberg [6] was used to control the False Discovery Rate (FDR). Both procedures are conservative however the Holm procedure is more conservative than the FDR procedure. The advantage of controlling the FWER is that any genes identified as differentially expressed are highly likely to be so, however the disadvantage is that it is easier to omit genes that are differentially expressed.

Disjoint genes, that is genes for which all the values for one yield type are higher than all the values for the other yield type, were also identified. The number of disjoint genes was counted, and the distribution of this number was calculated under random permutation of the group (low/high yield) labels. If the observed number of disjoint genes is extreme on this permutation distribution, than the data provide evidence of a statistically significant number of disjoint genes.

### Results

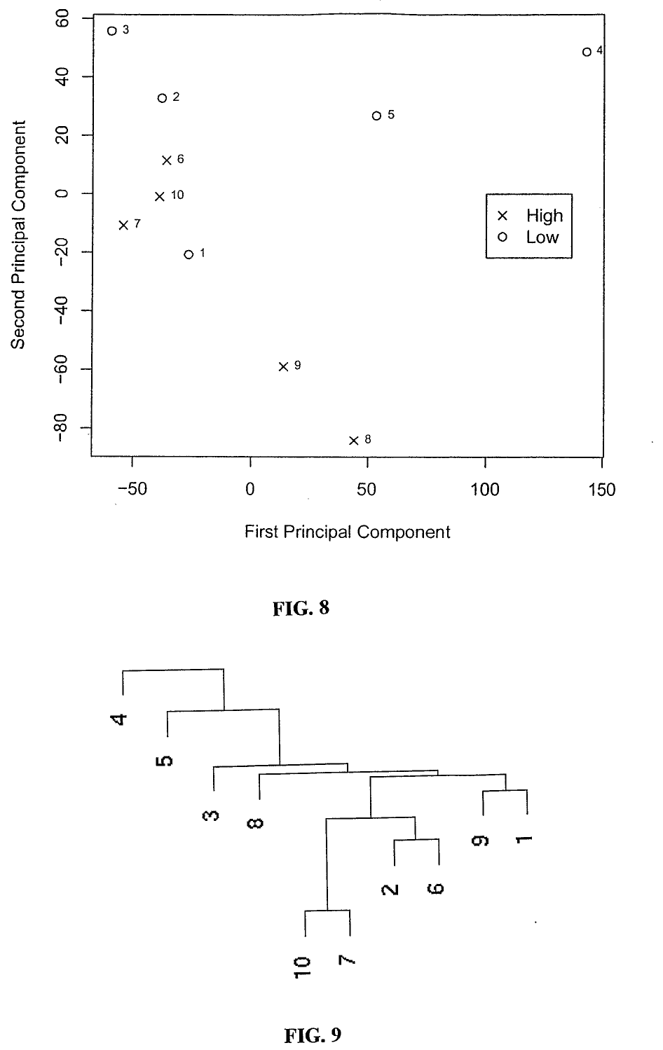

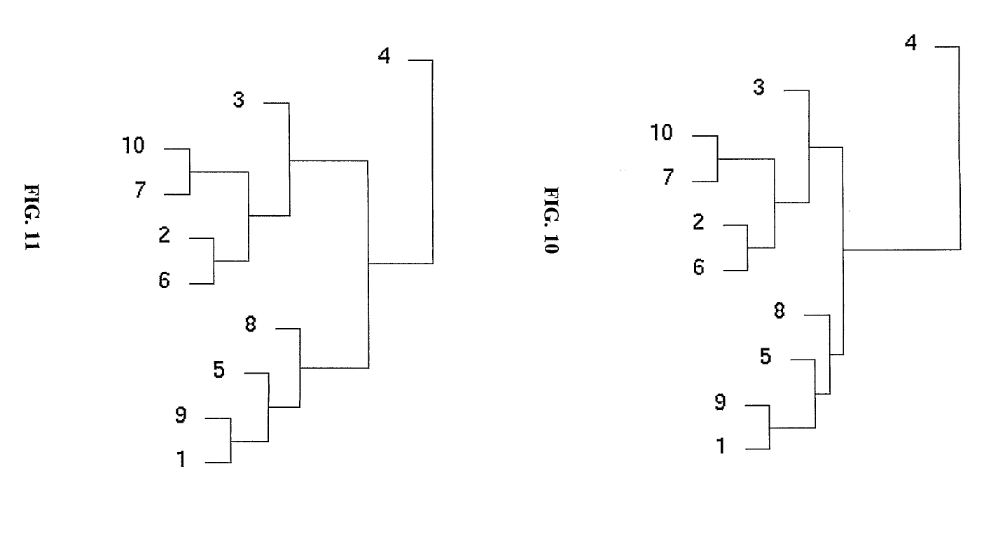

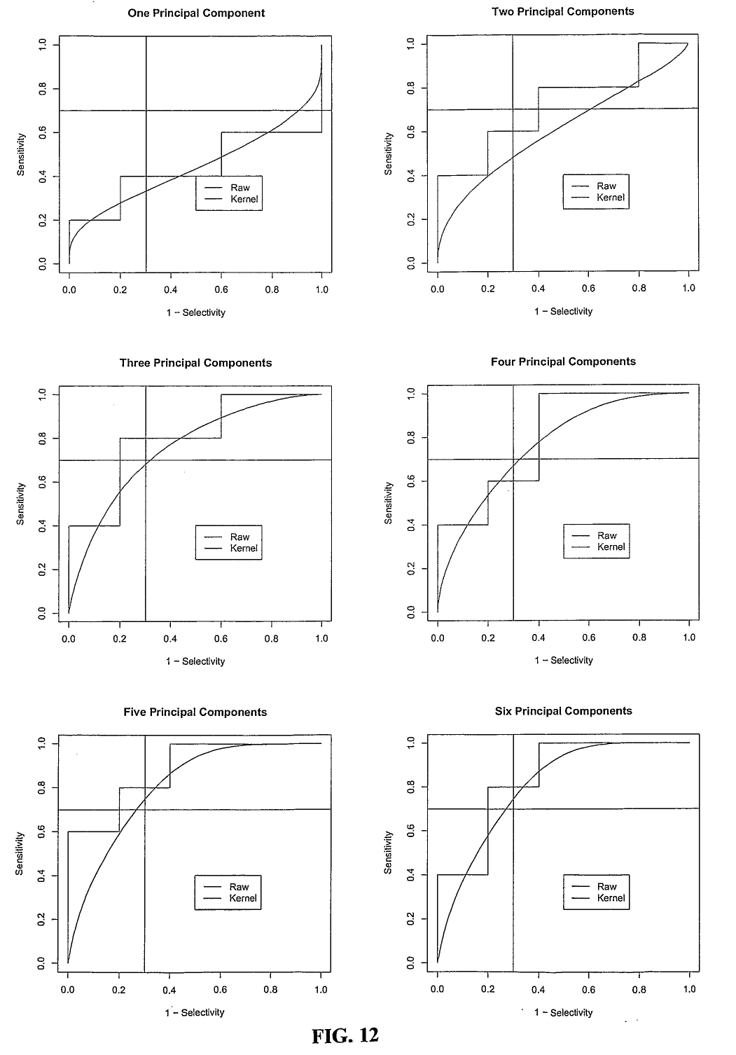

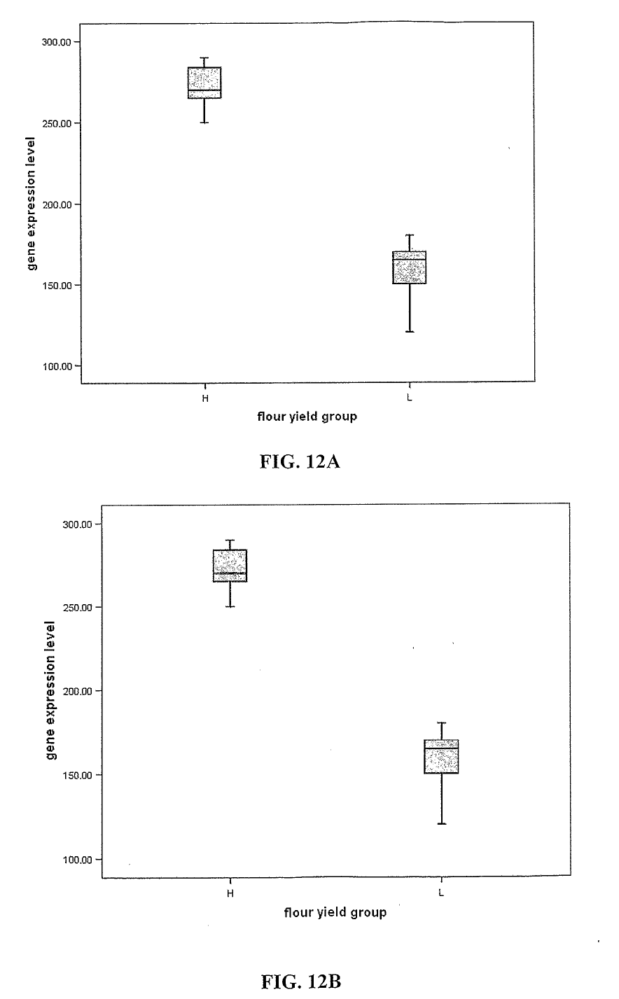

FIG. 8 shows the varieties plotted in the space of their first two principal components. There is no separation between the low yield and high yield varieties in the first principal component, however there is some separation in the second principal component. Cluster dendograms based on the single linkage, average linkage and complete linkage algorithms are shown in FIGS. 9, 10 and 11 respectively. None of the dendograms exhibit any clustering of the varieties by yield. The ROC curves based on one, two, three, four, five and six principal components are included in FIG. 12. The best sensitivities and selectivities and largest areas under the curves are obtained when at least three principal components are included. This suggests that the differences between the low yield and high yield varieties consist of small changes in a large number of genes rather than large changes in a small number of genes. Overall, the best result for sensitivity and selectivity is 0.8 for both values, based on either three or six principal components. The support vector machine method was also used to classify the wheat varieties, but the results of this analysis were inferior to the results given by the ROC curves.

From the analysis of the data using linear models, one statistically significant gene based on the Holm and FDR corrections was detected—Ta.28688.1.A1_at.

In summary analysis and identification of the 14 dpa gene was carried out as follows. Data from the arrays was analysed using currently accepted procedures for Affymetrix GeneChip® arrays (RMA—robust multi-array average (Irizarry et al., 2003)). Probe level data was background corrected, normalised between arrays and gene level data (expression values) summarised from the probe data. Probe data was checked for equivalent distribution between the Affymetrix GeneChip® arrays in the experiment (Box and whisker plots, kernel density estimates) and data from each chip compared to the median of all chips (MA plots) to detect any anomalies in hybridisation behaviour between samples. A statistical test (t-test) was then carried out on the log 2 transformed values from each expressed gene and the p-values adjusted by multiplication by the number of genes tested (Holm correction).

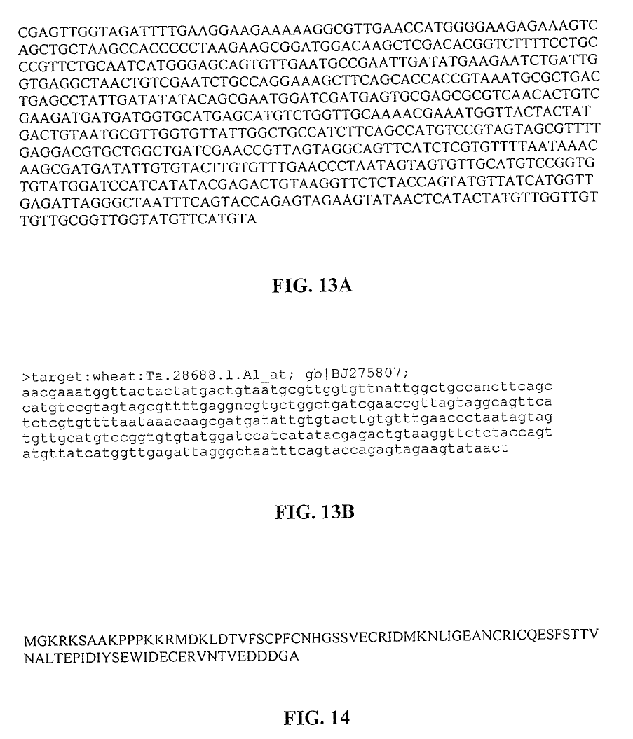

It was determined that 1,014 genes were found to be expressed disjointly (no overlap in gene expression, FIG. 12A) between the low and high flour yielding groups. One gene, termed “14dpa gene”, appears to be down regulated in the high milling-yield varieties. The gene identified with the corrected p value of 0.04 corresponded to the affy-probes (Table 7) that were designed over the target Ta.28688.1.A1_at (FIG. 13B). There was a 12.8 fold difference in the ‘expression values’ obtained from the Affymetrix arrays for gene Ta.28688 between the high and low milling yield varieties.

### Conclusion

The differences in gene expression between the low yield and high yield wheat varieties consist of small differences in a large number of genes rather than large differences in a small number of genes.

After conservative adjustment of p values, there is only one gene, Ta.28688.1.A1_at, for which a statistically significant difference between the low yield and high yield varieties could be detected.

### Example 3

Measurements of flour yield made on wheat varieties at two sites for comparison with gene expression analysis experiments using DNA microarrays from samples of developing seed at 30 days post anthesis.

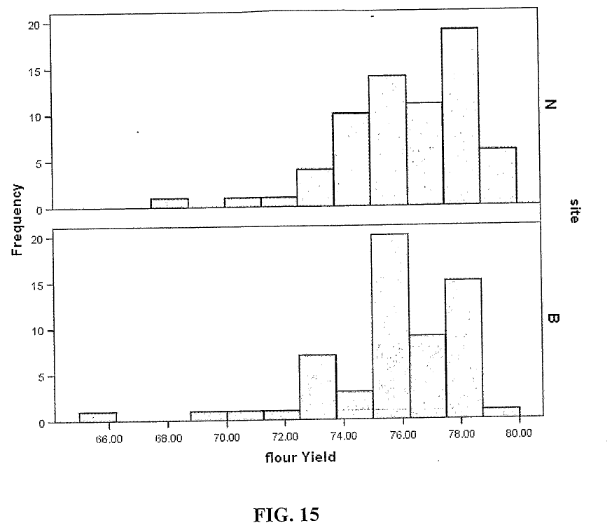

From 200 varieties of wheat grown in trials, 154 varieties provided sufficient seed material to enable small scale milling trials which provided data on flour yield from the site at Narrabri and 97 varieties at Biloela. Seventy one varieties classed as ‘hard wheats’ were selected for analysis for potential inclusion in microarray gene expression analysis studies. Fifty-five varieties provided flour yield data from both sites. The distribution of flour yield measurements is presented in FIG. 15.

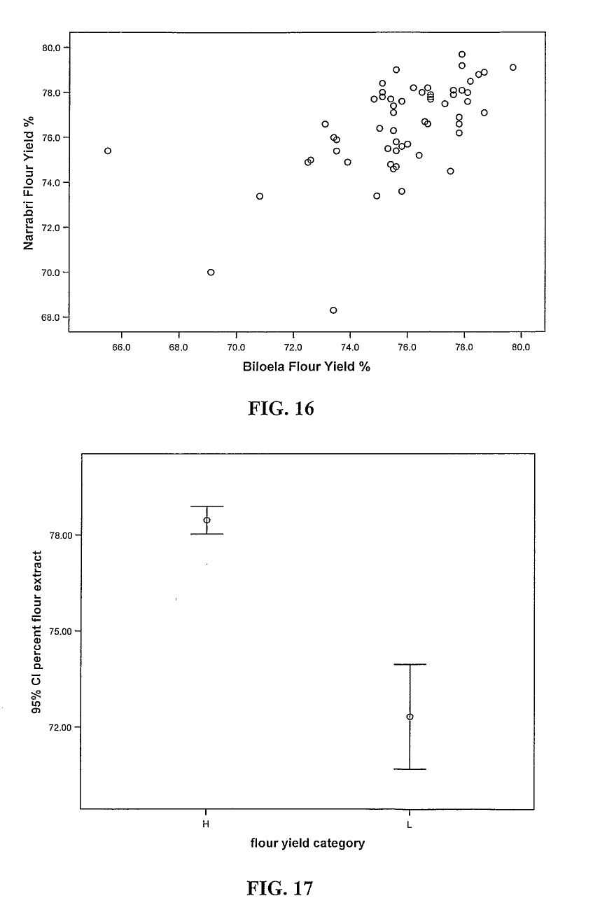

The flour yield from the wheat varieties was similar at the two sites with a correlation coefficient of r=0.59 between sites. A scatter plot comparing yield at the two sites is shown in FIG. 16. The difference in flour yield between sites (Table 4) was found to be statistically significant as was importantly the difference in flour yield between genotypes (varieties) (Table 5). However no significant interaction was found between these two factors (Table 5).

From the highest yielding and lowest yielding wheat varieties, RNA was extracted for gene expression analysis from samples of developing seed at 30 days post anthesis. Ten wheat varieties were selected that had RNA of sufficient quality and quantity and that came from the extremes of the distribution of flour yield. The yields from these varieties at the two experimental sites are indicated in Table 6. A 95% confidence interval about the mean of flour yield for the two classes of wheat (high—H and low—L) indicates that the two classes are widely separated in their flour yield properties and are thus likely to be quite distinct genetically for this trait (FIG. 17).

### Example 4

A statistical analysis of expression differences between low yield and high yield wheat varieties at 30 days post anthesis.

### Introduction

The present inventors have investigated gene expression in wheat seeds using Affymetrix wheat chips. This report provides a statistical analysis of these investigations. The experiment was designed to compare gene expression between wheat varieties with low flour yield and high flour yield. Affymetrix expression data is available for ten varieties, five with low flour yield and five with high flour yield. This experiment follows an earlier experiment that used wheat seeds from a different time point. The results of this experiment are generally similar to those of the earlier experiment.

An assessment of the quality of the data indicated that the data from each of the chips was suitable for analysis. A series of analyses were performed to investigate overall differences in expression between the low and high yield varieties as well as differences in individual genes. There is some evidence of small differences in a large number of genes. The results of this study were also combined with the results of the earlier study. When the studies are combined there is little evidence of differential expression between the low yield and high yield varieties.

### Data Screening

High quality data is essential for obtaining meaningful results from gene expression studies. Both RNA quality and the quality of the chip and its processing influence the final data. RNA quality can be assessed by the RNA Integrity Number (RIN) [5]. The RIN is a score produced by the Agilent bioanalyzer system that is designed to measure RNA degradation. The scores range from 1, indicating severe degradation to 10, indicating no degradation. For this experiment, the RINs ranged from 7.4 to 8.4 which is an average to above average result.

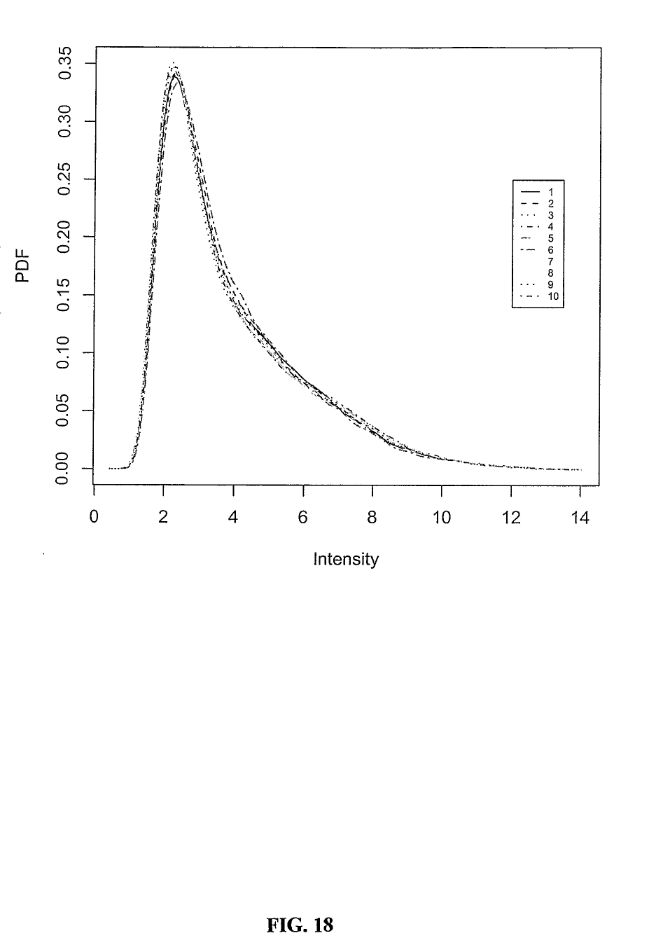

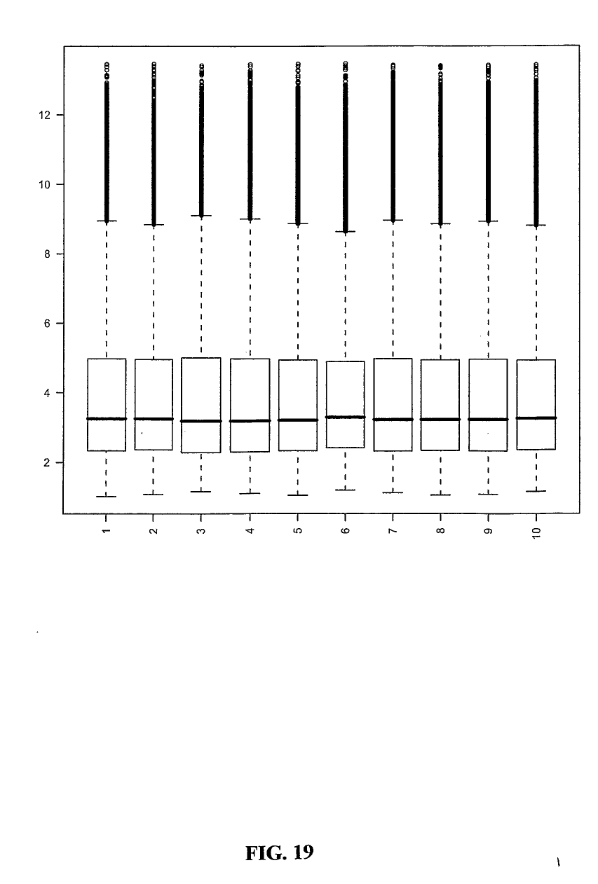

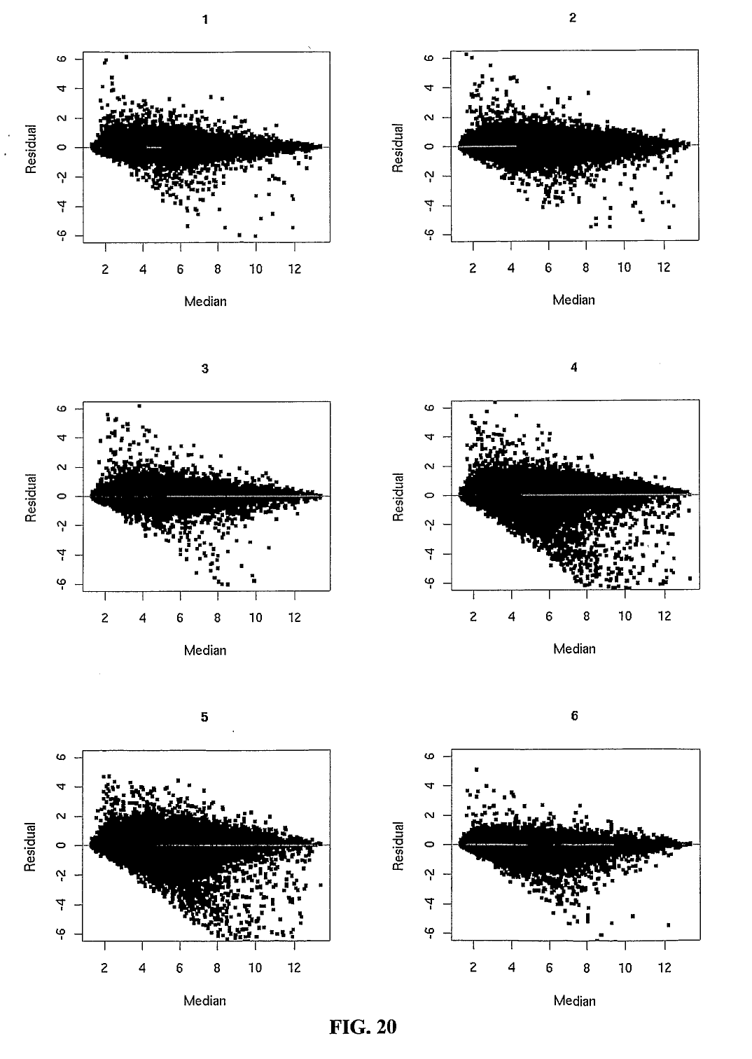

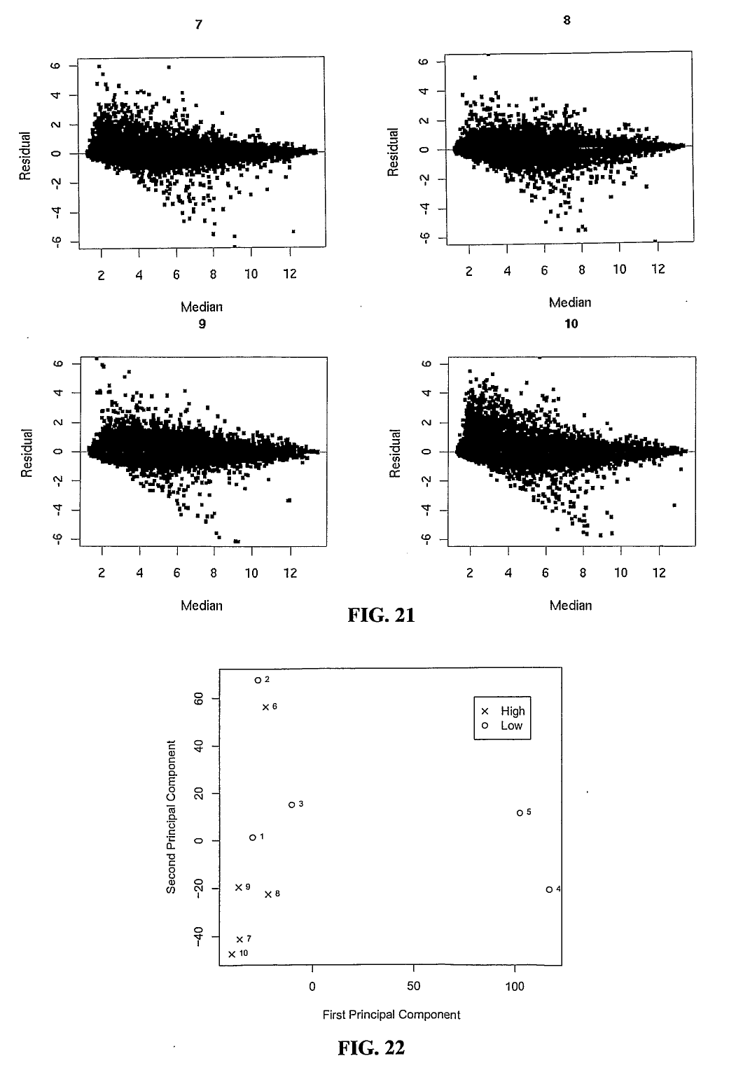

The distributions of the processed expression values can also be examined for quality. After processing of the chips using the RMA algorithm [3], the kernel density estimates of expression (see FIG. 18) and the box and whisker plots (see FIG. 19) were used to display the distributions. Although RMA forces probe level expression estimates to have the same distribution on each chip, this is not the case for gene level expression estimates. Nevertheless, the distributions of gene level expression estimates for each of the chips are all very similar. No chip has an atypical distribution. MA plots were also used to examine the expression values (see FIGS. 0 and 21). The MA plot for a given chip is a comparison for each gene of the median across all chips with the difference between the expression of the chip and the median across all chips. Ideally, the points of the MA plot are evenly scattered about the horizontal line through zero. Although there is some evidence of non-linearity for chips 4 and 5, overall the MA plots indicate that the data is suitable for analysis.

### Data Analysis

### Methods

The first analyses were designed to examine any structure in the data. The varieties were plotted in the space of their first two principal components and cluster analysis was performed. The results of cluster analyses can be easily biased by the choice of clustering algorithm. To avoid bias, the clustering was conducted three times, each time using a different algorithm. Cluster dendograms based on the single linkage, average linkage and complete linkage algorithms were constructed.

The ability to use the gene expression data to distinguish between the low yield and high yield varieties is summarised by Receiver Operator Characteristic (ROC) curves. These curves illustrate the relationship between correctly identifying high yield samples as high yield and correctly identifying low yield samples as low yield. We will use the terms sensitivity and selectivity to describe this relationship. Usually, these terms are applied to positive and negative samples, e.g. samples that are either positive or negative for a disease. For the purposes of this report we will take the high yield varieties to be ‘positive’ and the low yield varieties to be ‘negative’. Thus the sensitivity is the percentage of high yield varieties correctly identified as high yield by the gene expression data and selectivity is the percentage of low yield varieties correctly identified as low yield by the gene expression data. ROC curves have received a great deal of attention in the biostatistical literature. Most techniques for the estimation of ROC curves have addressed problems in which diagnosis is based on a single measurement (for example an antibody titre). The techniques are based on estimates of the distribution of the measurement within the two groups to be differentiated, and a smoothly varying decision threshold.

All of this statistical apparatus is available to us, once the discriminant function score has been calculated. A decision process based on a varying threshold and fixed discriminant function scores can be motivated by the discussion of the effect of prior probability (see above). We consider a random variable X, which represents the value of the discriminant function score, and a threshold c such that observations with values of X below c are classified as negative, and observations with X greater than c are classified as positive. Now we may define the sensitivity and selectivity as functions of c as follows:

$$Sensitivity ( c ) = 1 - {\int }_{- ∞}^{o} {f}_{2} ( X ) d X , \\ Selectivity ( c ) = {\int }_{- ∞}^{o} {f}_{1} ( X ) d X ;$$

where f1(X) is the probability density function of X for normal individuals, and f2(X) is the probability density function of X for affected individuals. Estimation of the ROC is then reduced to the problem of estimating the distribution of the discriminant function score for negative and positive subjects (f1(X) and f2(X) respectively). Two methods were used for this process:

A method based on the empirical distribution function of X, and which produces a raw ROC curve. This has the disadvantage that it is a step function—changing whenever the value of c crosses a value actually present in the data.

An approach using a kernel density estimate of the distribution of the score in each group, with a bandwidth chosen by the method of Lloyd. The kernel density approach produces a smooth estimate of the ROC, but is not dependent on the assumption of normality.

The values of the discriminant function scores used in this analysis were obtained using cross-validation. That is, rather than use the discriminant function scores obtained from a discriminant analysis with all the data, the scores for each observation were obtained by dropping that observation, fitting the discriminant function, and then calculating the discriminant function score for the dropped observation. This process is likely to be more conservative, and lead to more ‘honest’ estimates of the ROC.

As well as the analysis of overall differences between the high and low yield varieties, differences between the varieties for individual genes were also investigated. A linear model was constructed to compare gene expression between the low yield and high yield wheat varieties. The empirical Bayes procedure [4] was applied to this model and p-values of differential expression were calculated for each of the genes. The p-values were adjusted for multiple comparisons using two procedures. Holms method [2] was used to provide strong control of the Family Wise Type 1 Error Rate. In addition, the method of Benjamin and Hochberg [6] was used to control the False Discovery Rate (FDR). Both procedures are conservative however the Holm procedure is more conservative than the FDR procedure. The advantage of controlling the FWER is that any genes identified as differentially expressed are highly likely to be so, however the disadvantage is that it is easier to omit genes that are differentially expressed.

A second linear model, that included the data from the first experiment, was constructed. As per the original linear model the p-values were adjusted using the FDR and Holm procedures.

Disjoint genes, that is genes for which all the values for one yield type are higher than all the values for the other yield type, were also identified. The sets of disjoint genes identified by the first and second experiments were compared and a Fisher test was performed to test for independence.

### Results

FIG. 22 shows the varieties plotted in the space of their first two principal components. There is no separation between the low yield and high yield varieties in either the first or second principal component. Note that varieties 4 and 5 differ significantly from the others on the first principal component. This may indicate differential expression between these two varieties and the others or perhaps a quality issue (as per the nonlinearity of the MA plots for these varieties).

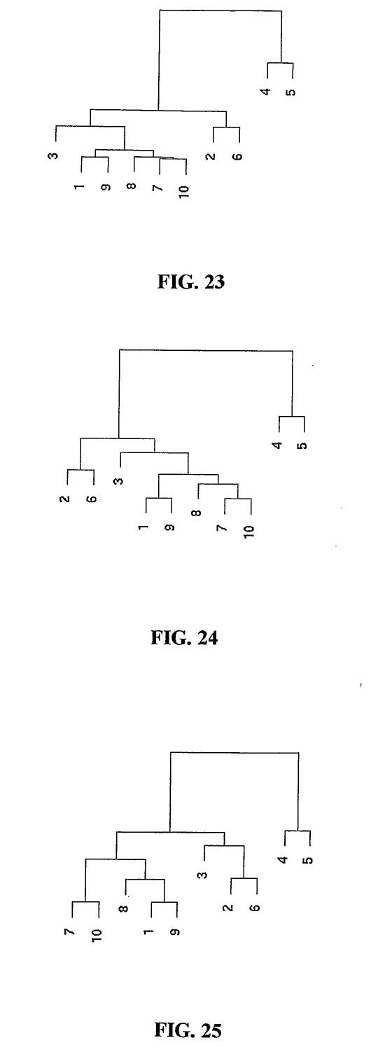

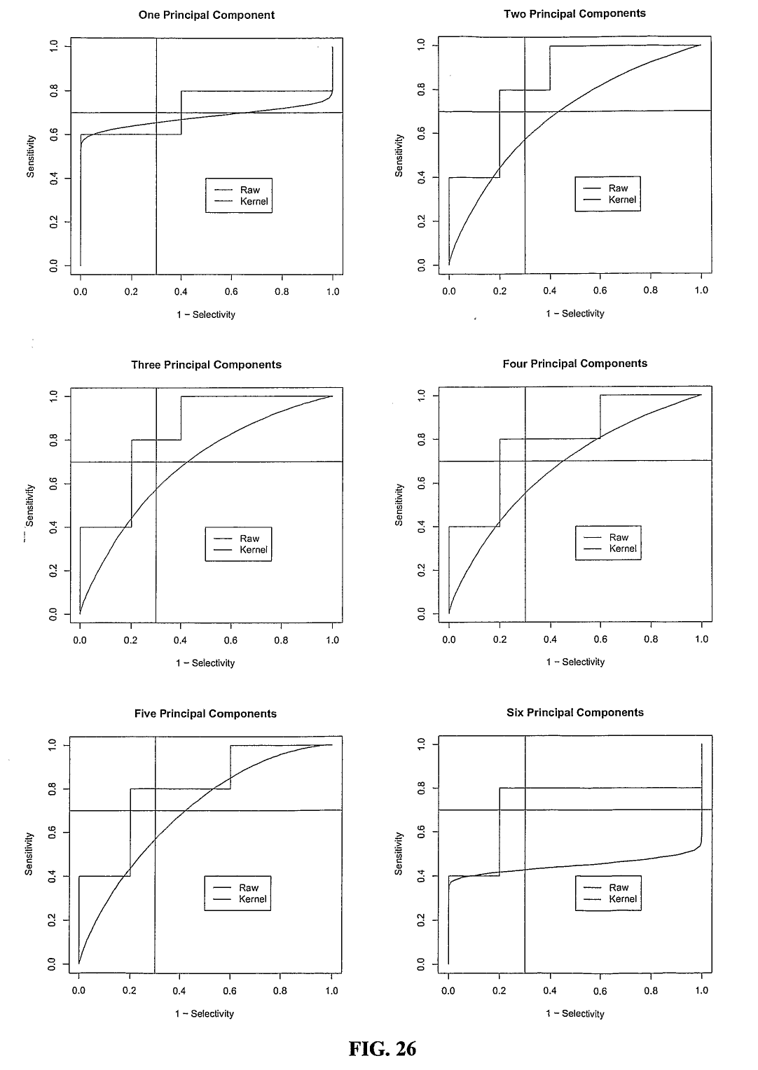

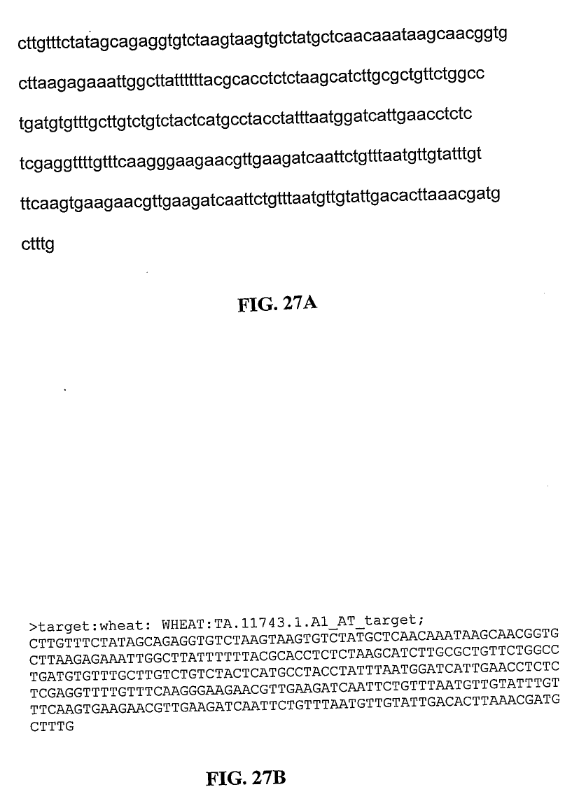

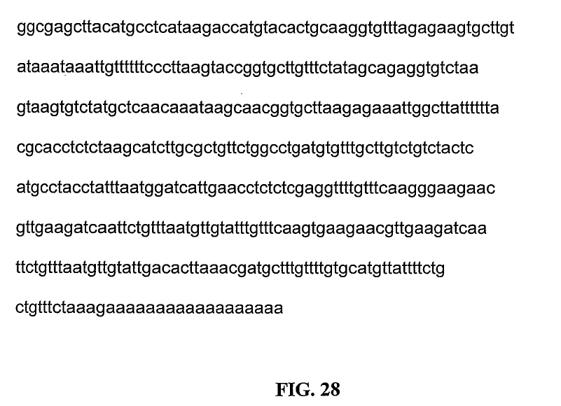

Cluster dendograms based on the single linkage, average linkage and complete linkage algorithms are shown in FIGS. 23, 24 and 25 respectively. None of the dendograms exhibit any clustering of the varieties by yield. As per the principal component plots there is some evidence of a difference between varieties 4 and 5 and the other varieties. The ROC curves based on one, two, three, four, five and six principal components are included in FIG. 26. None of the ROC curves indicate substantial differences between the low yield and high yield wheat varieties. The best sensitivity and selectivity was 0.8 for both values based on six principal components. The support vector machine method was also used to classify the wheat varieties, but the results of this analysis were inferior to the results given by the ROC curves.

From the analysis of the data using linear models, one statistically significant gene based on the Holm and FDR corrections was detected—Ta.11743.1.A1 at.

In summary Affymetrix GeneChip® Wheat Genome Arrays were interrogated with probes derived from RNA samples that were extracted from developing seed samples at 30 days after anthesis (dpa) of high and low milling varieties, and candidate genes showing significant difference in expression profile were identified.

Based on the flour yield measurement from 80 wheat varieties, 10 wheat varieties were chosen based on their flour yield measurements; 5 each for high flour yielding and low flour yielding varieties. Developing wheat seeds of plants at 30 dpa were harvested, RNA extracted and purified and the yield and quality checked by Bioanalyser. The RNA was then labelled and hybridised to Affymetrix GeneChip® Wheat Genome Arrays and the data analysed to identify genes in rank order that showed significant differences in transcript level between the high milling group and the low milling group of wheat varieties.

Analysis and identification of the 30 dpa gene was carried out as follows. Data from the arrays was analysed using currently accepted procedures for Affymetrix GeneChip® arrays (RMA—robust multi-array average (Irizarry et al., 2003)). Probe level data was background corrected, normalised between arrays and gene level data (expression values) summarised from the probe data. Probe data was checked for equivalent distribution between the Affymetrix GeneChip® arrays in the experiment (Box and whisker plots, kernel density estimates) and data from each chip compared to the median of all chips (MA plots) to detect any anomalies in hybridisation behaviour between samples. A statistical test (t-test) was then carried out on the log 2 transformed values from each expressed gene and the p-values adjusted by multiplication by the number of genes tested (Holm correction).

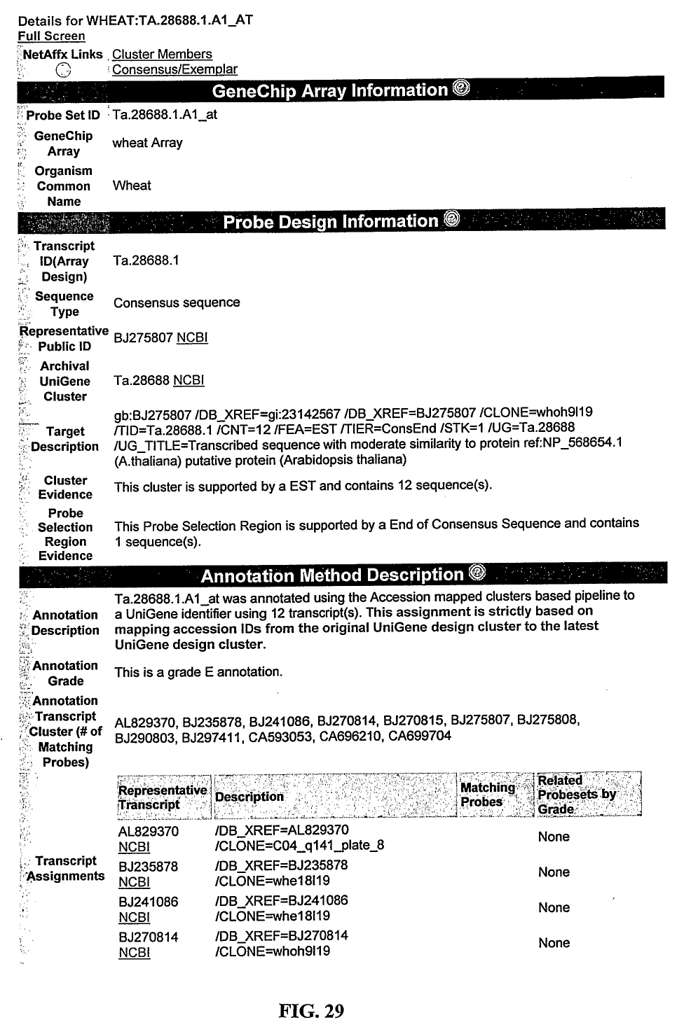

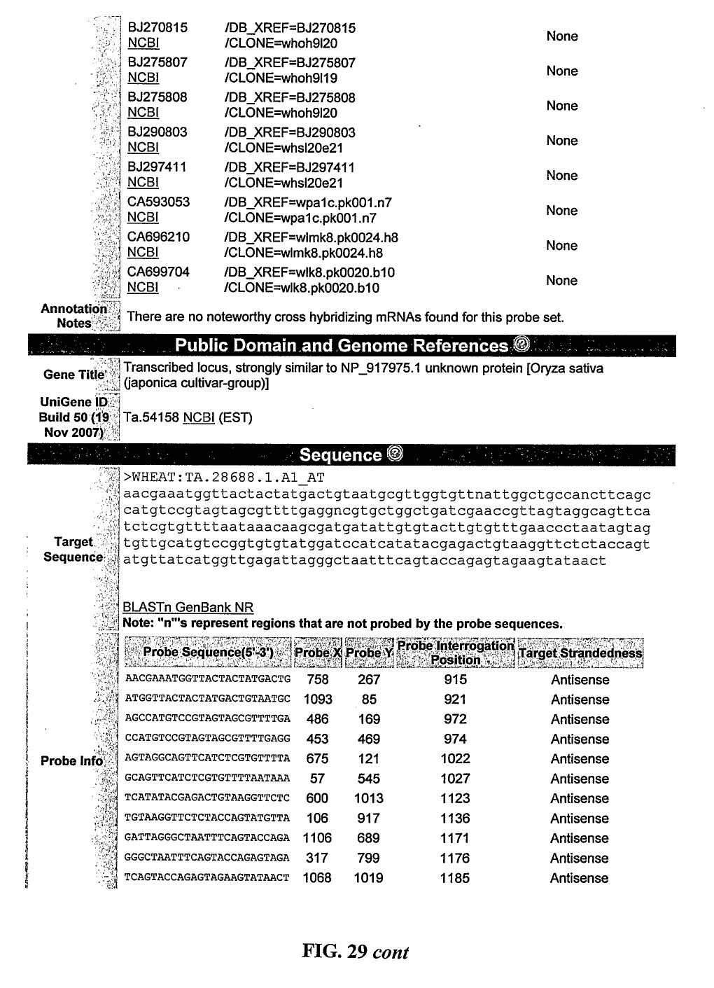

We determined that 1,038 genes were found to be expressed disjointly (no overlap in gene expression, FIG. 12B) between the low and high flour yielding groups. One gene, that we termed “30dpa gene”, appears to be down regulated in the high milling-yield varieties. The 30dpa gene was found to have a significantly different expression level at a corrected 0.05 level. The Holm correction method adjusts for the fact that a large number of genes were tested for expression level differences. The gene identified with the corrected p value of 0.04 corresponded to the affy-probes (Table 8) that were designed over the target Ta.11743.1.A1_at (FIG. 29B). For the 30dpa gene the fold difference of the gene expression values was 3.1 fold larger in the low yield varieties compared to the high yield varieties.

### Conclusion

1. The differences in gene expression between the low yield and high yield wheat varieties consist of small differences in a large number of genes rather than large differences in a small number of genes.

2. After conservative adjustment of p values, there is only one gene for which a statistically significant difference between the low yield and high yield varieties could be detected.

### Example 5

### Characterisation of the 14 dpa Gene

The sequence for the candidate gene corresponding to the target “Ta.28688.1.A1_at” present on the Affymetrix GeneChip® Wheat Genome Array was obtained through the NetAffx website http://www.affymetrix.com/analysis/netaffx/index.affx (FIG. 29). The “Ta.28688.1.A1_at” also shows similarity to the transcribed locus NP_{—}001060360.1 from rice which corresponds to the locus NC_{—}008400 on the rice genome, and to the transcribed locus AT5G46030 hypothetical protein from Arabidopsis which corresponds to the locus NC_{—}003076 on the Arabidopsis genome. However, the target “Ta.28688.1.A1_at” shows similarity an annotated transcribed locus from yeast (transcribed locus NP_{—}012762.1) which corresponds to locus NC_{—}001143 and is thought to be a transcription elongation factor that contains a conserved zinc finger domain and is implicated in the maintenance of proper chromatin structure in actively transcribed regions.

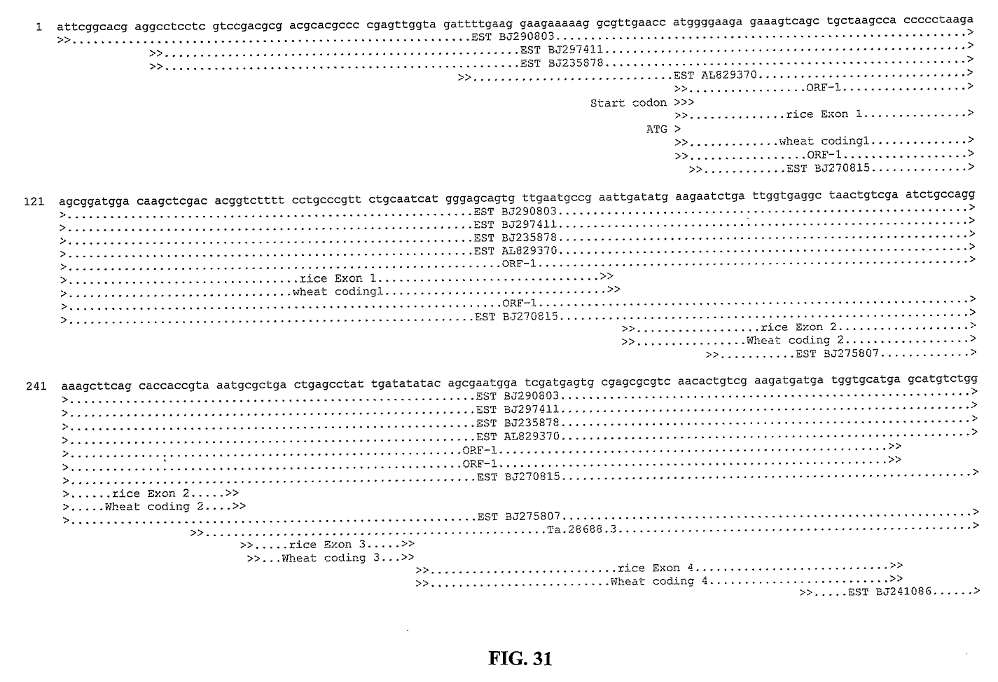

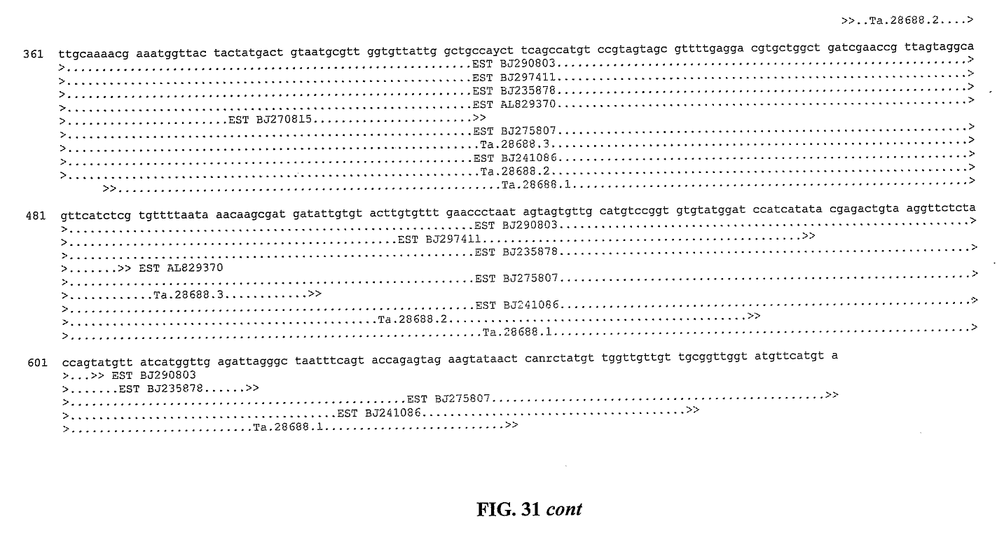

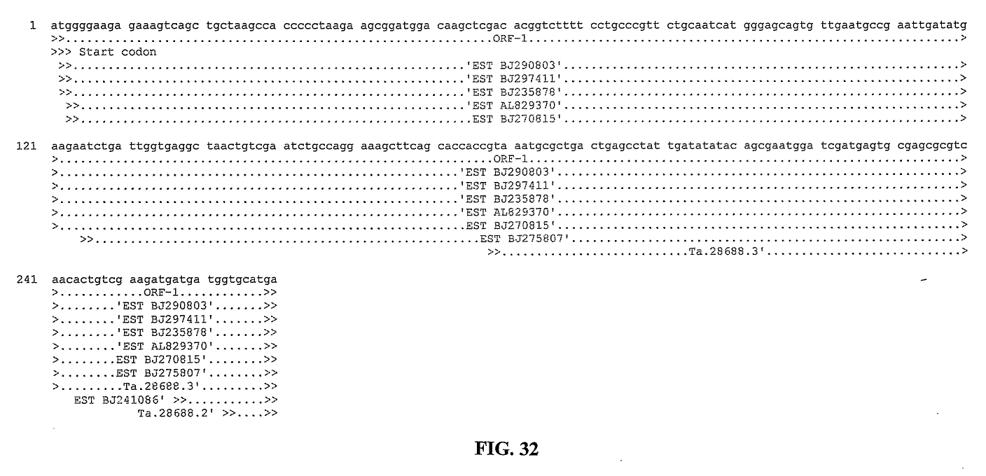

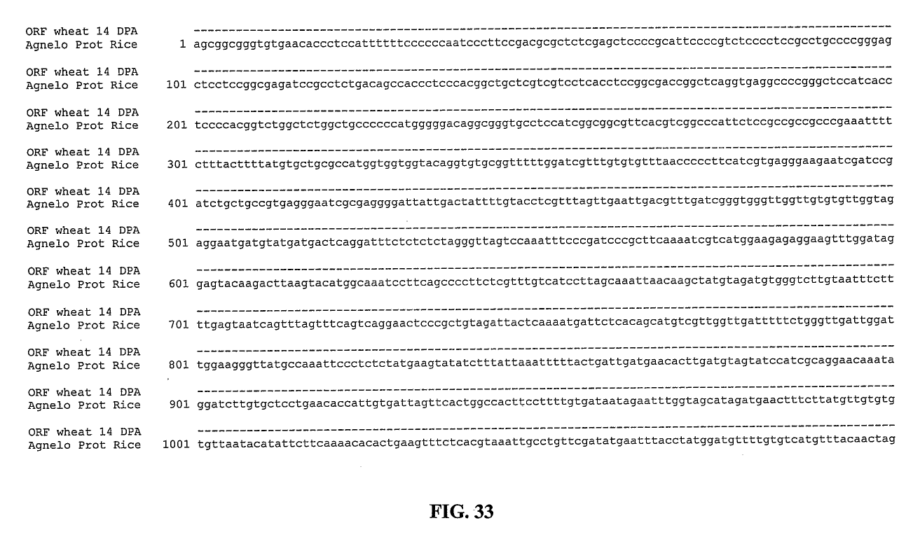

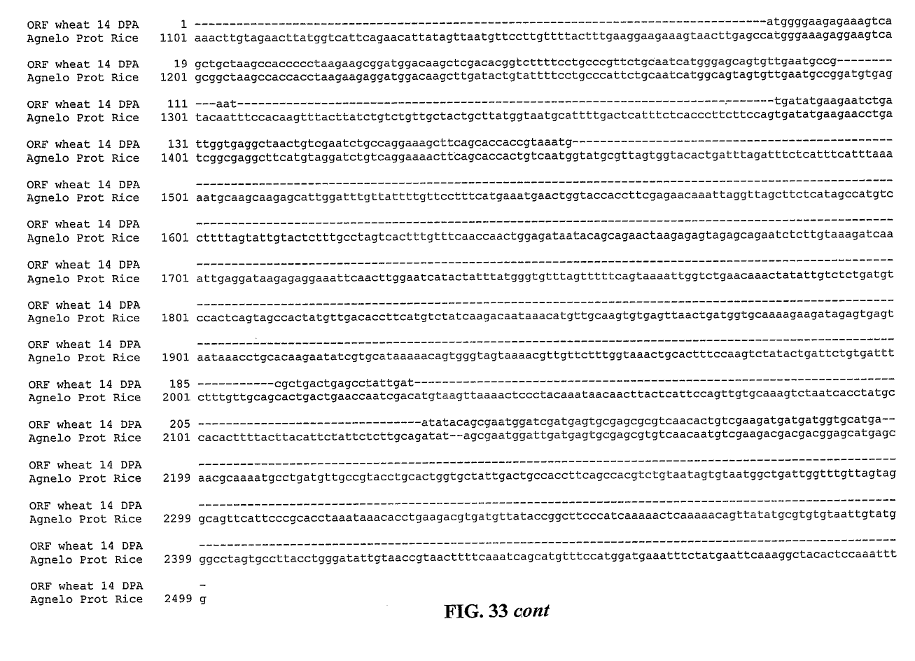

The target “Ta.28688.1.A1_at” is an EST from wheat. The target “Ta.28688.1.A1_at” clusters with other incomplete EST's from wheat and an alignment of all these ESTs is shown in FIG. 30. The EST alignment was used to generate a consensus sequence (FIG. 31), and this consensus sequence was used to predict an open reading frame which is shown in FIG. 32. The open reading frame shown in FIG. 8 was aligned with the rice genomic DNA sequence corresponding to NC_{—}008400 to predict possible intron exon boundaries, and was found to contain four exons (FIG. 33).

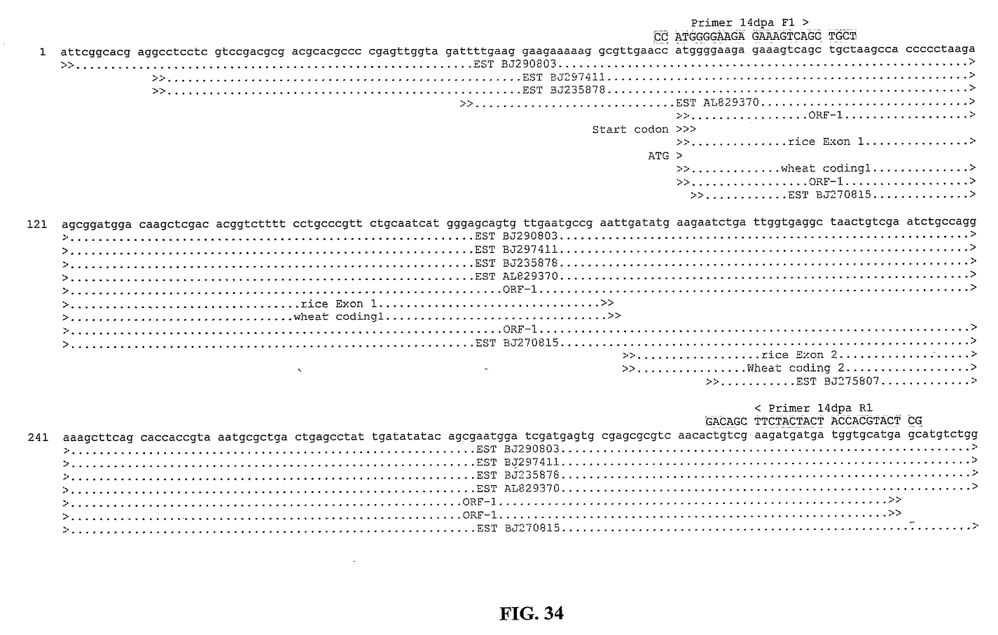

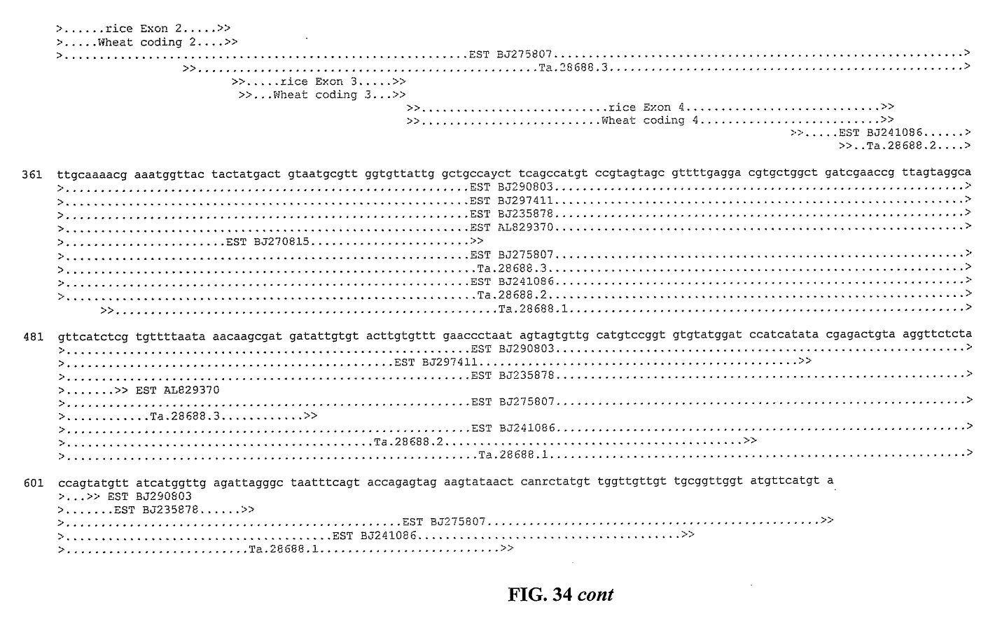

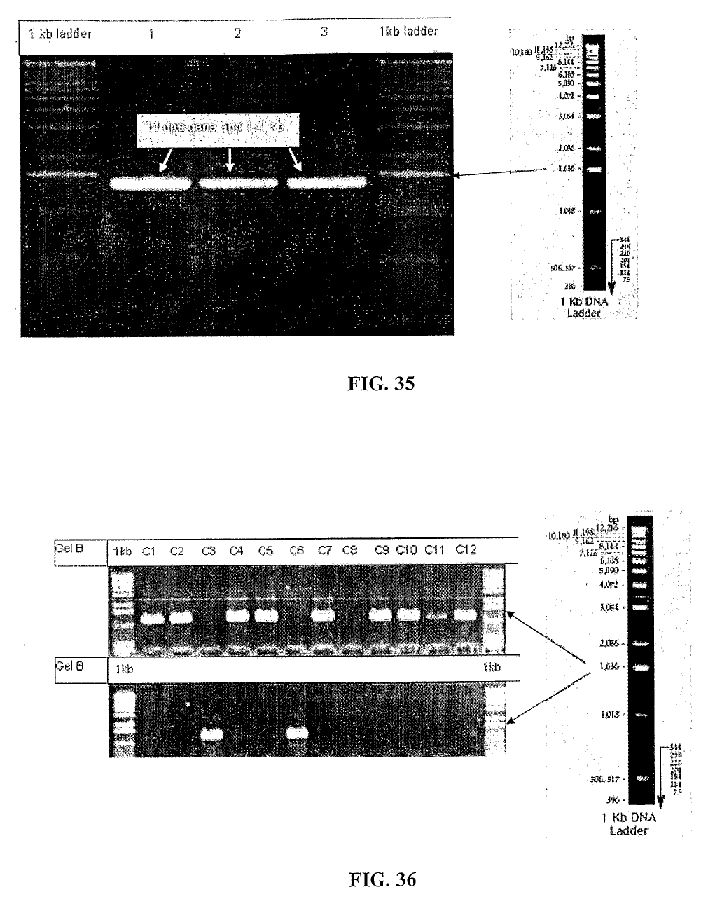

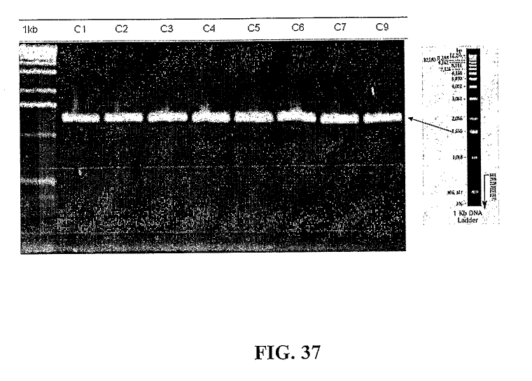

The consensus sequence as shown in FIG. 31 was used to design PCR primers (Primer14dpaF1 and Primed 4dpaR1) to amplify the 14 dpa gene in wheat (FIG. 34). The 14 dpa gene sequence was successfully amplified by PCR using the primers Primer14dpaF1 and Primer14dpaR1 in combination with wheat genomic DNA from the variety Bob white (FIG. 35). The PCR fragments were purified and ligated into pGEM3zf (Promega, USA) TA vector followed by cloning into JM109 cells (Promega, USA). Twelve white colonies (C1 to C12) were selected and screened by PCR using M13F+Primer 14dpaF1 (Gel A) and M13F+Primer 14dpaF1 (Gel B), to identify recombinant colonies (FIG. 36). Of the twelve colonies, six colonies C1, C2, C3, C4, C5, C6. C7 and C9 identified as recombinant were subjected to PCR (M13F and M13R) to amplify the 14 dpa gene, and the amplified product (FIG. 37) was subjected to sequencing.

### Example 6

### Isolated Wheat 14 dpa Gene Sequences

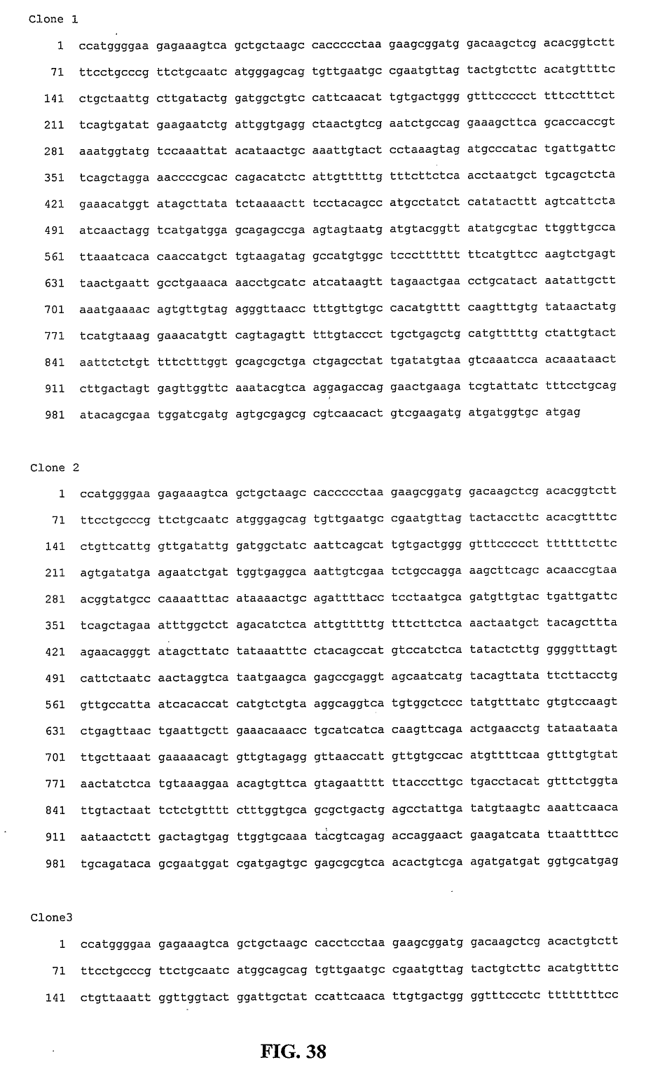

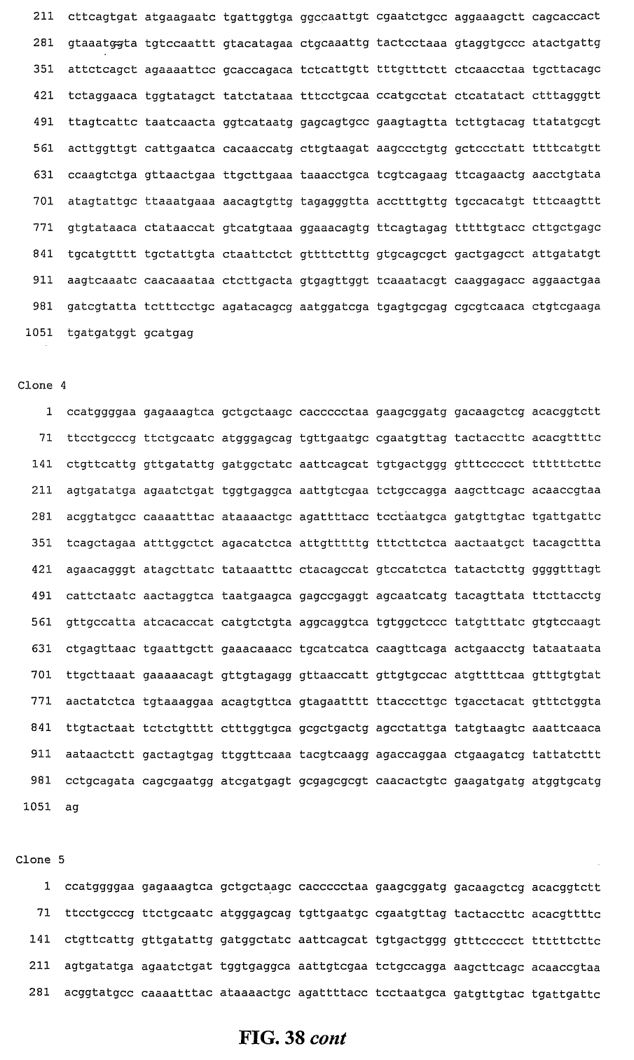

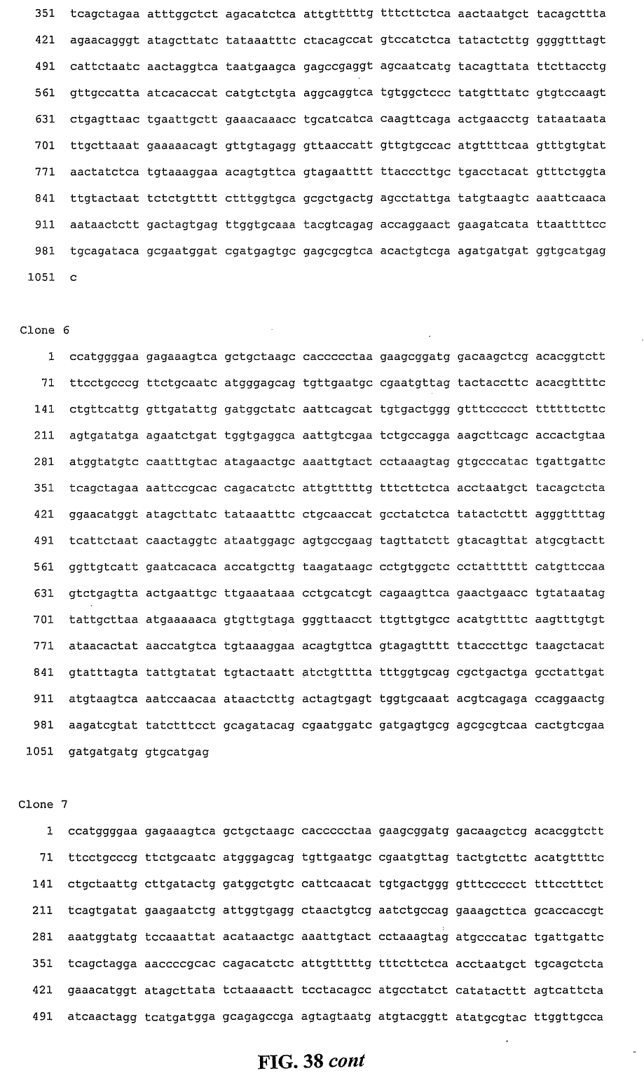

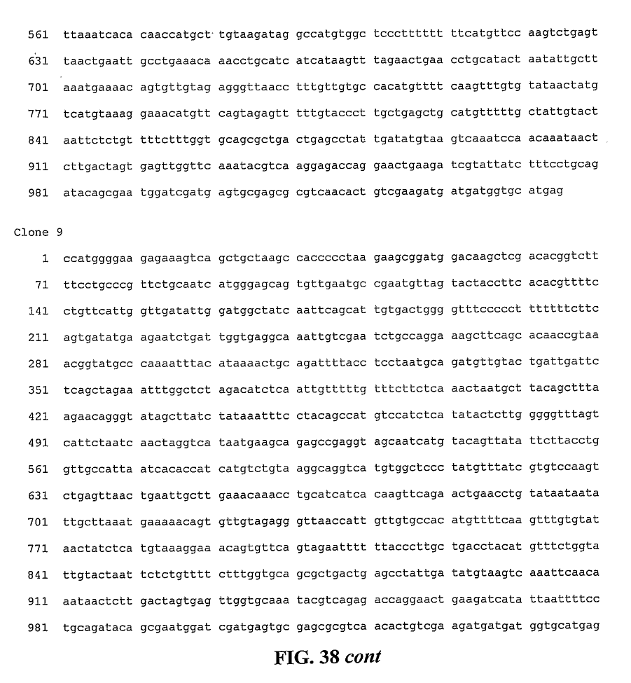

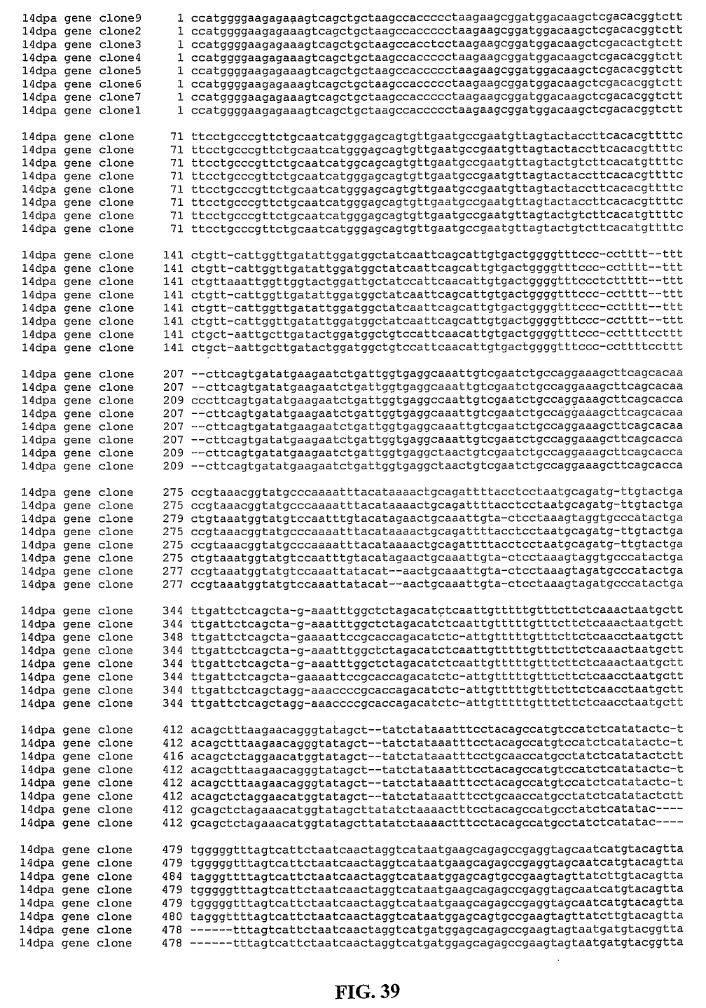

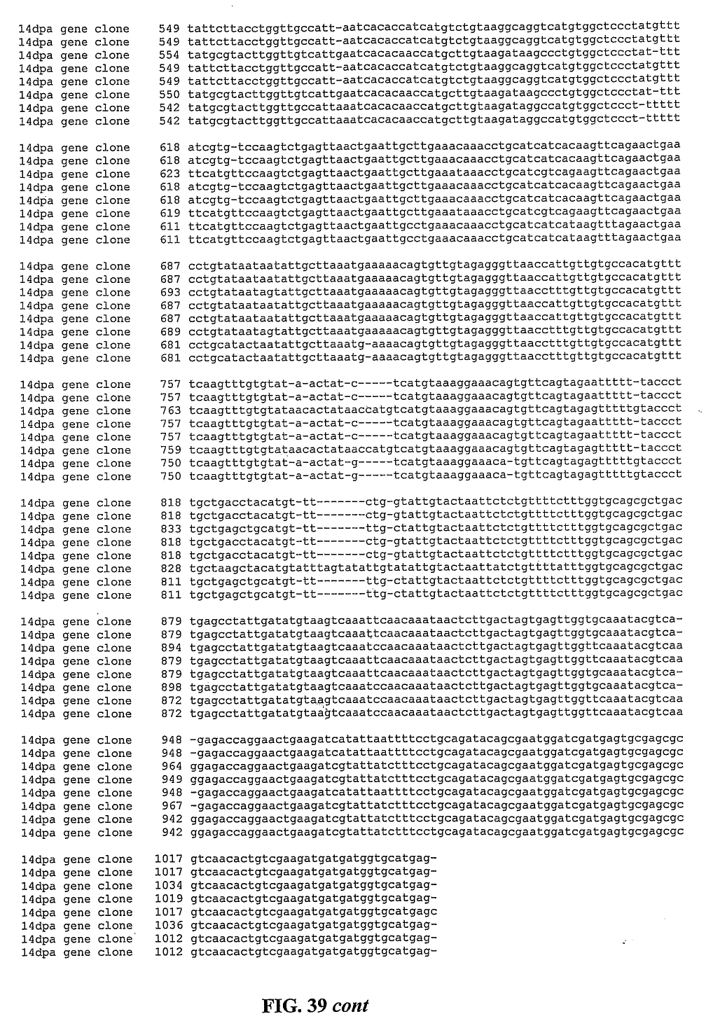

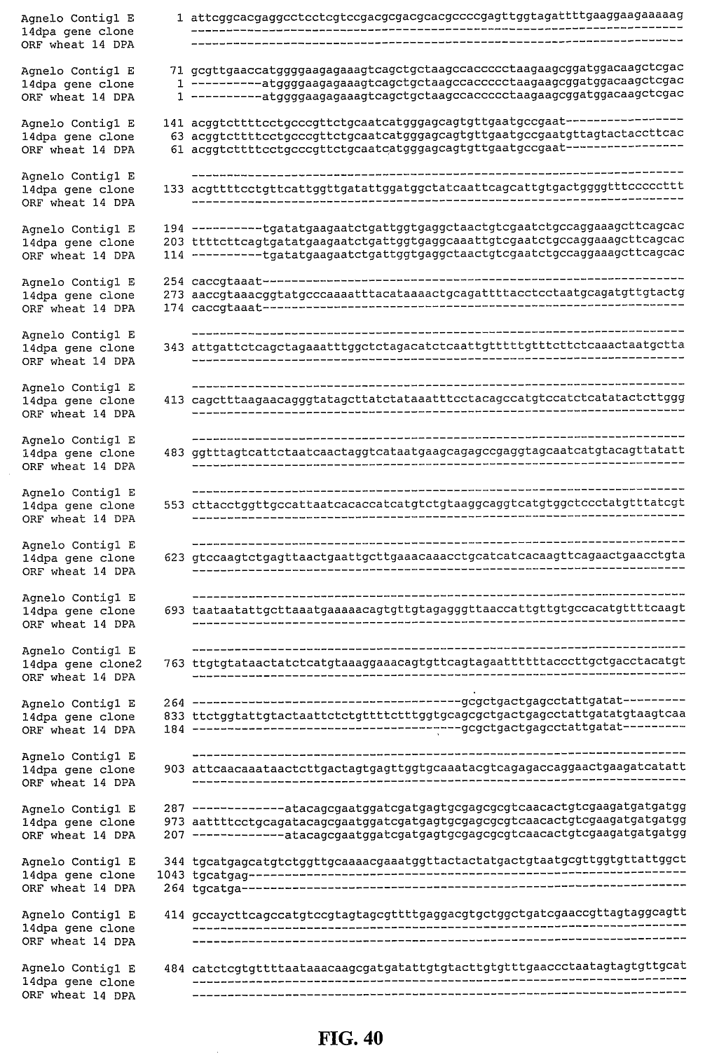

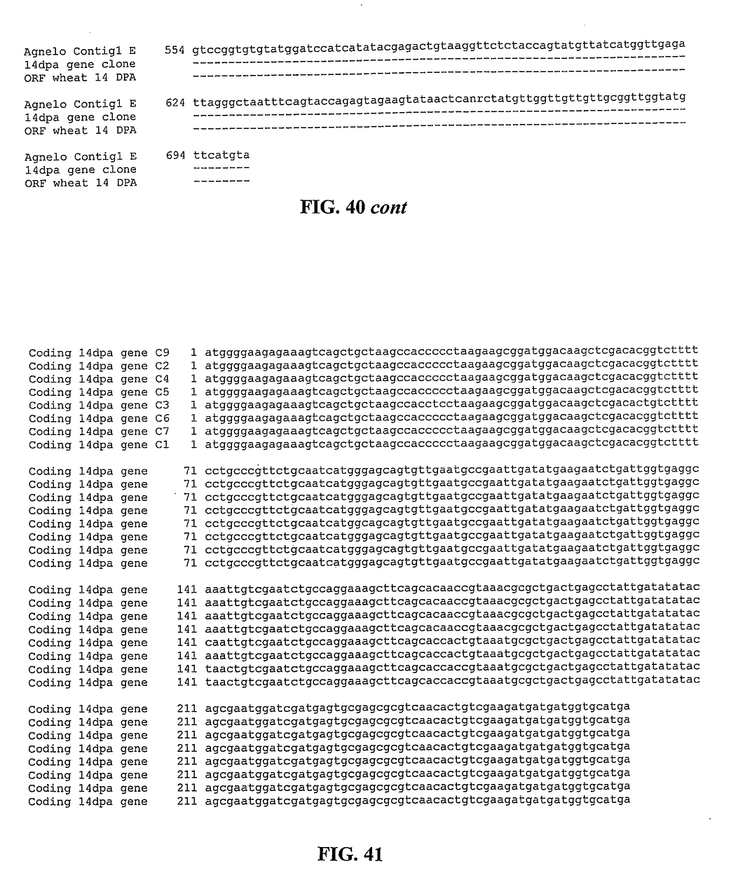

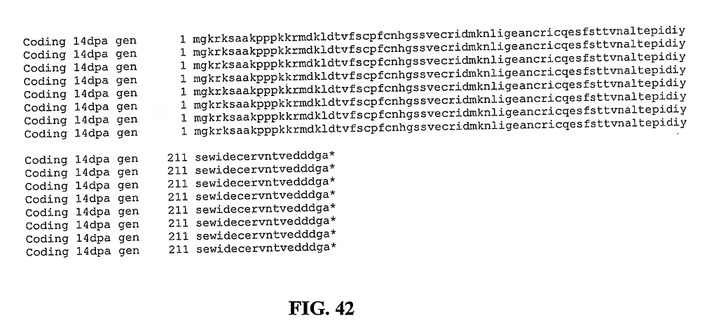

The sequences corresponding to clones C1, C2, C3, C4, C5, C6. C7 and C9 are shown in FIG. 38. An alignment of these gene sequences indicate high homology although some differences were observed (FIG. 39). These sequences are gene sequences and thus have the intron and exon sequences. To identify the intron sequences, the sequence corresponding to C2 was aligned to the consensus sequences of EST to target “Ta.28688.1.A1_at” (FIG. 31) and the predicted coding sequence of the 14 dpa gene (FIG. 32), and the resulting alignment is shown in FIG. 40. Based on the data in FIG. 40, the intron-exon boundaries for sequences of all clones (C1, C2, C3, C4, C5, C6. C7 and C9) were noted, and the exon sequences deleted to yield corresponding coding sequences. The coding sequences for all clones (C1, C2, C3, C4, C5, C6. C7 and C9) when aligned showed high homology with some differences (FIG. 41). The coding sequences were translated and aligned to show a perfect match (FIG. 42) indicating that the nucleotide differences in the corresponding coding sequences (FIG. 41) are all in the wobble-position and that the protein sequence is under high evolutionary pressure to maintain its sequence.

### Example 7

### Comparison Between Wheat 14dpa Gene Sequences and Other Plant Coding Sequences

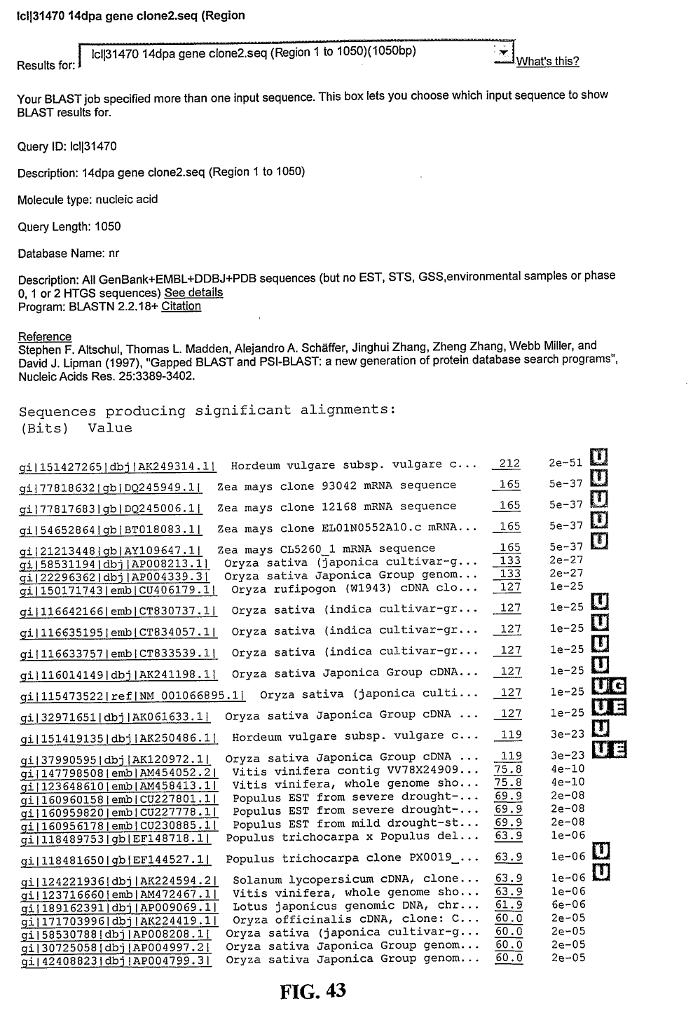

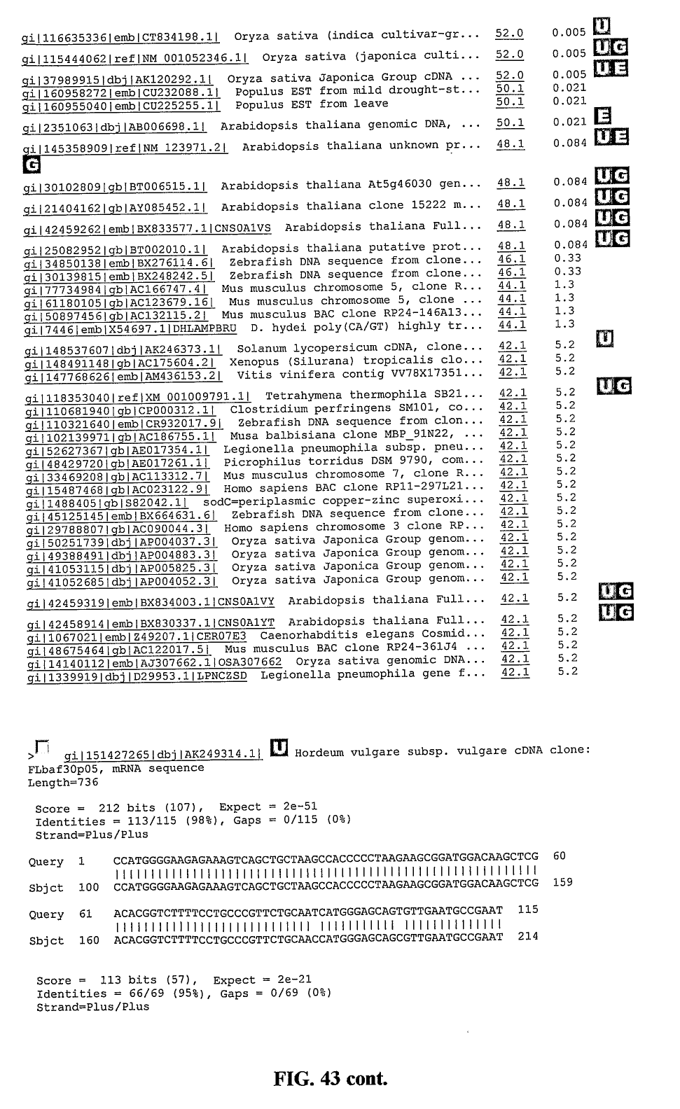

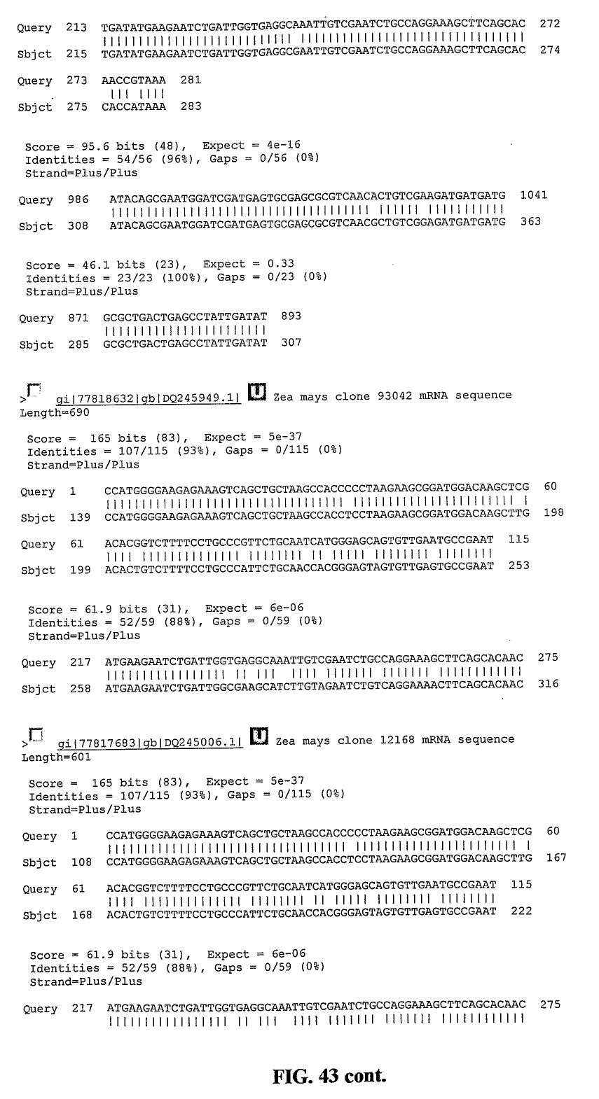

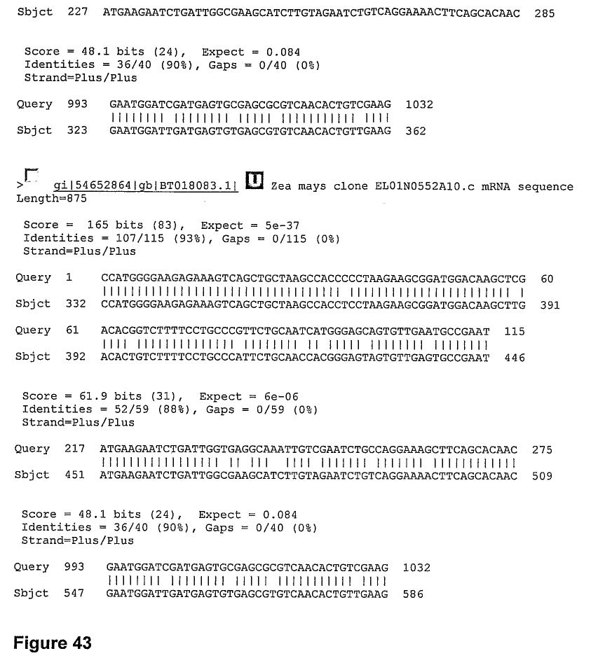

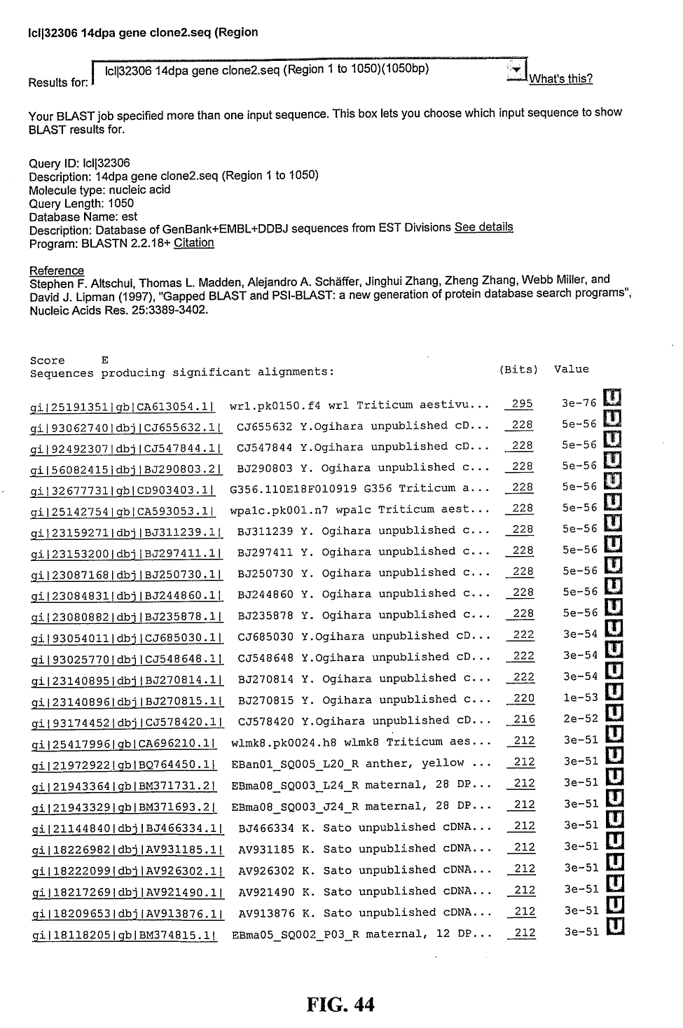

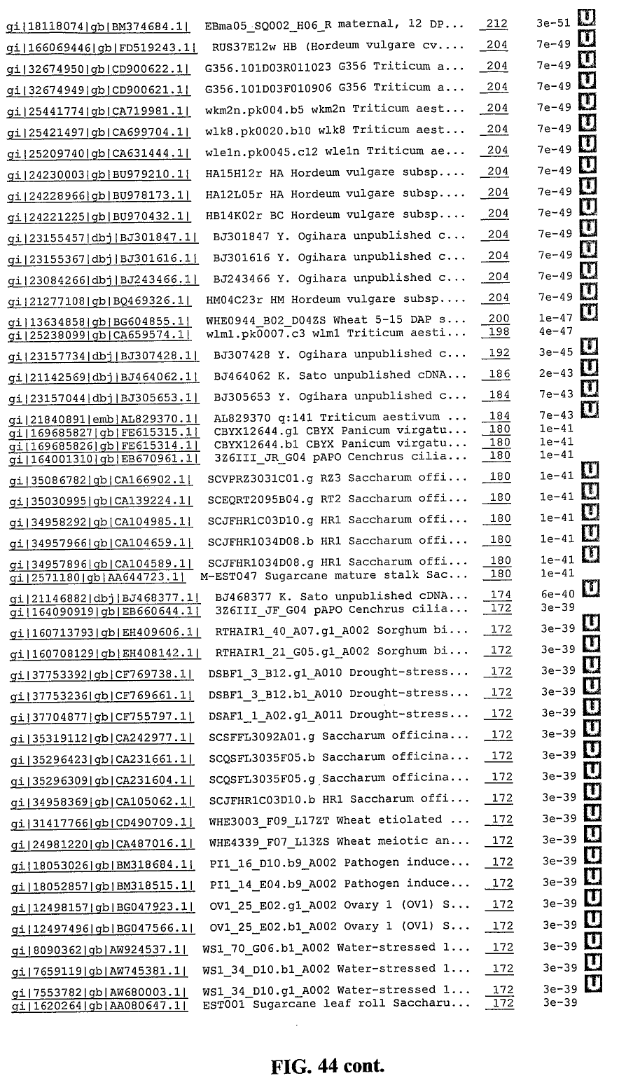

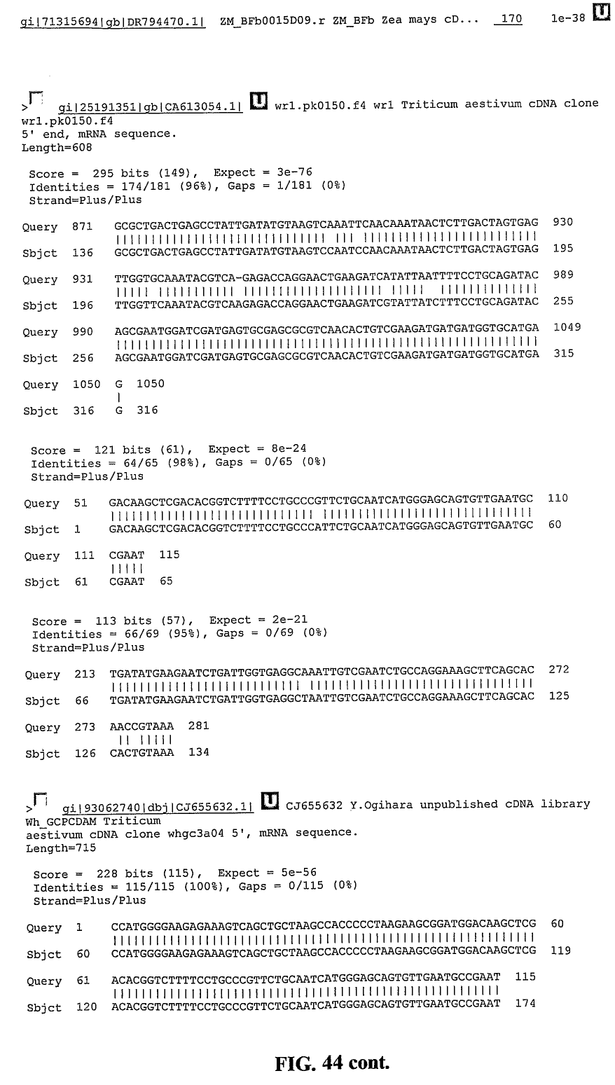

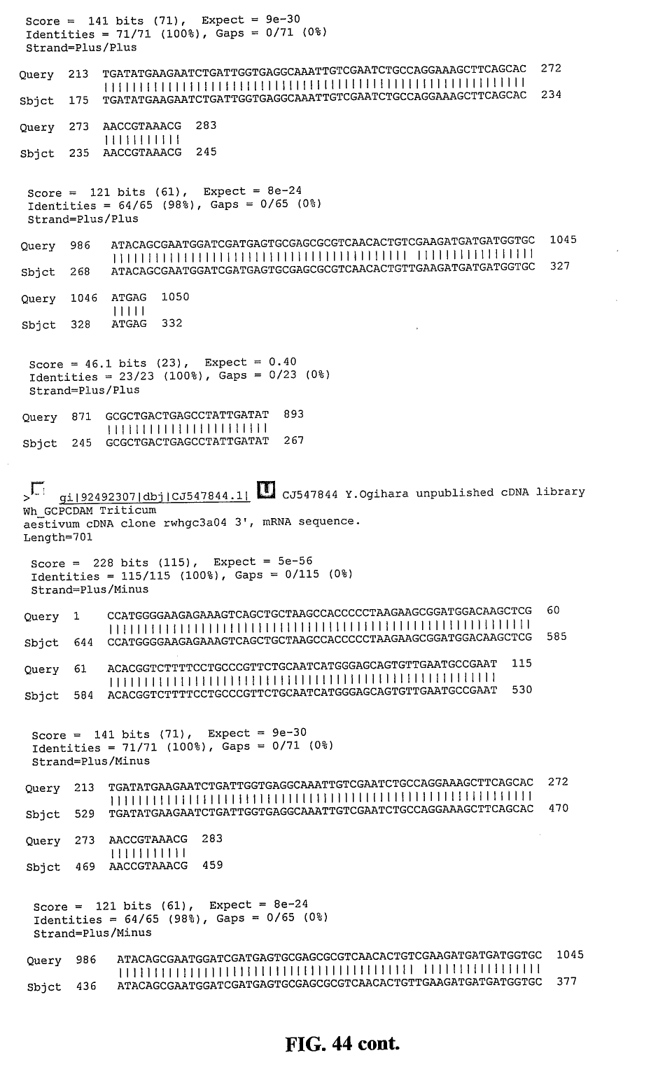

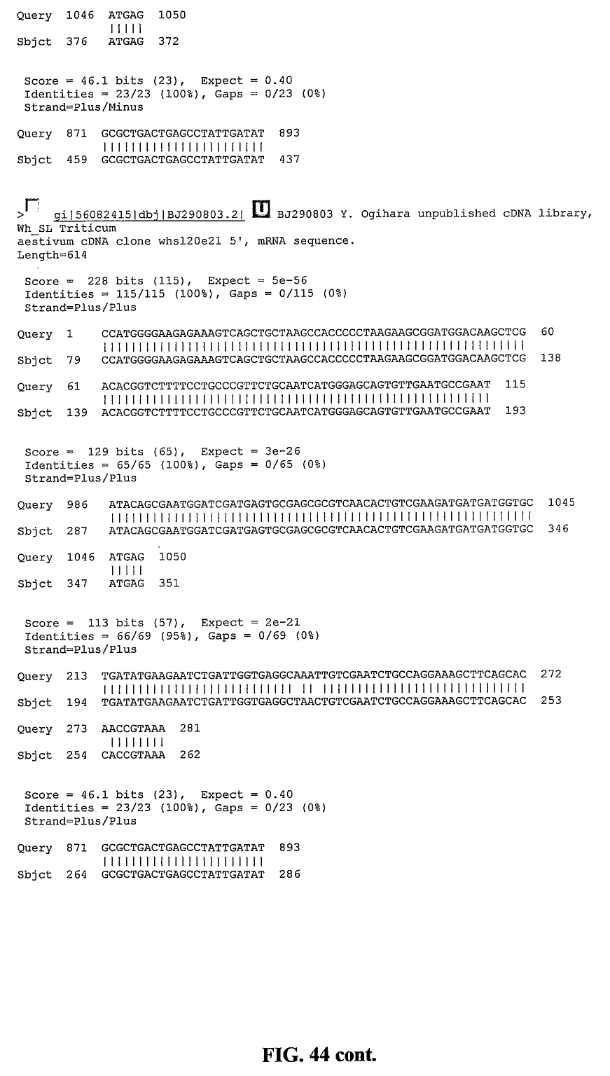

The isolated wheat genomic sequence of the 14dpa gene corresponding to Clone 2 was subjected to BLAST analysis on the NCBI portal for non redundant DNA (nr-DNA) and for ESTs.: http://blast.ncbi.nlm.nih.gov/Blast.cgi. Results of nr-DNA BLAST analysis with alignments for the first 4 hits are shown in FIG. 43. Results of the ESTBLAST analysis with alignments for the first 4 hits are shown in FIG. 44.

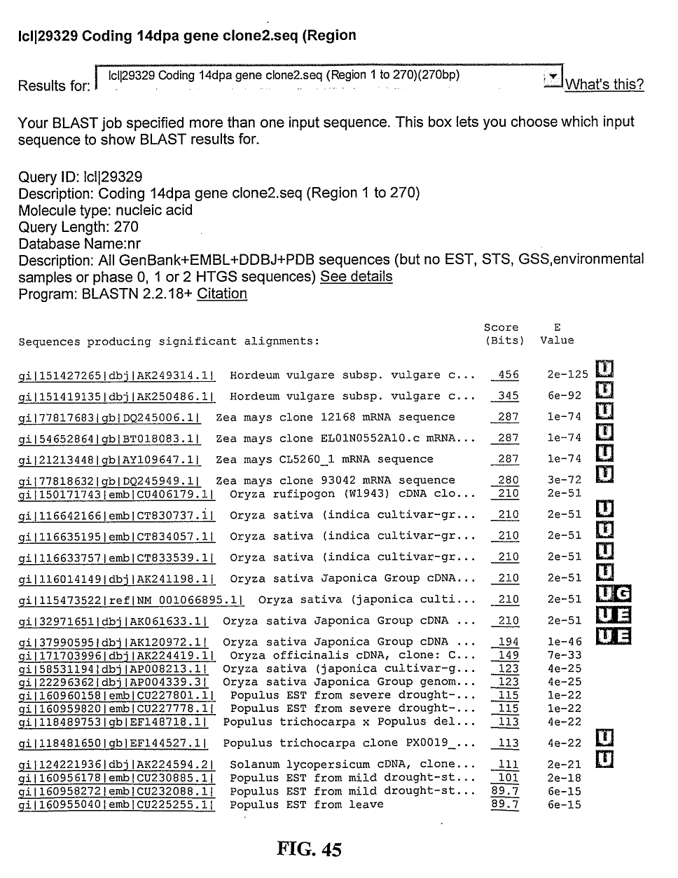

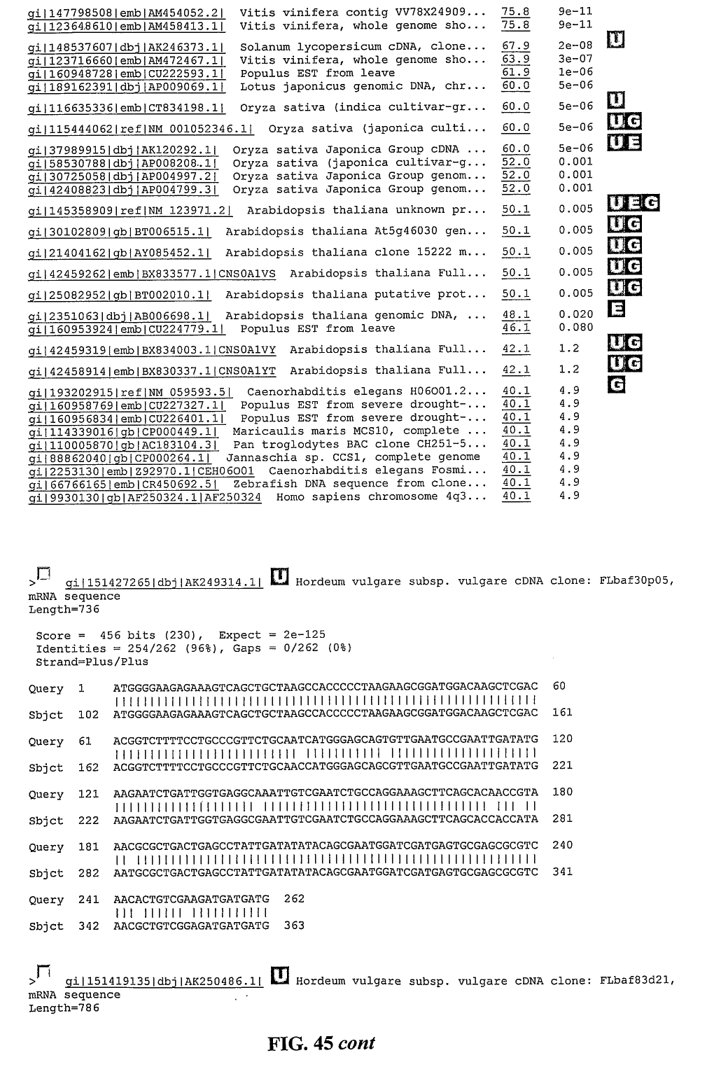

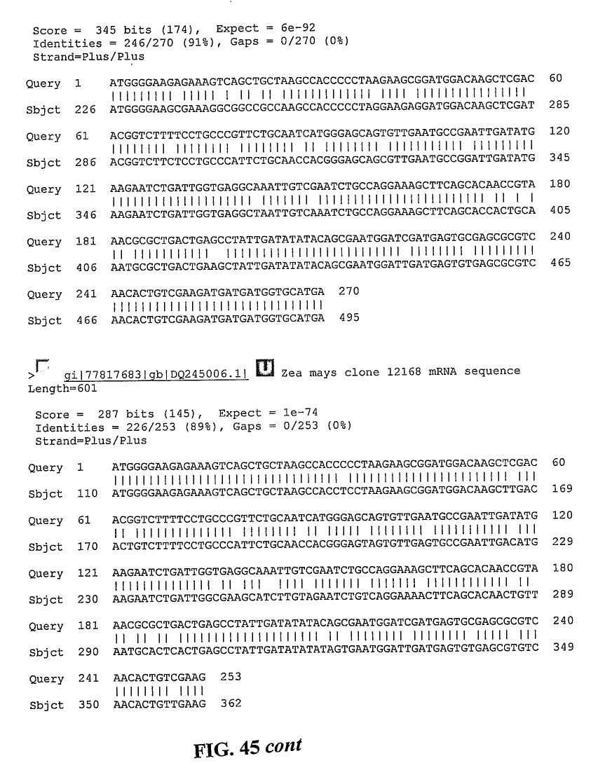

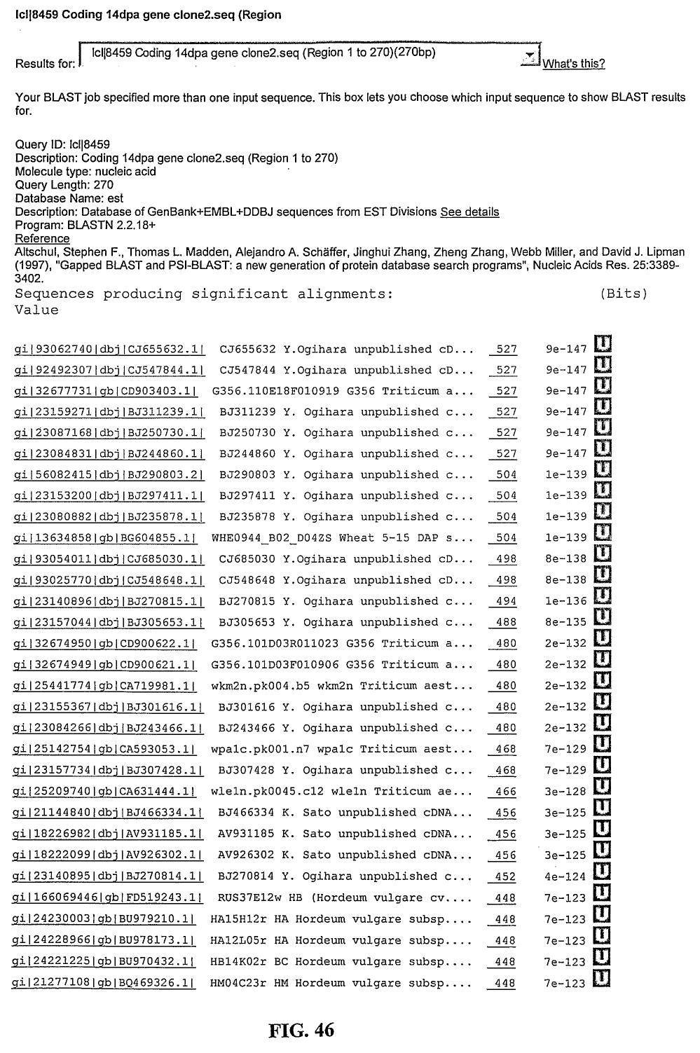

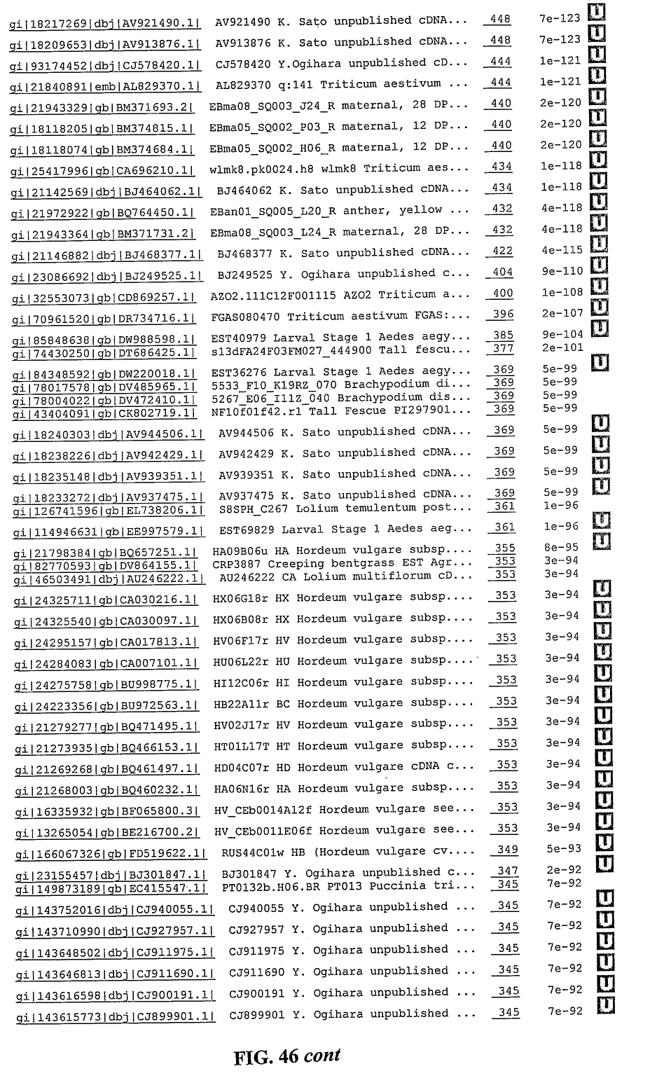

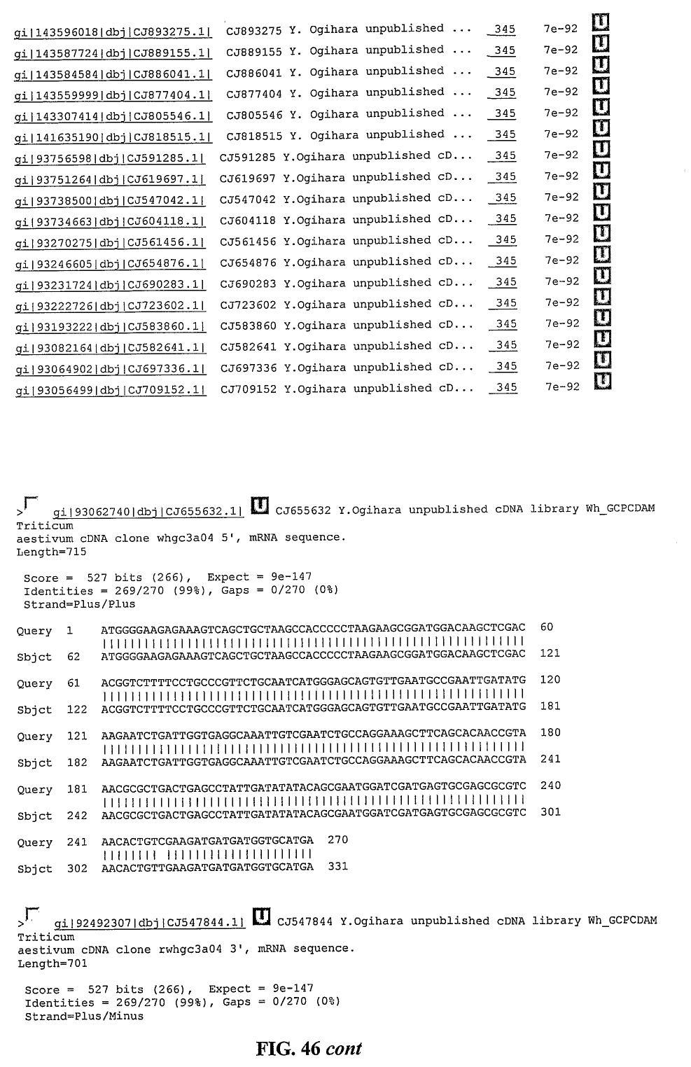

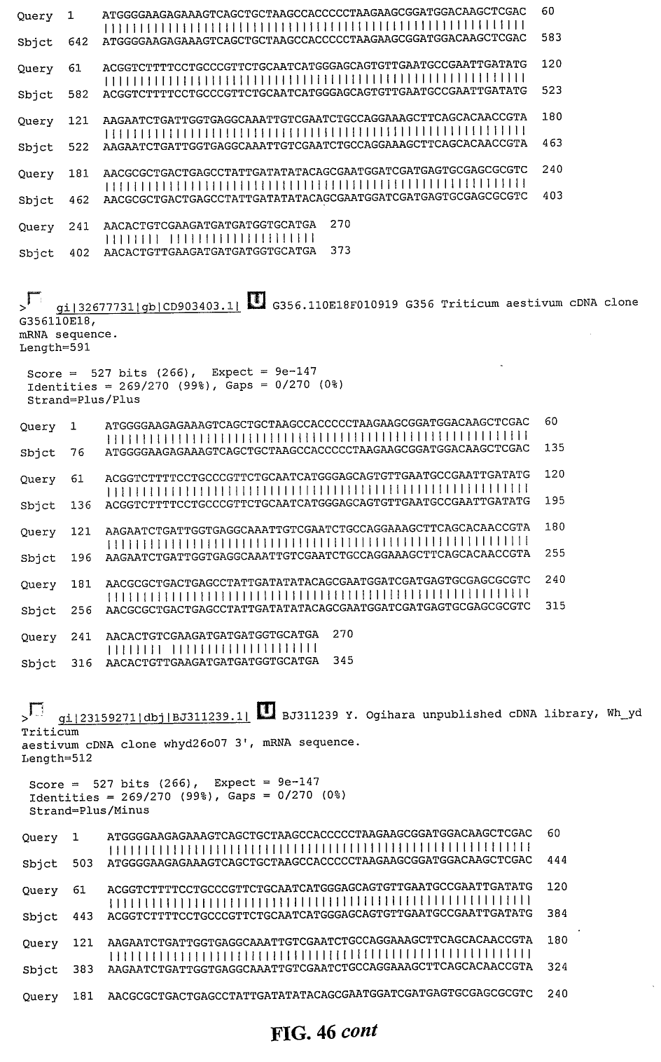

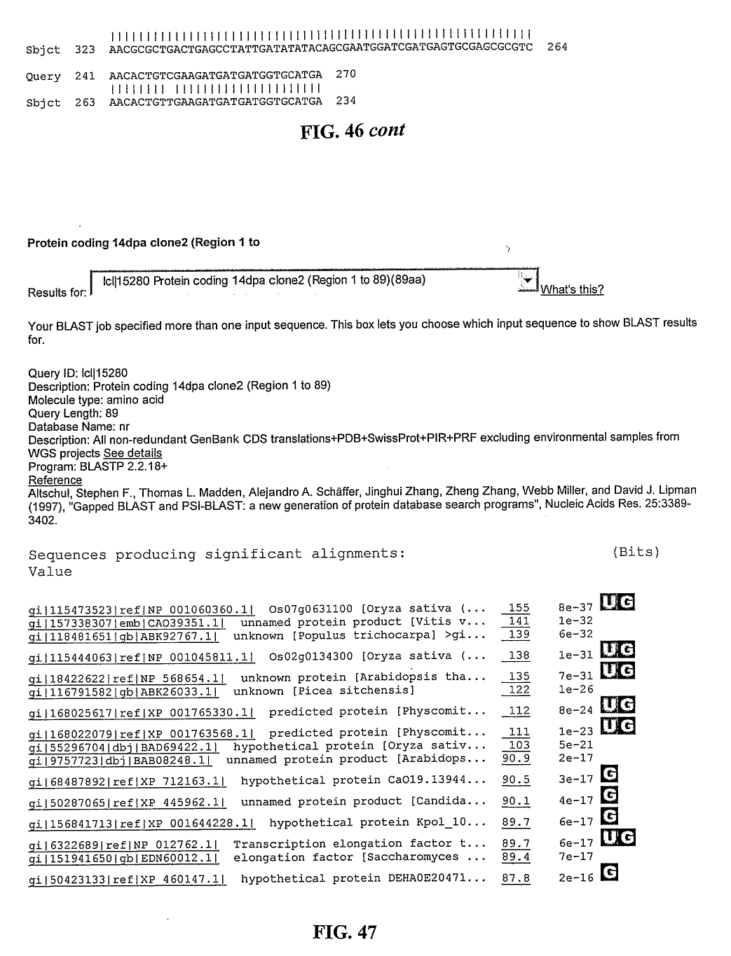

Similarly the coding sequence (exons only) of the 14 dpa gene (clone 2), corresponding to the isolated wheat genomic sequence Clone 2 was subjected to BLAST analysis on the NCBI portal for non redundant DNA (nr-DNA) and for ESTs.: http://blast.ncbi.nlm.nih.gov/Blast.cgi. Results of nr-DNA BLAST analysis with alignments for the first 4 hits are shown in FIG. 45. Results of the ESTBLAST analysis with alignments for the first 4 hits are shown in FIG. 46.

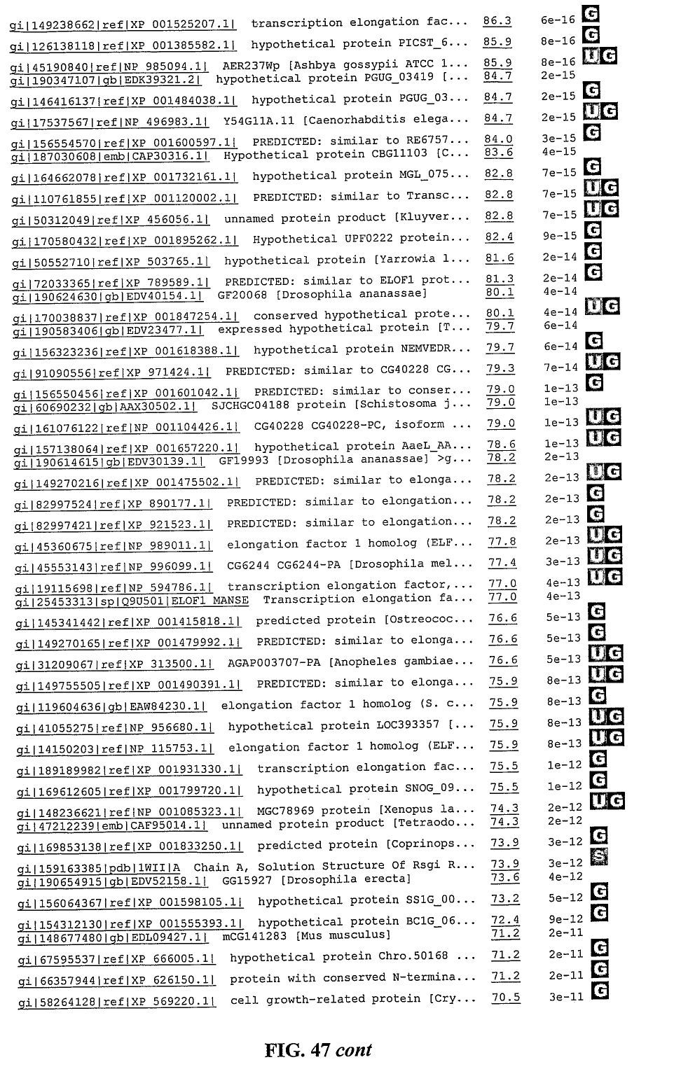

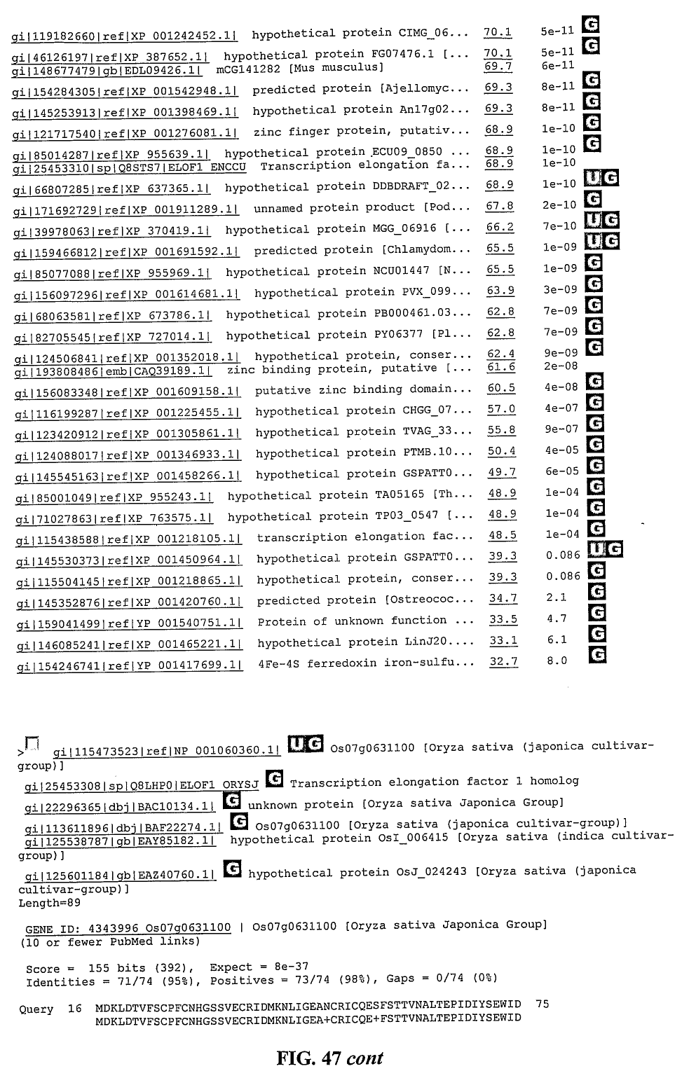

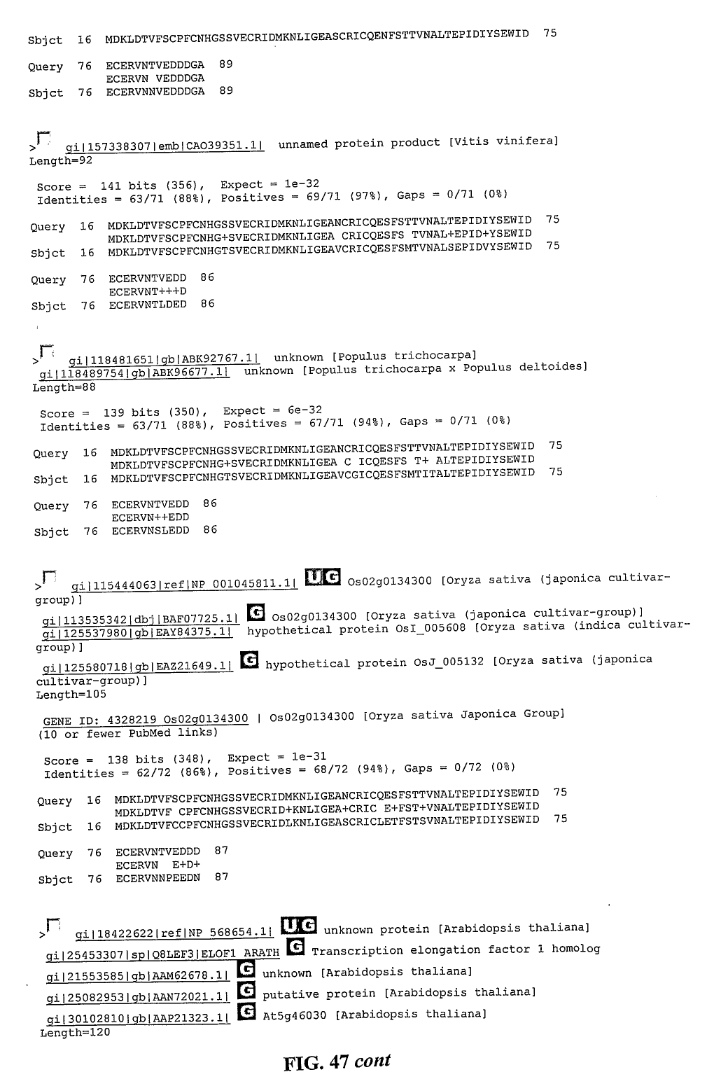

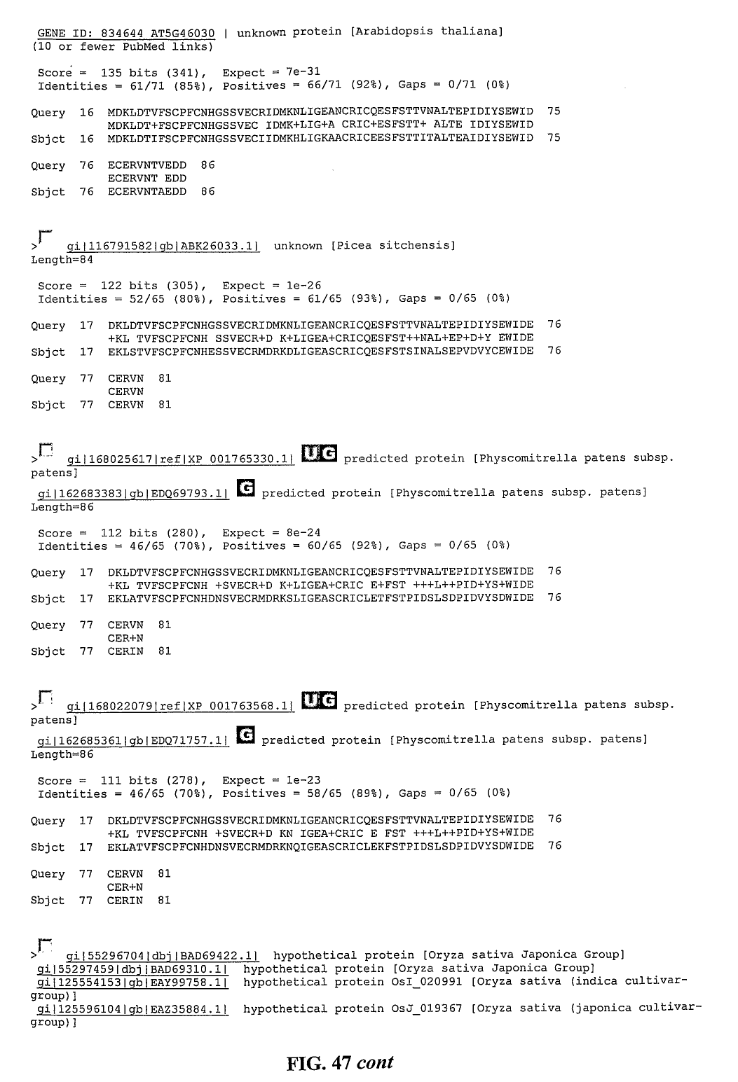

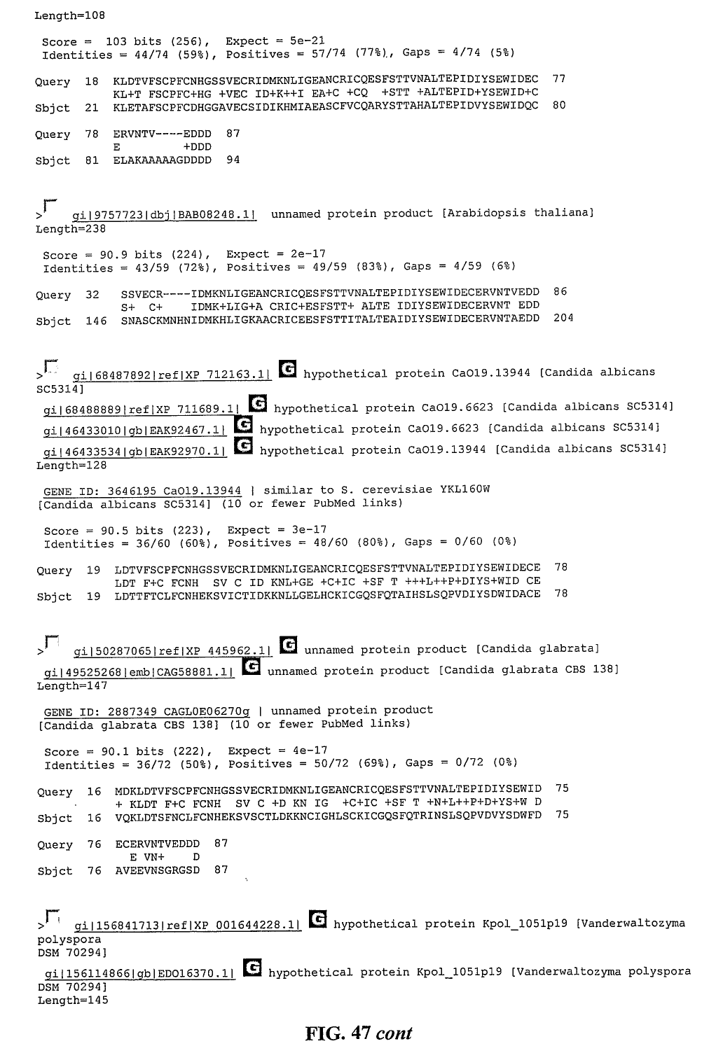

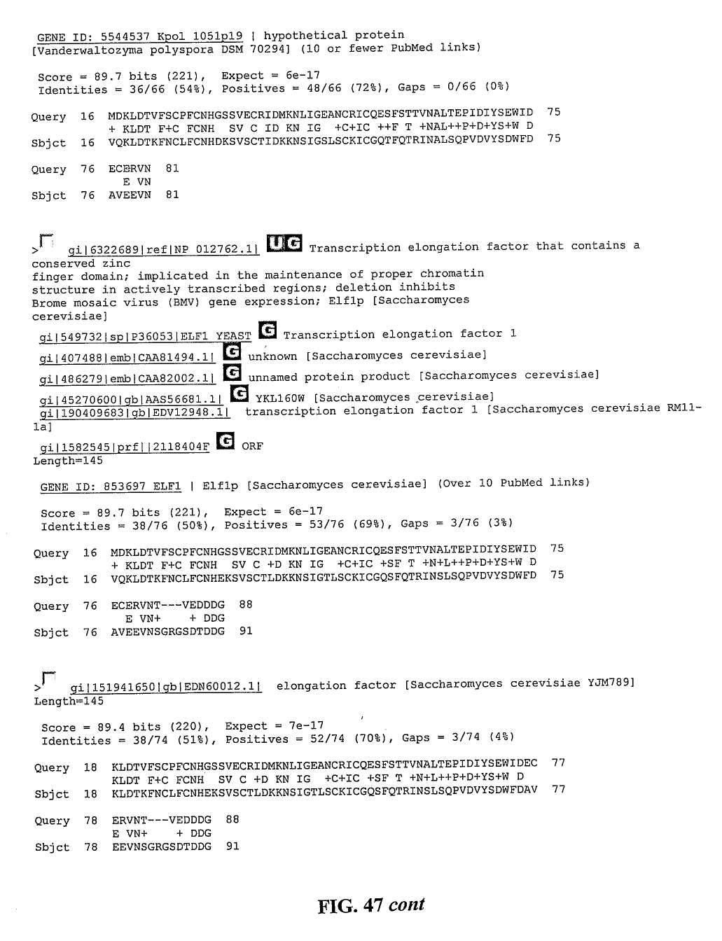

Similarly the translated amino acid sequence of 14 dpa coding sequence of clone 2 was subjected to BLASTp for non-redundant protein sequences. Results of nr-protein sequences with alignments for the first 4 hits are shown in FIG. 47.

### Example 8

### Characterisation of the 30 dpa Gene

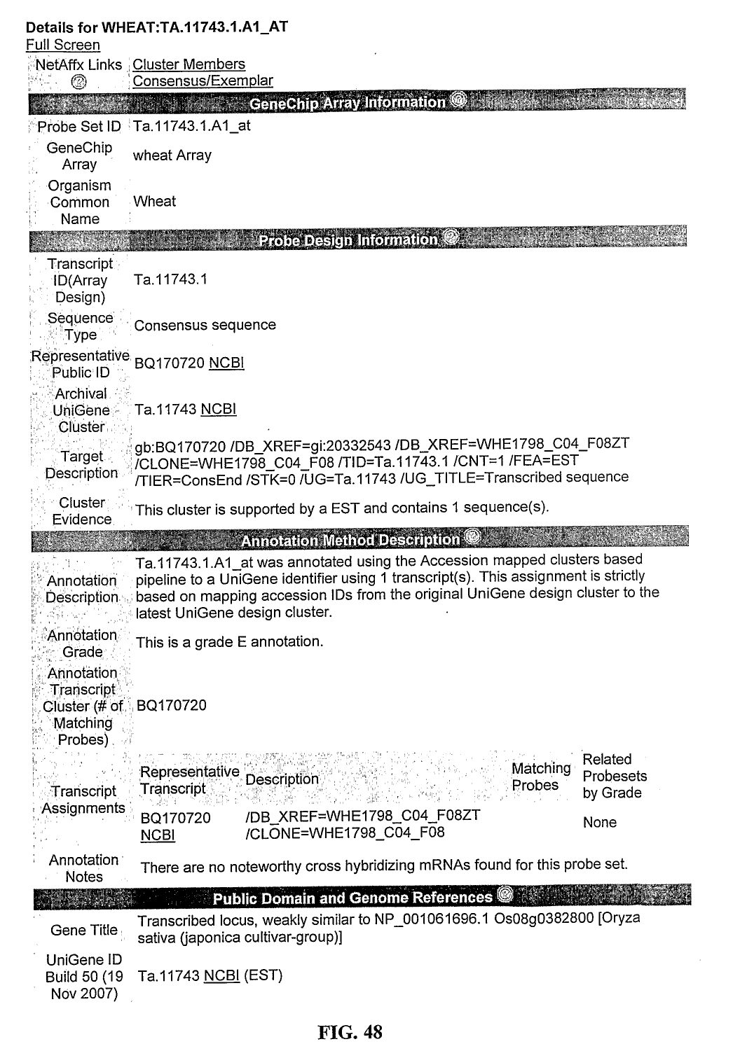

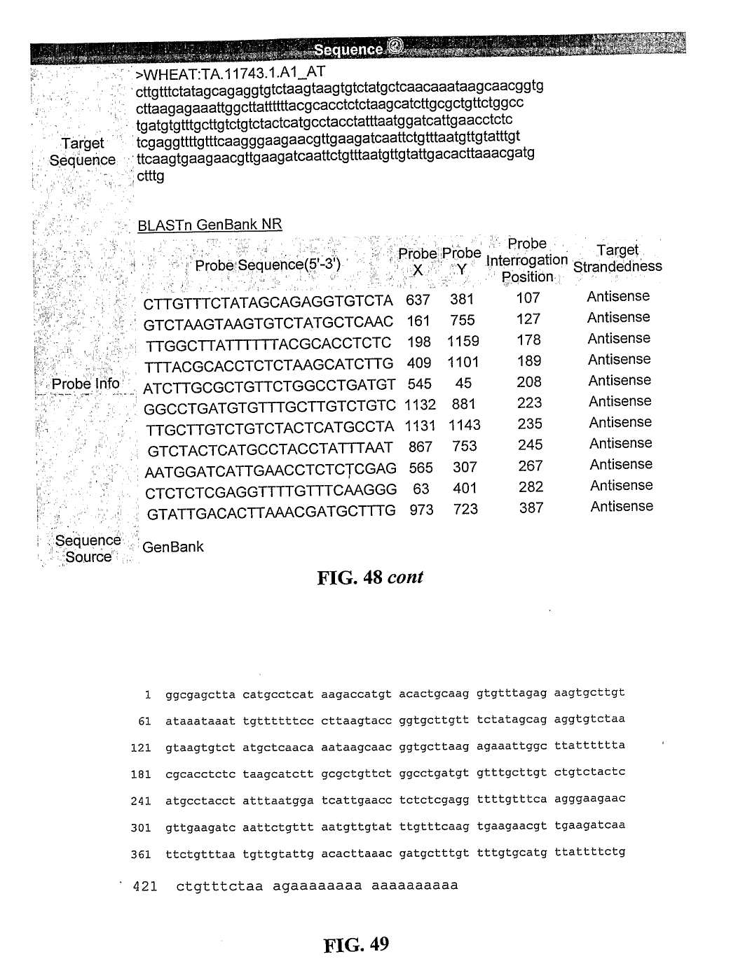

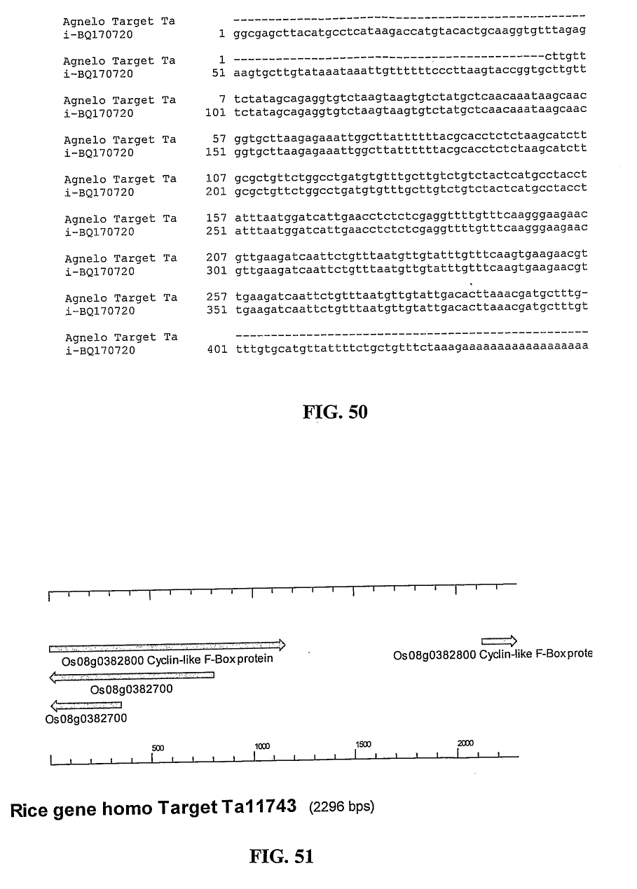

The sequence for the candidate gene corresponding to the target “Ta.11743.1.A1_at” present on the Affymetrix GeneChip® Wheat Genome Array was obtained through the NetAffx web site (http://www.affymetrix.com/analysis/netaffx/index.affx.,) (FIG. 48). The target “Ta.11743.1.A1_at” shows no similarity to any nr-DNA but matches perfectly one EST with locus BQ170720 (FIG. 49), not surprisingly as this is the EST that contains the target “Ta.11743.1.A1_at” (FIG. 50).

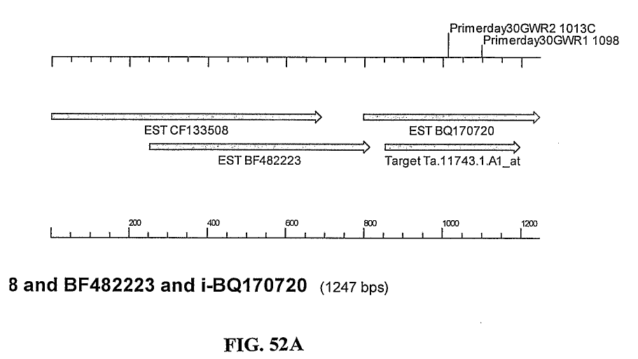

However, the target “Ta.11743.1.A1_at” shows weak similarity to a transcribed locus on the rice genome NP_{—}001061696.1. The weak similarity (13 for 27 bp) of the target “Ta.11743.1.A1_at” to the transcribed locus NP_{—}001061696.1 corresponds to the locus NC_{—}008401 on the rice genome. This region shows three open reading frames; one in the sense strand and two in the complement strand. The open reading frame corresponding to the sense strand spans 1 bp to 1163 bp and 2125 by to 2296 bp, indicating the presence of one intron (FIG. 51). This open reading frame is noted to be a “Cyclin-like F-box domain” containing protein, where the F-box domain is approximately 50 amino acids long, and is usually found in the N-terminal half of a variety of proteins. Two motifs that are commonly found associated with the F-box domain are the leucine rich repeats and the WD repeat. The F-box domain has a role in mediating protein-protein interactions in a variety of contexts, such as polyubiquitination, transcription elongation, centromere binding and translational repression.

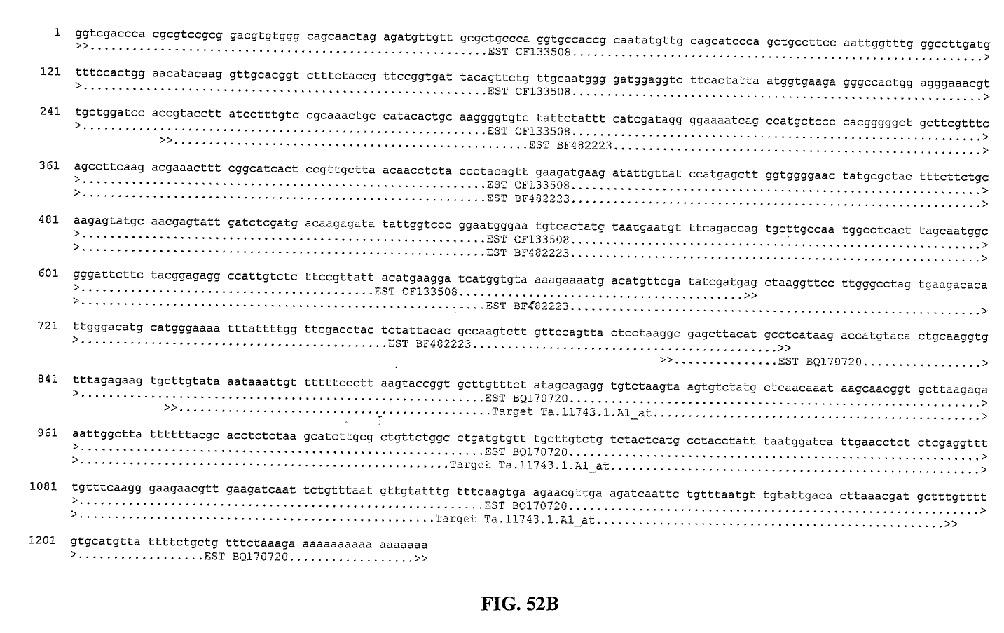

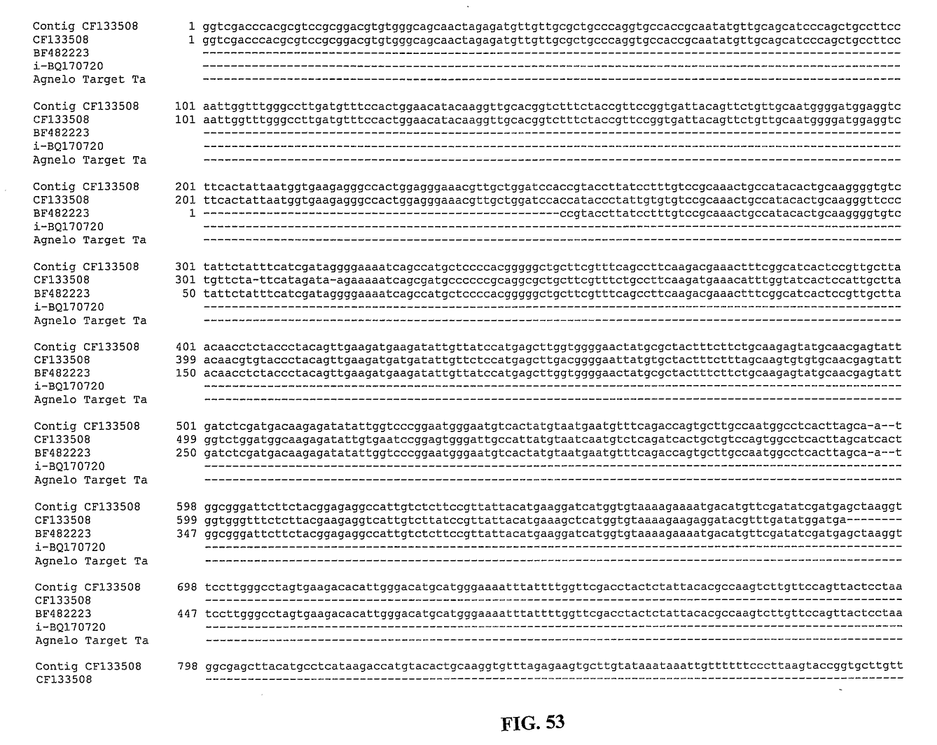

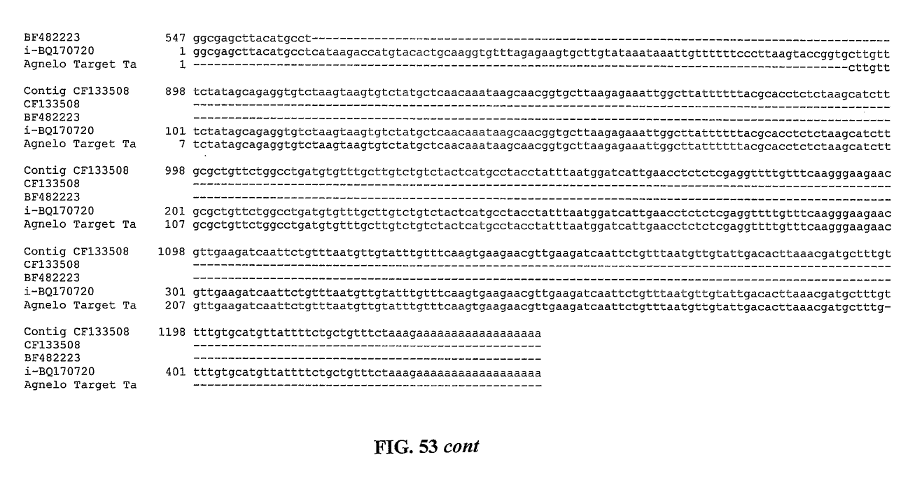

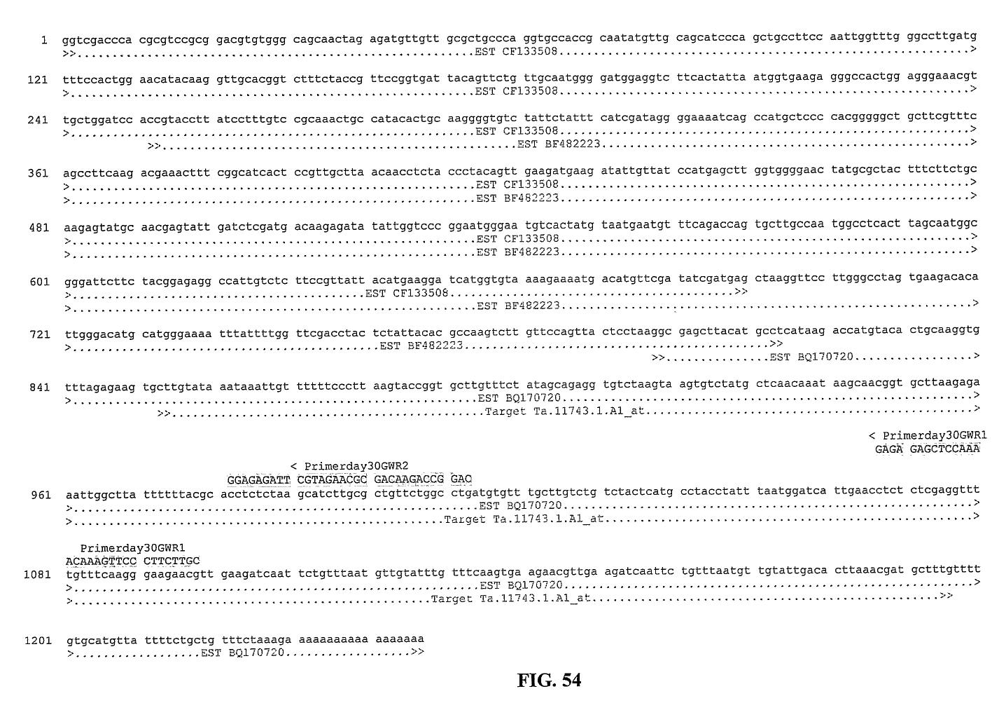

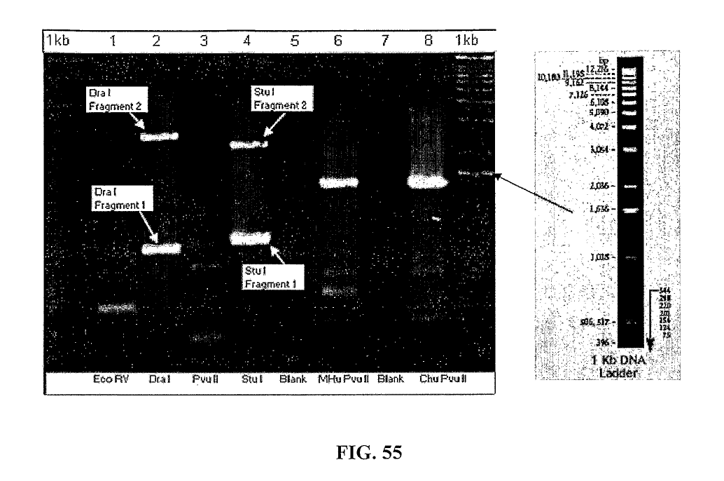

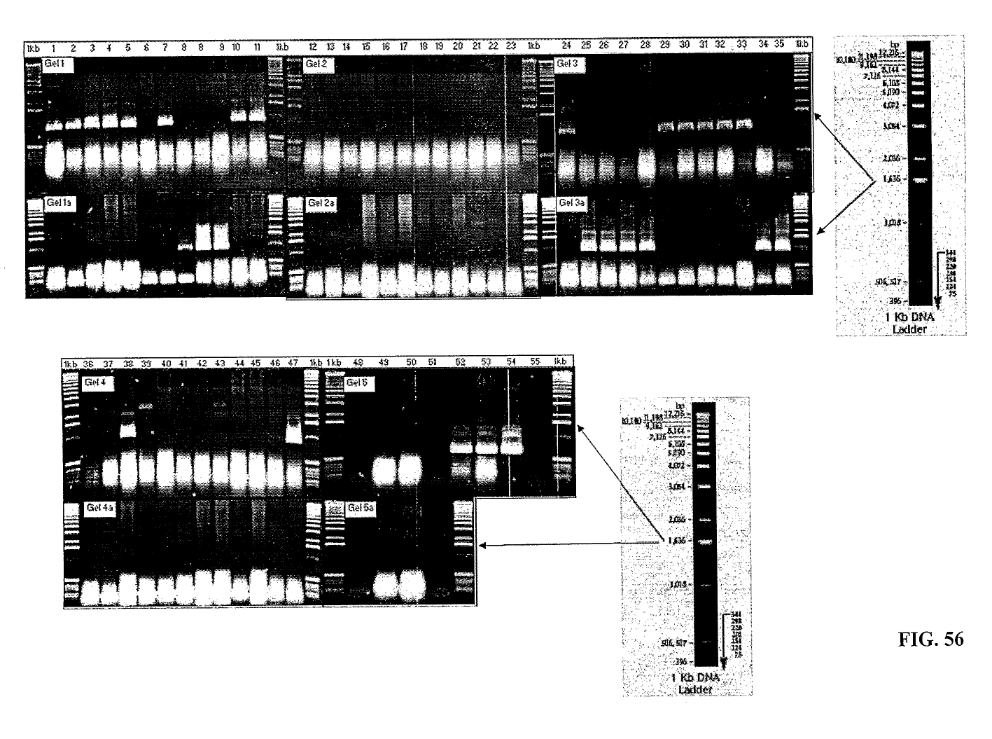

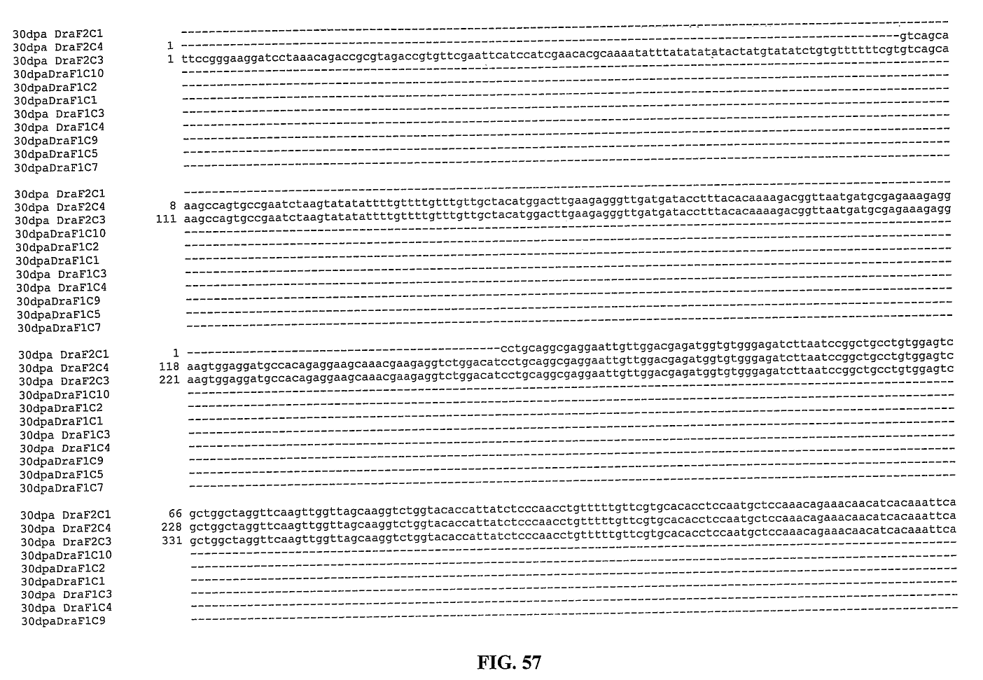

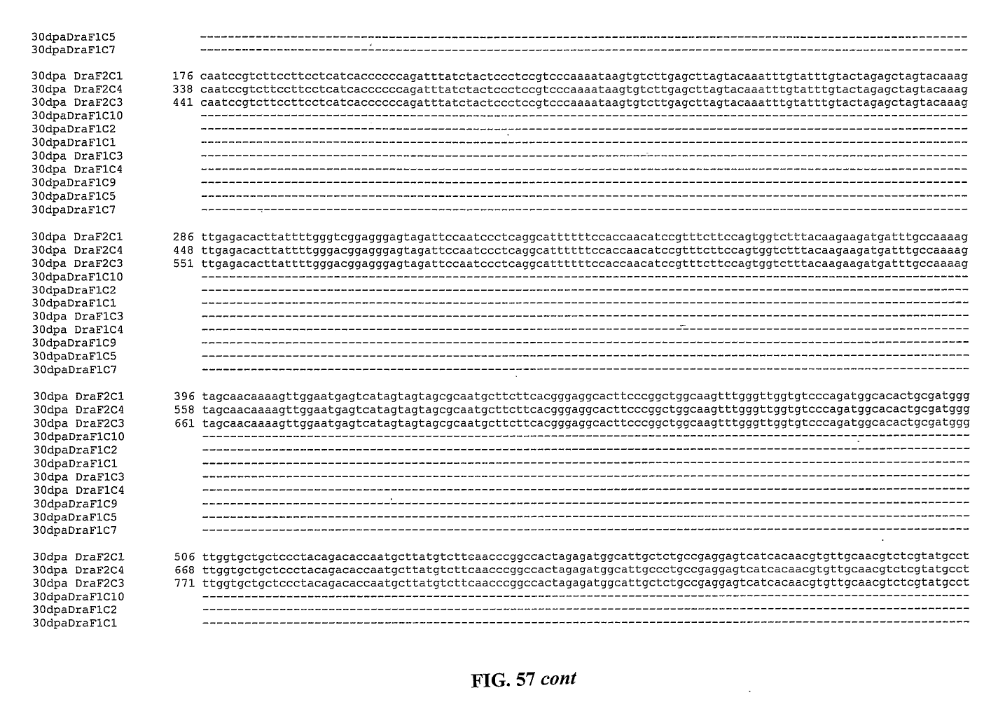

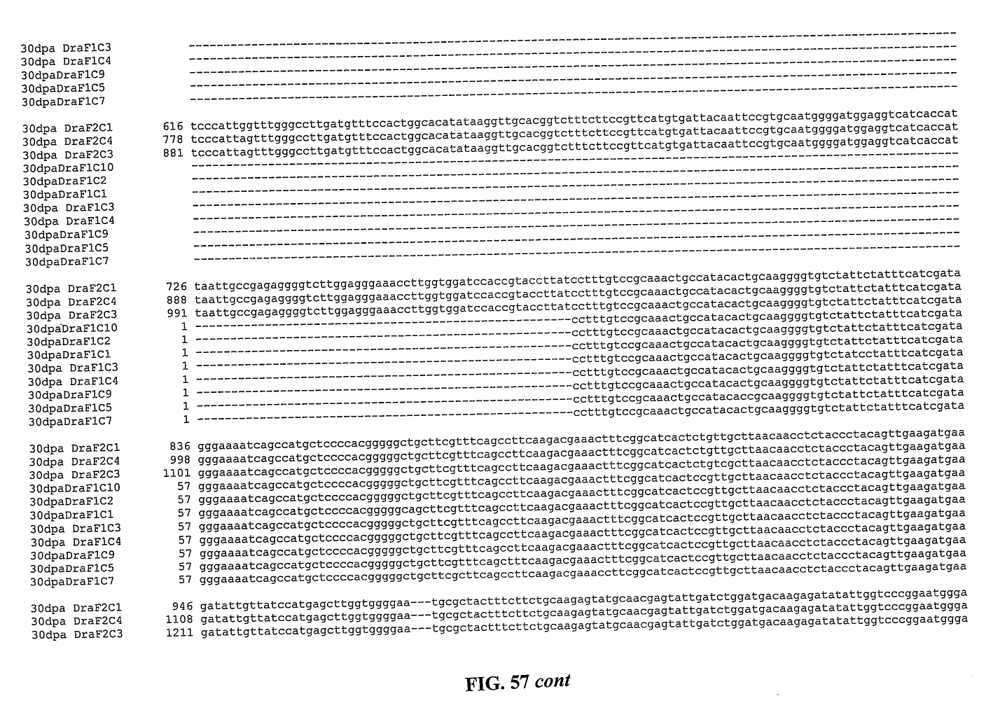

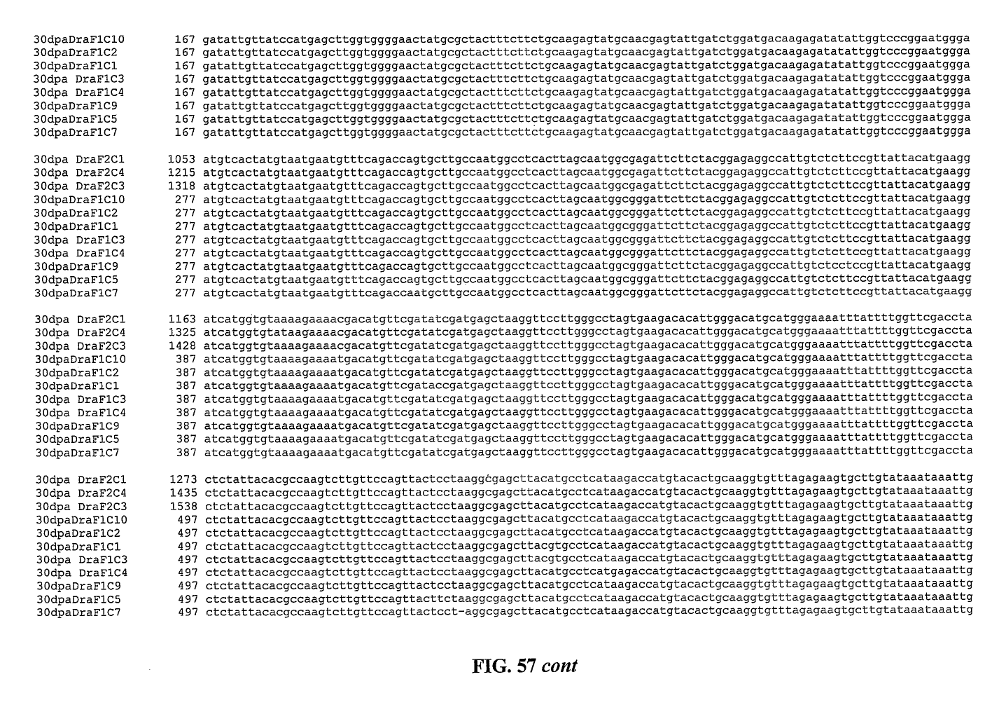

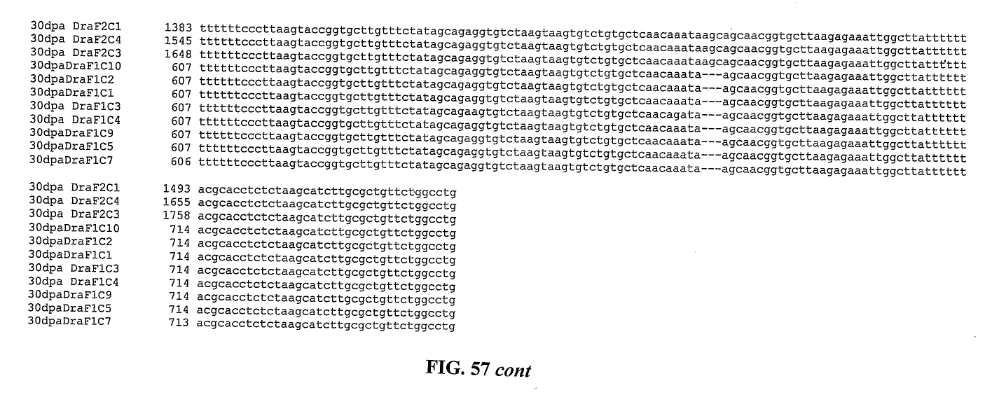

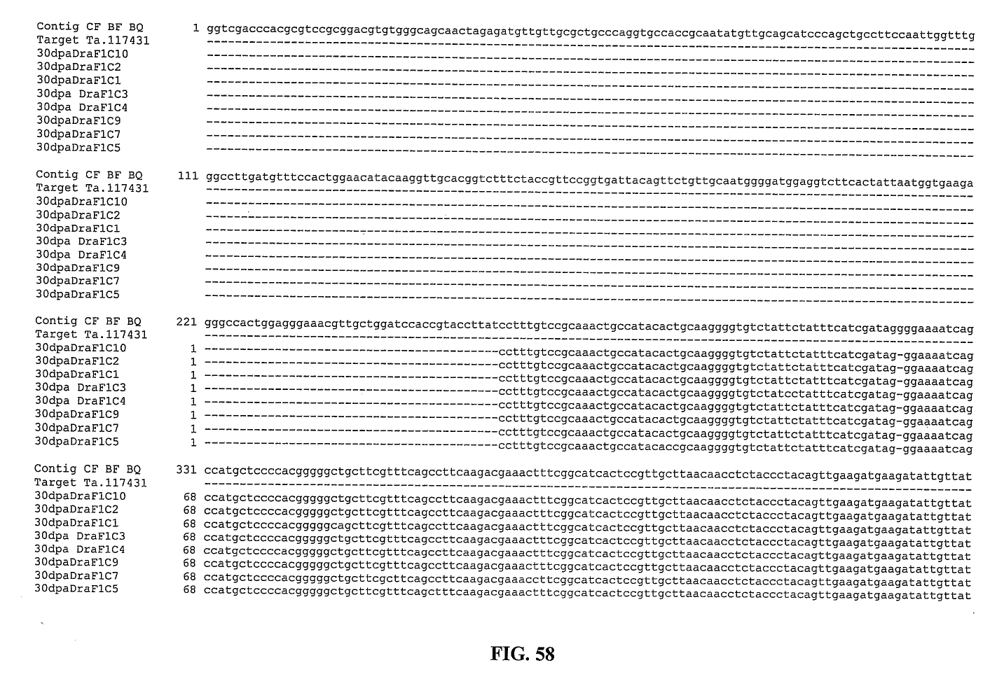

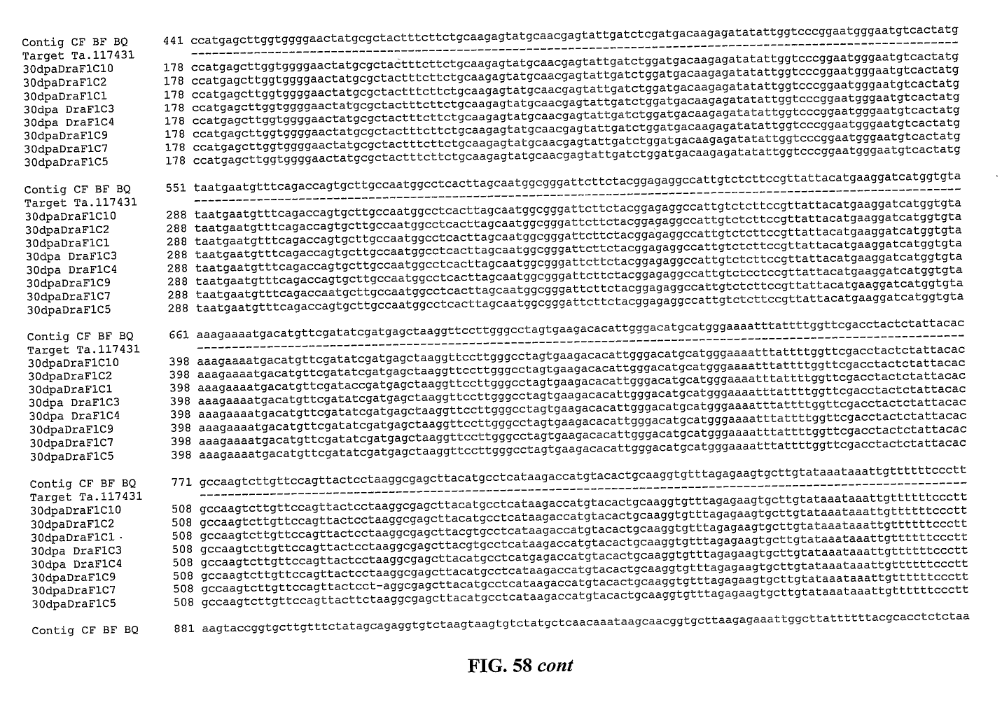

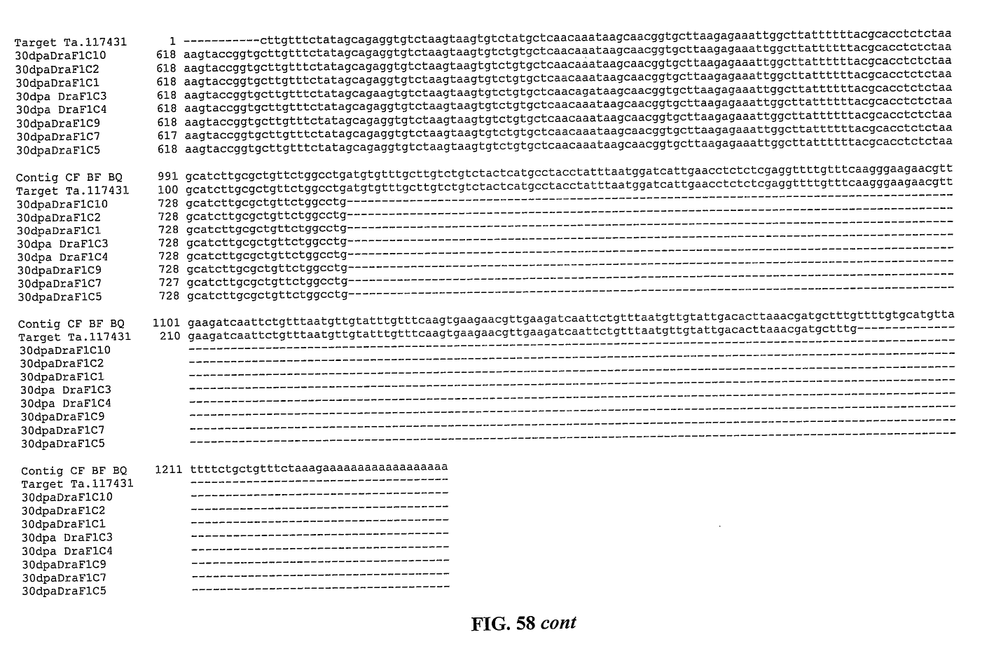

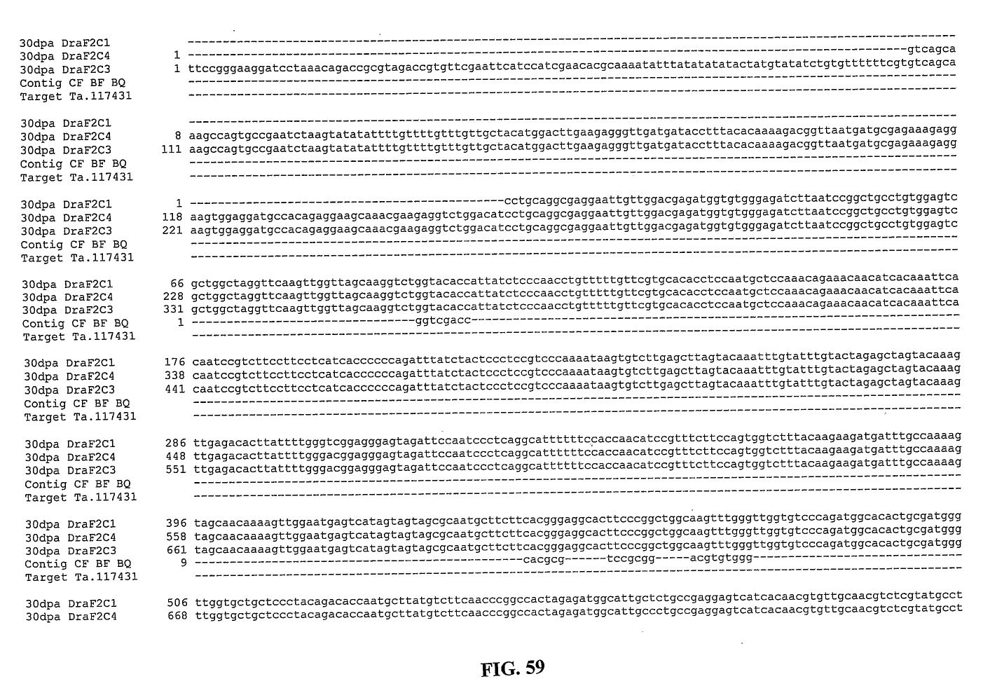

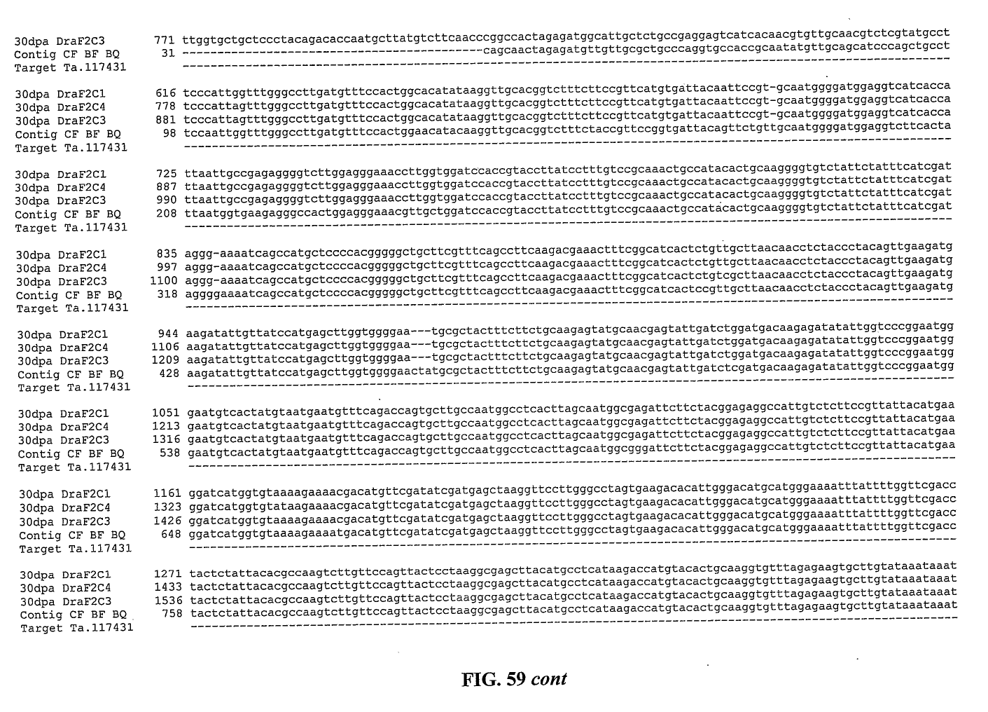

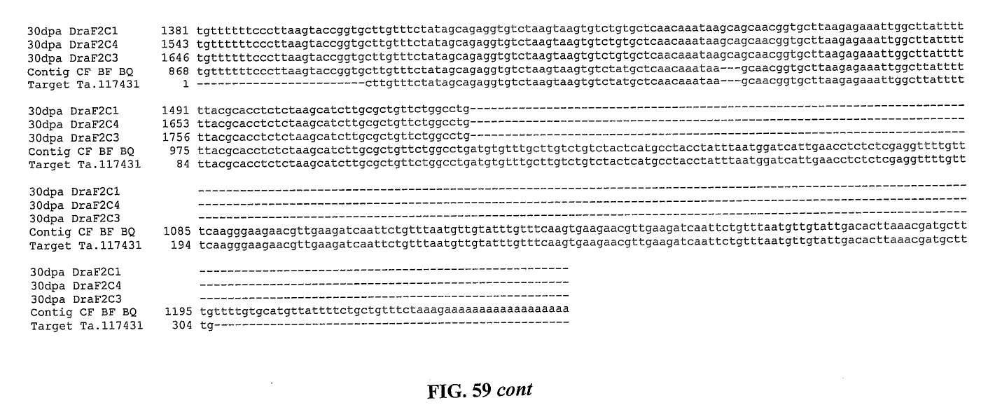

The 5′ end of the EST BQ170720 which contains the target “Ta.11743.1.A1_at” shows a perfect match over 17 by towards the 3′-end end of another wheat ESTs with locus ID BF482223. The EST BF482223 in turn shows strong similarity with another wheat EST with locus ID CF133508 (FIGS. 52A and B). The alignment between the “Ta.11743.1.A1_at”, BQ170720, BF482223 and CF133508 is, contig EST-CFBFBQ shown as a consensus sequence in FIG. 53. The consensus sequence as shown in FIG. 53 was used to design PCR primers (Primerday30GMR1 and Primer day 30GMR2) to amplify the 30 dpa gene fragments from wheat (FIG. 54). These primers were designed to isolate the upstream sequence which would correspond to the remainder of the gene sequence. The isolation of the upstream sequence was carried out using the Universal GenomeWalker kit (Clonetech, USA). Wheat genomic DNA was isolated and digested separately with Eco RV, Dra I, Pvu II and Stu I restriction to yield blunt ended fragments. Adaptors provided by the supplier of the kit were then ligated to both end of these blunt DNA fragments to obtain corresponding GenomeWalker libraries, namely, Eco RV-library, Dra I Library, Pvu II-library and Stu I-library. Primary PCR was carried out using primers Primer30GWR1 (CGTTCTTCCCTTGAAACAAAACCTCGAGAGAG) and AP1 (TAATACGACTCACTATAGGGC) using manufacturer's recommendations. The primary PCR was diluted to fifty times and 2 ul of this was taken in the secondary PCR (nested PCT) using primers Primer30GWR2 (CAGGCCAGAACAGCGCAAGATGCTTAGAGAGG) and AP2 (ACTATAGGGCACGCGTGGT) using manufacturer's recommendations. Two PCR fragments each were amplified with the Dra I and the Stu I libraries, and labelled as DraF1 and DraF2 fragments and StuF1 and StuF2 fragments. The approximate sizes of the DraF1 and DraF2 fragments are 0.7 Kb and 2.6 Kb respectively and StuF1 and StuF2 fragments are 0.8 Kb and 2.6 Kb respectively (FIG. 55). The Dra and Stu-fragments were ligated into pGEMT-easy, a TA cloning vector (Promega, USA), and cloned into JM109 cells (Promega, USA). Several white colonies were screened by PCR using Primer30GWR2 (CAGGCCAGAACAGCGCAAGATGCTTAGAGAGG) and M13 reverse (CACACCGGAAACAGCTATGACC) (FIG. 56) to identify recombinant colonies. Eight colonies each corresponding to the DraF1 and StuF1, and four colonies each corresponding to DraF2 and StuF2 were selected for plasmid preparation and sequencing. Sequence of clones corresponding to DraF1 (C1 to C8) and DraF2 (C1, C3, C4) and were found to show high homology to each other (FIG. 57). All 30dpa DraF1 fragments show high homology to the contig EST-CFBFBQ (CF133508, BF482223 and BQ170720) and the target Ta.11743.1 sequence (FIG. 58). Similarly, all 30dpa DraF2 fragments show high homology to the contig EST-CFBFBQ (CF133508, BF482223 and BQ170720) and the target Ta.11743.1 sequence (FIG. 59).

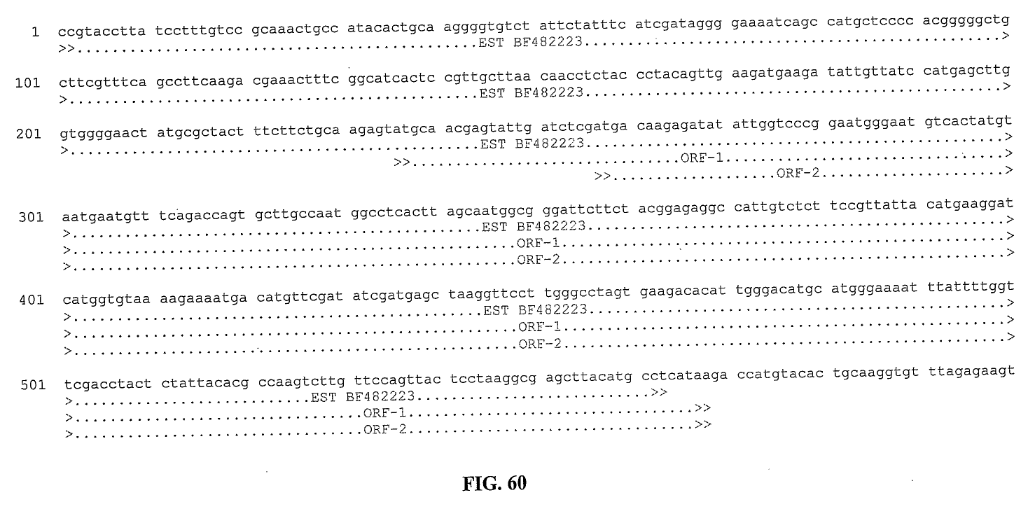

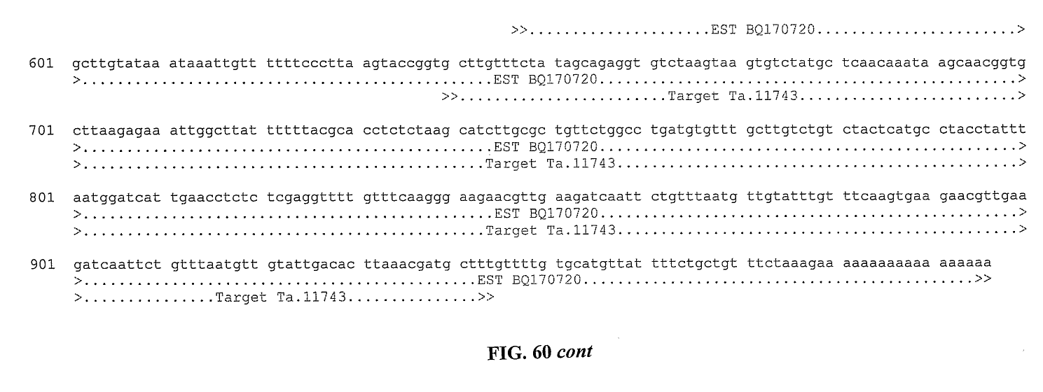

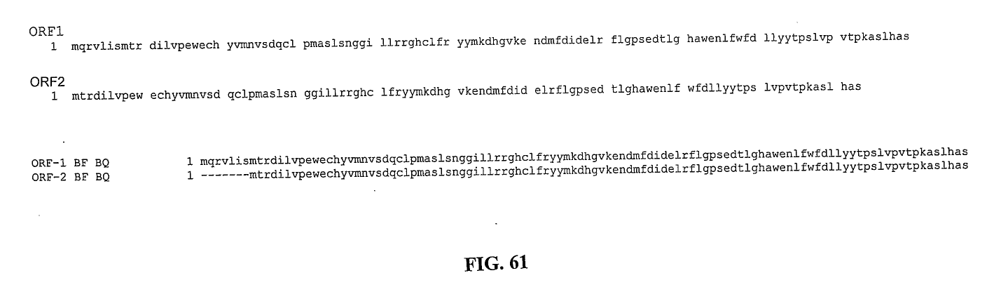

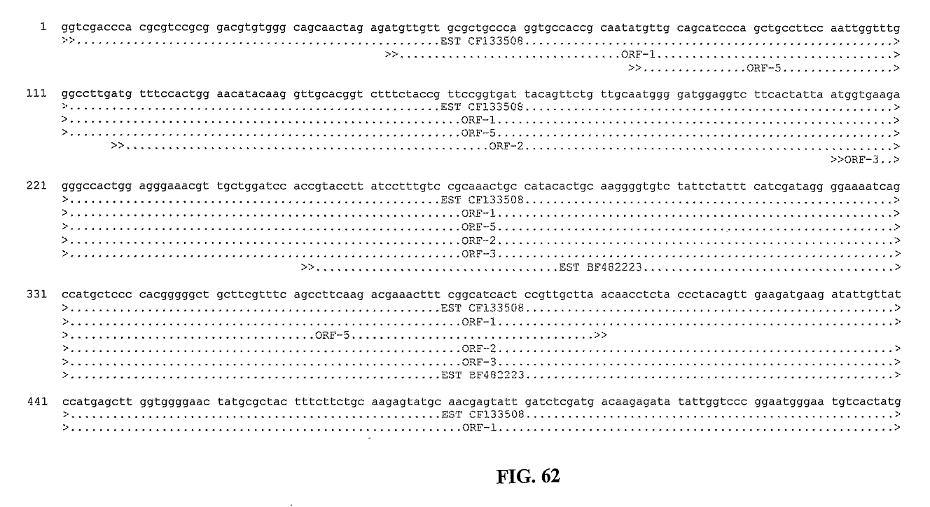

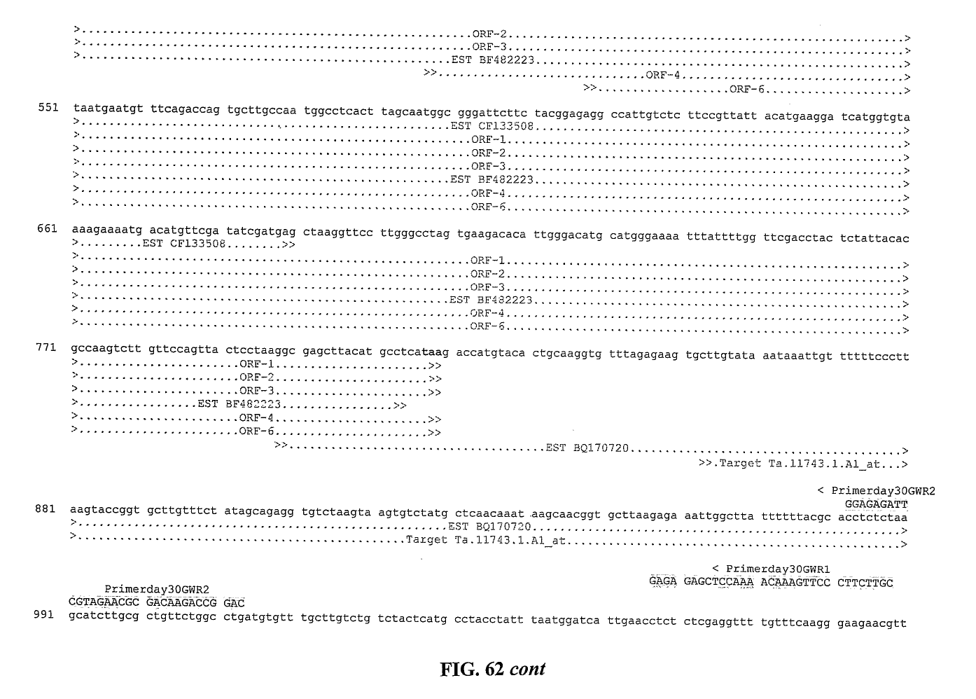

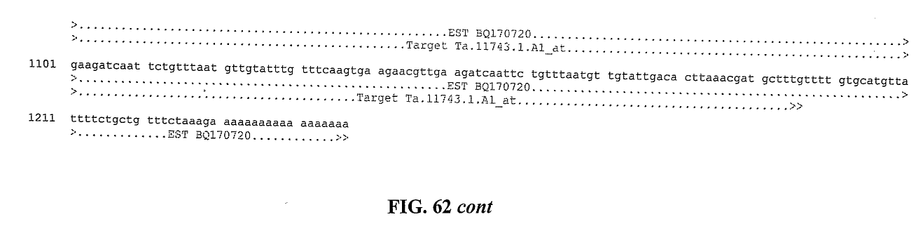

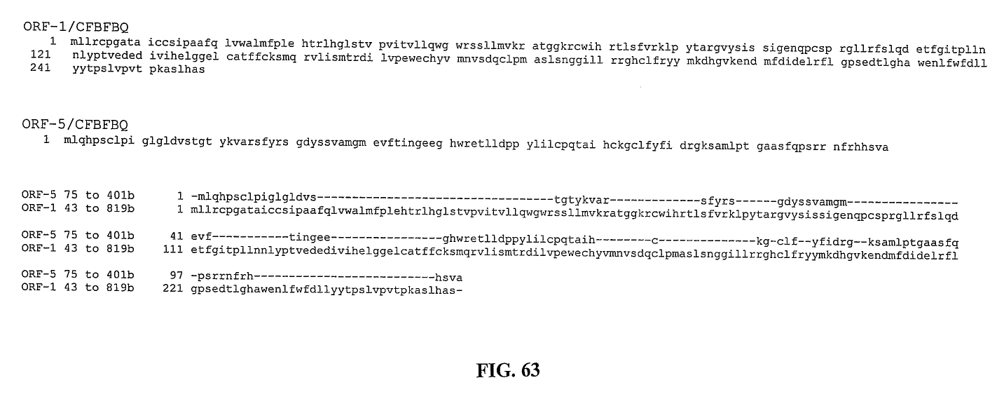

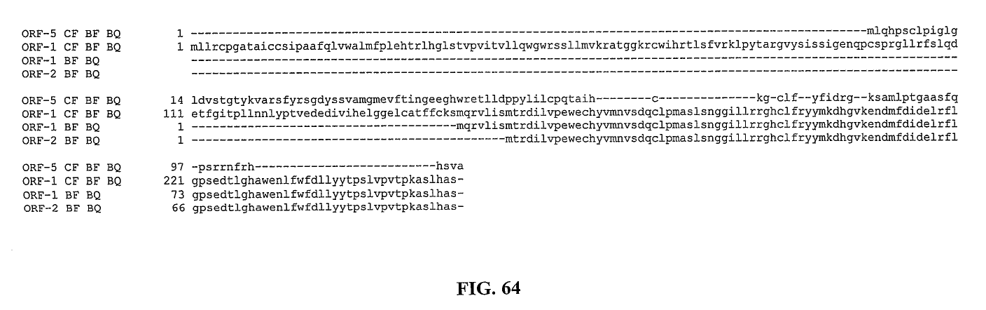

The next step was to determine the open reading frame/s (ORFs) on the DraF2 fragments. To ascertain this, we first determined the ORFs on the contig EST-BFBQ (BF482223 and BQ170720) and the contig EST-CFBFBQ (CF133508, BF482223 and BQ170720). Two ORFs, ORF-1/BFBQ and ORF-2/BFBQ were found on the contig EST-BFBQ (FIG. 60). The ORF-1/BFBQ is longer than the ORF-2/BFBQ but showed complete homology to each other at the overlap as they were in frame to each other and showed good homology (FIG. 61). The contig EST-CFBFBQ showed a total of 6 ORF in the sense direction ORF-1/CFBFBQ, ORF-2/CFBFBQ, ORF-3/CFBFBQ, ORF-4/CFBFBQ, ORF-5/CFBFBQ and ORF-6/CFBFBQ (FIG. 62). The OFR-1/CFBFBQ was found to be in frame and was longer than the ORF-2/CFBFBQ, ORF-3/CFBFBQ, ORF-4/CFBFBQ and ORF-6/CFBFBQ. The amino acid sequence and the alignments of ORF-1/CFBFBQ and ORF-5/CFBFBQ are shown in FIG. 63 where both the ORFs show partial homology as they are not in frame to each other. The alignments of ORFs on contig EST-BFBQ and Contig EST CFBFBQ indicates that ORF-1/CFBFBQ, ORF-1/BFBQ and ORF-2/BFBQ show high homology and that ORF-1/CFBFBQ is a longer protein sequence and is in frame with the other two EST (FIG. 64).

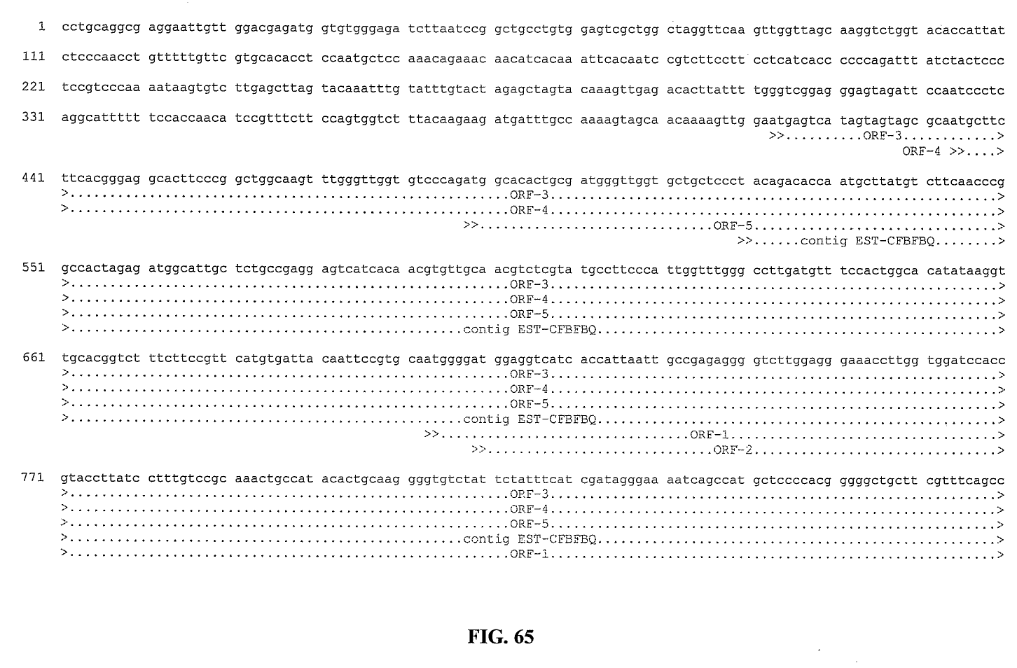

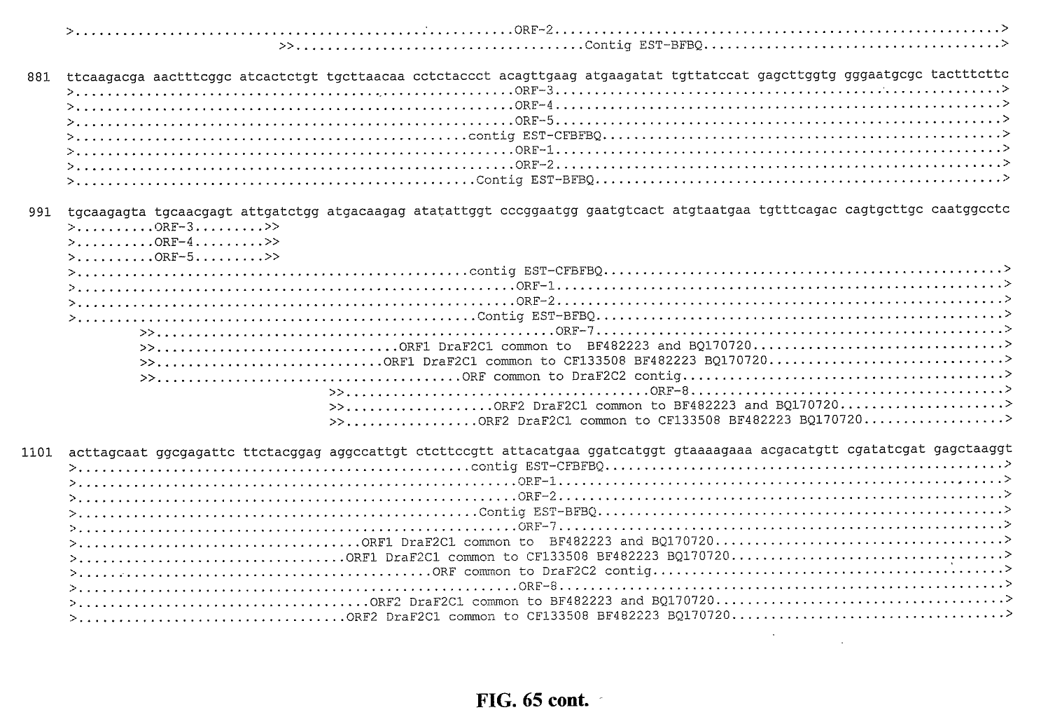

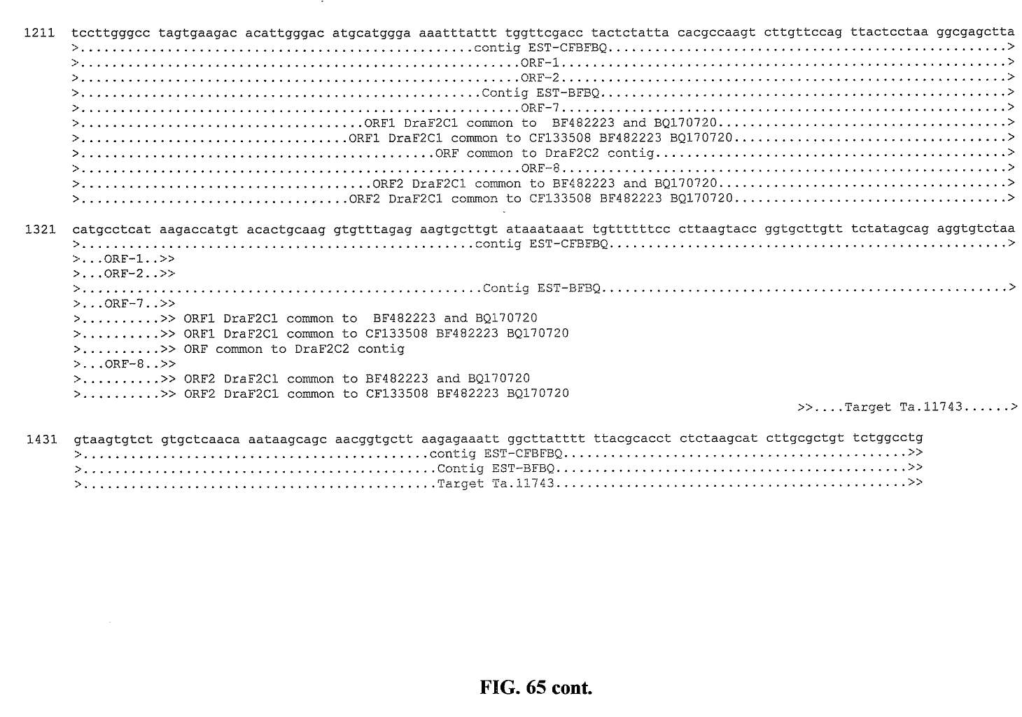

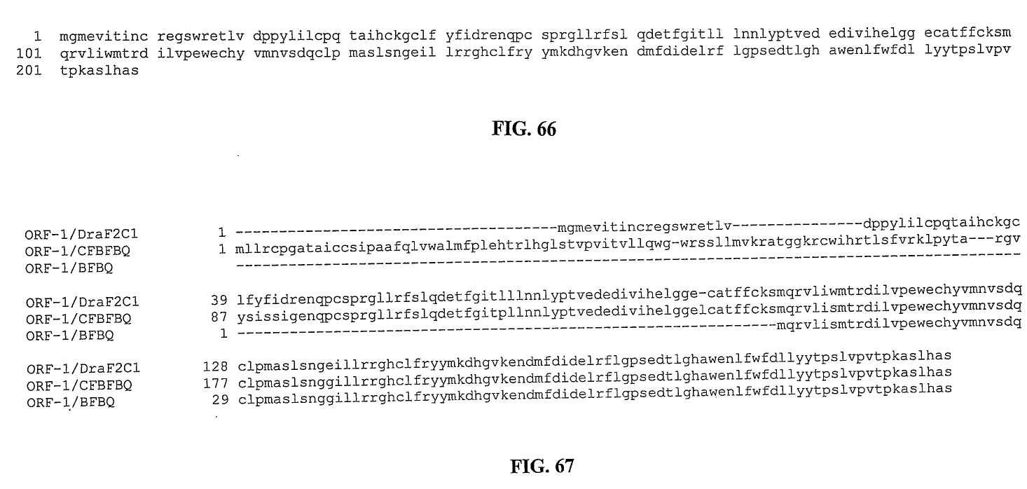

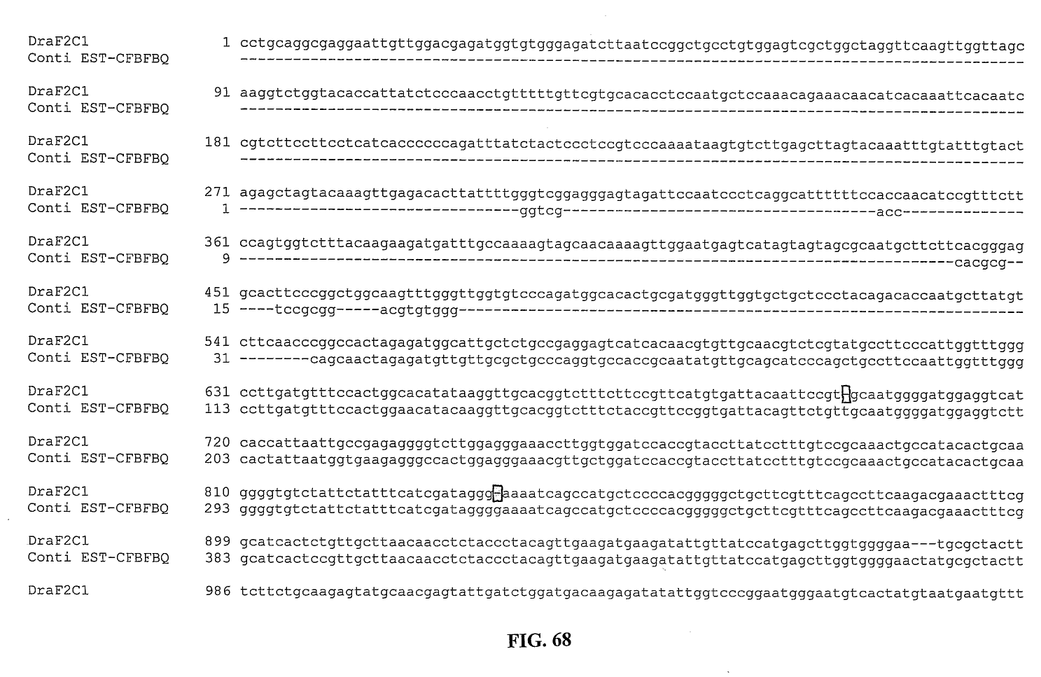

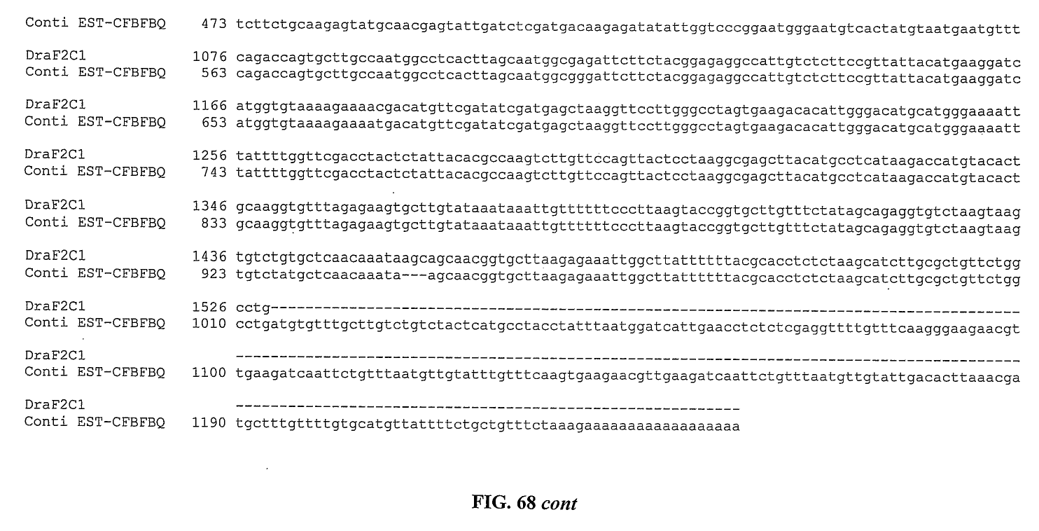

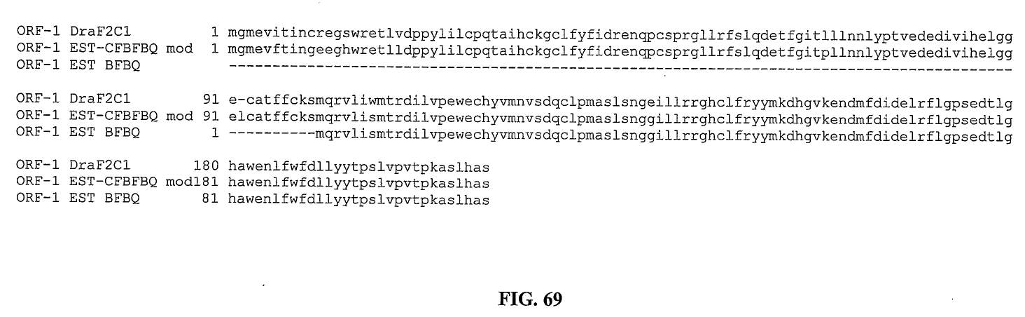

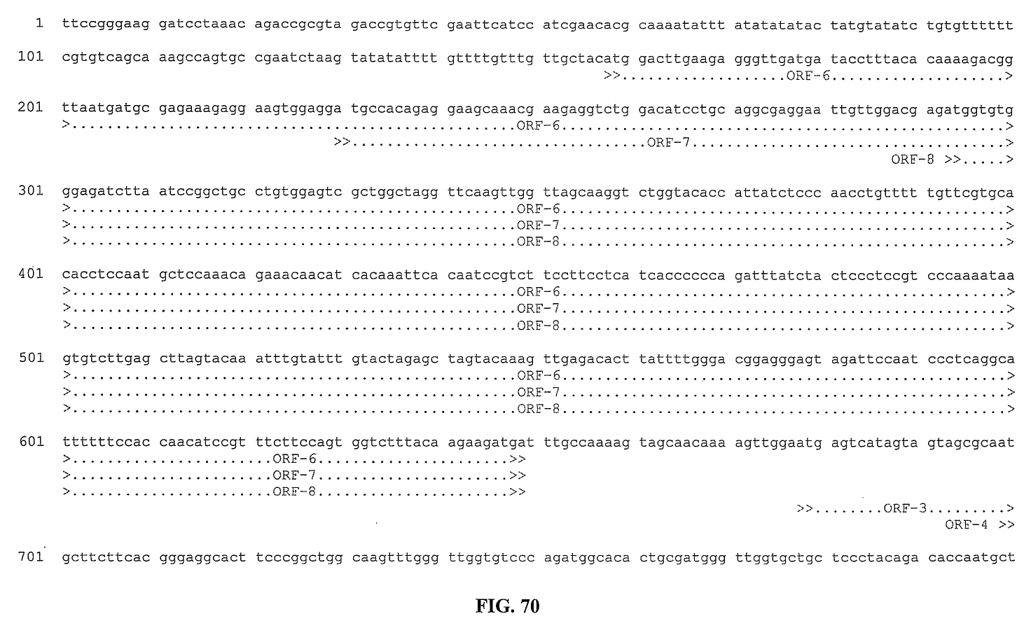

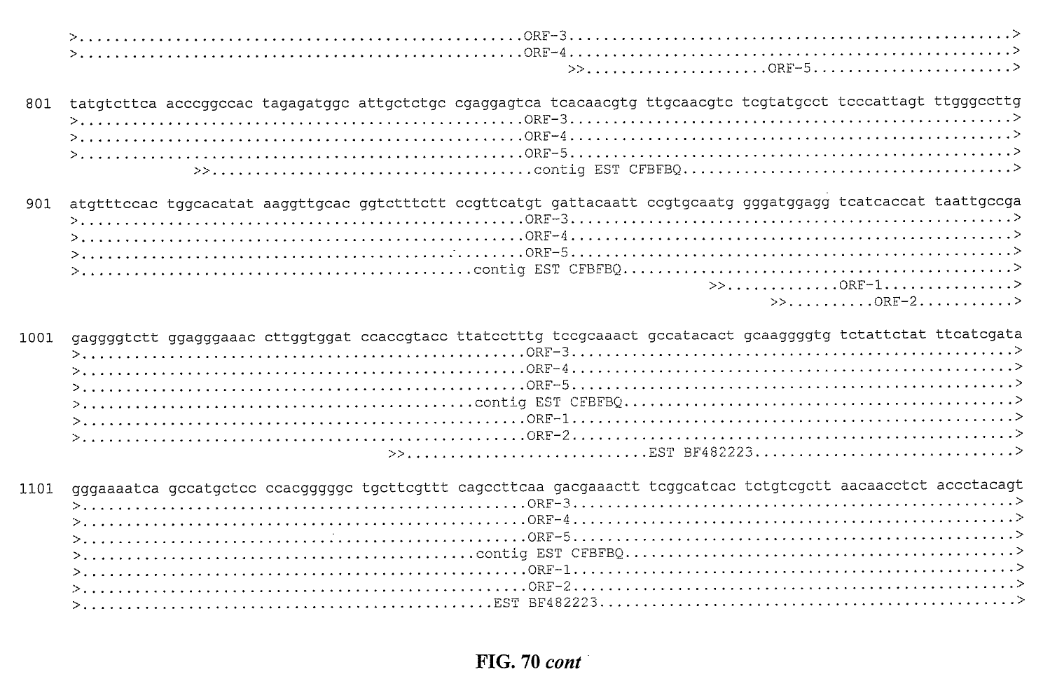

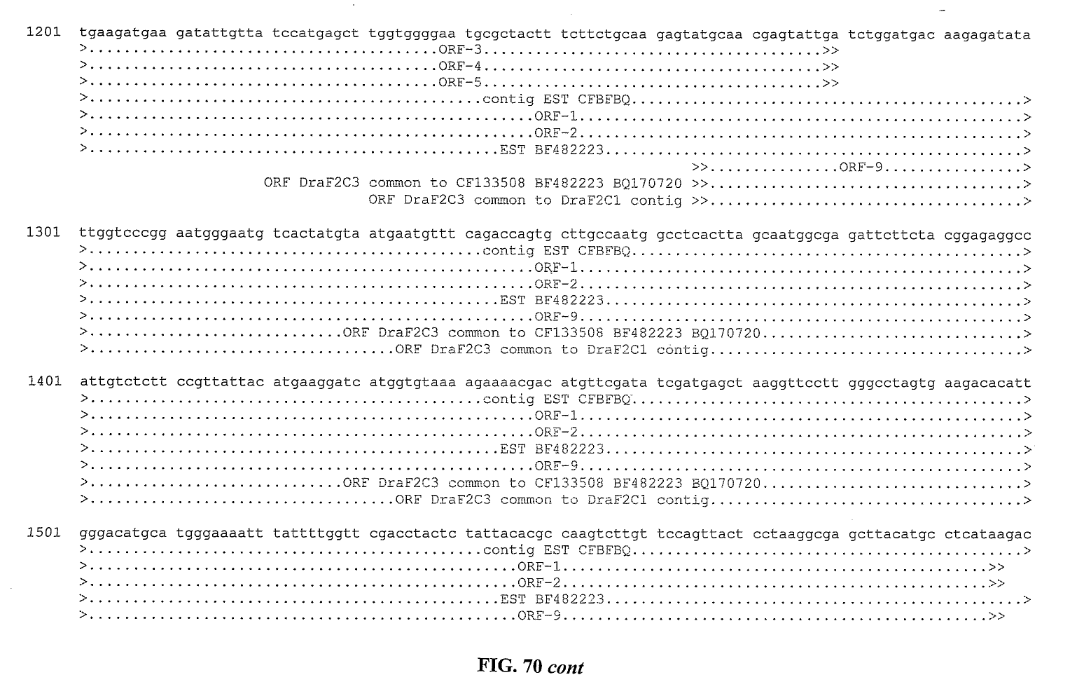

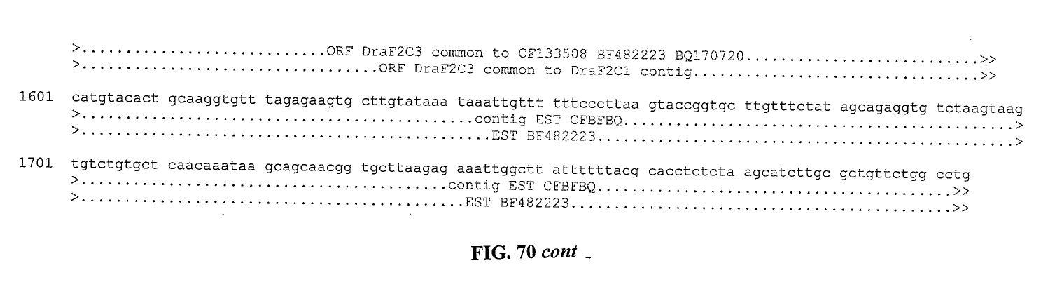

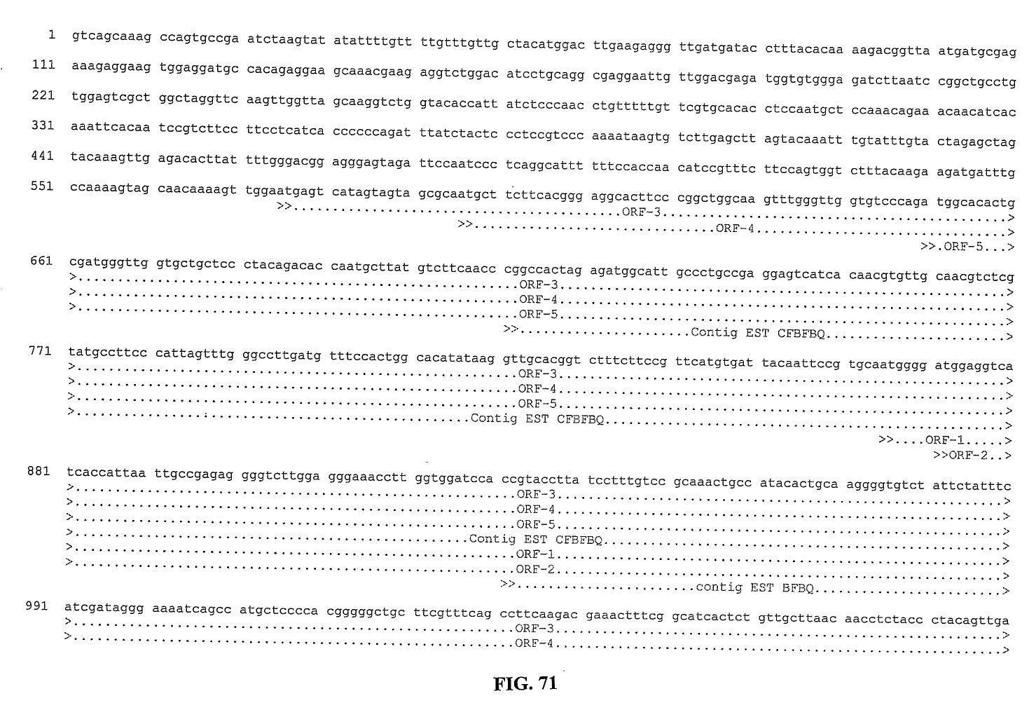

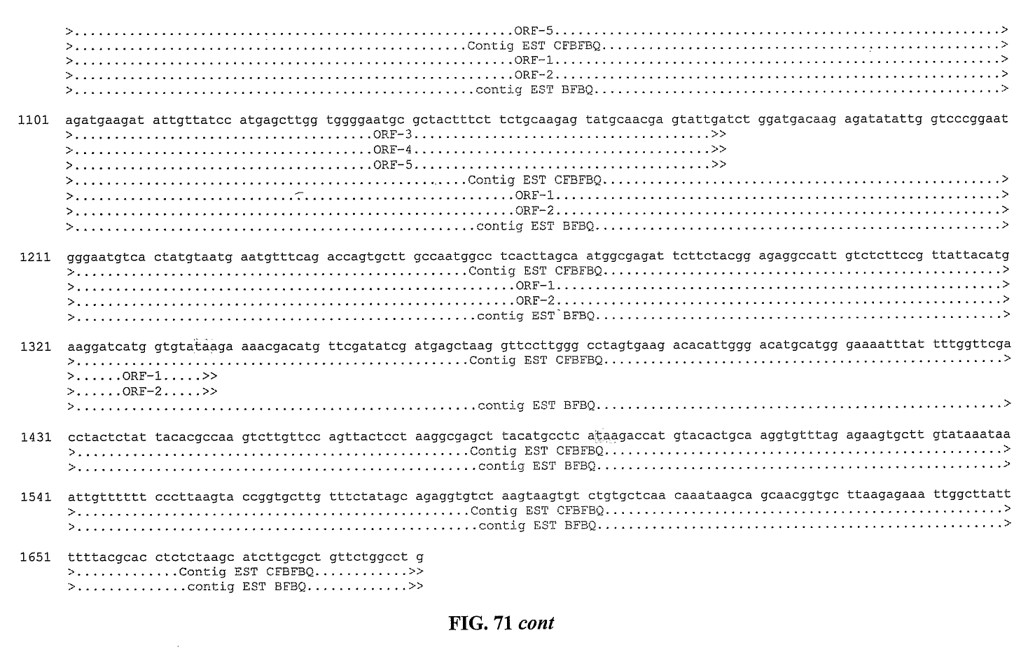

The next step was to determine the ORFs on the three DraF2 fragments, compare these to each other and to the ORFs on the contig EST-CFBFBQ. The DraF2C1 fragment shows a number of ORFs (FIG. 65), and where ORF-1/DraF2C1 is the longest and is in frame with the ORFs found on contig EST-BFBQ (ORF-1/BFBQ and ORF-2/BFBQ) and contig EST-CFBFBQ (ORF-5.CFBFBQ). The amino acid sequence of the ORF1-DraF2C1 is shown in FIG. 66 and the level of homology with ORF-1/BFBQ from contig EST-BFBQ, and between ORF-1/CFBFBQ form contig EST-CFBFBQ is shown in FIG. 67. As shown in FIG. 67, the homology between the ORF1-DraF2C1 and the ORF-1/CFBFBQ is significant for the entire length except the first part of the sequence. We feel this lack of homology in the first part of the gene is due to the presence of two indels in the contig EST-CFBFBQ (shown as a boxed region in FIG. 68) which are possible sequencing errors in the EST CF133508 and not in the DraF2C1 clone as we have checked our sequence for sequencing errors. Once these two indels are removed in the EST-CFBFBQ, an ORF matching the ORF1/DraF2C1 in sequence and length is located on the contig EST-CFBFBQ (FIG. 69). This result demonstrates that the ORF1/DraF2C1 located on the 30 dpa gene fragment DraF2C1 is most likely to be the true ORF. The ORF1/DraF2C1 is also located on the 30dpa DraF2C3 fragment (FIG. 70). However, on the 30dpa DraF2C4 fragment the, ORF1/DraF2C1 is present but in a truncated form due to a mutation in the gene (at position where T is replaced by A) leading to a stop codon being generated (see shaded regions) (FIG. 71).

### Example 9

### Isolated Wheat 30 dpa Gene Sequences

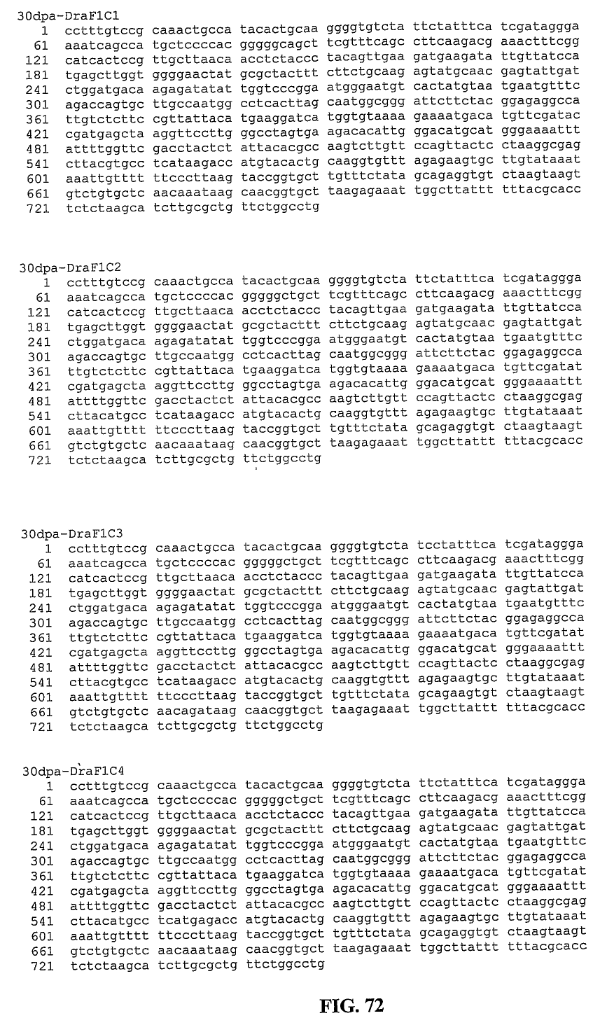

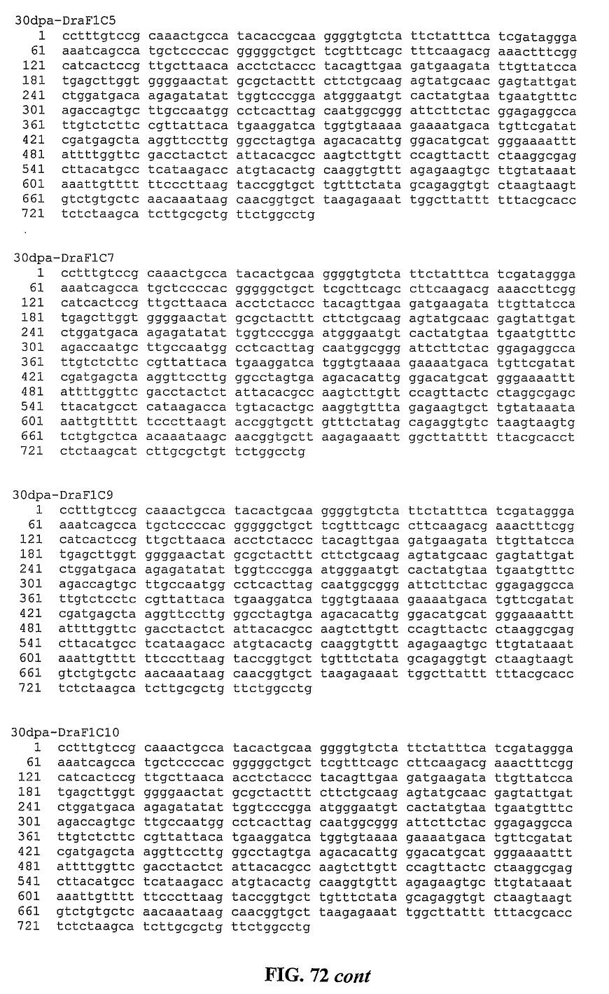

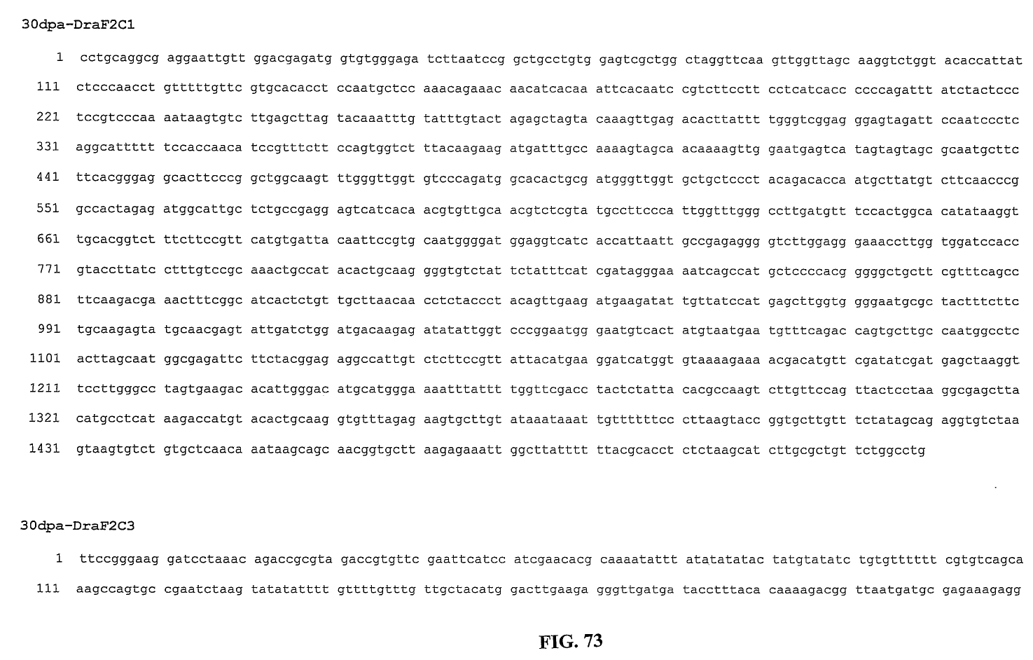

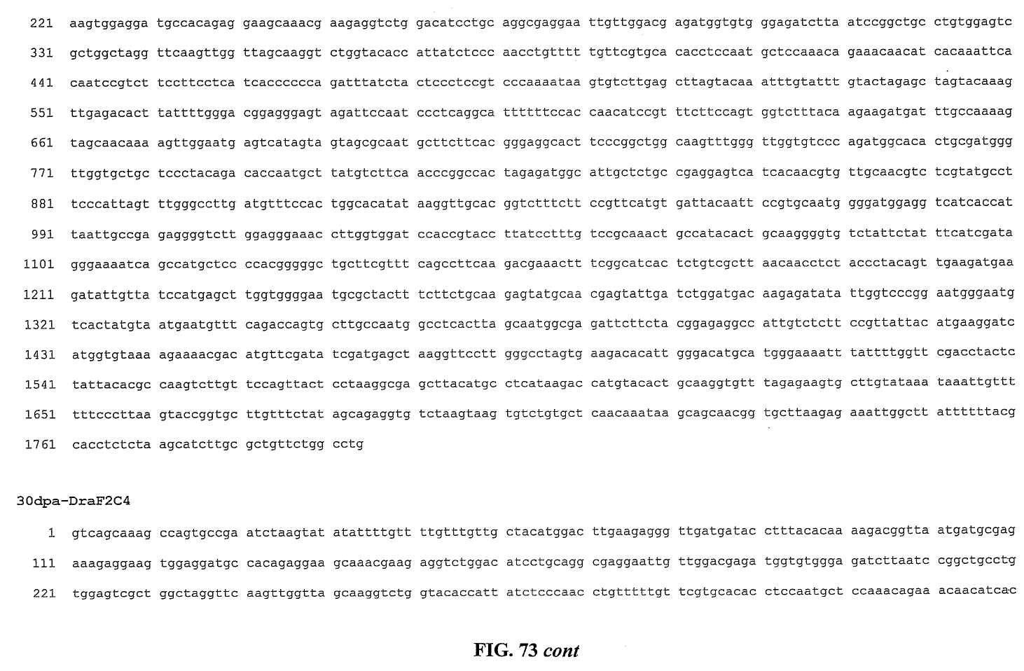

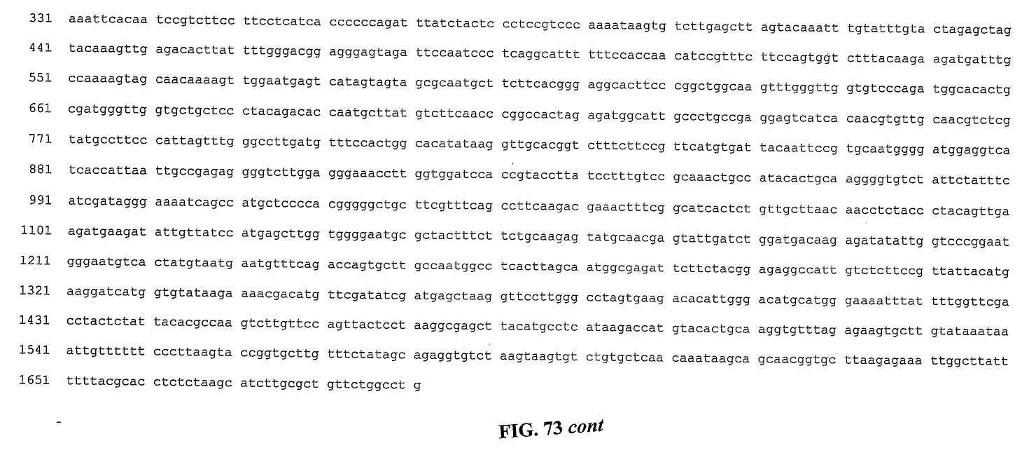

The 30dpa gene sequences were isolated as two fragments, DraF1 and DraF2 fragments. The sequences of variants of the DraF1 fragments DraF2-C1, DraF2-C2, DraF2-C3, DraF2-C4, DraF2-05, DraF2-C7 and DraF2-C10 are shown in FIG. 72. The sequence variants of 30dpa-DraF2 fragments DraF2C1, DraF2C3 and DraF2C4 are shown in FIG. 73.

### Example 10

### Comparison Between Wheat 30dpa Gene Sequences and Other Plant Coding Sequences

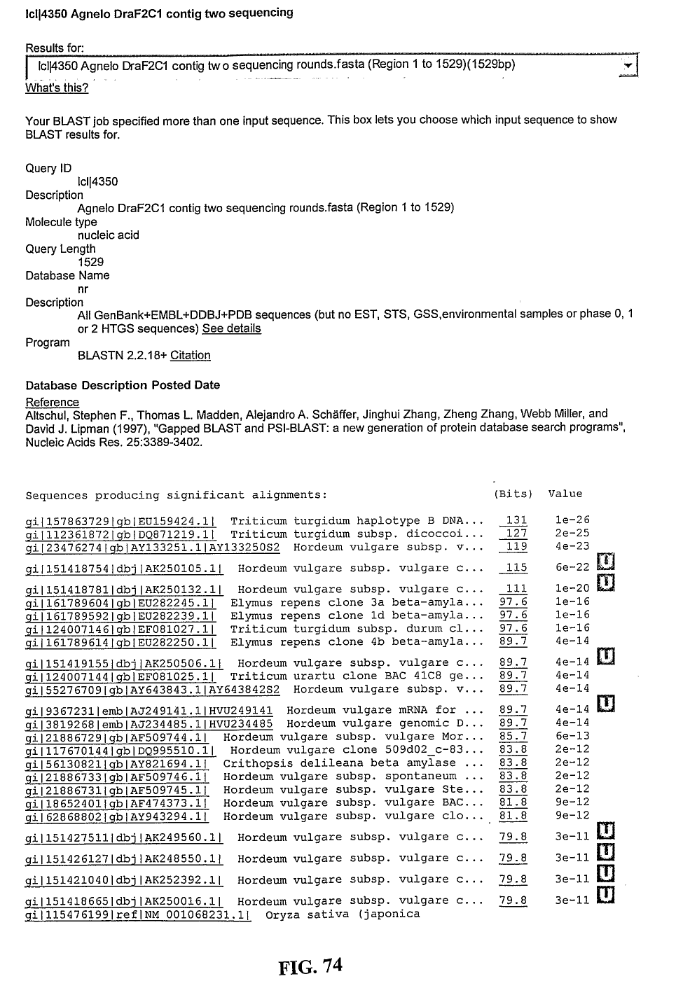

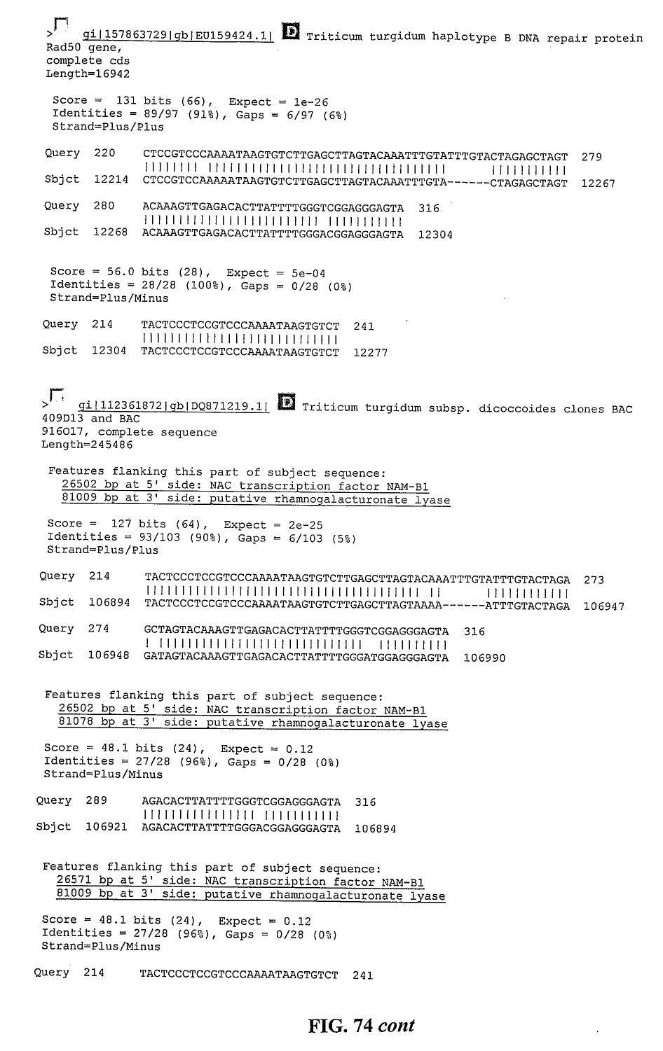

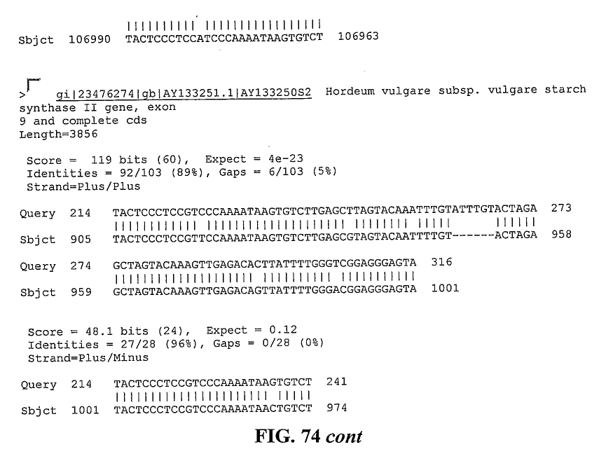

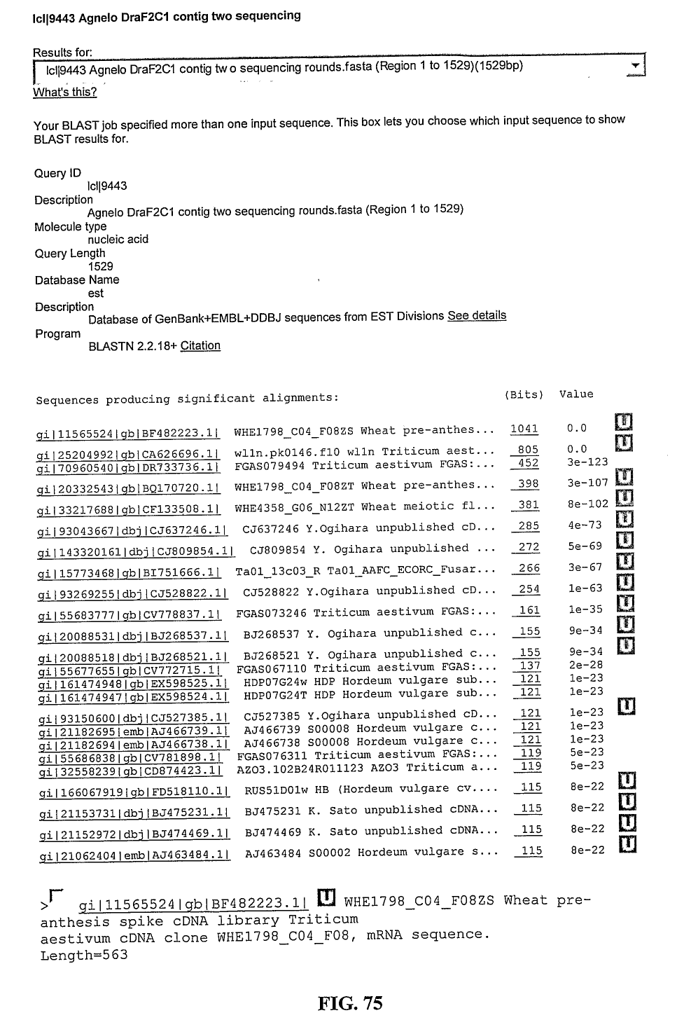

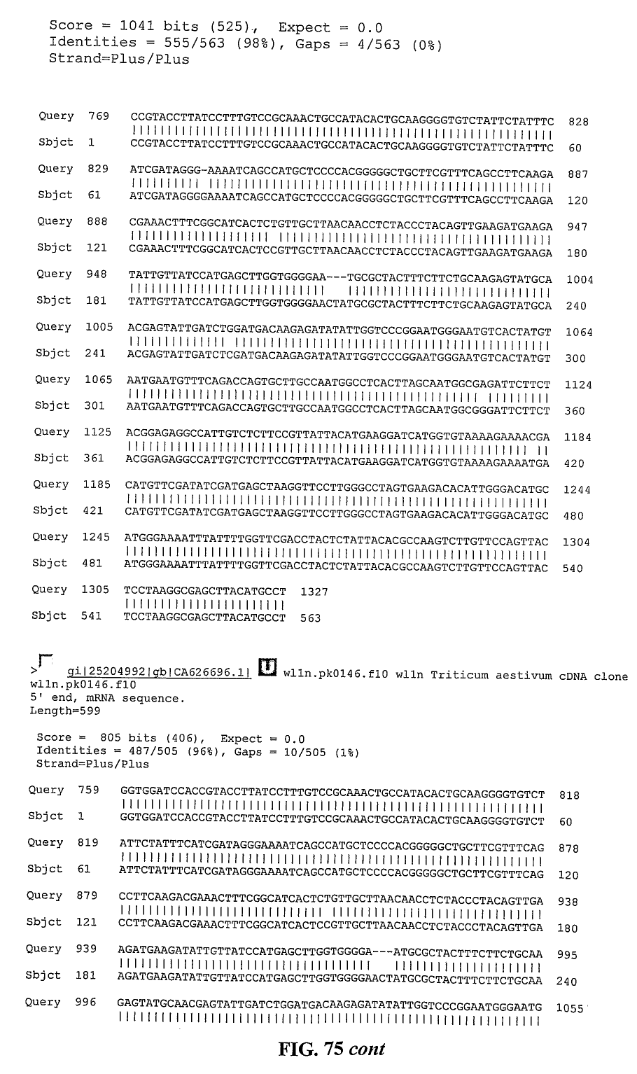

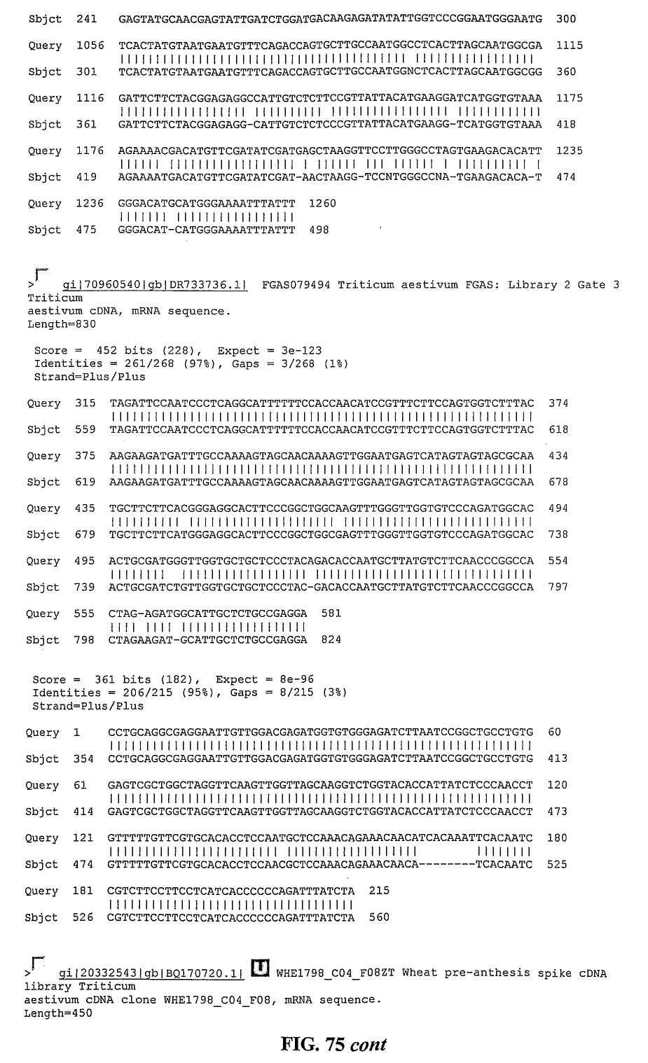

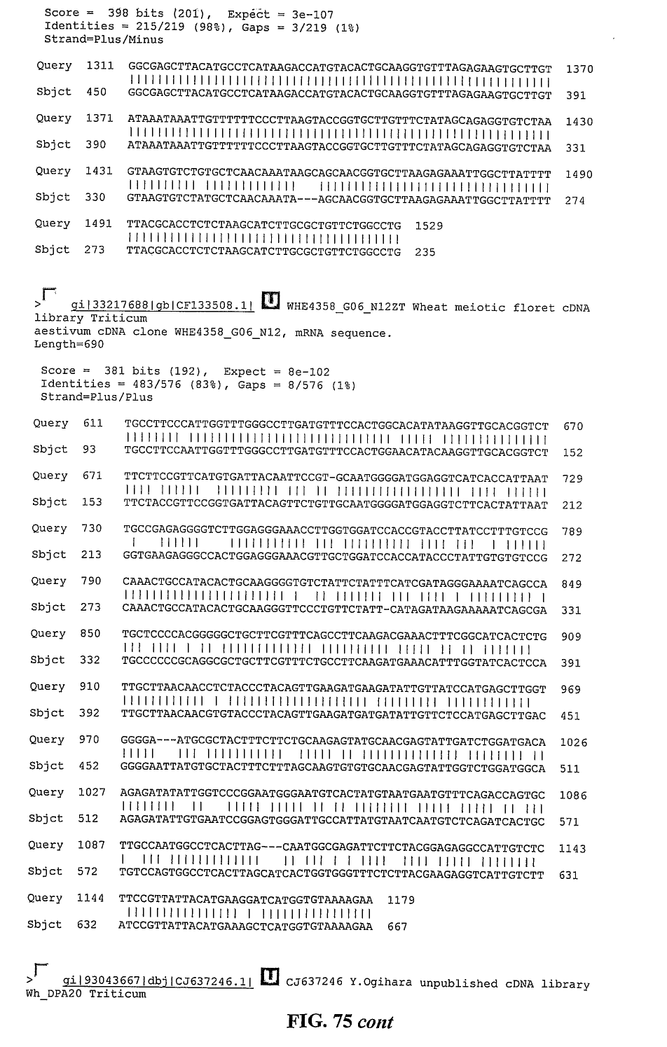

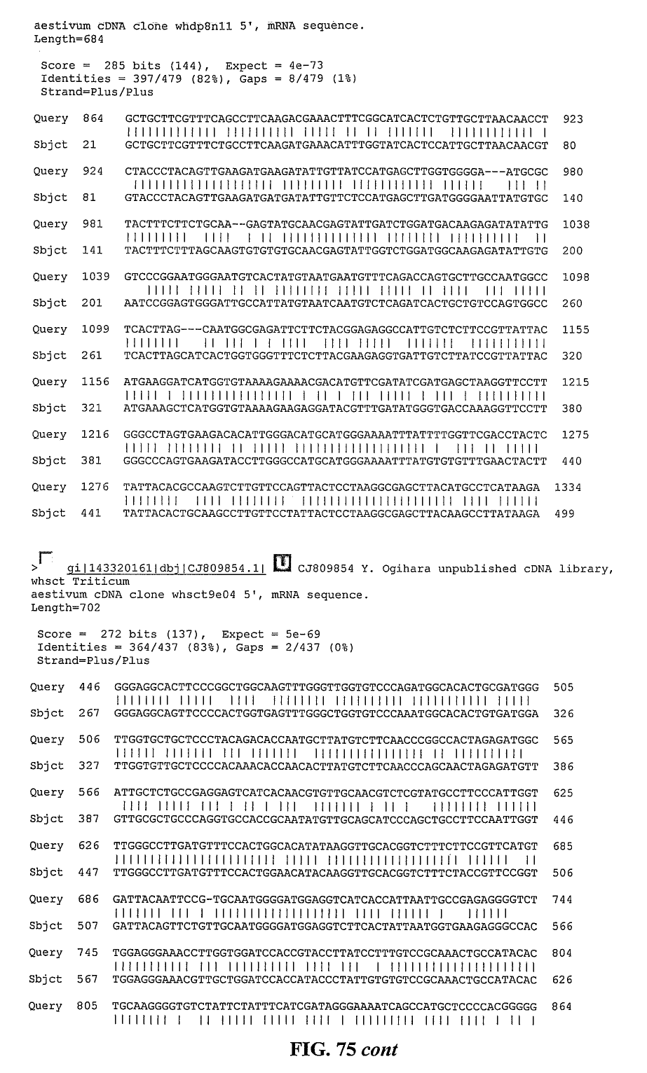

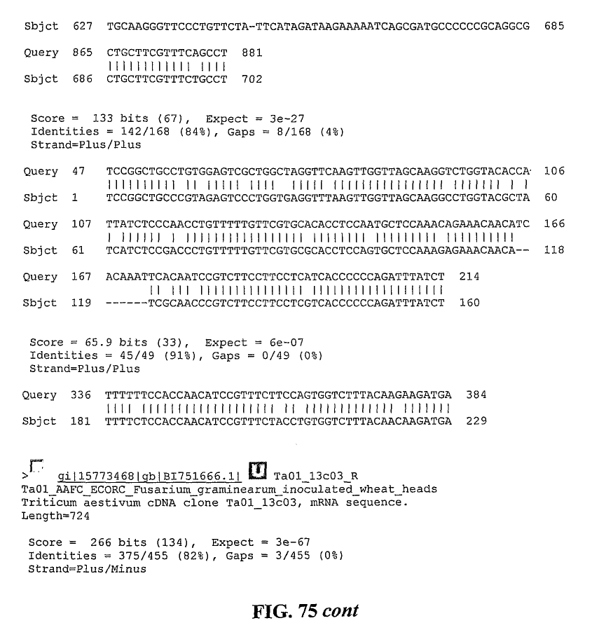

The isolated wheat genomic sequence of the 30dpa gene corresponding to DraF2C1 was subjected to BLAST analysis on the NCBI portal for non redundant DNA (nr-DNA) and for ESTs.: http://blast.ncbi.nlm.nih.gov/Blast.cgi. Results of nr-DNA BLAST analysis with alignments for the first few hits are shown in FIG. 74. Results of the ESTBLAST analysis with alignments for the first 4 hits are shown in FIG. 75.

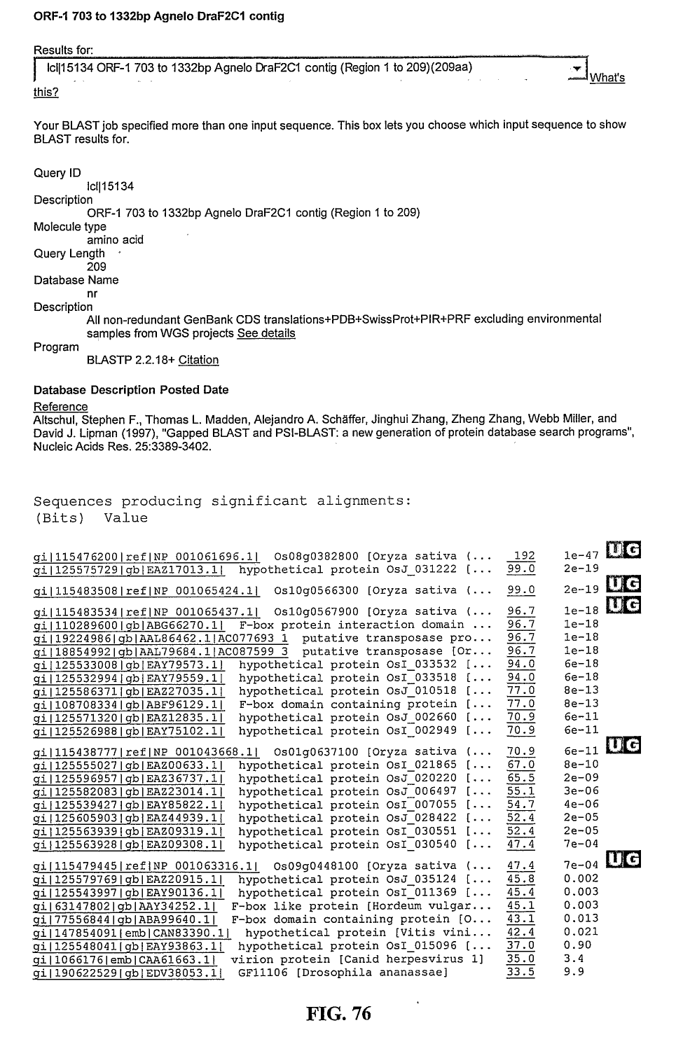

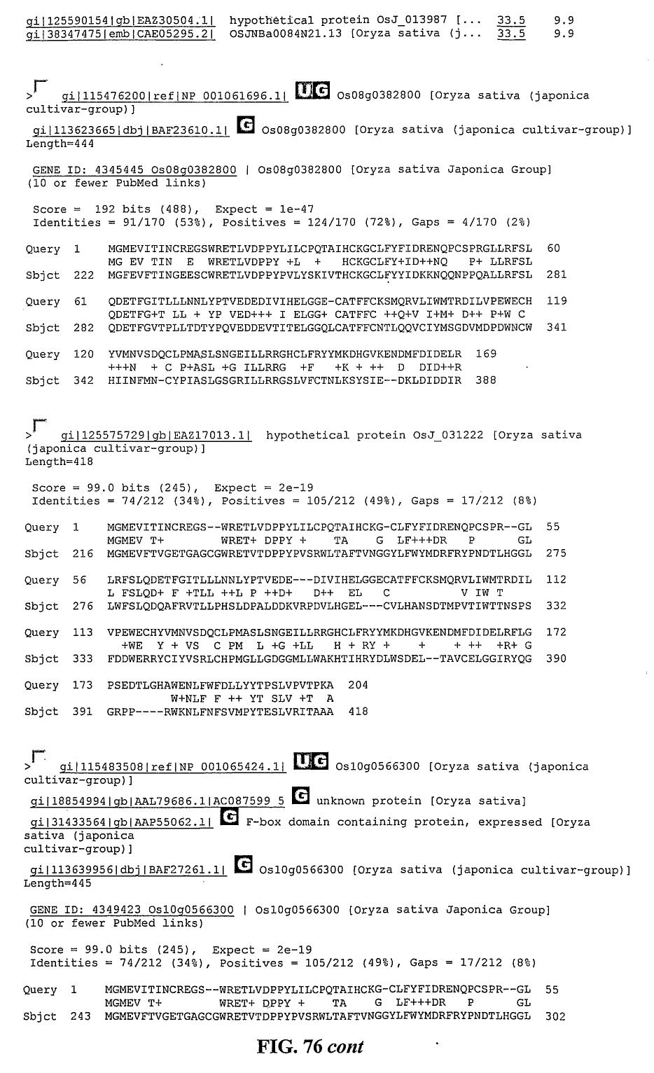

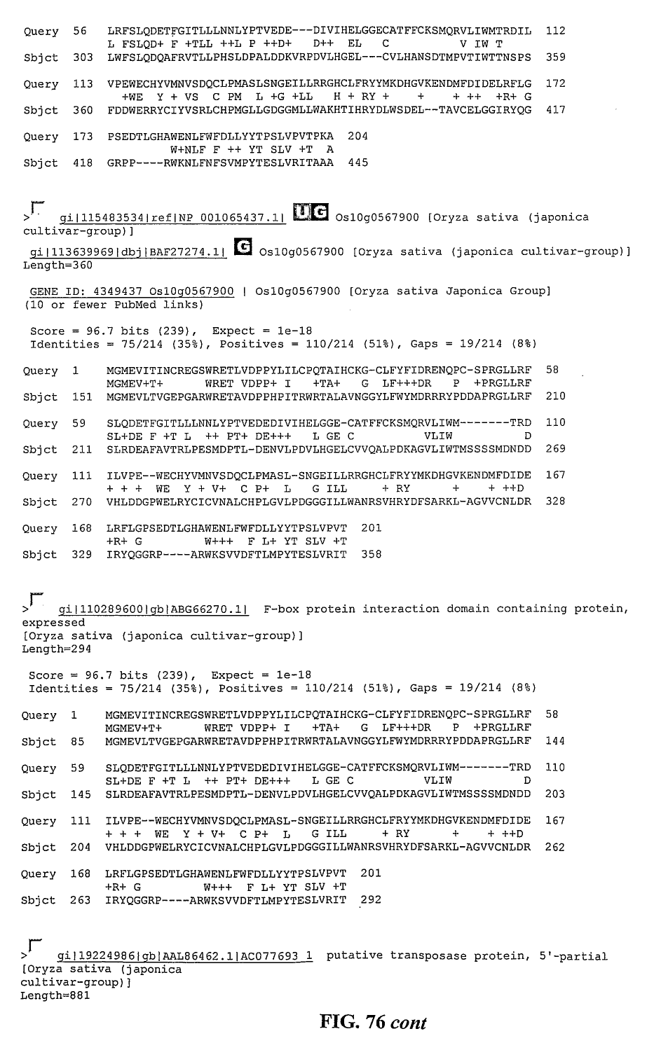

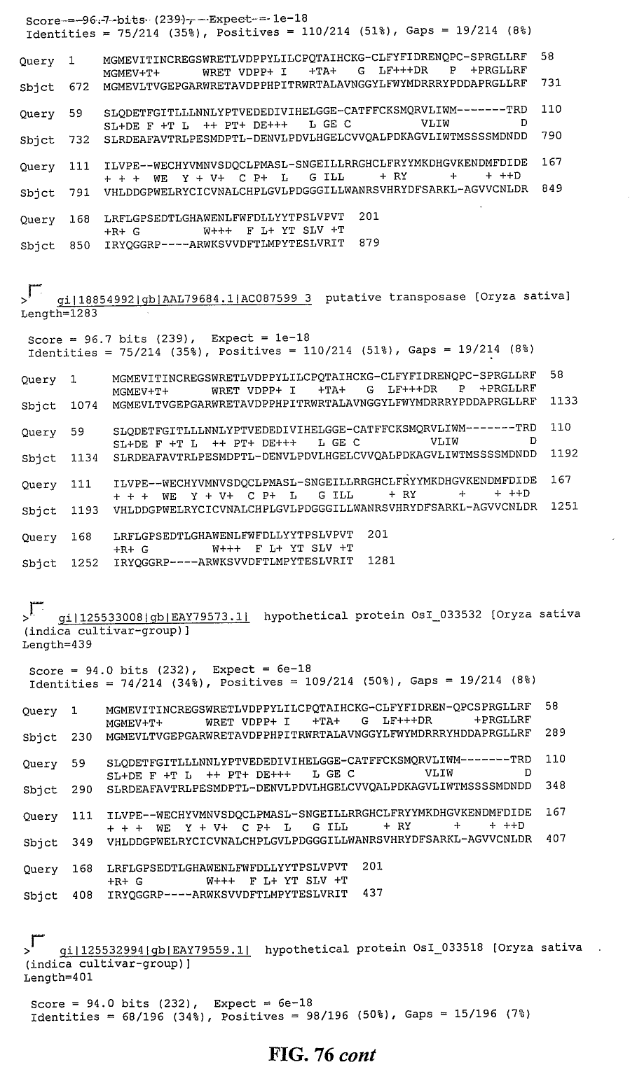

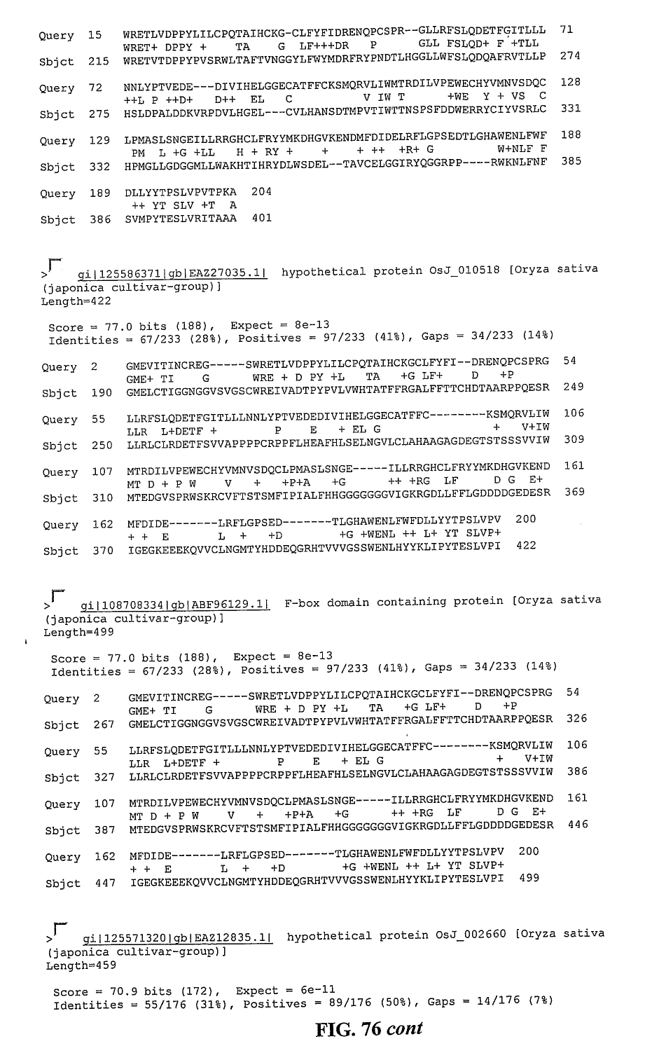

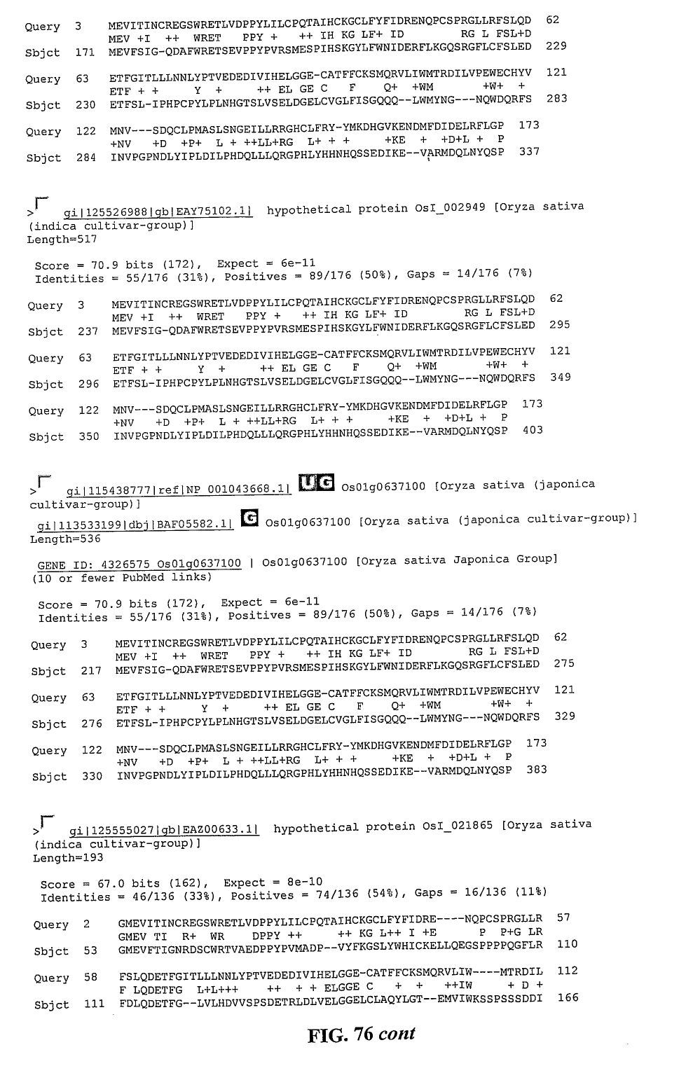

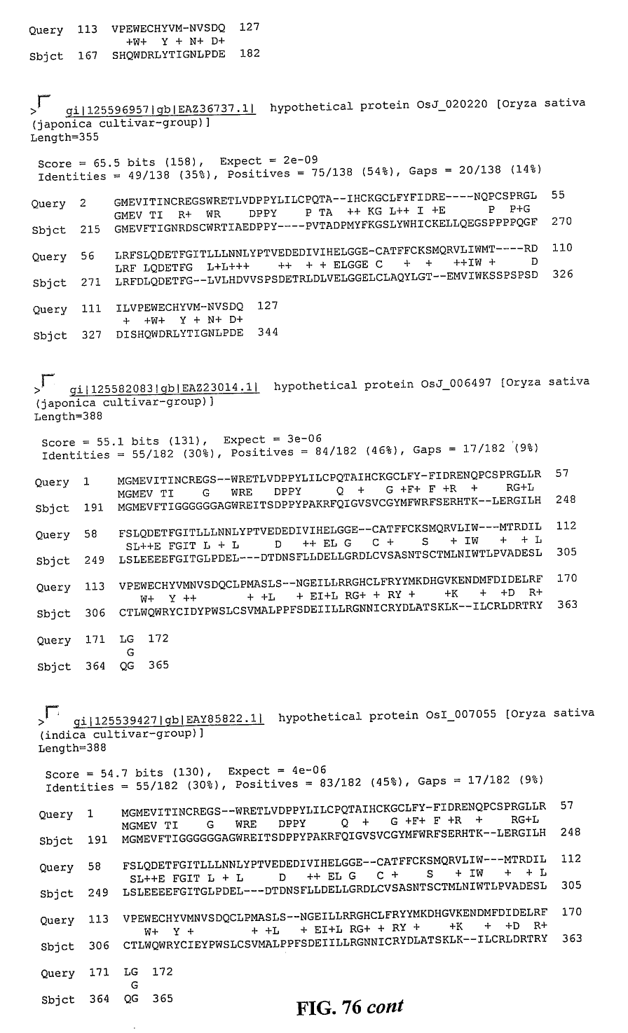

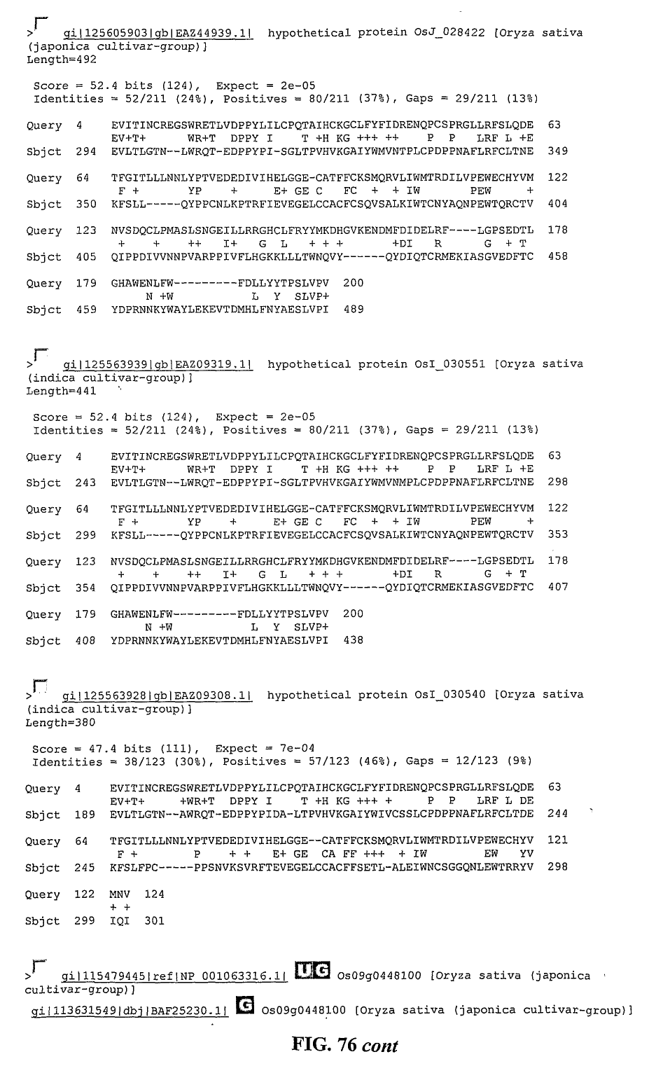

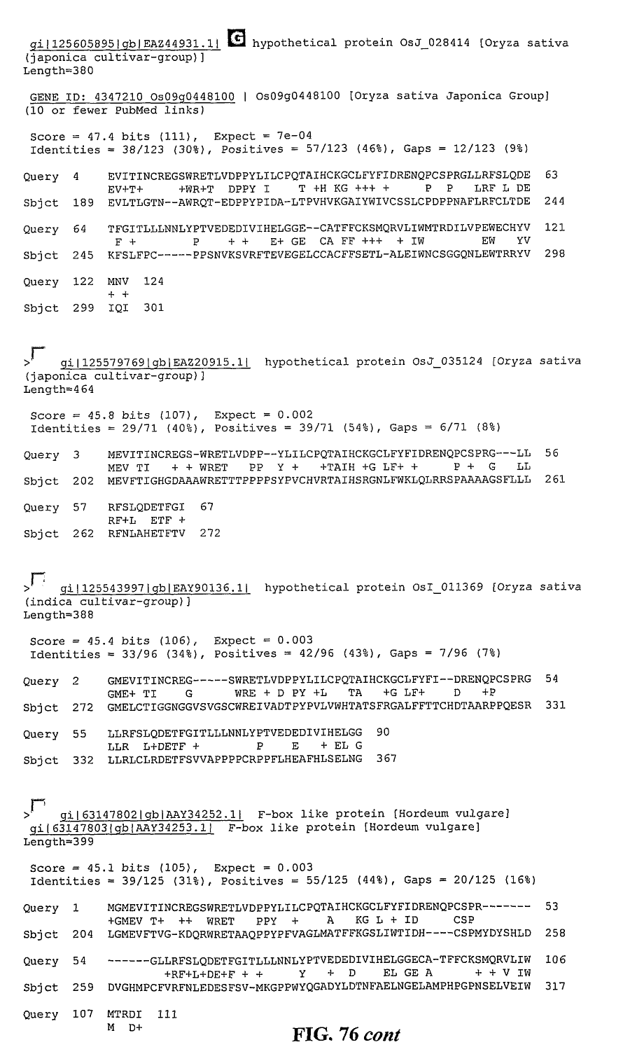

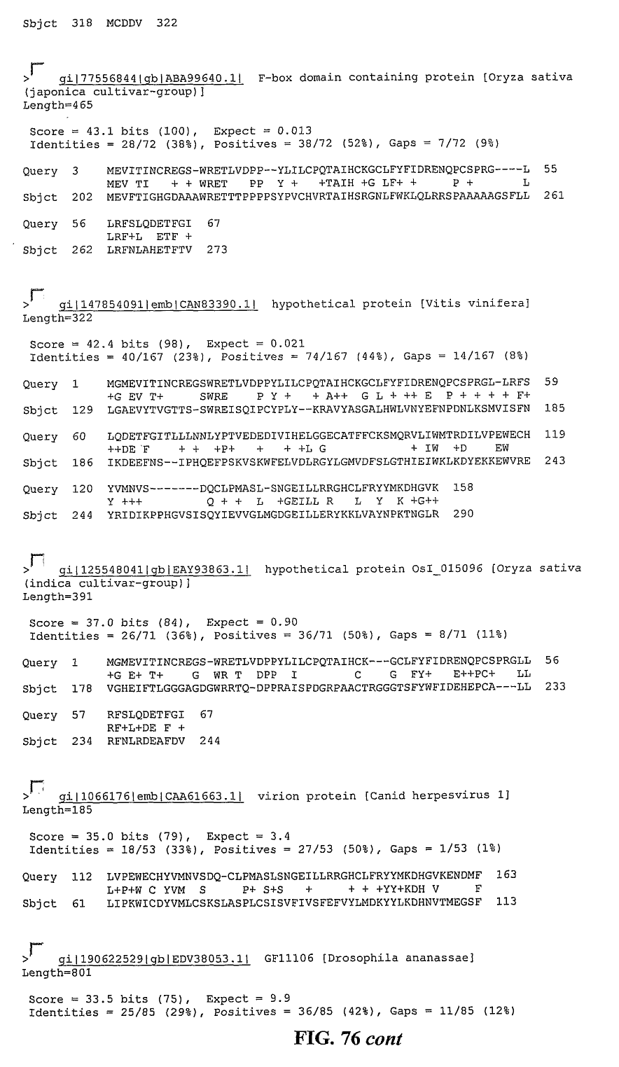

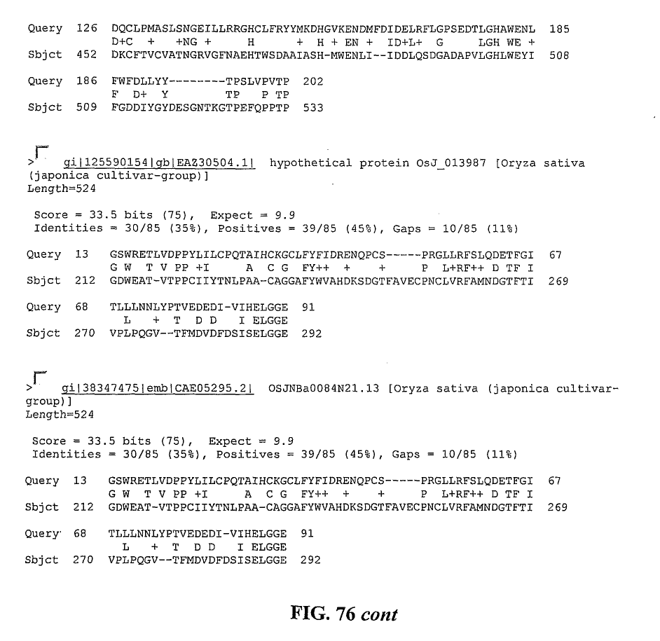

Similarly the translated amino acid sequence ORF-1/DraF2C1 located on the DraF2C1 fragment was to BLAST analysis on the NCBI portal for non redundant DNA (nr-DNA) and for ESTs.: http://blast.ncbi.nlm.nih.gov/Blast.cgi. Results of nr-DNA BLAST analysis with alignments for the first few hits are shown in FIG. 76.

### Example 11

### Construction of Genetically-Modified Wheat by Agrobacterium-Mediated Transformation

### Constructs

The construct pEvec202Nnos will be used to prepare all the constructs used for transformation of wheat.

Agrobacterium-Mediated Transformation of Barley and Rice

Transgenic wheat will be generated by Agrobacterium-mediated transformation of embryogenic callus. The embryo will be isolated from wheat seed under sterile conditions. Agrobacterium tumefaciens transformed with constructs will be grown overnight in MGL medium. For inoculation, an Eppendorf pipette will be used to place drops of the Agrobacterium culture on the cut side of the immature embryos. After incubation of the plates for about two days in the dark at 24° C., the embryos will be transferred into plates containing BCI-DM medium supplemented with hygromycin and timentin. After about six weeks of dark incubation, with transfers in fresh medium every two weeks, the embryogenic callus produced will be transferred to FHG medium supplemented with hygromycin. Regenerated shoots will be transferred into BCI medium for development of roots before transfer in soil. Detection of green fluorescence from GFP will be carried out using a compound microscope equipped with an attachment for fluorescence observations.

To determine presence of transgene, PCR screening of transgenic plants will be carried out using purified genomic DNA. All hygromycin-resistant plants will be screened for, the gfp-nos sequence by PCR (such as according to Furtado, A. and Henry, R. J. (2006), Plant Biotechnology Journal 3: 421-434). Southern-blot hybridisation will be carried out essentially according to established procedures (Maniatis et al., 1982). Genomic DNA from non-transformed or transformed plants will be digested with Hind III and checked for digestion before resolving on an agarose gel, followed by transfer onto a nylon membrane (Nylon-hybond, Roche, Germany). Hybridisation will be carried out using Dig-labelled probe corresponding to the gfp gene, followed by signal development using the Dig-detection system (Roche, Germany).

### Example 12

### Construction of Genetically-Modified Wheat by Particle Bombardment

### Constructs Used for Particle Bombardment

In one approach, the plasmid pAGN, a pGEM3Zf+ based vector (Promega Corporation, MI, USA) and containing a synthetic variant of the green fluorescent protein gene (gfpS65T) (Patterson et al., 1997) and nos terminator sequence, will be used as the cloning vector to generate the promoter construct. The promoter.gfp.nos construct will be prepared as a transcriptional fusion of the promoter with the gfpS65T henceforth referred as the gfp gene. The plasmid pAGN will also be used as the cloning vector to generate the gene constructs pUbi.gfp.nos, pCaMV35S.gfp.nos which contain the maize ubiquitin, the cauliflower mosaic virus 35S RNA promoter, linked to the gfp gene and nos terminator sequence. Plasmid pDP687 will be used as a control to check for successful particle-bombardment and viability of cells, and contains the cauliflower mosaic virus 35S RNA promoter (CaMV35S) which controls the constitutive expression of two genes, each encoding transcription factors which regulate synthesis of the red anthocyanin pigment.

Tissue preparation, particle bombardment and incubation conditions will be performed such as described in Furtado, A. and Henry, R. J. (2006), Plant Biotechnology Journal 3: 421-434.

In another approach, gene constructs will be prepared for genetic transformation of wheat by particle bombardment of immature wheat embryos. Gene constructs will be prepared to contain the gene of interest and the selectable marker gene on construct (strategy 1), or alternatively the gene of interest and the selectable marker gene were on separate gene constructs (strategy 2).

Strategy 1

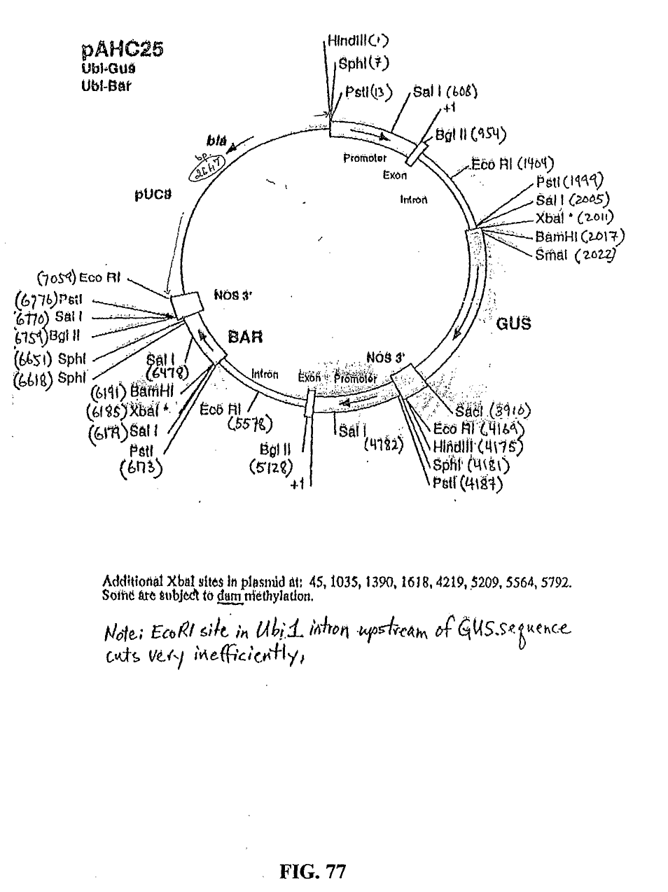

The vector pAHC25 (Christensen and Quail) contains two gene cassettes; one containing the GUS (UidA) gene under control of the Ubiquitin promoter from maize, and the other containing the bar gene under control of another Ubiquitin promoter (FIG. 77). The GUS gene will be excised out and the 30dpa-gene or 14dpa-gene will be directionally cloned to obtain the plasmid pUbi.30dpagene/14dpagene.nos-Ubi.bar.nos. The recombinant construct will be checked for correctness of sequence by sequencing. A Maxi-prep of the construct will be prepared using commercially available kits (Promega, USA) and the plasmid will be prepared at a final construct of about 1 ug/ul for use in the genetic transformation of wheat by particle bombardment.

Strategy 2

Two vectors will be used in this strategy with the aim of co-bombardment using an equal mixture of two gene constructs.

### a) Gene Construct Containing the Selectable Marker Gene.

The construct pAHC25 (FIG. 77) will be used to derive the construct pUbi.bar.nos as follows. The plasmid pAHC25 will be digested with the restriction enzyme Hind III to yield two fragments one of which contains the gene cassette Ubi.bar.nos linked to the rest of the plasmid. The fragment containing the bar gene will be re-ligated to obtain the gene construct pUbi.bar.nos.

### b) Gene Construct Containing the Gene of Interest.

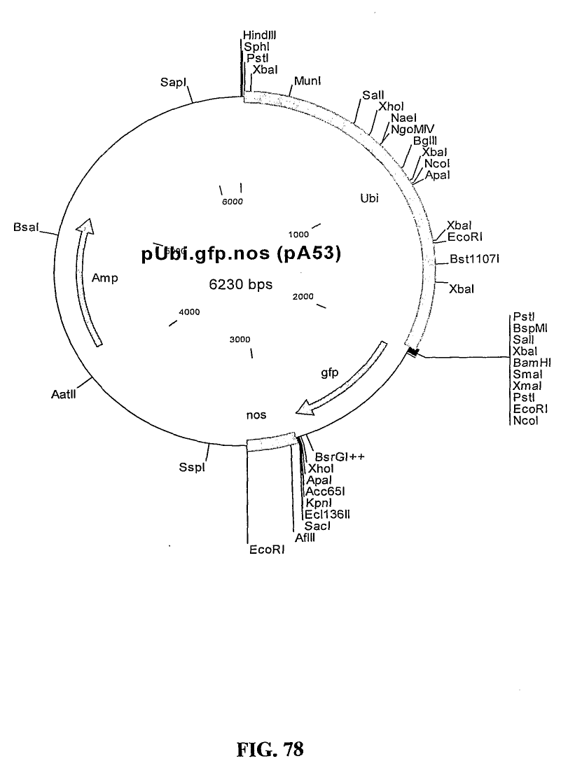

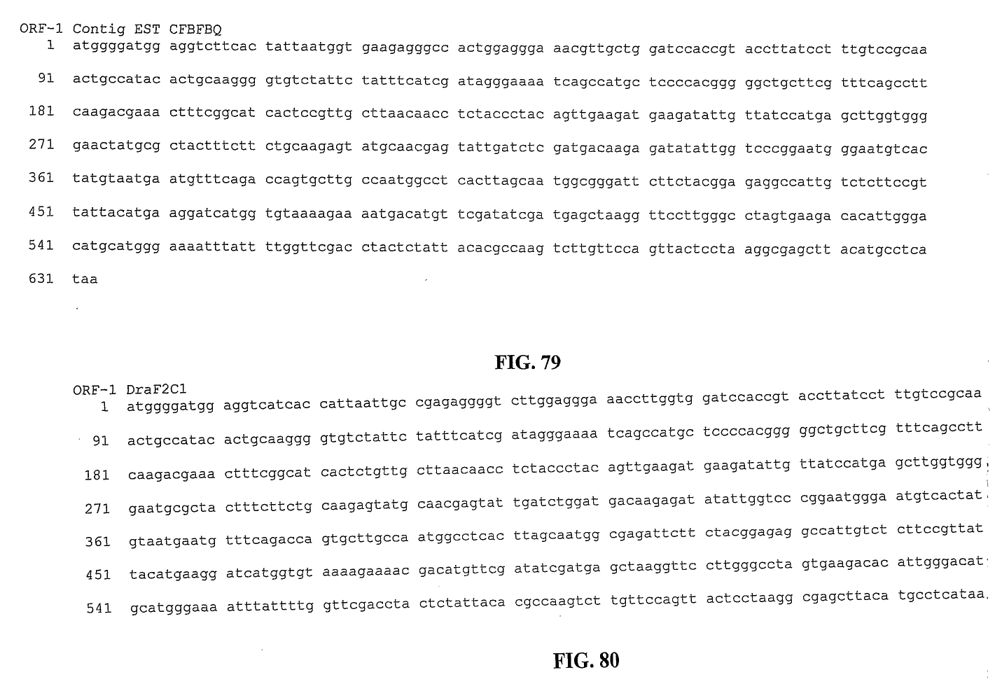

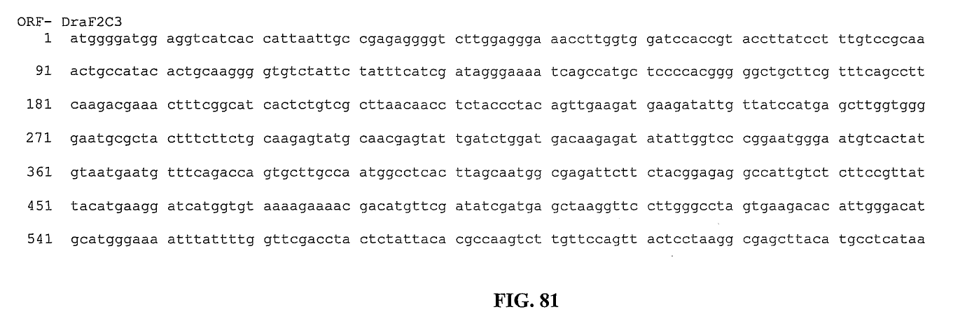

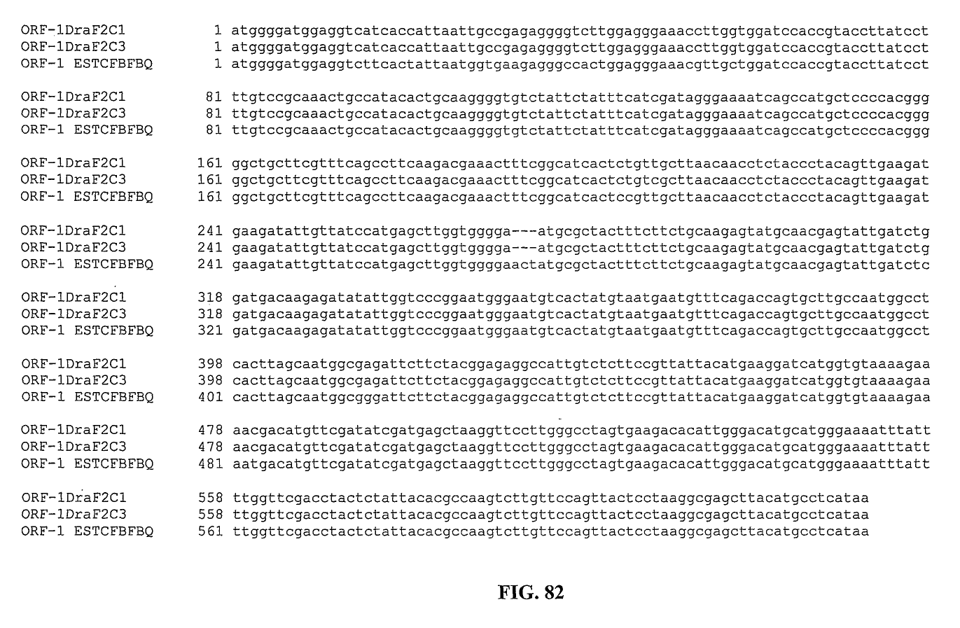

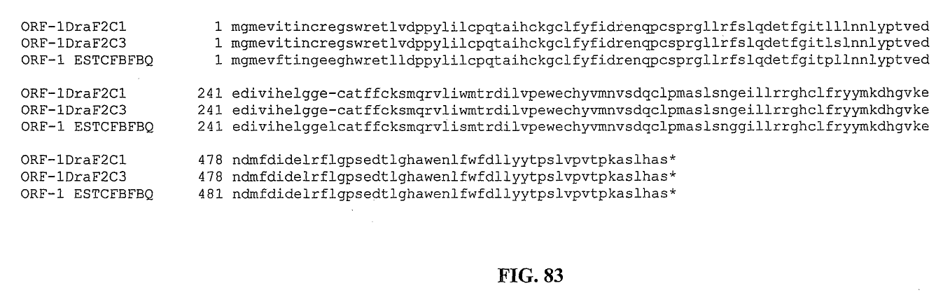

The plasmid pGEM3zf (Promega, USA) will be used as a base vector to generate the vector pUbi.gfp.nos. The plasmid pUbi.gfp.nos (FIG. 78) contains the Ubiquitin promoter from maize, the green fluorescent protein gene and the nos terminator sequence. The gfp gene will be replaced with the 30dpa-gene or 14 dpa gene using standard molecular biology techniques to yield the vector pUbi.30dpagene/14dpagene.nos.

### Example 13

### Biolistic Transformation of Wheat (Triticum aestivum L.)

Gene constructs prepared as shown in Example 12 will be used for the genetic transformation of wheat by particle bombardment of immature zygotic embryos. The transformation procedure is outlined as a schematic in below. The transformation procedure can be carried out as outlined in the following steps.

### Growing Donor Plant Material.

Wheat plants (Triticum aestivum L.) will be grown in the glasshouse to obtain immature embryos. Seeds of Bobwhite MPB26 (126 ‘Bobwhite” accession) will be used to generate wheat plants with the following growth regime;

### Sowing

Five seeds can be sowed in pots (27 cms upper diameter, vol 8.1 L) containing potting mix (equal parts (1:1:1) peat moss, perlite and vermiculite containing Dolomite-for pH, micromax-trace elements and osmocote exact-bulk nutrient).

### Sowing to 6 Weeks

Grow seedlings and plants at 24° C., with less than 70% humidity and 12 h day-length (these conditions may not be tightly controlled).

### 6 Weeks to Harvest

Grow plants at 24° C. with less than 70% humidity and 14 h day-length to stimulate flowering. This regime ensures that flowering takes place within 2 weeks.

### Harvesting of Explant Tissue.

### Method

1. Donor wheat plants will be identified with developing wheat caryopsis 14 to 18 DPA.
2. Developing caryopsis will be removed from the heads and any plant material found adhering to them such as glumes, anthers etc.
3. The immature caryopsis will be sterilised for 20 min as using sodium hypochlorite (0.8% available chlorine).
4. The surface sterilized immature caryopsis will be transferred into a sterile petri plate (10 cm×1.4 cm ht).
   Dissection of Immature Embryos from Wheat Caryopsis

### Method

1. A small batch of steralised immature embryos in sterile Petri plates will be taken.
2. Using a Stereo-microscope, the embryo axis will be excised while the immature embryo is in the caryopsis.
3. 16 to 25 embryos with their scutellum side facing up (away from the medium) will be placed in an array of 4×4 or 5×5 respectively in the centre of a petri plate containing solid osmotic medium (E3-Maltose medium).
4. The plates containing the embryos in the laminar flow will be incubated for two hrs, after which bombardment should be carried out within an hour.

### Preparation Before Using the PDS-1000 Gun

Steps in Brief with Details Outlined Below
1. Bombardment parameters for gap distance between rupture disk retaining cap and microcarrier launch assembly will be selected/adjusted. Placement of stopping screen support in proper position inside fixed nest of microcarrier launch assembly. Make sure that the distance between the stop screen and the explant material is 6 cms.
2. Helium supply (200 PSI in excess of desired rupture disc burst pressure) will be checked. If using rupture discs of 900 PSI, the working helium pressure will be adjusted to 1100 PSI.
3. The following will be cleaned/sterilized:

Equipment: rupture disk retaining cap, microcarrier launch assembly

Consumables: macrocarriers/macrocarrier holders.
4. Sterile microcarriers (gold particles 0.6 μm in diameter).
5. Microcarriers with DNA will be coated and load onto sterile macrocarrier/macrocarrier holder the day of experiment.

Sterilization of Gold Particles

Gold particles of varying diameters in microns will be obtained. Although particle sizes from 600 to 1200 microns have been used, the following procedures use 0.6 μm gold particles (Finer and McMullen, 1990; Finer et al., 1992).

### Method

1. In a 1.5 ml eppendorf tube 40 mg gold (0.6 μm in diameter) in 1 ml of 95% ethanol will be resuspended.
2. Incubation for 20 minutes at room temperature will proceed.
3. The mixture will be centrifuged for 1 to 2 minutes at 12,000 rpm in a table-top centrifuge.
4. The supernatant will be discarded and washed 3 times in 500 μl of sterile distilled water.
5. Finally, the tungsten or gold particles will be suspended in 1 ml of sterile distilled water to obtain the sterile gold-prep.
6. 50 μl of evenly dispersed sterile gold-prep will be transferred into eppendorf tubes and these can be stored at 4° C. for use up to 4 weeks

### Preparation of Gold-Plasmid Mixture

### Method

1. 50 μl (2 mg) of sterile Gold-prep (40 mg/ml) will be taken in an eppendorf tube and ensure the particles are evenly dispersed into a fine dispersion.
2. Then 5 μl of plasmid DNA (1 μg/μl) will be added. If more than one plasmid is to be used (for co-bombardments) then 5 μl of each plasmid (1 μg/μl) will be taken.
3. Before adding the CaCl₂ solution, even dispersion of the gold-DNA solution by finger-mixing will be ensured.
4. 25 μl CaCl₂ (2.5 M) will be immediately added and finger-mixing will be carried for even dispersion of Ca-DNA-coated gold particles.
5. Then 10 μl spermidine (0.1 M) will be added and again finger-mixed.
6. The tube will be incubated on ice for 5 minutes and then 50 μl of supernatant will be discarded.
7. Then 1 ml of 100% ethanol will be added and kept on ice for 1 minute.
8. Centrifugation will take place at 12,000 RPM for 2 min and the supernatant will be discarded.
9. 1 ml of 100% ethanol will be added, then finger-mixed and after centrifugation will take place 12,000 rpm for 2 min remove as much supernatant as possible.

110 μl of 100% ethanol will be added and finger mixed to resuspend particles. The mixture may be kept on ice and can be used for bombardment within 1 or 2 hrs. This preparation will contain 2 mg of gold in 110 μl. 10 μl (182 μg gold particles) of the above Ca-gold-DNA suspension for each bombardment will be used.

### Tissue Culture and Selection to Obtain Transgenic Wheat Plant

### Method

This procedure is based on the use of the bar gene as the selectable marker gene and the use of the herbicide Phosphinothricin (PPT).

1. Eighteen to 24 hrs after bombardment, bombarded immature embryos will be transferred to callus induction medium (E3call-Ind). The plates will be sealed and incubated for 14 days in dark at 25° C.
2. Plates will be monitored every 3 days to check for contamination and the recovery of uncontaminated material.
3. Take the bombardment-control-plate for GUS histochemical assay (GUS staining) if bombarded with the GUS construct (Ubi.gus.nos) or for GFP expression if bombarded with the GFP construct (Ubi.gfp.nos).
4. The proliferating callus will be transferred from the experimental plates on to selection medium containing 5 mg/l PPT+250 mg/l Cefotaxim. The plates will be sealed and incubated at 25° C. in light (16 h) and dark (8 h) cycles until the somatic embryos show signs of greening.
5. The plates will be transferred under direct light at 25° C. in light (16 h) and dark (8 h) cycles to enhance the germination of the somatic embryos into shoots and roots. The tissue will be transferred to fresh medium every 10 days.
6. PPT-resistant shoots will be transferred into rooting medium (RMed, containing 5 mg/l PPT and 250 mg/l Cefotaxim. Make sure to take shoots with as little callus as possible. This way there will be one shoot per plate. The plates will be sealed with Parafilm and incubatde at 25° C. in light (16 h) and dark (8 h) cycles till well developed roots can be seen. The culture will be subcultured every 10 days.
7. Those shoots with well developed roots will be transferred in to tissue culture vessels containing rooting medium (RMed, containing 5 mg/l PPT and 250 mg/l Cefotaxim. Incubated at 25° C. in light (16 h) and dark (8 h) cycles for further development of roots
8. Plants with well developed roots will be transferred into small pots containing potting mix and transfer to the glasshouse and watered regularly so as to increase survival.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

[1] W. S. Cleveland. Robust locally weighted regression and smoothing scatterplots. J. Amer. Statist. Assoc, 74:829-836, 1979.
[2] S. Holm. A simple sequentially rejective multiple test procedure. Scandinavian Journal of Statistics, 6:65-70, 1979.
[3] R. Irizarry, B. Hobbs, F. Collin, Y. Beazer-Barclay, K. Antonellis, U. Scherf, and T. Speed. Exploration, normalization, and summaries of high density oligonucleotide array probe level data. Biostatistics, 4:249 264, 2003.
[4] I. Lonnstedt and T. Speed. Replicated microarray data. Statistica Sinica, pages 31-46, 2002.
[5] 0. Mueller, S. Lightfoot, and A. Schroeder. RNA integrity number (RN)—standardization of RNA quality control. Technical Report 5989-1165EN, Agilent Technologies, May 2004.
[6] Y. Benjamini and Y. Hochberg. Controlling the false discovery rate: a practical and powerful approach to multiple testing. Journal of the Royal Statistical Society Series

### Tables

TABLE 1
Mean flour yield of wheat varieties grown at two sites -
Biloela (B) and Narrabri (N) for 14 dpa sample.

| | | | Std. |
|---|---|---|---|
| site | Mean | N | Deviation |
| B | 75.6627 | 59 | 2.43918 |
| N | 76.2224 | 67 | 2.13618 |
| Total | 75.9603 | 126 | 2.29099 |

TABLE 2
Tests of significance of the effects of site, genotype (variety)
and their interaction on flour yield of wheat for 14 dpa sample.

| | Type III Sum | | | | |
|---|---|---|---|---|---|
| Source | of Squares | df | Mean Square | F | Sig. |
| Model | 727671.675^{a} | 124 | 5868.320 | 19399.40 | .000 |
| site | 17.250 | 1 | 17.250 | 57.024 | .017 |
| variety | 523.052 | 66 | 7.925 | 26.198 | .037 |
| site * variety | 122.456 | 56 | 2.187 | 7.229 | .129 |
| Error | .605 | 2 | .303 | | |
| Total | 727672.280 | 126 | | | |

TABLE 3
Flour yield from 10 varieties of wheat chosen as low
or high yielding varieties based on yield measurements
from 80 varieties at two sites for 14 dpa sample.

| | | Yield Class | | |
|---|---|---|---|---|
| | AUS | (high = H, | Flour Yield | Flour Yield |
| Wheat variety | number | low = L) | Narrabri (%) | Biloela (%) |
| RHODESIAN | 1075 | L | 74.9 | 73.9 |
| NW51A | 14996 | L | 71.4 | — |
| W216 | 19310 | L | 74.9 | 72.5 |
| RUFUS | 33374 | L | 70.0 | 69.1 |
| YUKURI | 33375 | L | 73.4 | 70.8 |
| FRONTANA | 2451 | H | 79.2 | 77.9 |
| JING HONG | 17863 | H | 78.8 | 78.5 |
| NO. 1 | | | | |
| JANZ | 24794 | H | 78.1 | 77.9 |
| SATURNO | 24431 | H | 78.5 | 78.2 |
| ELLISON | 33371 | H | 79.7 | 77.9 |

TABLE 4
Mean flour yield of wheat varieties grown at two sites -
Biloela (B) and Narrabri (N) for 30 dpa sample.

| | | | Std. |
|---|---|---|---|
| site | Mean | N | Deviation |
| B | 75.6627 | 59 | 2.43918 |
| N | 76.2224 | 67 | 2.13618 |
| Total | 75.9603 | 126 | 2.29099 |

TABLE 5
Tests of significance of the effects of site, genotype (variety)
and their interaction on flour yield of wheat for 30 dpa sample.

| | Type III Sum | | | | |
|---|---|---|---|---|---|
| Source | of Squares | df | Mean Square | F | Sig. |
| Model | 727671.675^{a} | 124 | 5868.320 | 19399.40 | .000 |
| site | 17.250 | 1 | 17.250 | 57.024 | .017 |
| variety | 523.052 | 66 | 7.925 | 26.198 | .037 |
| site * variety | 122.456 | 56 | 2.187 | 7.229 | .129 |
| Error | .605 | 2 | .303 | | |
| Total | 727672.280 | 126 | | | |

TABLE 6
Flour yield from 10 varieties of wheat chosen as low
or high yielding varieties based on yield measurements
from 80 varieties at two sites for 30 dpa sample.

| | | Yield Class | | |
|---|---|---|---|---|
| | AUS | (high = H, | Flour Yield | Flour Yield |
| Wheat variety | number | low = L) | Narrabri (%) | Biloela (%) |
| RHODESIAN | 1075 | L | 74.9 | 73.9 |
| NW51A | 14996 | L | 71.4 | — |
| W216 | 19310 | L | 74.9 | 72.5 |
| RUFUS | 33374 | L | 70.0 | 69.1 |
| YUKURI | 33375 | L | 73.4 | 70.8 |
| FRONTANA | 2451 | H | 79.2 | 77.9 |
| JING HONG | 17863 | H | 78.8 | 78.5 |
| NO. 1 | | | | |
| JANZ | 24794 | H | 78.1 | 77.9 |
| SATURNO | 24431 | H | 78.5 | 78.2 |
| ELLISON | 33371 | H | 79.7 | 77.9 |

TABLE 7
AffyMatrix probes designed to target Ta.28688

| Probe Name | Nucleotide Sequence |
|---|---|
| >Ta.28688.1.A1_at*probe1 | AACGAAATGGTTACTACTATGACTG |
| >Ta.28688.1.A1_at*probe2 | ATGGTTACTACTATGACTGTAATGC |
| >Ta.28688.1.A1_at*probe3 | AGCCATGTCCGTAGTAGCGTTTTGA |
| >Ta.28688.1.A1_at*probe4 | CCATGTCCGTAGTAGCGTTTTGAGG |
| >Ta.28688.1.A1_at*probe5 | AGTAGGCAGTTCATCTCGTGTTTTA |
| >Ta.28688.1.A1_at*probe6 | GCAGTTCATCTCGTGTTTTAATAAA |
| >Ta.28688.1.A1_at*probe7 | TCATATACGAGACTGTAAGGTTCTC |
| >Ta.28688.1.A1_at*probe8 | TGTAAGGTTCTCTACCAGTATGTTA |
| >Ta.28688.1.A1_at*probe9 | GATTAGGGCTAATTTCAGTACCAGA |
| >Ta.28688.1.A1_at* | GGGCTAATTTCAGTACCAGAGTAGA |
| probe10 | |
| >Ta.28688.1.A1_at* | TCAGTACCAGAGTAGAAGTATAACT |
| probe11 | |

TABLE 8
AffyMatrix probes designed to target Ta.11743

| Probe Name | Nucleotide Sequence |
|---|---|
| >Ta.11743.1.A1_at*probe1 | CTTGTTTCTATAGCAGAGGTGTCTA |
| >Ta.11743.1.A1_at*probe2 | GTCTAAGTAAGTGTCTATGCTCAAC |
| >Ta.11743.1.A1_at*probe3 | TTGGCTTATTTTTTACGCACCTCTC |
| >Ta.11743.1.A1_at*probe4 | TTTACGCACCTCTCTAAGCATCTTG |
| >Ta.11743.1.A1_at*probe5 | ATCTTGCGCTGTTCTGGCCTGATGT |
| >Ta.11743.1.A1_at*probe6 | GGCCTGATGTGTTTGCTTGTCTGTC |
| >Ta.11743.1.A1_at*probe7 | TTGCTTGTCTGTCTACTCATGCCTA |
| >Ta.11743.1.A1_at*probe8 | GTCTACTCATGCCTACCTATTTAAT |
| >Ta.11743.1.A1_at*probe9 | AATGGATCATTGAACCTCTCTCGAG |
| >Ta.11743.1.A1_at* | CTCTCTCGAGGTTTTGTTTCAAGGG |
| probe10 | |
| >Ta.11743.1.A1_at* | GTATTGACACTTAAACGATGCTTTG |
| probe11 | |

1. A method of selecting a grain or a grain-producing plant with improved flour yield, said method including the step of determining a relative amount of an isolated nucleic acid associated with or linked to improved flour yield present in the grain or grain-producing plant to determine whether or not the grain or grain-producing plant has a predisposition to improved flour yield, wherein said isolated nucleic acid encodes a polypeptide which regulates transcription.

2. The method of claim 1, wherein said isolated nucleic acid encodes a polypeptide which regulates transcription elongation.

3. The method of claim 1, wherein the isolated nucleic acid associated with or linked to improved flour yield encodes a polypeptide comprising an amino acid sequence selected from the group consisting of SEQ ID NO:49 and SEQ ID NO:190, or a fragment thereof.

4. The method of claim 1, wherein the isolated nucleic acid associated with or linked to improved flour yield comprises a nucleotide sequence selected from the group consisting of SEQ ID NO:26 and SEQ ID NO:285, or a fragment thereof.

5. The method of claim 1, wherein the fragment comprises a nucleotide sequence selected from the group consisting of SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

6. The method of claim 1, wherein the isolated nucleic acid associated with or linked to improved flour yield is a variant having at least 70% sequence identity to a nucleotide sequence selected from the group consisting of SEQ ID NO:26 and SEQ ID NO:285.

7. The method of claim 1, wherein the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

8. The method of claim 1, wherein the grain or the grain-producing plant has a grain comprising at least an endosperm and a bran layer.

9. The method of claim 1, wherein the grain or the grain-producing plant is wheat.

10. The method of claim 1, wherein the grain or the grain-producing plant has a reduced relative amount of the isolated nucleic acid associated with or linked to improved flour yield when compared to a reference sample.

11. A method of determining whether a grain or a grain-producing plant is genetically predisposed to improved flour yield, said method including the step of detecting an isolated nucleic acid associated with or linked to improved flour yield to thereby determine whether the grain or grain-producing plant is genetically predisposed to improved flour yield, wherein said isolated nucleic acid encodes a polypeptide which regulates transcription

12. The method of claim 11, wherein said polypeptide regulates transcription elongation.

13. The method of claim 11, wherein the isolated nucleic acid associated with or linked to improved flour yield comprises a nucleotide sequence selected from the group consisting of SEQ ID NO:26 and SEQ ID NO:285, or a fragment thereof.

14. The method of claim 11, wherein the fragment comprises a nucleotide sequence selected from the group consisting of SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

15. The method claim 11, wherein the isolated nucleic acid associated with or linked to improved flour yield is a variant having at least 70% sequence identity to a nucleotide sequence selected from the group consisting of SEQ ID NO:26 and SEQ ID NO:285.

16. The method of claim 11, wherein the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

17. The method of claim 11, wherein the grain or the grain-producing plant has a grain comprising at least an endosperm and a bran layer.

18. The method of claim 11, wherein the grain or the grain-producing plant is wheat.

19. A method of milling flour, said method including the step of selecting a millable grain or a millable grain-producing plant according to the method of claim 1 for subsequent milling of said grain to produce a flour.

20. The method of claim 19, wherein the millable grain or the millable grain producing plant has a grain comprising at least an endosperm and a bran layer.

21. The method of claim 19, wherein the millable grain or the millable grain-producing plant is wheat.

22. A method of identifying one or more plant genetic loci which is/are associated with improved flour yield of a grain or a grain-producing plant, said method including the step of determining whether one or more plant genetic loci is/are associated with or linked to flour milling yield, wherein said one or more plant genetic loci encodes a polypeptide which regulates transcription.

23. The method of claim 22, wherein said polypeptide regulates transcription elongation.

24. The method of claim 22, wherein the one or more plant genetic loci is a polymorphism of a nucleotide sequence selected from the group consisting of SEQ ID NO: 15 and SEQ ID NO:159.

25. A method of producing a grain-producing plant with improved flour yield, said method including the step of selectively modulating a gene associated with or linked to improved flour yield, so that the relative amount of said gene associated with or linked to improved flour yield is lower or higher than in a grain-producing plant where said gene has not been selectively modulated, wherein said gene encodes a polypeptide which regulates transcription.

26. The method of claim 25, wherein said polypeptide regulates transcription elongation.

27. The method of claim 25, wherein selective modulation is down-regulation of the gene associated with or linked to improved flour yield.

28. The method of claim 25, wherein the gene encodes a polypeptide with an amino acid sequence selected from the group consisting of SEQ ID NO:49 and SEQ ID NO:190, or a fragment thereof.

29. The method of claim 25, wherein the gene comprises a nucleotide sequence selected from the group consisting of SEQ ID NO:26 and SEQ ID NO:285.

30. The method of claim 25, wherein the gene comprises a nucleotide sequence which is a variant having at least 70% sequence identity to SEQ ID NO:26 and SEQ ID NO:285.

31. The method of claim 25, wherein the gene comprises a nucleotide sequence which is a variant selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

32. The method of claim 25, the grain or the grain-producing plant has a grain comprising at least an endosperm and a bran layer.

33. The method of claim 25, wherein the grain or the grain-producing plant is wheat.

34. A grain-producing plant having improved flour yield produced according to the method of claim 25.

35. The grain-producing plant of claim 34, which is wheat.

36. A method of milling flour, said method including the step of obtaining a grain from a grain-producing plant produced according to the method of claim 25 for subsequent milling to produce a flour.

37. A genetic construct when used to improve grain flour yield comprising an isolated nucleic acid associated with or linked to improved flour yield, wherein said isolated nucleic acid encodes a polypeptide which regulates transcription.

38. The genetic construct of claim 37, wherein said polypeptide regulates transcription elongation.

39. The genetic construct of claim 37, wherein the isolated nucleic acid associated with or linked to improved flour yield encodes a polypeptide with an amino acid sequence selected from the group consisting of SEQ ID NO:49 and SEQ ID NO:190, or a fragment thereof.

40. The genetic construct of claim 37, wherein the isolated nucleic acid associated with or linked to improved flour yield comprises a nucleotide sequence selected from the group consisting of SEQ ID NO:26 and SEQ ID NO:285, or a fragment thereof.

41. The genetic construct of claim 37, wherein the fragment comprises a nucleotide sequence selected from the group consisting of SEQ ID NO:162, SEQ ID NO:163, SEQ ID NO:164, SEQ ID NO:165, SEQ ID NO:166, SEQ ID NO:167, SEQ ID NO:168 and SEQ ID NO:169.

42. The genetic construct of claim 37, wherein the isolated nucleic acid associated with or linked to improved flour yield is a variant having at least 70% sequence identity to a nucleotide sequence selected from the group consisting of SEQ ID NO:26 and SEQ ID NO:285.

43. The genetic construct of claim 37, wherein the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.

44. A grain-producing plant with improved flour yield wherein a gene associated with or linked to improved flour yield is selectively modulated to have a lower relative amount of the gene associated with or linked to improved flour yield than in a plant where the gene has not been modulated, wherein said genes encodes a polypeptide which regulates transcription.

45. The grain-producing plant of claim 44, wherein the polypeptide regulates transcription elongation.

46. The grain-producing plant of claim 44, wherein the gene associated with or linked to improved flour yield encodes a polypeptide with an amino acid sequence selected from the group consisting of SEQ ID NO:49 and SEQ ID NO:190, or a fragment thereof.

47. The grain-producing plant of claim 44, wherein the gene associated with or linked to improved flour yield comprises a nucleotide sequence selected from the group consisting of SEQ ID NO:26 and SEQ ID NO:285, or a fragment thereof.

48. The grain-producing plant of claim 44, wherein the gene associated with or linked to improved flour yield comprises a nucleotide sequence which is a variant having at least 70% sequence identity to SEQ ID NO:26 and SEQ ID NO:285.

49. The grain-producing plant of claim 44, wherein the variant comprises a nucleotide sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48 and SEQ ID NO:194.


SEQUENCE LISTING (316 sequences)

SEQ ID NO: 1 - DNA, 661 bp, Triticum sp.
cgagttggtagattttgaaggaagaaaaaggcgttgaaccatggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccatcttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtatgttatcatggttgagattagggctaatttcagtaccagagtagaagtataactcatactatgttggttgttgttgcggttggtatgttcatgta

SEQ ID NO: 2 - protein, 89 aa, Triticum sp.
MGKRKSAAKPPPKKRMDKLDTVFSCPFCNHGSSVECRIDMKNLIGEANCRICQESFSTTVNALTEPIDIYSEWIDECERVNTVEDDDGA

SEQ ID NO: 3 - DNA, 305 bp, Triticum sp.
cttgtttctatagcagaggtgtctaagtaagtgtctatgctcaacaaataagcaacggtgcttaagagaaattggcttattttttacgcacctctctaagcatcttgcgctgttctggcctgatgtgtttgcttgtctgtctactcatgcctacctatttaatggatcattgaacctctctcgaggttttgtttcaagggaagaacgttgaagatcaattctgtttaatgttgtatttgtttcaagtgaagaacgttgaagatcaattctgtttaatgttgtattgacacttaaacgatgctttg

SEQ ID NO: 4 - DNA, 450 bp, Triticum sp.
ggcgagcttacatgcctcataagaccatgtacactgcaaggtgtttagagaagtgcttgtataaataaattgttttttcccttaagtaccggtgcttgtttctatagcagaggtgtctaagtaagtgtctatgctcaacaaataagcaacggtgcttaagagaaattggcttattttttacgcacctctctaagcatcttgcgctgttctggcctgatgtgtttgcttgtctgtctactcatgcctacctatttaatggatcattgaacctctctcgaggttttgtttcaagggaagaacgttgaagatcaattctgtttaatgttgtatttgtttcaagtgaagaacgttgaagatcaattctgtttaatgttgtattgacacttaaacgatgctttgttttgtgcatgttattttctgctgtttctaaagaaaaaaaaaaaaaaaaaa

SEQ ID NO: 5 - DNA, 295 bp, Triticum sp.
  misc_feature (41)..(41): n is a, c, g, or t
aacgaaatggttactactatgactgtaatgcgttggtgttnattggctgccancttcagccatgtccgtagtagcgttttgaggncgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtatgttatcatggttgagattagggctaatttcagtaccagagtagaagtataact

SEQ ID NO: 6 - DNA, 280 bp, Triticum sp.
  misc_feature (43)..(43): n is a, c, g, or t
gtgcatgagcatgtctggttgcaaaatgaaatggttattactnctatgactgtaatgcgttggtgttattggctgccaccttcagccatnnctgtagtnagcnttatggctgatcgaaccgttagtaggcagttcatcctgcgtttaataaacaagcgatgatattgtgtacttgtgtttgaaccttaatagtagtgttgcatgtccgatgtgtatggatccatcatatatgagactgtaaggttggttctcttccagtatgttatcatggttgagatta

SEQ ID NO: 7 - DNA, 344 bp, Triticum sp.
  misc_feature (22)..(22): n is a, c, g, or t
atggtgcatgagcatgtctggntgcaaaacgaaatggttactactatgactgtaatgcgttggtgttattggctgccatcttcagccatgtccgtagtagcgttttgaggacgtgctggctgatcgaaccgttagtaggcagttcntctcgtgttttaataaacaagcgatgatattgtgtacttgtgtttgaaccctaatagtagtgttgcatgtccggtgtgtatggatccatcatatncgagactgtaaggttctctaccagtatgttatcatggttgagattagggctaatttcagtaccanagtagaagtataactcangctatgttggttgttgttgc

SEQ ID NO: 8 - DNA, 301 bp, Triticum sp.
  misc_feature (217)..(217): n is a, c, g, or t
gtaaatgcgctgactgagcctattgatatatacagcgaatggatcgatgagtgcgagcgcgtcaacactgtcgaagatgatgatggtgcatgagcatgtctggttgcaaaatgaaatggttattactcctatgactgtaatgcgttggtgttattggctgccaccttcagccatgtctgtagtagcgttatggctgatcgaaccgttagtaggcagntcatcctgcgtttaataaacaagcgatgatattgtgtacttgcgtttgaaccttaatagtagtgttgcatgtccagggttggtt

SEQ ID NO: 9 - DNA, 497 bp, Triticum sp.
  misc_feature (459)..(459): n is a, c, g, or t
atctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccatcttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtatgttatcatggttgagattagggctaatttcagtaccagagtagaagtataactcanactatgttggttgttgttgcggttggtatgttcatgta

SEQ ID NO: 10 - DNA, 334 bp, Triticum sp.
  misc_feature (259)..(259): n is a, c, g, or t
ggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatganggtgcatgagcatgtctggttgcaaaacgaaatggttactactatgactgtaatgcgttggtgttattggctgcc

SEQ ID NO: 11 - DNA, 569 bp, Triticum sp.
  misc_feature (545)..(545): n is a, c, g, or t
gcctcctcgtccgacgcgacgcaccgccccgagttggtagattttgaaggaagaaaaaggcgttgaaccatggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccatcttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtnccggtgtgtatggatccatcatat

SEQ ID NO: 12 - DNA, 611 bp, Triticum sp.
gaggaattcggcacgaggcacgccccgagttggtagattttgaaggaagaaaaaggcgttgaaccatggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccatcttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtatgttatcatggttgagatta

SEQ ID NO: 13 - DNA, 614 bp, Triticum sp.
  misc_feature (43)..(43): n is a, c, g, or t
attcggcacgaggcctcctcgtccgacgcgacgcacgccccgngttggtagattttgaaggaagaaaaaggcgttgaaccatggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccatcttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtaaaaaaaaa

SEQ ID NO: 14 - DNA, 474 bp, Triticum sp.
  misc_feature (377)..(377): n is a, c, g, or t
gaagcctcgtccgacgcgccgcacgccccgtagcttttgaaggaagaaaaagaggttgaaccatggggaagagtaaagtcagccgctaatgccaccccctaagaagcggatggacaagctcgacacggtcttttcctgcccattctgcaatcatgggagcagtgttgaatgccgaattgatatgaagaatctgattggtgaggctaattgtcgaatctgccaggaaagcttcagcaccactgtaaatgcgctgactgagcctattgatatatacagcgaatggatcgatcagtgcgagcgcgtcaacactgtcgaagatgatgattgtgcatgagcatgtctggttgcaaaacgaaatggttactactatgactgtnatgcgttggttttattggctgccaccttcagccatgtctgtagtagcgttttgaggtcgtgctggctgatcgaaccgtttgtaggcagttcatctcg

SEQ ID NO: 15 - DNA, 701 bp, Triticum sp.
  misc_feature (663)..(663): n is a, c, g, or t
attcggcacgaggcctcctcgtccgacgcgacgcacgccccgagttggtagattttgaaggaagaaaaaggcgttgaaccatggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatgagcatgtctggttgcaaaacgaaatggttactactatgactgtaatgcgttggtgttattggctgccaycttcagccatgtccgtagtagcgttttgaggacgtgctggctgatcgaaccgttagtaggcagttcatctcgtgttttaataaacaagcgatgatattgtgtacttgtgtttgaaccctaatagtagtgttgcatgtccggtgtgtatggatccatcatatacgagactgtaaggttctctaccagtatgttatcatggttgagattagggctaatttcagtaccagagtagaagtataactcanrctatgttggttgttgttgcggttggtatgttcatgta

SEQ ID NO: 16 - DNA, 270 bp, Triticum sp.
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggctaactgtcgaatctgccaggaaagcttcagcaccaccgtaaatgcgctgactgagcctattgatatatacagcgaatggatcgatgagtgcgagcgcgtcaacactgtcgaagatgatgatggtgcatga

SEQ ID NO: 17 - DNA, 113 bp, Oryza sp.
atggggaagagaaagtcagctgctaagccaccccctaagaagcggatggacaagctcgacacggtcttttcctgcccgttctgcaatcatgggagcagtgttgaatgccgaat

SEQ ID NO: 18 - DNA, 70 bp, Oryza sp.
tgatatgaagaatctgattggtgaggctaactgtcgaatctgccaggaaagcttcagcaccaccgtaaat

SEQ ID NO: 19 - DNA, 23 bp, Oryza sp.
gcgctgactgagcctattgatat

SEQ ID NO: 20 - DNA, 64 bp, Oryza sp.
atacagcgaatggatcgatgagtgcgagcgcgtcaacactgtcgaagatgatgatggtgcatga

SEQ ID NO: 21 - DNA, 40 bp, Triticum sp.
atggggaagagaaagtcagctgctaagccaccccctaaga

SEQ ID NO: 22 - DNA, 47 bp, Triticum sp.
tgatatgaagaatctgattggtgaggctaactgtcgaatctgccagg

SEQ ID NO: 23 - DNA, 22 bp, Triticum sp.
cgctgactgagcctattgatat

SEQ ID NO: 24 - DNA, 64 bp, Triticum sp.; identical to SEQ ID NO: 20

SEQ ID NO: 25 - DNA, 3 bp, Triticum sp.
atg

SEQ ID NO: 26 - DNA, 270 bp, Triticum sp.; identical to SEQ ID NO: 16

SEQ ID NO: 27 - DNA, 2499 bp, Oryza sp.
agcggcgggtgtgaacaccctccattttttccccccaatcccttccgacgcgctctcgagctccccgcattccccgtctcccctccgcctgccccgggagctcctccggcgagatccgcctctgacagccaccctcccacggctgctcgtcgtcctcacctccggcgaccggctcaggtgaggccccgggctccatcacctccccacggtctggctctggctgccccccatgggggacaggcgggtgcctccatcggcggcgttcacgtcggcccattctccgccgccgcccgaaattttctttacttttatgtgctgcgccatggtggtggtacaggtgtgcggtttttggatcgtttgtgtgtttaacccccttcatcgtgagggaagaatcgatccgatctgctgccgtgagggaatcgcgaggggattattgactattttgtacctcgtttagttgaattgacgtttgatcgggtgggttggttgtgtgttggtagaggaatgatgtatgatgactcaggatttctctctctagggttagtccaaatttcccgatcccgcttcaaaatcgtcatggaagagaggaagtttggataggagtacaagacttaagtacatggcaaatccttcagccccttctcgtttgtcatccttagcaaattaacaagctatgtagatgtgggtcttgtaatttcttttgagtaatcagtttagtttcagtcaggaactcccgctgtagattactcaaaatgattctcacagcatgtcgttggttgatttttctgggttgattggattggaagggttatgccaaattccctctctatgaagtatatctttattaaatttttactgattgatgaacacttgatgtagtatccatcgcaggaacaaataggatcttgtgctcctgaacaccattgtgattagttcactggccacttccttttgtgataatagaatttggtagcatagatgaactttcttatgttgtgtgtgttaatacatattcttcaaaacacactgaagtttctcacgtaaattgcctgttcgatatgaatttacctatggatgttttgtgtcatgtttacaactagaaacttgtagaacttatggtcattcagaacattatagttaatgttccttgttttactttgaaggaagaaagtaacttgagccatgggaaagaggaagtcagcggctaagccaccacctaagaagaggatggacaagcttgatactgtattttcctgcccattctgcaatcatggcagtagtgttgaatgccggatgtgagtacaatttccacaagtttacttatctgtctgttgctactgcttatggtaatgcattttgactcatttctcacccttcttccagtgatatgaagaacctgatcggcgaggcttcatgtaggatctgtcaggaaaacttcagcaccactgtcaatggtatgcgttagtggtacactgatttagatttctcatttcatttaaaaatgcaagcaagagcattggatttgttattttgttcctttcatgaaatgaactggtaccaccttcgagaacaaattaggttagcttctcatagccatgtccttttagtattgtactctttgcctagtcactttgtttcaaccaactggagataatacagcagaactaagagagtagagcagaatctcttgtaaagatcaaattgaggataagagaggaaattcaacttggaatcatactatttatgggtgtttagtttttcagtaaaattggtctgaacaaactatattgtctctgatgtccactcagtagccactatgttgacaccttcatgtctatcaagacaataaacatgttgcaagtgtgagttaactgatggtgcaaaagaagatagagtgagtaataaacctgcacaagaatatcgtgcataaaaacagtgggtagtaaaacgttgttctttggtaaactgcactttccaagtctatactgattctgtgatttctttgttgcagcactgactgaaccaatcgacatgtaagttaaaactccctacaaataacaacttactcattccagttgtgcaaagtctaatcacctatgccacacttttacttacattctattctcttgcagatatagcgaatggattgatgagtgcgagcgtgtcaacaatgtcgaagacgacgacggagcatgagcaacgcaaaatgcctgatgttgccgtacctgcactggtgctattgactgccaccttcagccacgtctgtaatagtgtaatggctgattggtttgttagtaggcagttcattcccgcacctaaataaacacctgaagacgtgatgttataccggcttcccatcaaaaactcaaaaacagttatatgcgtgtgtaattgtatgggcctagtgccttacctgggatattgtaaccgtaacttttcaaatcagcatgtttccatggatgaaatttctatgaattcaaaggctacactccaaatttg

SEQ ID NO: 28 - DNA, 25 bp, Triticum sp.
aacgaaatggttactactatgactg

SEQ ID NO: 29 - DNA, 25 bp, Triticum sp.
atggttactactatgactgtaatgc

SEQ ID NO: 30 - DNA, 25 bp, Triticum sp.
agccatgtccgtagtagcgttttga

SEQ ID NO: 31 - DNA, 25 bp, Triticum sp.
ccatgtccgtagtagcgttttgagg

SEQ ID NO: 32 - DNA, 25 bp, Triticum sp.
agtaggcagttcatctcgtgtttta

SEQ ID NO: 33 - DNA, 25 bp, Triticum sp.
gcagttcatctcgtgttttaataaa

SEQ ID NO: 34 - DNA, 25 bp, Triticum sp.
tcatatacgagactgtaaggttctc

SEQ ID NO: 35 - DNA, 25 bp, Triticum sp.
tgtaaggttctctaccagtatgtta

SEQ ID NO: 36 - DNA, 25 bp, Triticum sp.
gattagggctaatttcagtaccaga

SEQ ID NO: 37 - DNA, 25 bp, Triticum sp.
gggctaatttcagtaccagagtaga

SEQ ID NO: 38 - DNA, 25 bp, Triticum sp.
tcagtaccagagtagaagtataact

SEQ ID NO: 39 - DNA, 26 bp, Triticum sp.
ccatggggaagagaaagtcagctgct

SEQ ID NO: 40 - DNA, 28 bp, Triticum sp.
gacagcttctactactaccacgtactcg

SEQ ID NO: 41 - DNA, 1045 bp, Triticum sp.
ccatggggaagagaaagtcagctgctaagccaccccctaagaagcggatggacaagctcgacacggtcttttcctgcccgttctgcaatcatgggagcagtgttgaatgccgaatgttagtactgtcttcacatgttttcctgctaattgcttgatactggatggctgtccattcaacattgtgactggggtttcccccttttcctttcttcagtgatatgaagaatctgattggtgaggctaactgtcgaatctgccaggaaagcttcagcaccaccgtaaatggtatgtccaaattatacataactgcaaattgtactcctaaagtagatgcccatactgattgattctcagctaggaaaccccgcaccagacatctcattgtttttgtttcttctcaacctaatgcttgcagctctagaaacatggtatagcttatatctaaaactttcctacagccatgcctatctcatatactttagtcattctaatcaactaggtcatgatggagcagagccgaagtagtaatgatgtacggttatatgcgtacttggttgccattaaatcacacaaccatgcttgtaagataggccatgtggctcccttttttttcatgttccaagtctgagttaactgaattgcctgaaacaaacctgcatcatcataagtttagaactgaacctgcatactaatattgcttaaatgaaaacagtgttgtagagggttaacctttgttgtgccacatgttttcaagtttgtgtataactatgtcatgtaaaggaaacatgttcagtagagtttttgtacccttgctgagctgcatgtttttgctattgtactaattctctgttttctttggtgcagcgctgactgagcctattgatatgtaagtcaaatccaacaaataactcttgactagtgagttggttcaaatacgtcaaggagaccaggaactgaagatcgtattatctttcctgcagatacagcgaatggatcgatgagtgcgagcgcgtcaacactgtcgaagatgatgatggtgcatgag

SEQ ID NO: 42 - DNA, 1050 bp, Triticum sp.
ccatggggaagagaaagtcagctgctaagccaccccctaagaagcggatggacaagctcgacacggtcttttcctgcccgttctgcaatcatgggagcagtgttgaatgccgaatgttagtactaccttcacacgttttcctgttcattggttgatattggatggctatcaattcagcattgtgactggggtttccccctttttttcttcagtgatatgaagaatctgattggtgaggcaaattgtcgaatctgccaggaaagcttcagcacaaccgtaaacggtatgcccaaaatttacataaaactgcagattttacctcctaatgcagatgttgtactgattgattctcagctagaaatttggctctagacatctcaattgtttttgtttcttctcaaactaatgcttacagctttaagaacagggtatagcttatctataaatttcctacagccatgtccatctcatatactcttgggggtttagtcattctaatcaactaggtcataatgaagcagagccgaggtagcaatcatgtacagttatattcttacctggttgccattaatcacaccatcatgtctgtaaggcaggtcatgtggctccctatgtttatcgtgtccaagtctgagttaactgaattgcttgaaacaaacctgcatcatcacaagttcagaactgaacctgtataataatattgcttaaatgaaaaacagtgttgtagagggttaaccattgttgtgccacatgttttcaagtttgtgtataactatctcatgtaaaggaaacagtgttcagtagaattttttacccttgctgacctacatgtttctggtattgtactaattctctgttttctttggtgcagcgctgactgagcctattgatatgtaagtcaaattcaacaaataactcttgactagtgagttggtgcaaatacgtcagagaccaggaactgaagatcatattaattttcctgcagatacagcgaatggatcgatgagtgcgagcgcgtcaacactgtcgaagatgatgatggtgcatgag

SEQ ID NO: 43 - DNA, 1067 bp, Triticum sp.
ccatggggaagagaaagtcagctgctaagccacctcctaagaagcggatggacaagctcgacactgtcttttcctgcccgttctgcaatcatggcagcagtgttgaatgccgaatgttagtactgtcttcacatgttttcctgttaaattggttggtactggattgctatccattcaacattgtgactggggtttccctcttttttttcccttcagtgatatgaagaatctgattggtgaggccaattgtcgaatctgccaggaaagcttcagcaccactgtaaatggtatgtccaatttgtacatagaactgcaaattgtactcctaaagtaggtgcccatactgattgattctcagctagaaaattccgcaccagacatctcattgtttttgtttcttctcaacctaatgcttacagctctaggaacatggtatagcttatctataaatttcctgcaaccatgcctatctcatatactctttagggttttagtcattctaatcaactaggtcataatggagcagtgccgaagtagttatcttgtacagttatatgcgtacttggttgtcattgaatcacacaaccatgcttgtaagataagccctgtggctccctattttttcatgttccaagtctgagttaactgaattgcttgaaataaacctgcatcgtcagaagttcagaactgaacctgtataatagtattgcttaaatgaaaaacagtgttgtagagggttaacctttgttgtgccacatgttttcaagtttgtgtataacactataaccatgtcatgtaaaggaaacagtgttcagtagagtttttgtacccttgctgagctgcatgtttttgctattgtactaattctctgttttctttggtgcagcgctgactgagcctattgatatgtaagtcaaatccaacaaataactcttgactagtgagttggttcaaatacgtcaaggagaccaggaactgaagatcgtattatctttcctgcagatacagcgaatggatcgatgagtgcgagcgcgtcaacactgtcgaagatgatgatggtgcatgag

SEQ ID NO: 44 - DNA, 1052 bp, Triticum sp.
ccatggggaagagaaagtcagctgctaagccaccccctaagaagcggatggacaagctcgacacggtcttttcctgcccgttctgcaatcatgggagcagtgttgaatgccgaatgttagtactaccttcacacgttttcctgttcattggttgatattggatggctatcaattcagcattgtgactggggtttccccctttttttcttcagtgatatgaagaatctgattggtgaggcaaattgtcgaatctgccaggaaagcttcagcacaaccgtaaacggtatgcccaaaatttacataaaactgcagattttacctcctaatgcagatgttgtactgattgattctcagctagaaatttggctctagacatctcaattgtttttgtttcttctcaaactaatgcttacagctttaagaacagggtatagcttatctataaatttcctacagccatgtccatctcatatactcttgggggtttagtcattctaatcaactaggtcataatgaagcagagccgaggtagcaatcatgtacagttatattcttacctggttgccattaatcacaccatcatgtctgtaaggcaggtcatgtggctccctatgtttatcgtgtccaagtctgagttaactgaattgcttgaaacaaacctgcatcatcacaagttcagaactgaacctgtataataatattgcttaaatgaaaaacagtgttgtagagggttaaccattgttgtgccacatgttttcaagtttgtgtataactatctcatgtaaaggaaacagtgttcagtagaattttttacccttgctgacctacatgtttctggtattgtactaattctctgttttctttggtgcagcgctgactgagcctattgatatgtaagtcaaattcaacaaataactcttgactagtgagttggttcaaatacgtcaaggagaccaggaactgaagatcgtattatctttcctgcagatacagcgaatggatcgatgagtgcgagcgcgtcaacactgtcgaagatgatgatggtgcatgag

SEQ ID NO: 45 - DNA, 1051 bp, Triticum sp.
ccatggggaagagaaagtcagctgctaagccaccccctaagaagcggatggacaagctcgacacggtcttttcctgcccgttctgcaatcatgggagcagtgttgaatgccgaatgttagtactaccttcacacgttttcctgttcattggttgatattggatggctatcaattcagcattgtgactggggtttccccctttttttcttcagtgatatgaagaatctgattggtgaggcaaattgtcgaatctgccaggaaagcttcagcacaaccgtaaacggtatgcccaaaatttacataaaactgcagattttacctcctaatgcagatgttgtactgattgattctcagctagaaatttggctctagacatctcaattgtttttgtttcttctcaaactaatgcttacagctttaagaacagggtatagcttatctataaatttcctacagccatgtccatctcatatactcttgggggtttagtcattctaatcaactaggtcataatgaagcagagccgaggtagcaatcatgtacagttatattcttacctggttgccattaatcacaccatcatgtctgtaaggcaggtcatgtggctccctatgtttatcgtgtccaagtctgagttaactgaattgcttgaaacaaacctgcatcatcacaagttcagaactgaacctgtataataatattgcttaaatgaaaaacagtgttgtagagggttaaccattgttgtgccacatgttttcaagtttgtgtataactatctcatgtaaaggaaacagtgttcagtagaattttttacccttgctgacctacatgtttctggtattgtactaattctctgttttctttggtgcagcgctgactgagcctattgatatgtaagtcaaattcaacaaataactcttgactagtgagttggtgcaaatacgtcagagaccaggaactgaagatcatattaattttcctgcagatacagcgaatggatcgatgagtgcgagcgcgtcaacactgtcgaagatgatgatggtgcatgagc

SEQ ID NO: 46 - DNA, 1069 bp, Triticum sp.
ccatggggaagagaaagtcagctgctaagccaccccctaagaagcggatggacaagctcgacacggtcttttcctgcccgttctgcaatcatgggagcagtgttgaatgccgaatgttagtactaccttcacacgttttcctgttcattggttgatattggatggctatcaattcagcattgtgactggggtttccccctttttttcttcagtgatatgaagaatctgattggtgaggcaaattgtcgaatctgccaggaaagcttcagcaccactgtaaatggtatgtccaatttgtacatagaactgcaaattgtactcctaaagtaggtgcccatactgattgattctcagctagaaaattccgcaccagacatctcattgtttttgtttcttctcaacctaatgcttacagctctaggaacatggtatagcttatctataaatttcctgcaaccatgcctatctcatatactctttagggttttagtcattctaatcaactaggtcataatggagcagtgccgaagtagttatcttgtacagttatatgcgtacttggttgtcattgaatcacacaaccatgcttgtaagataagccctgtggctccctattttttcatgttccaagtctgagttaactgaattgcttgaaataaacctgcatcgtcagaagttcagaactgaacctgtataatagtattgcttaaatgaaaaacagtgttgtagagggttaacctttgttgtgccacatgttttcaagtttgtgtataacactataaccatgtcatgtaaaggaaacagtgttcagtagagttttttacccttgctaagctacatgtatttagtatattgtatattgtactaattatctgttttatttggtgcagcgctgactgagcctattgatatgtaagtcaaatccaacaaataactcttgactagtgagttggtgcaaatacgtcagagaccaggaactgaagatcgtattatctttcctgcagatacagcgaatggatcgatgagtgcgagcgcgtcaacactgtcgaagatgatgatggtgcatgag

SEQ ID NO: 47 - DNA, 1045 bp, Triticum sp.; identical to SEQ ID NO: 41

SEQ ID NO: 48 - DNA, 1050 bp, Triticum sp.; identical to SEQ ID NO: 42

SEQ ID NO: 49 - protein, 89 aa, Triticum sp.; identical to SEQ ID NO: 2

SEQ ID NO: 50 - protein, 89 aa, Triticum sp.; identical to SEQ ID NO: 2

SEQ ID NO: 51 - protein, 89 aa, Triticum sp.; identical to SEQ ID NO: 2

SEQ ID NO: 52 - protein, 89 aa, Triticum sp.; identical to SEQ ID NO: 2

SEQ ID NO: 53 - protein, 89 aa, Triticum sp.; identical to SEQ ID NO: 2

SEQ ID NO: 54 - protein, 89 aa, Triticum sp.; identical to SEQ ID NO: 2

SEQ ID NO: 55 - protein, 89 aa, Triticum sp.; identical to SEQ ID NO: 2

SEQ ID NO: 56 - protein, 89 aa, Triticum sp.; identical to SEQ ID NO: 2

SEQ ID NO: 57 - DNA, 115 bp, Triticum sp.
ccatggggaagagaaagtcagctgctaagccaccccctaagaagcggatggacaagctcgacacggtcttttcctgcccgttctgcaatcatgggagcagtgttgaatgccgaat

SEQ ID NO: 58 - DNA, 69 bp, Triticum sp.
tgatatgaagaatctgattggtgaggcaaattgtcgaatctgccaggaaagcttcagcacaaccgtaaa

SEQ ID NO: 59 - DNA, 56 bp, Triticum sp.
atacagcgaatggatcgatgagtgcgagcgcgtcaacactgtcgaagatgatgatg

SEQ ID NO: 60 - DNA, 23 bp, Triticum sp.
gcgctgactgagcctattgatat

SEQ ID NO: 61 - DNA, 115 bp, Hordeum vulgare
ccatggggaagagaaagtcagctgctaagccaccccctaagaagcggatggacaagctcgacacggtcttttcctgcccgttctgcaaccatgggagcagcgttgaatgccgaat

SEQ ID NO: 62 - DNA, 69 bp, Hordeum vulgare
tgatatgaagaatctgattggtgaggcgaattgtcgaatctgccaggaaagcttcagcaccaccataaa

SEQ ID NO: 63 - DNA, 56 bp, Hordeum vulgare
atacagcgaatggatcgatgagtgcgagcgcgtcaacgctgtcggagatgatgatg

SEQ ID NO: 64 - DNA, 23 bp, Hordeum vulgare
gcgctgactgagcctattgatat

SEQ ID NO: 65 - DNA, 115 bp, Triticum sp.; identical to SEQ ID NO: 57

SEQ ID NO: 66 - DNA, 40 bp, Triticum sp.
gaatggatcgatgagtgcgagcgcgtcaacactgtcgaag

SEQ ID NO: 67 - DNA, 115 bp, Zea mays
ccatggggaagagaaagtcagctgctaagccacctcctaagaagcggatggacaagcttgacactgtcttttcctgcccattctgcaaccacgggagtagtgttgagtgccgaat

SEQ ID NO: 68 - DNA, 59 bp, Zea mays
atgaagaatctgattggcgaagcatcttgtagaatctgtcaggaaaacttcagcacaac

SEQ ID NO: 69 - DNA, 40 bp, Zea mays
gaatggattgatgagtgtgagcgtgtcaacactgttgaag

SEQ ID NO: 70 - DNA, 115 bp, Zea mays; identical to SEQ ID NO: 67

SEQ ID NO: 71 - DNA, 40 bp, Zea mays; identical to SEQ ID NO: 69

SEQ ID NO: 72 - DNA, 182 bp, Triticum sp.
gcgctgactgagcctattgatatgtaagtcaaatccaacaaataactcttgactagtgagttggttcaaatacgtcaaggagaccaggaactgaagatcgtattatctttcctgcagatacagcgaatggatcgatgagtgcgagcgcgtcaacactgtcgaagatgatgatggtgcatgag

SEQ ID NO: 73 - DNA, 71 bp, Triticum sp.
tgatatgaagaatctgattggtgaggctaactgtcgaatctgccaggaaagcttcagcaccaccgtaaatg

SEQ ID NO: 74 - DNA, 65 bp, Triticum sp.
gacaagctcgacacggtcttttcctgcccgttctgcaatcatgggagcagtgttgaatgccgaat

SEQ ID NO: 75 - DNA, 181 bp, Triticum aestivum
gcgctgactgagcctattgatatgtaagtccaatccaacaaataactcttgactagtgagttggttcaaatacgtcaagagaccaggaactgaagatcgtattatctttcctgcagatacagcgaatggatcgatgagtgcgagcgcgtcaacactgtcgaagatgatgatggtgcatgag

SEQ ID NO: 76 - DNA, 71 bp, Triticum aestivum
tgatatgaagaatctgattggtgaggctaattgtcgaatctgccaggaaagcttcagcaccactgtaaatg

SEQ ID NO: 77 - DNA, 65 bp, Triticum aestivum
gacaagctcgacacggtcttttcctgcccattctgcaatcatgggagcagtgttgaatgccgaat

SEQ ID NO: 78 - DNA, 65 bp, Triticum sp.
atacagcgaatggatcgatgagtgcgagcgcgtcaacactgtcgaagatgatgatggtgcatgag

SEQ ID NO: 79 - DNA, 69 bp, Triticum sp.
tgatatgaagaatctgattggtgaggctaactgtcgaatctgccaggaaagcttcagcaccaccgtaaa

SEQ ID NO: 80 - DNA, 23 bp, Triticum sp.
gcgctgactgagcctattgatat

SEQ ID NO: 81 - DNA, 115 bp, Triticum aestivum; identical to SEQ ID NO: 57

SEQ ID NO: 82 - DNA, 65 bp, Triticum aestivum
atacagcgaatggatcgatgagtgcgagcgcgtcaacactgttgaagatgatgatggtgcatgag

SEQ ID NO: 83 - DNA, 69 bp, Triticum aestivum; identical to SEQ ID NO: 58

SEQ ID NO: 84 - DNA, 23 bp, Triticum aestivum
gcgctgactgagcctattgatat

SEQ ID NO: 85 - DNA, 115 bp, Triticum aestivum; identical to SEQ ID NO: 57

SEQ ID NO: 86 - DNA, 65 bp, Triticum aestivum; identical to SEQ ID NO: 82

SEQ ID NO: 87 - DNA, 69 bp, Triticum aestivum; identical to SEQ ID NO: 58

SEQ ID NO: 88 - DNA, 23 bp, Triticum aestivum
gcgctgactgagcctattgatat

SEQ ID NO: 89 - DNA, 71 bp, Triticum sp.; identical to SEQ ID NO: 73

SEQ ID NO: 90 - DNA, 65 bp, Triticum sp.; identical to SEQ ID NO: 78

SEQ ID NO: 91 - DNA, 23 bp, Triticum sp.
gcgctgactgagcctattgatat

SEQ ID NO: 92 - DNA, 115 bp, Triticum aestivum; identical to SEQ ID NO: 57

SEQ ID NO: 93 - DNA, 71 bp, Triticum aestivum; identical to SEQ ID NO: 73

SEQ ID NO: 94 - DNA, 65 bp, Triticum aestivum; identical to SEQ ID NO: 78

SEQ ID NO: 95 - DNA, 23 bp, Triticum aestivum
gcgctgactgagcctattgatat

SEQ ID NO: 96 - DNA, 262 bp, Triticum sp.
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggcaaattgtcgaatctgccaggaaagcttcagcacaaccgtaaacgcgctgactgagcctattgatatatacagcgaatggatcgatgagtgcgagcgcgtcaacactgtcgaagatgatgatg

SEQ ID NO: 97 - DNA, 270 bp, Triticum sp.
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggcaaattgtcgaatctgccaggaaagcttcagcacaaccgtaaacgcgctgactgagcctattgatatatacagcgaatggatcgatgagtgcgagcgcgtcaacactgtcgaagatgatgatggtgcatga

SEQ ID NO: 98 - DNA, 253 bp, Triticum sp.
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggcaaattgtcgaatctgccaggaaagcttcagcacaaccgtaaacgcgctgactgagcctattgatatatacagcgaatggatcgatgagtgcgagcgcgtcaacactgtcgaag

SEQ ID NO: 99 - DNA, 262 bp, Hordeum vulgare
atggggaagagaaagtcagctgctaagccaccccctaagaagcggatggacaagctcgacacggtcttttcctgcccgttctgcaaccatgggagcagcgttgaatgccgaattgatatgaagaatctgattggtgaggcgaattgtcgaatctgccaggaaagcttcagcaccaccataaatgcgctgactgagcctattgatatatacagcgaatggatcgatgagtgcgagcgcgtcaacgctgtcggagatgatgatg

SEQ ID NO: 100 - DNA, 270 bp, Hordeum vulgare
atggggaagcgaaaggcggccgccaagccaccccctaggaagaggatggacaagctcgatacggtcttctcctgcccattctgcaaccacgggagcagcgttgaatgccggattgatatgaagaatctgattggtgaggctaattgtcaaatctgccaggaaagcttcagcaccactgcaaatgcgctgactgaagctattgatatatacagcgaatggattgatgagtgtgagcgcgtcaacactgtcgaagatgatgatggtgcatga

SEQ ID NO: 101 - DNA, 253 bp, Zea mays
atggggaagagaaagtcagctgctaagccacctcctaagaagcggatggacaagcttgacactgtcttttcctgcccattctgcaaccacgggagtagtgttgagtgccgaattgacatgaagaatctgattggcgaagcatcttgtagaatctgtcaggaaaacttcagcacaactgttaatgcactcactgagcctattgatatatatagtgaatggattgatgagtgtgagcgtgtcaacactgttgaag

SEQ ID NO: 102 - DNA, 270 bp, Triticum sp.; identical to SEQ ID NO: 97

SEQ ID NO: 103 - DNA, 270 bp, Triticum aestivum
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggcaaattgtcgaatctgccaggaaagcttcagcacaaccgtaaacgcgctgactgagcctattgatatatacagcgaatggatcgatgagtgcgagcgcgtcaacactgttgaagatgatgatggtgcatga

SEQ ID NO: 104 - DNA, 270 bp, Triticum aestivum; identical to SEQ ID NO: 103

SEQ ID NO: 105 - DNA, 270 bp, Triticum aestivum; identical to SEQ ID NO: 103

SEQ ID NO: 106 - DNA, 270 bp, Triticum aestivum; identical to SEQ ID NO: 103

SEQ ID NO: 107 - protein, 74 aa, Triticum sp.
MDKLDTVFSCPFCNHGSSVECRIDMKNLIGEANCRICQESFSTTVNALTEPIDIYSEWIDECERVNTVEDDDGA

SEQ ID NO: 108 - protein, 71 aa, Triticum sp.
MDKLDTVFSCPFCNHGSSVECRIDMKNLIGEANCRICQESFSTTVNALTEPIDIYSEWIDECERVNTVEDD

SEQ ID NO: 109 - protein, 72 aa, Triticum sp.
MDKLDTVFSCPFCNHGSSVECRIDMKNLIGEANCRICQESFSTTVNALTEPIDIYSEWIDECERVNTVEDDD

SEQ ID NO: 110 - protein, 65 aa, Triticum sp.
DKLDTVFSCPFCNHGSSVECRIDMKNLIGEANCRICQESFSTTVNALTEPIDIYSEWIDECERVN

SEQ ID NO: 111 - protein, 65 aa, Triticum sp.; identical to SEQ ID NO: 110

SEQ ID NO: 112 - protein, 55 aa, Triticum sp.
SSVECRIDMKNLIGEANCRICQESFSTTVNALTEPIDIYSEWIDECERVNTVEDD

SEQ ID NO: 113 - protein, 74 aa, Triticum sp.; identical to SEQ ID NO: 107

SEQ ID NO: 114 - protein, 60 aa, Triticum sp.
LDTVFSCPFCNHGSSVECRIDMKNLIGEANCRICQESFSTTVNALTEPIDIYSEWIDECE

SEQ ID NO: 115 - protein, 66 aa, Triticum sp.
MDKLDTVFSCPFCNHGSSVECRIDMKNLIGEANCRICQESFSTTVNALTEPIDIYSEWIDECERVN

SEQ ID NO: 116 - protein, 73 aa, Triticum sp.
MDKLDTVFSCPFCNHGSSVECRIDMKNLIGEANCRICQESFSTTVNALTEPIDIYSEWIDECERVNTVEDDDG

SEQ ID NO: 117 - protein, 71 aa, Triticum sp.
KLDTVFSCPFCNHGSSVECRIDMKNLIGEANCRICQESFSTTVNALTEPIDIYSEWIDECERVNTVEDDDG

SEQ ID NO: 118 - protein, 74 aa, Oryza sativa
MDKLDTVFSCPFCNHGSSVECRIDMKNLIGEASCRICQENFSTTVNALTEPIDIYSEWIDECERVNNVEDDDGA

SEQ ID NO: 119 - protein, 71 aa, Vitis vinifera
MDKLDTVFSCPFCNHGTSVECRIDMKNLIGEAVCRICQESFSMTVNALSEPIDVYSEWIDECERVNTLDED

SEQ ID NO: 120 - protein, 71 aa, Populus sp.
MDKLDTVFSCPFCNHGTSVECRIDMKNLIGEAVCGICQESFSMTITALTEPIDIYSEWIDECERVNSLEDD

SEQ ID NO: 121 - protein, 72 aa, Oryza sativa
MDKLDTVFCCPFCNHGSSVECRIDLKNLIGEASCRICLETFSTSVNALTEPIDIYSEWIDECERVNNPEEDN

SEQ ID NO: 122 - protein, 71 aa, Arabidopsis thaliana
MDKLDTIFSCPFCNHGSSVECIIDMKHLIGKAACRICEESFSTTITALTEAIDIYSEWIDECERVNTAEDD

SEQ ID NO: 123 - protein, 65 aa, Picea sitchensis
EKLSTVFSCPFCNHESSVECRMDRKDLIGEASCRICQESFSTSINALSEPVDVYCEWIDECERVN

SEQ ID NO: 124 - protein, 65 aa, Physcomitrella patens
EKLATVFSCPFCNHDNSVECRMDRKSLIGEASCRICLETFSTPIDSLSDPIDVYSDWIDECERIN

SEQ ID NO: 125 - protein, 65 aa, Physcomitrella patens
EKLATVFSCPFCNHDNSVECRMDRKNQIGEASCRICLEKFSTPIDSLSDPIDVYSDWIDECERIN

SEQ ID NO: 126 - protein, 74 aa, Oryza sativa
KLETAFSCPFCDHGGAVECSIDIKHMIAEASCFVCQARYSTTAHALTEPIDVYSEWIDQCELAKAAAAAGDDDD

SEQ ID NO: 127 - protein, 74 aa, Oryza sativa; identical to SEQ ID NO: 126

SEQ ID NO: 128 - protein, 74 aa, Oryza sativa; identical to SEQ ID NO: 126

SEQ ID NO: 129 - protein, 74 aa, Oryza sativa; identical to SEQ ID NO: 126

SEQ ID NO: 130 - protein, 59 aa, Arabidopsis thaliana
SNASCKMNHNIDMKHLIGKAACRICEESFSTTITALTEAIDIYSEWIDECERVNTAEDD

SEQ ID NO: 131 - protein, 60 aa, Candida albicans
LDTTFTCLFCNHEKSVICTIDKKNLLGELHCKICGQSFQTAIHSLSQPVDIYSDWIDACE

SEQ ID NO: 132 - protein, 60 aa, Candida albicans; identical to SEQ ID NO: 131

SEQ ID NO: 133 - protein, 60 aa, Candida albicans; identical to SEQ ID NO: 131

SEQ ID NO: 134 - protein, 60 aa, Candida albicans; identical to SEQ ID NO: 131

SEQ ID NO: 135 - protein, 60 aa, Candida albicans; identical to SEQ ID NO: 131

SEQ ID NO: 136 - protein, 72 aa, Candida glabrata
VQKLDTSFNCLFCNHEKSVSCTLDKKNCIGHLSCKICGQSFQTRINSLSQPVDVYSDWFDAVEEVNSGRGSD

SEQ ID NO: 137 - protein, 66 aa, Vanderwaltozyma polyspora
VQKLDTKFNCLFCNHDKSVSCTIDKKNSIGSLSCKICGQTFQTRINALSQPVDVYSDWFDAVEEVN

SEQ ID NO: 138 - protein, 76 aa, Saccharomyces cerevisiae
VQKLDTKFNCLFCNHEKSVSCTLDKKNSIGTLSCKICGQSFQTRINSLSQPVDVYSDWFDAVEEVNSGRGSDTDDG

SEQ ID NO: 139 - protein, 74 aa, Saccharomyces cerevisiae
KLDTKFNCLFCNHEKSVSCTLDKKNSIGTLSCKICGQSFQTRINSLSQPVDVYSDWFDAVEEVNSGRGSDTDDG

SEQ ID NO: 140 - DNA, 25 bp, Triticum sp.
cttgtttctatagcagaggtgtcta

SEQ ID NO: 141 - DNA, 25 bp, Triticum sp.
gtctaagtaagtgtctatgctcaac

SEQ ID NO: 142 - DNA, 25 bp, Triticum sp.
ttggcttattttttacgcacctctc

SEQ ID NO: 143 - DNA, 25 bp, Triticum sp.
tttacgcacctctctaagcatcttg

SEQ ID NO: 144 - DNA, 25 bp, Triticum sp.
atcttgcgctgttctggcctgatgt

SEQ ID NO: 145 - DNA, 25 bp, Triticum sp.
ggcctgatgtgtttgcttgtctgtc

SEQ ID NO: 146 - DNA, 25 bp, Triticum sp.
ttgcttgtctgtctactcatgccta

SEQ ID NO: 147 - DNA, 25 bp, Triticum sp.
gtctactcatgcctacctatttaat

SEQ ID NO: 148 - DNA, 25 bp, Triticum sp.
aatggatcattgaacctctctcgag

SEQ ID NO: 149 - DNA, 25 bp, Triticum sp.
ctctctcgaggttttgtttcaaggg

SEQ ID NO: 150 - DNA, 25 bp, Triticum sp.
gtattgacacttaaacgatgctttg

SEQ ID NO: 151 - DNA, 32 bp, Triticum sp.
cgttcttcccttgaaacaaaacctcgagagag

SEQ ID NO: 152 - DNA, 32 bp, Triticum sp.
caggccagaacagcgcaagatgcttagagagg

SEQ ID NO: 153 - DNA, 21 bp, Triticum sp.
taatacgactcactatagggc

SEQ ID NO: 154 - DNA, 19 bp, Triticum sp.
actatagggcacgcgtggt

SEQ ID NO: 155 - DNA, 1247 bp, Triticum sp.
ggtcgacccacgcgtccgcggacgtgtgggcagcaactagagatgttgttgcgctgcccaggtgccaccgcaatatgttgcagcatcccagctgccttccaattggtttgggccttgatgtttccactggaacatacaaggttgcacggtctttctaccgttccggtgattacagttctgttgcaatggggatggaggtcttcactattaatggtgaagagggccactggagggaaacgttgctggatccaccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctatgctcaacaaataagcaacggtgcttaagagaaattggcttattttttacgcacctctctaagcatcttgcgctgttctggcctgatgtgtttgcttgtctgtctactcatgcctacctatttaatggatcattgaacctctctcgaggttttgtttcaagggaagaacgttgaagatcaattctgtttaatgttgtatttgtttcaagtgaagaacgttgaagatcaattctgtttaatgttgtattgacacttaaacgatgctttgttttgtgcatgttattttctgctgtttctaaagaaaaaaaaaaaaaaaaaa

SEQ ID NO: 156 - DNA, 690 bp, Triticum sp.
ggtcgacccacgcgtccgcggacgtgtgggcagcaactagagatgttgttgcgctgcccaggtgccaccgcaatatgttgcagcatcccagctgccttccaattggtttgggccttgatgtttccactggaacatacaaggttgcacggtctttctaccgttccggtgattacagttctgttgcaatggggatggaggtcttcactattaatggtgaagagggccactggagggaaacgttgctggatccaccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgag

SEQ ID NO: 157 - DNA, 563 bp, Triticum sp.
ccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcct

SEQ ID NO: 158 - DNA, 450 bp, Triticum sp.; identical to SEQ ID NO: 4

SEQ ID NO: 159 - DNA, 1529 bp, Triticum sp.
cctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggtcggagggagtagattccaatccctcaggcattttttccaccaacatccgtttcttccagtggtctttacaagaagatgatttgccaaaagtagcaacaaaagttggaatgagtcatagtagtagcgcaatgcttcttcacgggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcagcaacggtgcttaagagaaattggcttattttttacgcacctctctaagcatcttgcgctgttctggcctg

SEQ ID NO: 160 - DNA, 1691 bp, Triticum sp.
gtcagcaaagccagtgccgaatctaagtatatattttgttttgtttgttgctacatggacttgaagagggttgatgatacctttacacaaaagacggttaatgatgcgagaaagaggaagtggaggatgccacagaggaagcaaacgaagaggtctggacatcctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggacggagggagtagattccaatccctcaggcattttttccaccaacatccgtttcttccagtggtctttacaagaagatgatttgccaaaagtagcaacaaaagttggaatgagtcatagtagtagcgcaatgcttcttcacgggaggcacttcccggctggcaagtttgggttggtgtcccagatggcacactgcgatgggttggtgctgctccctacagacaccaatgcttatgtcttcaacccggccactagagatggcattgccctgccgaggagtcatcacaacgtgttgcaacgtctcgtatgccttcccattagtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtataagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcagcaacggtgcttaagagaaattggcttattttttacgcacctctctaagcatcttgcgctgttctggcctg

SEQ ID NO: 161 - DNA, 1794 bp, Triticum sp.
ttccgggaaggatcctaaacagaccgcgtagaccgtgttcgaattcatccatcgaacacgcaaaatatttatatatatactatgtatatctgtgttttttcgtgtcagcaaagccagtgccgaatctaagtatatattttgttttgtttgttgctacatggacttgaagagggttgatgatacctttacacaaaagacggttaatgatgcgagaaagaggaagtggaggatgccacagaggaagcaaacgaagaggtctggacatcctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggacggagggagtagattccaatccctcaggcattttttccaccaacatccgtttcttccagtggtctttacaagaagatgatttgccaaaagtagcaacaaaagttggaatgagtcatagtagtagcgcaatgcttcttcacgggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattagtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgtcgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcagcaacggtgcttaagagaaattggcttattttttacgcacctctctaagcatcttgcgctgttctggcctg

SEQ ID NO: 162 - DNA, 750 bp, Triticum sp.
cctttgtccgcaaactgccatacactgcaaggggtgtctattctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 163 - DNA, 750 bp, Triticum sp.; identical to SEQ ID NO: 162

SEQ ID NO: 164 - DNA, 750 bp, Triticum sp.
cctttgtccgcaaactgccatacactgcaaggggtgtctattctatttcatcgatagggaaaatcagccatgctccccacgggggcagcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgataccgatgagctaaggttccttgggcctagtgaagacacattgggacatgcatgggaaaatttattttggttcgacctactctattacacgccaagtcttgttccagttactcctaaggcgagcttacgtgcctcataagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 165 - DNA, 750 bp, Triticum sp.
cctttgtccgcaaactgccatacactgcaaggggtgtctatcctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacgtgcctcataagaccatgtacactgcaaggtgtttagagaagtgcttgtataaataaattgttttttcccttaagtaccggtgcttgtttctatagcagaagtgtctaagtaagtgtctgtgctcaacagataagcaacggtgcttaagagaaattggcttattttttacgcacctctctaagcatcttgcgctgttctggcctg

SEQ ID NO: 166 - DNA, 750 bp, Triticum sp.
cctttgtccgcaaactgccatacactgcaaggggtgtctattctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcatgagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 167 - DNA, 750 bp, Triticum sp.
cctttgtccgcaaactgccatacactgcaaggggtgtctattctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcctccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 168 - DNA, 750 bp, Triticum sp.
cctttgtccgcaaactgccatacaccgcaaggggtgtctattctatttcatcgatagggaaaatcagccatgctccccacgggggctgcttcgtttcagctttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttacttctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 169 - DNA, 749 bp, Triticum sp.
cctttgtccgcaaactgccatacactgcaaggggtgtctattctatttcatcgatagggaaaatcagccatgctccccacgggggctgcttcgcttcagccttcaagacgaaaccttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccaatgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcaacggtgcttaagagaaattggcttattttttacgcacctctctaagcatcttgcgctgttctggcctg

SEQ ID NO: 170 - DNA, 996 bp, Triticum sp.
ccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctatgctcaacaaataagcaacggtgcttaagagaaattggcttattttttacgcacctctctaagcatcttgcgctgttctggcctgatgtgtttgcttgtctgtctactcatgcctacctatttaatggatcattgaacctctctcgaggttttgtttcaagggaagaacgttgaagatcaattctgtttaatgttgtatttgtttcaagtgaagaacgttgaagatcaattctgtttaatgttgtattgacacttaaacgatgctttgttttgtgcatgttattttctgctgtttctaaagaaaaaaaaaaaaaaaaaa

SEQ ID NO: 171 - DNA, 333 bp, Triticum sp.
atgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 172 - DNA, 312 bp, Triticum sp.
atgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 173 - protein, 110 aa, Triticum sp.
MQRVLISMTRDILVPEWECHYVMNVSDQCLPMASLSNGGILLRRGHCLFRYYMKDHGVKENDMFDIDELRFLGPSEDTLGHAWENLFWFDLLYYTPSLVPVTPKASLHAS

SEQ ID NO: 174 - protein, 103 aa, Triticum sp.
MTRDILVPEWECHYVMNVSDQCLPMASLSNGGILLRRGHCLFRYYMKDHGVKENDMFDIDELRFLGPSEDTLGHAWENLFWFDLLYYTPSLVPVTPKASLHAS

SEQ ID NO: 175 - DNA, 633 bp, Triticum sp.
atggggatggaggtcttcactattaatggtgaagagggccactggagggaaacgttgctggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 176 - DNA, 702 bp, Triticum sp.
atgtttccactggaacatacaaggttgcacggtctttctaccgttccggtgattacagttctgttgcaatggggatggaggtcttcactattaatggtgaagagggccactggagggaaacgttgctggatccaccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 177 - DNA, 609 bp, Triticum sp.
atggtgaagagggccactggagggaaacgttgctggatccaccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctcgatgacaagagatatattggtcccggaatgggaatgtcactatgtaatgaatgtttcagaccagtgcttgccaatggcctcacttagcaatggcgggattcttctacggagaggccattgtctcttccgttattacatgaaggatcatggtgtaaaagaaaatgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 178 - DNA, 333 bp, Triticum sp.; identical to SEQ ID NO: 171

SEQ ID NO: 179 - DNA, 327 bp, Triticum sp.
atgttgcagcatcccagctgccttccaattggtttgggccttgatgtttccactggaacatacaaggttgcacggtctttctaccgttccggtgattacagttctgttgcaatggggatggaggtcttcactattaatggtgaagagggccactggagggaaacgttgctggatccaccgtaccttatcctttgtccgcaaactgccatacactgcaaggggtgtctattctatttcatcgataggggaaaatcagccatgctccccacgggggctgcttcgtttcagccttcaagacgaaactttcggcatcactccgttgcttaa

SEQ ID NO: 180 - DNA, 312 bp, Triticum sp.; identical to SEQ ID NO: 172

SEQ ID NO: 181 - protein, 258 aa, Triticum sp.
MLLRCPGATAICCSIPAAFQLVWALMFPLEHTRLHGLSTVPVITVLLQWGWRSSLLMVKRATGGKRCWIHRTLSFVRKLPYTARGVYSISSIGENQPCSPRGLLRFSLQDETFGITPLLNNLYPTVEDEDIVIHELGGELCATFFCKSMQRVLISMTRDILVPEWECHYVMNVSDQCLPMASLSNGGILLRRGHCLFRYYMKDHGVKENDMFDIDELRFLGPSEDTLGHAWENLFWFDLLYYTPSLVPVTPKASLHAS

SEQ ID NO: 182 - protein, 108 aa, Triticum sp.
MLQHPSCLPIGLGLDVSTGTYKVARSFYRSGDYSSVAMGMEVFTINGEEGHWRETLLDPPYLILCPQTAIHCKGCLFYFIDRGKSAMLPTGAASFQPSRRNFRHHSVA

SEQ ID NO: 183 - DNA, 333 bp, Triticum sp.
atgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 184 - DNA, 333 bp, Triticum sp.; identical to SEQ ID NO: 183

SEQ ID NO: 185 - DNA, 333 bp, Triticum sp.; identical to SEQ ID NO: 183

SEQ ID NO: 186 - DNA, 312 bp, Triticum sp.
atgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 187 - DNA, 312 bp, Triticum sp.; identical to SEQ ID NO: 186

SEQ ID NO: 188 - DNA, 333 bp, Triticum sp.; identical to SEQ ID NO: 183

SEQ ID NO: 189 - DNA, 312 bp, Triticum sp.; identical to SEQ ID NO: 186

SEQ ID NO: 190 - protein, 209 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPVTPKASLHAS

SEQ ID NO: 191 - DNA, 1529 bp, Triticum sp.; identical to SEQ ID NO: 159

SEQ ID NO: 192 - protein, 210 aa, Triticum sp.
MGMEVFTINGEEGHWRETLLDPPYLILCPQTAIHCKGCLFYFIDRENQPCSPRGLLRFSLQDETFGITPLLNNLYPTVEDEDIVIHELGGELCATFFCKSMQRVLISMTRDILVPEWECHYVMNVSDQCLPMASLSNGGILLRRGHCLFRYYMKDHGVKENDMFDIDELRFLGPSEDTLGHAWENLFWFDLLYYTPSLVPVTPKASLHAS

SEQ ID NO: 193 - DNA, 1794 bp, Triticum sp.; identical to SEQ ID NO: 161

SEQ ID NO: 194 - DNA, 630 bp, Triticum sp.
atggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgtcgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 195 - DNA, 624 bp, Triticum sp.
atggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgtcgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 196 - DNA, 603 bp, Triticum sp.
atgagtcatagtagtagcgcaatgcttcttcacgggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattga

SEQ ID NO: 197 - DNA, 582 bp, Triticum sp.
atgcttcttcacgggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattga

SEQ ID NO: 198 - DNA, 528 bp, Triticum sp.
atggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattga

SEQ ID NO: 199 - DNA, 492 bp, Triticum sp.
atggacttgaagagggttgatgatacctttacacaaaagacggttaatgatgcgagaaagaggaagtggaggatgccacagaggaagcaaacgaagaggtctggacatcctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggacggagggagtagattccaatccctcaggcattttttccaccaacatccgtttcttccagtggtctttacaagaagatga

SEQ ID NO: 200 - DNA, 420 bp, Triticum sp.
atgccacagaggaagcaaacgaagaggtctggacatcctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggacggagggagtagattccaatccctcaggcattttttccaccaacatccgtttcttccagtggtctttacaagaagatga

SEQ ID NO: 201 - DNA, 357 bp, Triticum sp.
atggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatctactccctccgtcccaaaataagtgtcttgagcttagtacaaatttgtatttgtactagagctagtacaaagttgagacacttattttgggacggagggagtagattccaatccctcaggcattttttccaccaacatccgtttcttccagtggtctttacaagaagatga

SEQ ID NO: 202 - DNA, 333 bp, Triticum sp.; identical to SEQ ID NO: 183

SEQ ID NO: 203 - DNA, 333 bp, Triticum sp.; identical to SEQ ID NO: 183

SEQ ID NO: 204 - DNA, 333 bp, Triticum sp.; identical to SEQ ID NO: 183

SEQ ID NO: 205 - DNA, 97 bp, Triticum sp.
ctccgtcccaaaataagtgtcttgagcttagtacaaatttgtatttgtactagagctagtacaaagttgagacacttattttgggtcggagggagta

SEQ ID NO: 206 - DNA, 28 bp, Triticum sp.
tactccctccgtcccaaaataagtgtct

SEQ ID NO: 207 - DNA, 28 bp, Triticum sp.
agacacttattttgggtcggagggagta

SEQ ID NO: 208 - DNA, 91 bp, Triticum turgidum
ctccgtccaaaaataagtgtcttgagcttagtacaaatttgtactagagctagtacaaagttgagacacttattttgggacggagggagta

SEQ ID NO: 209 - DNA, 28 bp, Triticum turgidum
tactccctccgtcccaaaataagtgtct

SEQ ID NO: 210 - DNA, 97 bp, Triticum turgidum
tactccctccgtcccaaaataagtgtcttgagcttagtaaaaatttgtactagagatagtacaaagttgagacacttattttgggatggagggagta

SEQ ID NO: 211 - DNA, 28 bp, Triticum turgidum
agacacttattttgggacggagggagta

SEQ ID NO: 212 - DNA, 28 bp, Triticum turgidum
tactccctccatcccaaaataagtgtct

SEQ ID NO: 213 - DNA, 97 bp, Hordeum vulgare
tactccctccgttccaaaataagtgtcttgagcgtagtacaattttgtactagagctagtacaaagttgagacagttattttgggacggagggagta

SEQ ID NO: 214 - DNA, 28 bp, Hordeum vulgare
tactccctccgtcccaaaataactgtct

SEQ ID NO: 215 - DNA, 559 bp, Triticum sp.
ccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcct

SEQ ID NO: 216 - DNA, 502 bp, Triticum sp.
ggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattt

SEQ ID NO: 217 - DNA, 267 bp, Triticum sp.
tagattccaatccctcaggcattttttccaccaacatccgtttcttccagtggtctttacaagaagatgatttgccaaaagtagcaacaaaagttggaatgagtcatagtagtagcgcaatgcttcttcacgggaggcacttcccggctggcaagtttgggttggtgtcccagatggcacactgcgatgggttggtgctgctccctacagacaccaatgcttatgtcttcaacccggccactagagatggcattgctctgccgagga

SEQ ID NO: 218 - DNA, 215 bp, Triticum sp.
cctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatcta

SEQ ID NO: 219 - DNA, 219 bp, Triticum sp.
ggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcagcaacggtgcttaagagaaattggcttattttttacgcacctctctaagcatcttgcgctgttctggcctg

SEQ ID NO: 220 - DNA, 569 bp, Triticum sp.
tgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaa

SEQ ID NO: 221 - DNA, 471 bp, Triticum sp.
gctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataaga

SEQ ID NO: 222 - DNA, 436 bp, Triticum sp.
gggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagcct

SEQ ID NO: 223 - DNA, 168 bp, Triticum sp.
tccggctgcctgtggagtcgctggctaggttcaagttggttagcaaggtctggtacaccattatctcccaacctgtttttgttcgtgcacacctccaatgctccaaacagaaacaacatcacaaattcacaatccgtcttccttcctcatcaccccccagatttatct

SEQ ID NO: 224 - DNA, 49 bp, Triticum sp.
ttttttccaccaacatccgtttcttccagtggtctttacaagaagatga

SEQ ID NO: 225 - DNA, 563 bp, Triticum aestivum; identical to SEQ ID NO: 157

SEQ ID NO: 226 - DNA, 498 bp, Triticum aestivum
  misc_feature (342)..(342): n is a, c, g, or t
ggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactccgttgcttaacaacctctaccctacagttgaagatgaagatattgttatccatgagcttggtggggaactatgcgctactttcttctgcaagagtatgcaacgagtattgatctggatgacaagagatatattggtcccggaatgggaatgtcactatgtaatgaatgtttcagaccagtgcttgccaatggnctcacttagcaatggcgggattcttctacggagaggcattgtctctcccgttattacatgaaggtcatggtgtaaaagaaaatgacatgttcgatatcgataactaaggtccntgggccnatgaagacacatgggacatcatgggaaaatttattt

SEQ ID NO: 227 - DNA, 266 bp, Triticum aestivum
tagattccaatccctcaggcattttttccaccaacatccgtttcttccagtggtctttacaagaagatgatttgccaaaagtagcaacaaaagttggaatgagtcatagtagtagcgcaatgcttcttcatgggaggcacttcccggctggcgagtttgggttggtgtcccagatggcacactgcgatctgttggtgctgctccctacgacaccaatgcttatgtcttcaacccggccactagaagatgcattgctctgccgagga

SEQ ID NO: 228 - DNA, 207 bp, Triticum aestivum
cctgcaggcgaggaattgttggacgagatggtgtgggagatcttaatccggctgcctgtggagtcgctggctaggttcaagttggttagcaaggtctggtacaccattatctcccaacctgtttttgttcgtgcacacctccaacgctccaaacagaaacaacatcacaatccgtcttccttcctcatcaccccccagatttatcta

SEQ ID NO: 229 - DNA, 216 bp, Triticum aestivum
ggcgagcttacatgcctcataagaccatgtacactgcaaggtgtttagagaagtgcttgtataaataaattgttttttcccttaagtaccggtgcttgtttctatagcagaggtgtctaagtaagtgtctatgctcaacaaataagcaacggtgcttaagagaaattggcttattttttacgcacctctctaagcatcttgcgctgttctggcctg

SEQ ID NO: 230 - DNA, 575 bp, Triticum aestivum
tgccttccaattggtttgggccttgatgtttccactggaacatacaaggttgcacggtctttctaccgttccggtgattacagttctgttgcaatggggatggaggtcttcactattaatggtgaagagggccactggagggaaacgttgctggatccaccataccctattgtgtgtccgcaaactgccatacactgcaagggttccctgttctattcatagataagaaaaatcagcgatgccccccgcaggcgctgcttcgtttctgccttcaagatgaaacatttggtatcactccattgcttaacaacgtgtaccctacagttgaagatgatgatattgttctccatgagcttgacggggaattatgtgctactttctttagcaagtgtgtgcaacgagtattggtctggatggcaagagatattgtgaatccggagtgggattgccattatgtaatcaatgtctcagatcactgctgtccagtggcctcacttagcatcactggtgggtttctcttacgaagaggtcattgtcttatccgttattacatgaaagctcatggtgtaaaagaa

SEQ ID NO: 231 - DNA, 479 bp, Triticum aestivum
gctgcttcgtttctgccttcaagatgaaacatttggtatcactccattgcttaacaacgtgtaccctacagttgaagatgatgatattgttctccatgagcttgatggggaattatgtgctactttctttagcaagtgtgtgtgcaacgagtattggtctggatggcaagagatattgtgaatccggagtgggattgccattatgtaatcaatgtctcagatcactgctgtccagtggcctcacttagcatcactggtgggtttctcttacgaagaggtgattgtcttatccgttattacatgaaagctcatggtgtaaaagaagaggatacgtttgatatgggtgaccaaaggttccttgggcccagtgaagataccttgggccatgcatgggaaaatttatgtgtgtttgaactactttattacactgcaagccttgttcctattactcctaaggcgagcttacaagccttataaga

SEQ ID NO: 232 - DNA, 436 bp, Triticum aestivum
gggaggcagttccccactggtgagtttgggctggtgtcccaaatggcacactgtgatggattggtgttgctccccacaaacaccaacacttatgtcttcaacccagcaactagagatgttgttgcgctgcccaggtgccaccgcaatatgttgcagcatcccagctgccttccaattggtttgggccttgatgtttccactggaacatacaaggttgcacggtctttctaccgttccggtgattacagttctgttgcaatggggatggaggtcttcactattaatggtgaagagggccactggagggaaacgttgctggatccaccataccctattgtgtgtccgcaaactgccatacacctgcttcgtttctgccttgcaagggttccctgttctattcatagataagaaaaatcagcgatgccccccgcaggcg

SEQ ID NO: 233 - DNA, 160 bp, Triticum aestivum
tccggctgcccgtagagtccctggtgaggtttaagttggttagcaaggcctggtacgctatcatctccgaccctgtttttgttcgtgcgcacctccagtgctccaaagagaaacaacatcgcaacccgtcttccttcctcgtcaccccccagatttatct

SEQ ID NO: 234 - DNA, 49 bp, Triticum aestivum
ttttctccaccaacatccgtttctacctgtggtctttacaacaagatga

SEQ ID NO: 235 - protein, 169 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELR

SEQ ID NO: 236 - protein, 204 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPVTPKA

SEQ ID NO: 237 - protein, 201 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPVT

SEQ ID NO: 238 - protein, 190 aa, Triticum sp.
WRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPVTPKA

SEQ ID NO: 239 - protein, 199 aa, Triticum sp.
GMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPV

SEQ ID NO: 240 - protein, 171 aa, Triticum sp.
MEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGP

SEQ ID NO: 241 - protein, 126 aa, Triticum sp.
GMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQ

SEQ ID NO: 242 - protein, 172 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLG

SEQ ID NO: 243 - protein, 197 aa, Triticum sp.
EVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPV

SEQ ID NO: 244 - protein, 121 aa, Triticum sp.
EVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNV

SEQ ID NO: 245 - protein, 65 aa, Triticum sp.
MEVITINCREGSWRETLVDPPYLILCPQTAIHCKGCLFYFIDRENQPCSPRGLLRFSLQDETFGI

SEQ ID NO: 246 - protein, 111 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDI

SEQ ID NO: 247 - protein, 158 aa, Triticum sp.
MGMEVITINCREGSWRETLVDPPYLILCPQTAIHCKGCLFYFIDRENQPCSPRGLLRFSLQDETFGITLLLNNLYPTVEDEDIVIHELGGECATFFCKSMQRVLIWMTRDILVPEWECHYVMNVSDQCLPMASLSNGEILLRRGHCLFRYYMKDHGVK

SEQ ID NO: 248 - protein, 67 aa, Triticum sp.
MGMEVITINCREGSWRETLVDPPYLILCPQTAIHCKGCLFYFIDRENQPCSPRGLLRFSLQDETFGI

SEQ ID NO: 249 - protein, 52 aa, Triticum sp.
LVPEWECHYVMNVSDQCLPMASLSNGEILLRRGHCLFRYYMKDHGVKENDMF

SEQ ID NO: 250 - protein, 77 aa, Triticum sp.
DQCLPMASLSNGEILLRRGHCLFRYYMKDHGVKENDMFDIDELRFLGPSEDTLGHAWENLFWFDLLYYTPSLVPVTP

SEQ ID NO: 251 - protein, 79 aa, Triticum sp.
GSWRETLVDPPYLILCPQTAIHCKGCLFYFIDRENQPCSPRGLLRFSLQDETFGITLLLNNLYPTVEDEDIVIHELGGE

SEQ ID NO: 252 - protein, 167 aa, Oryza sativa
MGFEVFTINGEESCWRETLVDPPYPVLYSKIVTHCKGCLFYYIDKKNQQNPPQALLRFSLQDETFGVTPLLTDTYPQVEDDEVTITELGGQLCATFFCNTLQQVCIYMSGDVMDPDWNCWHIINFMNCYPIASLGSGRILLRRGSLVFCTNLKSYSIEDKLDIDDIR

SEQ ID NO: 253 - protein, 203 aa, Oryza sativa
MGMEVFTVGETGAGCGWRETVTDPPYPVSRWLTAFTVNGGYLFWYMDRFRYPNDTLHGGLLWFSLQDQAFRVTLLPHSLDPALDDKVRPDVLHGELCVLHANSDTMPVTIWTTNSPSFDDWERRYCIYVSRLCHPMGLLGDGGMLLWAKHTIHRYDLWSDELTAVCELGGIRYQGGRPPRWKNLFNFSVMPYTESLVRITAAA

SEQ ID NO: 254 - protein, 203 aa, Oryza sativa; identical to SEQ ID NO: 253

SEQ ID NO: 255 - protein, 208 aa, Oryza sativa
MGMEVLTVGEPGARWRETAVDPPHPITRWRTALAVNGGYLFWYMDRRRYPDDAPRGLLRFSLRDEAFAVTRLPESMDPTLDENVLPDVLHGELCVVQALPDKAGVLIWTMSSSSMDNDDVHLDDGPWELRYCICVNALCHPLGVLPDGGGILLWANRSVHRYDFSARKLAGVVCNLDRIRYQGGRPARWKSVVDFTLMPYTESLVRIT

SEQ ID NO: 256 - protein, 208 aa, Oryza sativa; identical to SEQ ID NO: 255

SEQ ID NO: 257 - protein, 208 aa, Oryza sativa; identical to SEQ ID NO: 255

SEQ ID NO: 258 - protein, 208 aa, Oryza sativa; identical to SEQ ID NO: 255

SEQ ID NO: 259 - protein, 208 aa, Oryza sativa
MGMEVLTVGEPGARWRETAVDPPHPITRWRTALAVNGGYLFWYMDRRRYHDDAPRGLLRFSLRDEAFAVTRLPESMDPTLDENVLPDVLHGELCVVQALPDKAGVLIWTMSSSSMDNDDVHLDDGPWELRYCICVNALCHPLGVLPDGGGILLWANRSVHRYDFSARKLAGVVCNLDRIRYQGGRPARWKSVVDFTLMPYTESLVRIT

SEQ ID NO: 260 - protein, 187 aa, Oryza sativa
WRETVTDPPYPVSRWLTAFTVNGGYLFWYMDRFRYPNDTLHGGLLWFSLQDQAFRVTLLPHSLDPALDDKVRPDVLHGELCVLHANSDTMPVTIWTTNSPSFDDWERRYCIYVSRLCHPMGLLGDGGMLLWAKHTIHRYDLWSDELTAVCELGGIRYQGGRPPRWKNLFNFSVMPYTESLVRITAAA

SEQ ID NO: 261 - protein, 233 aa, Oryza sativa
GMELCTIGGNGGVSVGSCWREIVADTPYPVLVWHTATFFRGALFFTTCHDTAARPPQESRLLRLCLRDETFSVVAPPPPCRPPFLHEAFHLSELNGVLCLAHAAGAGDEGTSTSSSVVIWMTEDGVSPRWSKRCVFTSTSMFIPIALFHHGGGGGGGVIGKRGDLLFFLGDDDDGEDESRIGEGKEEEKQVVCLNGMTYHDDEQGRHTVVVGSSWENLHYYKLIPYTESLVPI

SEQ ID NO: 262 - protein, 233 aa, Oryza sativa; identical to SEQ ID NO: 261

SEQ ID NO: 263 - protein, 167 aa, Oryza sativa
MEVFSIGQDAFWRETSEVPPYPVRSMESPIHSKGYLFWNIDERFLKGQSRGFLCFSLEDETFSLIPHPCPYLPLNHGTSLVSELDGELCVGLFISGQQQLWMYNGNQWDQRFSINVPGPNDLYIPLDILPHDQLLLQRGPHLYHHNHQSSEDIKEVARMDQLNYQSP

SEQ ID NO: 264 - protein, 167 aa, Oryza sativa; identical to SEQ ID NO: 263

SEQ ID NO: 265 - protein, 167 aa, Oryza sativa; identical to SEQ ID NO: 263

SEQ ID NO: 266 - protein, 130 aa, Oryza sativa
GMEVFTIGNRDSCWRTVAEDPPYPVMADPVYFKGSLYWHICKELLQEGSPPPPQGFLRFDLQDETFGLVLHDVVSPSDETRLDLVELGGELCLAQYLGTEMVIWKSSPSSSDDISHQWDRLYTIGNLPDE

SEQ ID NO: 267 - protein, 130 aa, Oryza sativa
GMEVFTIGNRDSCWRTIAEDPPYPVTADPMYFKGSLYWHICKELLQEGSPPPPQGFLRFDLQDETFGLVLHDVVSPSDETRLDLVELGGELCLAQYLGTEMVIWKSSPSPSDDISHQWDRLYTIGNLPDE

SEQ ID NO: 268 - protein, 175 aa, Oryza sativa
MGMEVFTIGGGGGGAGWREITSDPPYPAKRFQIGVSVCGYMFWRFSERHTKLERGILHLSLEEEEFGITGLPDELDTDNSFLLDELLGRDLCVSASNTSCTMLNIWTLPVADESLCTLWQWRYCIDYPWSLCSVMALPPFSDEIILLRGNNICRYDLATSKLKILCRLDRTRYQG

SEQ ID NO: 269 - protein, 175 aa, Oryza sativa
MGMEVFTIGGGGGGAGWREITSDPPYPAKRFQIGVSVCGYMFWRFSERHTKLERGILHLSLEEEEFGITGLPDELDTDNSFLLDELLGRDLCVSASNTSCTMLNIWTLPVADESLCTLWQWRYCIEYPWSLCSVMALPPFSDEIILLRGNNICRYDLATSKLKILCRLDRTRYQG

SEQ ID NO: 270 - protein, 196 aa, Oryza sativa
EVLTLGTNLWRQTEDPPYPISGLTPVHVKGAIYWMVNTPLCPDPPNAFLRFCLTNEKFSLLQYPPCNLKPTRFIEVEGELCCACFCSQVSALKIWTCNYAQNPEWTQRCTVQIPPDIVVNNPVARPPIVFLHGKKLLLTWNQVYQYDIQTCRMEKIASGVEDFTCYDPRNNKYWAYLEKEVTDMHLFNYAESLVPI

SEQ ID NO: 271 - protein, 196 aa, Oryza sativa
EVLTLGTNLWRQTEDPPYPISGLTPVHVKGAIYWMVNMPLCPDPPNAFLRFCLTNEKFSLLQYPPCNLKPTRFIEVEGELCCACFCSQVSALKIWTCNYAQNPEWTQRCTVQIPPDIVVNNPVARPPIVFLHGKKLLLTWNQVYQYDIQTCRMEKIASGVEDFTCYDPRNNKYWAYLEKEVTDMHLFNYAESLVPI

SEQ ID NO: 272 - protein, 113 aa, Oryza sativa
EVLTLGTNAWRQTEDPPYPIDALTPVHVKGAIYWIVCSSLCPDPPNAFLRFCLTDEKFSLFPCPPSNVKSVRFTEVEGELCCACFFSETLALEIWNCSGGQNLEWTRRYVIQI

SEQ ID NO: 273 - protein, 113 aa, Oryza sativa; identical to SEQ ID NO: 272

SEQ ID NO: 274 - protein, 71 aa, Oryza sativa
MEVFTIGHGDAAAWRETTTPPPPSYPVCHVRTAIHSRGNLFWKLQLRRSPAAAAGSFLLLRFNLAHETFTV

SEQ ID NO: 275 - protein, 96 aa, Oryza sativa
GMELCTIGGNGGVSVGSCWREIVADTPYPVLVWHTATSFRGALFFTTCHDTAARPPQESRLLRLCLRDETFSVVAPPPPCRPPFLHEAFHLSELNG

SEQ ID NO: 276 - protein, 119 aa, Hordeum vulgare
LGMEVFTVGKDQRWRETAAQPPYPFVAGLMATFFKGSLIWTIDHCSPMYDYSHLDDVGHMPCFVRFNLEDESFSVMKGPPWYQGADYLDTNFAELNGELAMPHPGPNSELVEIWMCDDV

SEQ ID NO: 277 - protein, 72 aa, Oryza sativa
MEVFTIGHGDAAAWRETTTPPPPSYPVCHVRTAIHSRGNLFWKLQLRRSPAAAAAGSFLLLRFNLAHETFTV

SEQ ID NO: 278 - protein, 162 aa, Vitis vinifera
LGAEVYTVGTTSSWREISQIPCYPLYKRAVYASGALHWLVNYEFNPDNLKSMVISFNIKDEEFNSIPHQEFPSKVSKWFELVDLRGYLGMVDFSLGTHIEIWKLKDYEKKEWVREYRIDIKPPHGVSISQYIEVVGLMGDGEILLERYKKLVAYNPKTNGLR

SEQ ID NO: 279 - protein, 67 aa, Oryza sativa
VGHEIFTLGGGAGDGWRRTQDPPRAISPDGRPAACTRGGGTSFYWFIDEHEPCALLRFNLRDEAFDV

SEQ ID NO: 280 - protein, 53 aa, Canid herpesvirus 1
LIPKWICDYVMLCSKSLASPLCSISVFIVSFEFVYLMDKYYLKDHNVTMEGSF

SEQ ID NO: 281 - protein, 82 aa, Drosophila ananassae
DKCFTVCVATNGRVGFNAEHTWSDAAIASHMWENLIIDDLQSDGADAPVLGHLWEYIFGDDIYGYDESGNTKGTPEFQPPTP

SEQ ID NO: 282 - protein, 81 aa, Oryza sativa
GDWEATVTPPCIIYTNLPAACAGGAFYWVAHDKSDGTFAVECPNCLVRFAMNDGTFTIVPLPQGVTFMDVDFDSISELGGE

SEQ ID NO: 283 - protein, 81 aa, Oryza sativa; identical to SEQ ID NO: 282

SEQ ID NO: 284 - protein, 209 aa, Triticum sp.
MGMEVITINCREGSWRETLVDPPYLILCPQTAIHCKGCLFYFIDRENQPCSPRGLLRFSLQDETFGITLSLNNLYPTVEDEDIVIHELGGECATFFCKSMQRVLIWMTRDILVPEWECHYVMNVSDQCLPMASLSNGEILLRRGHCLFRYYMKDHGVKENDMFDIDELRFLGPSEDTLGHAWENLFWFDLLYYTPSLVPVTPKASLHAS

SEQ ID NO: 285 - DNA, 630 bp, Triticum sp.
atggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 286 - DNA, 624 bp, Triticum sp.
atggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataa

SEQ ID NO: 287 - DNA, 603 bp, Triticum sp.; identical to SEQ ID NO: 196

SEQ ID NO: 288 - DNA, 582 bp, Triticum sp.
atgcttcttcacgggaggcacttcccggctggcaagtttgggttggtgtcccagatggcacactgcgatgggttggtgctgctccctacagacaccaatgcttatgtcttcaacccggccactagagatggcattgctctgccgaggagtcatcacaacgtgttgcaacgtctcgtatgccttcccattggtttgggccttgatgtttccactggcacatataaggtgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatgcacggtctttcttccgttcatgtgattacaattccgtgcaatggggatggaggtcatcaccattaattgccgagaggggtcttggagggaaaccttggtggatccacc

SEQ ID NO: 289 - DNA, 528 bp, Triticum sp.; identical to SEQ ID NO: 198

SEQ ID NO: 290 - protein, 200 aa, Triticum sp.
MSHSSSAMLLHGRHFPAGKFGLVSQMAHCDGLVLLPTDTNAYVFNPATRDGIALPRSHHNVLQRLVCLPIGLGLDVSTGTYKVARSFFRSCDYNSVQWGWRSSPLIAERGLGGKPWWIHRTLSFVRKLPYTARGVYSISSIGKISHAPHGGCFVSAFKTKLSASLCCLTTSTLQLKMKILLSMSLVGNALLSSARVCNEY

SEQ ID NO: 291 - protein, 193 aa, Triticum sp.
MLLHGRHFPAGKFGLVSQMAHCDGLVLLPTDTNAYVFNPATRDGIALPRSHHNVLQRLVCLPIGLGLDVSTGTYKVARSFFRSCDYNSVQWGWRSSPLIAERGLGGKPWWIHRTLSFVRKLPYTARGVYSISSIGKISHAPHGGCFVSAFKTKLSASLCCLTTSTLQLKMKILLSMSLVGNALLSSARVCNEY

SEQ ID NO: 292 - protein, 175 aa, Triticum sp.
MAHCDGLVLLPTDTNAYVFNPATRDGIALPRSHHNVLQRLVCLPIGLGLDVSTGTYKVARSFFRSCDYNSVQWGWRSSPLIAERGLGGKPWWIHRTLSFVRKLPYTARGVYSISSIGKISHAPHGGCFVSAFKTKLSASLCCLTTSTLQLKMKILLSMSLVGNALLSSARVCNEY

SEQ ID NO: 293 - protein, 163 aa, Triticum sp.
MDLKRVDDTFTQKTVNDARKRKWRMPQRKQTKRSGHPAGEELLDEMVWEILIRLPVESLARFKLVSKVWYTIISQPVFVRAHLQCSKQKQHHKFTIRLPSSSPPRFIYSLRPKISVLSLVQICICTRASTKLRHLFWDGGSRFQSLRHFFHQHPFLPVVFTRR

SEQ ID NO: 294 - protein, 139 aa, Triticum sp.
MPQRKQTKRSGHPAGEELLDEMVWEILIRLPVESLARFKLVSKVWYTIISQPVFVRAHLQCSKQKQHHKFTIRLPSSSPPRFIYSLRPKISVLSLVQICICTRASTKLRHLFWDGGSRFQSLRHFFHQHPFLPVVFTRR

SEQ ID NO: 295 - protein, 118 aa, Triticum sp.
MVWEILIRLPVESLARFKLVSKVWYTIISQPVFVRAHLQCSKQKQHHKFTIRLPSSSPPRFIYSLRPKISVLSLVQICICTRASTKLRHLFWDGGSRFQSLRHFFHQHPFLPVVFTRR

SEQ ID NO: 296 - DNA, 603 bp, Triticum sp.; identical to SEQ ID NO: 196

SEQ ID NO: 297 - DNA, 582 bp, Triticum sp.; identical to SEQ ID NO: 197

SEQ ID NO: 298 - DNA, 528 bp, Triticum sp.; identical to SEQ ID NO: 198

SEQ ID NO: 299 - DNA, 492 bp, Triticum sp.; identical to SEQ ID NO: 199

SEQ ID NO: 300 - DNA, 420 bp, Triticum sp.; identical to SEQ ID NO: 200

SEQ ID NO: 301 - DNA, 357 bp, Triticum sp.; identical to SEQ ID NO: 201

SEQ ID NO: 302 - DNA, 1010 bp, Triticum sp.
tacagacaccaatgcttatgtcttcaacccggccactagagatggcattgctctgccgaggagtcatcacaacgtgttgcaacgtctcgtatgccttcccattggtttgggccttgatgtttccactggcacatataaggttgcacggtctttcttccgttcatgtgattacaattccgtgcaatggggatggaggtcatcaccattaattgccgagaggggtcttggagggaaaccttggtggatccaccgtaccttatcctttgtccgcaaactgccatacactgcaaggggtgtctattctatttcatcgatagggaaaatcagccatgctccccacgggggctgcttcgtttcagccttcaagacgaaactttcggcatcactctgttgcttaacaacctctaccctacagttgaagatgaagatattgttatccatgagcttggtggggaatgcgctactttcttctgcaagagtatgcaacgagtattgatctggatgacaagagatatattggtcccggaatgggaatgtcactatgtaatgaatgtttcagaccagtgcttgccaatggcctcacttagcaatggcgagattcttctacggagaggccattgtctcttccgttattacatgaaggatcatggtgtaaaagaaaacgacatgttcgatatcgatgagctaaggttccttgggcctagtgaagacacattgggacatgcatgggaaaatttattttggttcgacctactctattacacgccaagtcttgttccagttactcctaaggcgagcttacatgcctcataagaccatgtacactgcaaggtgtttagagaagtgcttgtataaataaattgttttttcccttaagtaccggtgcttgtttctatagcagaggtgtctaagtaagtgtctgtgctcaacaaataagcagcaacggtgcttaagagaaattggcttattttttacgcacctctctaagcatcttgcgctgttctggcctg

SEQ ID NO: 303 - DNA, 22 bp, Bacteriophage M13mp18
cacaccggaaacagctatgacc

SEQ ID NO: 304 - protein, 89 aa, Oryza sativa
GMEVITINCREGSWRETLVDPPYLILCPQTAIHCKGCLFYFIDRENQPCSPRGLLRFSLQDETFGITLLLNNLYPTVEDEDIVIHELGG

SEQ ID NO: 305 - DNA, 59 bp, Triticum sp.
atgaagaatctgattggtgaggcaaattgtcgaatctgccaggaaagcttcagcacaac

SEQ ID NO: 306 - DNA, 115 bp, Zea mays; identical to SEQ ID NO: 67

SEQ ID NO: 307 - DNA, 59 bp, Zea mays; identical to SEQ ID NO: 68

SEQ ID NO: 308 - DNA, 59 bp, Zea mays; identical to SEQ ID NO: 68

SEQ ID NO: 309 - DNA, 270 bp, Triticum sp.; identical to SEQ ID NO: 16

SEQ ID NO: 310 - DNA, 270 bp, Triticum sp.; identical to SEQ ID NO: 97

SEQ ID NO: 311 - DNA, 270 bp, Triticum sp.
atggggaagagaaagtcagctgctaagccacctcctaagaagcggatggacaagctcgacactgtcttttcctgcccgttctgcaatcatggcagcagtgttgaatgccgaattgatatgaagaatctgattggtgaggccaattgtcgaatctgccaggaaagcttcagcaccactgtaaatgcgctgactgagcctattgatatatacagcgaatggatcgatgagtgcgagcgcgtcaacactgtcgaagatgatgatggtgcatga

SEQ ID NO: 312 - DNA, 270 bp, Triticum sp.; identical to SEQ ID NO: 97

SEQ ID NO: 313 - DNA, 270 bp, Triticum sp.; identical to SEQ ID NO: 97

SEQ ID NO: 314 - DNA, 270 bp, Triticum sp.
atggggaagagaaagtcagctgctaagccaccccctaagaagcggatggacaagctcgacacggtcttttcctgcccgttctgcaatcatgggagcagtgttgaatgccgaattgatatgaagaatctgattggtgaggcaaattgtcgaatctgccaggaaagcttcagcaccactgtaaatgcgctgactgagcctattgatatatacagcgaatggatcgatgagtgcgagcgcgtcaacactgtcgaagatgatgatggtgcatga

SEQ ID NO: 315 - DNA, 270 bp, Triticum sp.; identical to SEQ ID NO: 16

SEQ ID NO: 316 - DNA, 270 bp, Triticum sp.; identical to SEQ ID NO: 97